(19) United States
(12) Patent Application Publication
(10) Pub. No.: US 2025/0312335 A1
(43) Pub. Date: Oct. 9, 2025

(54) FLT COMBINATION THERAPY FOR CANCER AND COMPOSITIONS THEREFOR

(71) Applicant: BIOMEA FUSION, INC., San Francisco, CA (US)

(72) Inventors: David Sperandio, San Carlos, CA (US); Yongli Su, Foster City, CA (US); Amna Trinity-Turjuman Adam, Redwood City, CA (US); James T. Palmer, Warrandyte (AU); Nan-Horng Lin, Vernon Hills, IL (US); Neil Howard Squires, San Francisco, CA (US); Ravindra B. Upasani, San Jose, CA (US); Solomon B. Ungashe, Sunnyvale, CA (US); Thomas Butler, Redwood City, CA (US); Thorsten A. Kirschberg, San Carlos, CA (US); Thu Phan, Fremont, CA (US); Xiaodong Wang, Millbrae, CA (US)

(21) Appl. No.: 18/865,235

(22) PCT Filed: May 16, 2023
(86) PCT No.: PCT/US2023/022402

§ 371 Date: Nov. 12, 2024

Related U.S. Application Data
(60) Provisional application No. 63/364,874, filed on May 17, 2022.

(51) Int. Cl. A61K 31/4965; A61K 31/497; A61K 31/5377; A61P 35/02

(52) U.S. Cl. CPC A61K 31/4965; A61K 31/497; A61K 31/5377; A61P 35/02

(57) ABSTRACT

Disclosed herein are combinations comprising an inhibitor of FLT3 and an inhibitor of menin. Also described are combinations of specific irreversible inhibitors of FLT3 and irreversible inhibitors of menin. Also described are methods of using the combinations for the treatment of proliferative diseases or conditions, including hematological malignancies and other diseases or conditions dependent on FLT3 activity.

### CROSS-REFERENCE TO RELATED APPLICATION

This application claims the benefit of U.S. provisional application No. 63/364,874 filed May 17, 2022, the entirety of which is incorporated by reference for all purposes.

### FIELD

Described herein are combination therapies comprising a FLT3 inhibitor and a menin inhibitor, compositions for the therapies, and methods of using such combinations to treat cancer.

### BACKGROUND

FMS-like tyrosine kinase 3 (FLT3) is a receptor tyrosine kinase that is expressed on normal hematopoietic stem/progenitor cells. Upon its activation by the FLT3 ligand (FL), FLT3 dimerizes and induces many signaling pathways related to hematopoietic cell survival and proliferation.

FLT3 is also often overexpressed in many acute leukemia cells, and mutation of the FLT3 gene is the most frequent generic alteration in acute myeloid leukemia. Genetic mutation of the FLT3 gene is identified in approximately one-third of newly diagnosed AML adult patients (Papaemmanuil et al., 2016, N Engl J Med. 374: 2209) where these mutations cause constitutive FLT3 activation. Genetic alterations of FLT3 have also been identified in other myeloid malignancies, such as myelodysplastic syndromes (MDS) and acute lymphocytic leukemia (ALL). Certain FLT3 mutations are either internal tandem duplicates (ITD) or point mutations in the tyrosine kinase domain. FLT3-ITD mutations are present in approximately 20% of AML patients, and point mutations are present in approximately 5%-10% of AML patients. Both mutations can constitutively activate FLT3 through ligand-independent autophosphorylation causing increased signaling and cellular proliferation, leading to survival of the leukemia cells (Kennedy et al., 2020, Front. Oncol. 10:612880; Kiyoi et al., 2020, Cancer Science 111:312). FLT3-ITD mutation is especially associated with a poor prognosis and high rate of relapse, and ITD mutations can be gained or lost during disease progression and/or relapse. For that reason, testing for FLT3-ITD in patients with AML is recommended by both the European Leukemia Net and Cancer Network Guidelines.

Early studied FLT3 inhibitors, referred to as first-generation FLT3 inhibitors, included multi-kinase inhibitors sorafenib, midostaurin, lestaurtinib, sunitinib, and tandutinib. These first-generation inhibitors lacked efficacy as a monotherapy, most likely due in part to their non-specific effects. While many of these first generation inhibitors have been abandoned as therapeutic agents for AML as monotherapy or in combination with chemotherapy, midostaurin (Rydapt) in combination with chemotherapy received FDA approval in 2017 for the adults with newly diagnosed FLT3-mutated AML.

Second-generation FLT3 inhibitors have greater specificity for FLT3 and are more potent. Second generation inhibitors include gilteritinib, crenolanib, and quizartinib. Gilteritinib and crenolanib are both Type I inhibitors, meaning that they can bind to both the inactive and active conformations of FLT3, while quizartinib is a Type II inhibitor that can only bind to the inactive conformation. In 2018, the FDA approved gilteritinib for relapsed or refractory AML with patients with FLT3 mutation. In a Phase III trial studying the effect of quizartinib in combination with induction or consolidation chemotherapy (QuANTUM-R) in patients with relapsed or refractory FLT3-ITD AML, quizartinib exhibited a survival benefit and a manageable safety profile (Cortes et al., 2019, Lancet Oncol., 20: 984). Currently, crenolanib is being studied in a Phase III clinical trial for the treatment of relapsed or refractory AML in patients with an FLT3 mutation.

An additional inhibitor of FLT3 includes the covalently-binding FLT3 inhibitor FF-10101, which has demonstrated activity against quizartinib-resistant AML (Yamaura et al., 2018, Blood, 131: 426) and is currently being studied in clinical trials for relapsed or refractory hematological malignancies, including AML. FF-10101 and other N-phenylpyrimidine-2-amine compounds are described in PCT Application WO 2013/157540 and U.S. Pat. No. 9,145,415 assigned to Fujifilm Corporation. PCT Application WO 2015/056683 and U.S. Pat. No. 9,701,644, also assigned to Fujifilm Corporation, describe crystalline forms of FF-10101. Additional patents and patent applications assigned to Fujifilm Corporation that describe FF-10101 and the use of FF-10101 for certain types of cancer include PCT Applications WO 2016/027904; WO 2020/075838; WO 2020/175629; and U.S. Pat. No. 9,987,278.

Hanmi Pharmaceutical. Co., Ltd describe pyrimidine-containing compounds and the use of these compounds in FLT3-mutated cancers in PCT Applications WO 2020/022600; WO 2020/171646; WO 2020/171649; and, WO 2020/262974. The pyrimidine-containing FLT3 inhibitor HM43239 is currently being studied in a Phase 1/2 clinical trial for patients with relapsed or refractory resistant AML (Daver et al. 2019, Blood, 134: 1331).

Genosco and Oscotec, Inc. describe pyridopyrimidine compounds and their use in the treatment of hematological malignancies in PCT Application WO 2013/142382 and U.S. Pat. No. 8,877,763. Crystal forms of the specific FLT3 inhibitor G-749 are described in WO 2020/040467 assigned to Oscotec, Inc. and oral pharmaceutical compositions of G-749 are described in WO 2020/159117 also assigned to Oscotec, Inc.

Biochemically, these inhibitors are reported to block auto-phosphorylation of FLT3 at TYR-589/591 and downstream signaling mediators signal transducer and activator of transcription 5 (STAT5) and extracellular-signal related kinase (ERK) (Wang et al., 2021, J. Med. Chem. 64(8):4870-4890. Resistance to FLT3 inhibitors has been reported in the clinic through secondary mutations in FLT3 including mutations in the tyrosine kinase domain in prior ITD-only mutant tumors (Heidel et al., 2006, Blood 107(1):293-300, Smith et al., 2012, Nature 485(7397):260-263).

Despite research in this area, there is still a need to deliver effective compounds for the inhibition of FLT3. Therefore, the object of the present invention is to provide inhibitors of FLT3, pharmaceutical compositions thereof, and methods for the inhibition of FLT3. Also of key importance is to develop inhibitors that will overcome the known resistance mechanisms of co-mutations ITD with XYZ that re-awaken the autophosphorylation capabilities of FLT3.

The Histone-lysine N-methyltransferase 2 (KMT2) family of proteins, which currently consists of at least 5 members, methylate lysine 4 on the histone H3 tails at important regulatory regions in the genome and thereby impart crucial functions through the modulation of chromatin structures and DNA accessibility (Morera, Lübbert, and Jung., 2016, Clin. Epigenetics 8, 57). These enzymes are known to play an important role in the regulation of gene expression during early development and hematopoiesis (Rao & Dou., Nat. Rev. Cancer 2015, 15, 334-346). The human KMT2 family was initially named the mixed-lineage leukemia (MLL) family, owing to the role of the first-found member in this disease, KMT2A, which is still commonly referred to as MLL1 or MLL in routine clinical practice.

KMT2A (MLL1) is frequently found to be cytogenetically targeted in several types of leukemia (e.g. ALL and AML), and in those cases where balanced chromosomal translocations are found, these typically target KMT2A (MLL1) and one of over 80 translocation partner genes that have been described to date (Winters and Bernt, 2017, Front. Pediatr. 5, 4). These chromosomal anomalies often result in the formation of fusion genes that encode fusion proteins which are believed to be causally related to the onset and/or progression of the disease. Inhibition of menin may be a promising strategy for treating MLL related diseases, including leukemia.

### SUMMARY

In one aspect, described herein are combination therapies comprising an inhibitor of FLT3 and an inhibitor of menin. In some embodiments, the inhibitors of FLT3 are irreversible inhibitors. In some embodiments, the inhibitors of FLT3 are reversible inhibitors. In some embodiments, the inhibitors of FLT3 are covalent inhibitors. In some embodiments, the inhibitors of menin are irreversible inhibitors. In some embodiments, the inhibitors of menin are reversible inhibitors. In some embodiments, the inhibitors of menin are covalent inhibitors.

In another aspect, described herein are methods for using such combinations of FLT3 and menin inhibitors in the treatment of diseases (including diseases wherein inhibition of FLT3 and/or menin provides therapeutic benefit to a patient having the disease). In certain embodiments, the FLT3 inhibitor and menin inhibitor are administered in separate compositions. For example, in certain embodiments, the FLT3 inhibitor is administered in a first composition according to a first schedule, and the menin inhibitor is administered in a second composition according to a second schedule. In other embodiments, the FLT3 inhibitor and the menin inhibitor are administered in the same composition. Accordingly, further described are pharmaceutical compositions that comprise an inhibitor of FLT3, an inhibitor of menin, and one or more pharmaceutically acceptable carriers, excipients, or diluents. In certain embodiments, the combinations and pharmaceutical compositions described herein are used for the treatment of hematological malignancies, including but not limited to, acute myeloid leukemia.

In some embodiments, the FLT3 inhibitor is a compound according to Formula (P-I) having the structure:

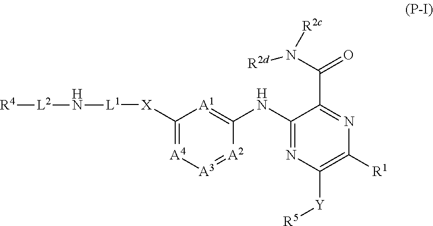

or a stereoisomer thereof, or a pharmaceutically acceptable salt thereof,
wherein: each A¹, A², A³, and A⁴ is independently —C(R⁷)═, or —N═; provided no more than two of A¹, A², A³, and A⁴ is N.
X is a single bond, —O—, or —NR^{2a}—;
Y is absent, —C(R^{2e}R^{2f}), —O—, or —NR^{2g}—;
L¹ is a single bond, substituted or unsubstituted C₁-C₄ alkylene, substituted or unsubstituted C₂-C₄ alkenylene; or substituted or unsubstituted C₂-C₄ alkynylene;
L² is a single bond, —C(O)-L³-NR^{2b}—, —S(O)-L³-NR^{2b}—, or —S(O)₂-L³-NR^{2b}—;
L³ is substituted or unsubstituted C₁-C₄ alkylene, substituted or unsubstituted C₂-C₄ alkenylene; or substituted or unsubstituted C₂-C₄ alkynylene;
Cy is substituted or unsubstituted cycloalkyl or substituted or unsubstituted heterocycloalkyl;
R¹ is H, halo, CN, substituted or unsubstituted C₁₋₆ alkyl, substituted or unsubstituted alkoxy, substituted or unsubstituted alkenyl, substituted or unsubstituted alkynyl, substituted or unsubstituted cycloalkyl, or substituted or unsubstituted heterocycloalkyl;
each R^{2a}, R^{2b}, R^{2c}, R^{2d}, R^{2e}, R^{2f}, and R^{2g}, is independently H or C₁-C₄ alkyl; and wherein R^{2c} and R^{2d} may join together to form a 4-6 membered heterocycloalkyl;
R⁴ is i) —C(O)—C(R^{6a})═C(R^{6b})(R^{6c}), ii) —S(O)—C(R^{6a})═C(R^{6b})(R^{6c}), iii) —S(O)₂—C(R^{6a})═C(R^{6b})(R^{6c}), or iv) substituted or unsubstituted epoxide;
R⁵ is H, Cy, CN, halo, substituted or unsubstituted C₁₋₆ alkyl, substituted or unsubstituted haloalkyl, substituted or unsubstituted C₁₋₆ alkoxy, substituted or unsubstituted haloalkoxy, or substituted or unsubstituted alkylamino;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, wherein the C₁₋₆ alkyl is unsubstituted or substituted with one or more groups selected from substituted or unsubstituted amino and substituted or unsubstituted heterocycloalkyl having 1-2 heteroatoms independently selected from nitrogen, oxygen, and sulfur; and
each R⁷ is independently H, halo, CN, substituted or unsubstituted C₁₋₆ alkyl, substituted or unsubstituted alkoxy, or substituted or unsubstituted heterocycloalkyl.

In some embodiments, the FLT3 inhibitor is a compound according to Formula (P4-I) having the structure:

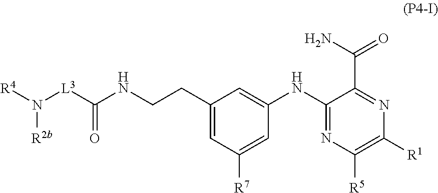

or a stereoisomer thereof, or a pharmaceutically acceptable salt thereof,

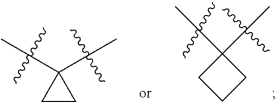 [SMILES: *C1(*)CC1 ; *C1(*)CCC1]

L³ is substituted or unsubstituted C₁-C₄ alkylene,
Cy is substituted or unsubstituted cycloalkyl or substituted or unsubstituted heterocycloalkyl;
R¹ is substituted or unsubstituted C₁₋₆ alkyl, substituted or unsubstituted alkoxy, substituted or unsubstituted cycloalkyl, or substituted or unsubstituted heterocycloalkyl;
R^{2b} is H or C₁-C₄ alkyl;
R⁴ is i) —C(O)—C(R^{6a})═C(R^{6b})(R^{6c}), ii) —S(O)—C(R^{6a})═C(R^{6b})(R^{6c}), iii) —S(O)₂—C(R^{6a})═C(R^{6b})(R^{6c}) or iv) substituted or unsubstituted epoxide;
R⁵ is Cy, substituted or unsubstituted C₁₋₆ alkyl, substituted or unsubstituted haloalkyl, substituted or unsubstituted C₁₋₆ alkoxy, substituted or unsubstituted haloalkoxy, or substituted or unsubstituted alkylamino;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, wherein the C₁₋₆ alkyl is unsubstituted or substituted with one or more groups selected from substituted or unsubstituted amino, and substituted or unsubstituted heterocycloalkyl having 1-2 heteroatoms independently selected from nitrogen, oxygen, and sulfur; and
each R⁷ is independently H, halo, CN, substituted or unsubstituted C₁₋₆ alkyl, substituted or unsubstituted alkoxy, or substituted or unsubstituted heterocycloalkyl.

In some embodiments, the FLT3 inhibitor is a compound according to Formula (P2-I) having the structure:

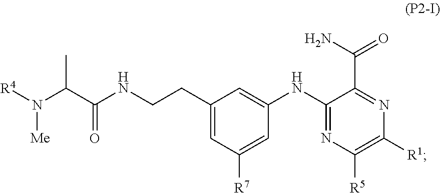

or a stereoisomer thereof, or a pharmaceutically acceptable salt thereof.

In some embodiments, the menin inhibitor is a compound according to Formula (I) having the structure:

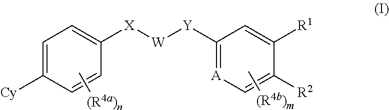

or a pharmaceutically acceptable salt thereof,
wherein:
A is C or N;
Cy is substituted or unsubstituted

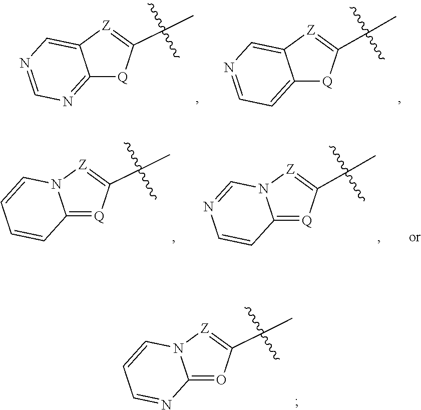

Q is N, —N(H)—, —O—, or —S—;
Z is —CR^{5a}═ or —N═;
X is —NR^{3a}—, —C(R^{3b})₂—, or —O—;
Y is a single bond, —NR^{3a}—, —C(R^{3b})₂—, or —O—;
W is —C(O)—, —S(O)—, or —S(O)₂—;
one of R¹ and R² is Cy²-N(H)C(O)—C(R^{6a})═(R^{6b})(R^{6c}) or CH₂—Cy²-N(H)C(O)—C(R^{6a})═C(R^{6b})(R^{6c}); and the other is H, C₁₋₆ alkyl, C₁₋₆ haloalkyl, halo, or CN;
Cy² is an optionally substituted group selected from phenyl, pyridyl, or a 4-7 membered heterocycloalkyl ring having 1-2 heteroatoms independently selected from nitrogen, oxygen, or sulfur;
each R^{3a} and R^{3b} is independently H or C₁₋₆ alkyl;
each R^{4a} and R^{4b} is independently H, halo, CN, OR, —N(R)₂, —C(O)N(R)₂, —NRC(O)R, —SO₂R, —C(O)R, —CO₂R, or an optionally substituted group selected from C₁₋₆ alkyl, C₃₋₇ cycloalkyl, a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur;
each R is independently H, or an optionally substituted group selected from C₁₋₆ aliphatic, phenyl, an 8-10 membered bicyclic aryl ring, a 4-7 membered saturated or partially unsaturated heterocyclic ring having 1-2 heteroatoms independently selected from nitrogen, oxygen, or sulfur, and a 5-6 membered heteroaryl ring having 1-4 heteroatoms independently selected from nitrogen, oxygen, or sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, or sulfur;
R^{5a} is H, C₁₋₆ alkyl, C₁₋₆ haloalkyl, halo, or CN;
each R^{6a} and R^{6b} is independently H or C₁₋₆ alkyl; or R^{6a} and R^{6b} are joined together to form a bond;
R^{6c} is H or substituted or unsubstituted C₁₋₆ alkyl;
m is 1, 2, or 3; and
n is 1, 2, 3, or 4.

In some embodiments, the menin inhibitors are compounds according to Formula:

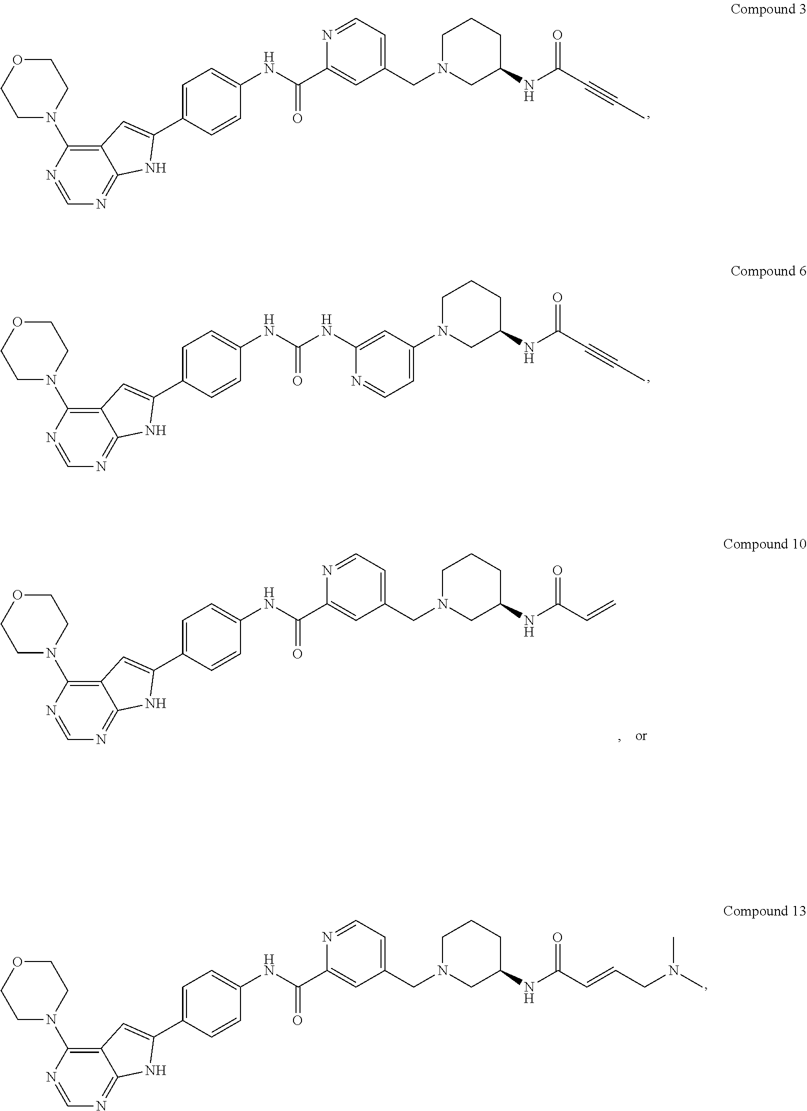 [SMILES: CC#CC(=O)N[C@@H]1CCCN(Cc2ccnc(C(=O)Nc3ccc(-c4cc5c(N6CCOCC6)ncnc5[nH]4)cc3)c2)C1 ; CC#CC(=O)N[C@@H]1CCCN(c2ccnc(NC(=O)Nc3ccc(-c4cc5c(N6CCOCC6)ncnc5[nH]4)cc3)c2)C1 ; C=CC(=O)N[C@@H]1CCCN(Cc2ccnc(C(=O)Nc3ccc(-c4cc5c(N6CCOCC6)ncnc5[nH]4)cc3)c2)C1 ; CN(C)C/C=C/C(=O)N[C@@H]1CCCN(Cc2ccnc(C(=O)Nc3ccc(-c4cc5c(N6CCOCC6)ncnc5[nH]4)cc3)c2)C1]

or a salt thereof.

In some embodiments, the menin inhibitors are compounds according to Formula:

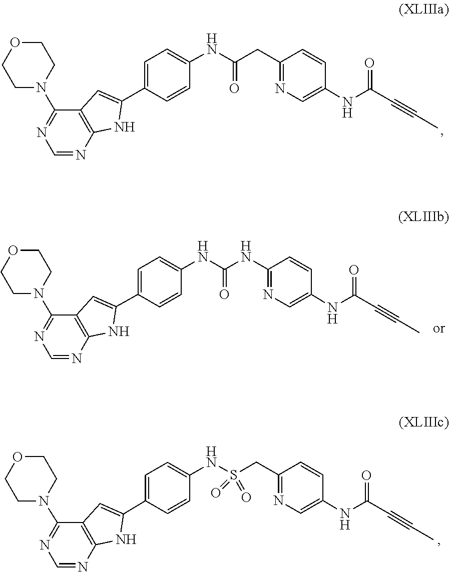 [SMILES: CC#CC(=O)Nc1ccc(CC(=O)Nc2ccc(-c3cc4c(N5CCOCC5)ncnc4[nH]3)cc2)nc1 ; CC#CC(=O)Nc1ccc(NC(=O)Nc2ccc(-c3cc4c(N5CCOCC5)ncnc4[nH]3)cc2)nc1 ; CC#CC(=O)Nc1ccc(CS(=O)(=O)Nc2ccc(-c3cc4c(N5CCOCC5)ncnc4[nH]3)cc2)nc1]

or a pharmaceutically acceptable salt thereof.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In certain embodiments, provided herein is a pharmaceutical composition comprising: i) a pharmaceutically acceptable carrier, diluent, and/or excipient; and ii) a FLT3 inhibitor and/or a menin inhibitor provided herein.

In some embodiments, provided herein are pharmaceutical compositions comprising a therapeutically effective amount of (i) a compound selected from Formula (P-I), (P2-I), and (P4-I); (ii) an inhibitor of menin, and (iii) a pharmaceutically acceptable carrier, excipient, or diluent. In some embodiments, provided herein are pharmaceutical compositions comprising a therapeutically effective amount of (i) a compound of Formula (P4-I); (ii) an inhibitor of menin, and (iii) a pharmaceutically acceptable carrier, excipient, or diluent.

In certain embodiments, provided herein is a pharmaceutical composition comprising a pharmaceutically acceptable carrier, diluent, and/or excipient and a FLT3 inhibitor for administration in combination with a pharmaceutical composition comprising a menin inhibitor and a pharmaceutically acceptable carrier, diluent, and/or excipient and a menin inhibitor.

In some embodiments, the pharmaceutical compositions described herein are formulated for a route of administration selected from oral administration, parenteral administration, buccal administration, nasal administration, topical administration, or rectal administration. In some embodiments, provided herein are methods for preventing, treating or ameliorating in a mammal a disease or condition that is related to the aberrant activity of FLT3, which comprises administering to the mammal an effective disease-treating or condition-treating amount of a combination comprising (i) a FLT3 inhibitor or a pharmaceutically acceptable salt thereof and (ii) an inhibitor of menin or a pharmaceutically acceptable salt thereof. In some embodiments, provided herein are methods for preventing, treating or ameliorating in a mammal a disease or condition that is related to the aberrant activity of FLT3, which comprises administering to the mammal an effective disease-treating or condition-treating amount of a combination comprising (i) a FLT3 inhibitor of Formula (P4-I) or a pharmaceutically acceptable salt thereof and (ii) an inhibitor of menin or a pharmaceutically acceptable salt thereof. In certain embodiments, the FLT3 inhibitor and the menin inhibitor are administered separately, each in its own composition and according to its own schedule. In other embodiments, the FLT3 inhibitor and the menin inhibitor are administered in the same composition on the same schedule. In other embodiments, provided herein are methods for preventing, treating or ameliorating in a mammal a disease or condition that is related to a mutation of the FLT3 gene, which comprises administering to the mammal an effective disease-treating or condition-treating amount of a combination described herein.

In some embodiments, the disease or condition is a hematologic malignancy, including, but not limited to leukemia, lymphoma, or multiple myeloma. In certain embodiments, the disease or condition is a leukemia, including, but not limited to, acute lymphocytic leukemia (ALL), acute myeloid leukemia (AML), chronic lymphocytic leukemia (CLL), chronic myeloid leukemia (CMIL), prolymphocytic leukemia (PLL), large granular lymphocytic (LGL), hairy cell leukemia (HCL), mast-cell leukemia (MCL) or myelodysplastic syndrome (MDS).

In certain embodiments, the disease or condition is acute myeloid leukemia (AML). In certain embodiments, the AML is FLT3 mutation-positive. In certain embodiments, the AML is newly diagnosed. In certain embodiments, the AML is FLT3 mutation-positive and newly diagnosed. In certain embodiments, the AML is relapsed or refractory. In certain embodiments, the AML is relapsed or refractory and is FLT3 mutation-positive.

In certain embodiments, the disease or condition is a lymphoma, including, but not limited to, non-Hodgkin's lymphoma or Hodgkin's lymphoma. In certain embodiments, the disease or condition is multiple myeloma.

In any of the aforementioned embodiments are some embodiments in which administration of the FLT3 inhibitor in combination with the menin inhibitor is enteral, parenteral, or both, and wherein (a) an effective amount of the FLT3 inhibitor and/or menin inhibitor is systemically administered to the mammal; (b) an effective amount of the FLT3 inhibitor and/or menin inhibitor is administered orally to the mammal; (c) an effective amount of the FLT3 inhibitor and/or menin inhibitor is intravenously administered to the mammal; (d) an effective amount of the FLT3 inhibitor and/or menin inhibitor is administered by inhalation; (e) an effective amount of the FLT3 inhibitor and/or menin inhibitor is administered by nasal administration; (f) an effective amount of the FLT3 inhibitor and/or menin inhibitor is administered by injection to the mammal; (g) an effective amount of the FLT3 inhibitor and/or menin inhibitor is administered topically (dermal) to the mammal; (h) an effective amount of the FLT3 inhibitor and/or menin inhibitor is administered by ophthalmic administration; or (i) an effective amount of the FLT3 inhibitor and/or menin inhibitor is administered rectally to the mammal.

In any of the aforementioned embodiments are some embodiments in which administration of the FLT3 inhibitor in combination with the menin inhibitor comprises single administrations of an effective amount of the FLT3 inhibitor and/or menin inhibitor including some embodiments in which (i) the FLT3 inhibitor and/or menin inhibitor is administered once; (ii) the FLT3 inhibitor and/or menin inhibitor is administered to the mammal multiple times over the span of one day; (iii) continually; or (iv) continuously.

In any of the aforementioned embodiments are some embodiments in which administration of the FLT3 inhibitor in combination with the menin inhibitor comprises multiple administrations of an effective amount of the FLT3 inhibitor and/or menin inhibitor, including some embodiments in which (i) the FLT3 inhibitor and/or menin inhibitor is administered in a single dose; (ii) the time between multiple administrations is every 6 hours; (iii) the FLT3 inhibitor and/or menin inhibitor is administered to the mammal every 8 hours. In some embodiments, the method comprises a drug holiday, wherein the administration of the FLT3 inhibitor and/or menin inhibitor is temporarily suspended or the dose of the FLT3 inhibitor and/or menin inhibitor being administered is temporarily reduced; at the end of the drug holiday, dosing of the compound is resumed. The length of the drug holiday can vary from 2 days to 1 year.

In any of the aforementioned embodiments involving the treatment of proliferative disorders, including cancer, for example, a hematological cancer, are some embodiments comprising administering at least one additional agent selected from the group consisting of alemtuzumab, azacitine, bortezomib, decitabine, everolimus, malademetan, palbociclib, ponatinib, venetoclax, and vorinostat. In other embodiments, the treatment of proliferative disorders, including cancer, for example, a hematological cancer, comprise the administration of at least one additional active agent selected from arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase irreversible inhibitors such as irinotecan or topotecan, tyrosine kinase irreversible inhibitors such as gefinitinib or imatinib, or agents to treat signs or symptoms induced by such therapy including allopurinol, filgrastim, granisetron/ondansetron/palonosetron, dronabinol.

In certain embodiments, provided herein are articles of manufacture including packaging material, a compound(s) or composition(s) thereof provided herein within the packaging material, and a label that indicates that the compound(s) or composition(s) is administered to treat a disease or condition.

In some embodiments, the compounds of Formula (P4-I) are irreversible inhibitors of FLT3 activity. In certain embodiments, such irreversible inhibitors have an IC₅₀ below 10 microM in enzyme assay. In some embodiments, a FLT3 inhibitor has an IC₅₀ of less than 1 microM, and in some embodiments, less than 0.25 microM or even less than 0.025 microM.

Other objects, features, and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### DETAILED DESCRIPTION OF THE PREFERRED EMBODIMENTS

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Use of the term “including” as well as other forms, such as “include”, “includes,” and “included,” is not limiting. Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In some embodiments, an alkyl comprises five to fifteen carbon atoms (e.g., C₅-C₁₅ alkyl). In certain embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl, or t-Bu), 3-methylhexyl, 2-methylhexyl, and the like. Unless stated otherwise specifically in the specification, an alkyl group is optionally substituted as defined and described below and herein.

The alkyl group could also be a “lower alkyl” having 1 to 6 carbon atoms.

As used herein, C₁-Cₓ includes C₁-C₂, C₁-C₃ . . . C₁-Cₓ.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In some embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted as defined and described below and herein.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In some embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted as defined and described below and herein.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted as defined and described below and herein.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted as defined and described below and herein.

“Alkynylene” or “alkynylene chain” refers to a straight or branched divalent hydrocarbon chain linking the rest of the molecule to a radical group, consisting solely of carbon and hydrogen, containing at least one triple bond and having from two to twelve carbon atoms, for example butyne. The alkynylene chain is attached to the rest of the molecule through a triple bond or a single bond and to the radical group through a triple bond or a single bond. The points of attachment of the alkynylene chain to the rest of the molecule and to the radical group can be through one carbon or any two carbons within the chain. Unless stated otherwise specifically in the specification, an alkynylene chain is optionally substituted as defined and described below and herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted as defined and described below and herein.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In some embodiments, a carbocyclyl comprises five to seven carbon atoms. The carbocyclyl is attached to the rest of the molecule by a single bond. Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted as defined and described below and herein.

“Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures, respectively, in which at least one hydrogen is replaced with a halogen atom. In certain embodiments in which two or more hydrogen atoms are replaced with halogen atoms, the halogen atoms are all the same as one another. In some embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to 14 ring atoms, such as three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

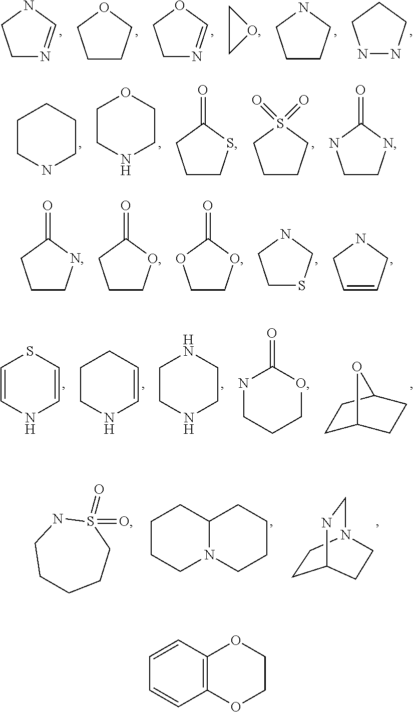

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some embodiments, heteroaryl rings have five, six, seven, eight, nine, or more than nine ring atoms. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted as defined and described below and herein.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Epoxide” refers to a three-membered cyclic ether. The epoxide is optionally substituted as defined and described below and herein.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S— radical.

“Sulfinyl” refers to the —S(═O)— radical.

“Sulfonyl” refers to the —S(═O)₂— radical.

“Amino” refers to the —NH₂ radical. A “substituted amino” refers to an “alkylamino” or “dialkylamino” group as defined herein.

“Cyano” refers to the —CN radical.

“Nitro” refers to the —NO₂ radical.

“Oxa” refers to the —O— radical.

“Oxo” refers to the ═O radical.

“Imino” refers to the ═NH radical.

“Thioxo” refers to the ═S radical.

An “alkoxy” group refers to a (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

As used herein, the terms “heteroalkyl,” “heteroalkenyl,” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger substructure.

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R.

As used herein, the term “trihalomethanesulfonyl” refers to a group of formula X₃CS(═O)₂— where X is a halogen.

“Cyanoalkyl” means an alkyl radical, as defined herein, substituted with at least one cyano group.

As used herein, the term “N-sulfonamido” or “sulfonylamino” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S)NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” or “alkylamino” refers to the —N(alkyl)ₓH_{y} group, where x and y are either (i) x=1 and y=1 or (ii) x=2 and y=0. When x=2, the alkyl groups, taken together with the N atom to which they are attached, can optionally form a cyclic ring system. In certain embodiments, the “alkylamino” group is a “dialkylamino” group wherein x=2 and y=0. In certain embodiments, the “dialkylamino” group is one wherein the alkyl groups are taken together with the N atom to which they are attached to form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

As described herein, compounds provided herein may be “optionally substituted”. In general, the term “substituted,” whether preceded by the term “optionally” or not, means that one or more hydrogens of a designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents provided herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR′, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each RT is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “nucleophile” or “nucleophilic” refers to an electron rich compound, or moiety thereof.

The term “electrophile”, or “electrophilic” refers to an electron poor or electron deficient molecule, or moiety thereof. Examples of electrophiles include, but in no way are limited to, Michael acceptor moieties.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

201 “Bioavailability” refers to the percentage of the weight of compounds disclosed herein, such as, compounds of any of Formula (P-1), (P2-1), (P4-I), or (I) dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein, such as, compounds of any of Formula (P-1), (P2-1), (P4-I), or (I) are absorbed into the general circulation when the pharmaceutical composition is taken orally as compared to intravenous injection.

“Blood plasma concentration” refers to the concentration of compounds disclosed herein, such as, compounds of any of Formula (P-1), (P2-1), (P4-I), or (I) in the plasma component of blood of a subject. It is understood that the plasma concentration of compounds of any of Formula (P-1), (P2-1), (P4-I), or (I) may vary significantly between subjects, due to variability with respect to metabolism and/or possible interactions with other therapeutic agents. In accordance with some embodiments disclosed herein, the blood plasma concentration of the compounds of any of Formula (P-1), (P2-1), (P4-I), or (I) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of any of Formula (P-1), (P2-1), (P4-I), or (I) may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of Formula (P-1), (P2-1), (P4-I), or (I), age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75-100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75-100 nucleic acids in length, over a region that is about 50 nucleic acids in length, or, where not specified, across the entire sequence of a polynucleotide sequence.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “irreversible inhibitor,” as used herein, refers to a compound that, upon contact with a target protein (e.g., FLT3) causes the formation of a new covalent bond with or within the protein, whereby one or more of the target protein's biological activities (e.g., phosphotransferase activity) is diminished or abolished notwithstanding the subsequent presence or absence of the irreversible inhibitor. In contrast, a reversible inhibitor compound upon contact with a target protein does not cause the formation of a new covalent bond with or within the protein and therefore can associate and dissociate from the target protein.

The term “irreversible inhibitor of FLT3 protein-protein interaction” as used herein, refers to an inhibitor of FLT3 that can form a covalent bond with an amino acid residue of FLT3. In one embodiment, the irreversible inhibitor of FLT3 can form a covalent bond with a Cys residue of FLT3; in particular embodiments, the irreversible inhibitor can form a covalent bond with a Cys 12 residue (or a homolog thereof) of FLT3.

The term “prophylactically effective amount,” as used herein, refers that amount of a composition applied to a patient that will relieve to some extent one or more of the symptoms of a disease, disease or condition being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

As used herein, the term “selective binding compound” refers to a compound that selectively binds to any portion of one or more target proteins.

As used herein, the term “selectively binds” refers to the ability of a selective binding compound to bind to a target protein, such as, for example, FLT3, with greater affinity than it binds to a non-target protein. In certain embodiments, specific binding refers to binding to a target with an affinity that is at least 10, 50, 100, 250, 500, 1000 or more times greater than the affinity for a non-target.

As used herein, the term “selective modulator” refers to a compound that selectively modulates a target activity relative to a non-target activity. In certain embodiments, specific modulater refers to modulating a target activity at least 10, 50, 100, 250, 500, 1000 times more than a non-target activity.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The term “subject” or “patient” as used herein, refers to an animal which is the object of treatment, observation, or experiment. By way of example only, a subject may be, but is not limited to, a mammal including, but not limited to, a human.

As used herein, the term “target activity” refers to a biological activity capable of being modulated by a selective modulator. Certain exemplary target activities include, but are not limited to, binding affinity, signal transduction, enzymatic activity, tumor growth, inflammation, or inflammation-related processes, and amelioration of one or more symptoms associated with a disease or condition.

As used herein, the term “target protein” refers to a molecule or a portion of a protein capable of being bound by a selective binding compound. In certain embodiments, a target protein is FLT3.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the term “FLT3” refers to fins-like tyrosine kinase 3. Synonyms include CD135, CD135 antigen, fetal liver kinase 2, FL cytokine receptor, FLK-2, FLK2, FLT3_HUMAN, fins-related tyrosine kinase 3, growth factor receptor tyrosine kinase type III, receptor-type tyrosine-protein kinase FLT3, stem cell tyrosine kinase 1, STK-1, and STK1. Human sequences include NM_004119 and NM_004119.2 (mRNA), and NP_004110 and NP_004110.2 (protein). Mouse sequences include NM_010229 and NM_010229.2 (mRNA) and NP_034359 and NP_034359.2 (protein).

As used herein, the IC₅₀ refers to an amount, concentration, or dosage of a particular test compound that achieves a 50% inhibition of a maximal response, such as inhibition of FLT3, in an assay that measures such response.

As used herein, EC₅₀ refers to a dosage, concentration, or amount of a particular test compound that elicits a dose-dependent response at 50% of maximal expression of a particular response that is induced, provoked or potentiated by the particular test compound.

### Combinations

Methods described herein include administering to a subject in need a composition containing a therapeutically effective amount of one or more FLT3 inhibitor compounds in combination with one or more menin inhibitors described herein.

In some embodiments, methods described herein can be used to treat a cancer, e.g., B-cell proliferative disorders, which include, but are not limited to diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, and lymphomatoid granulomatosis.

Symptoms, diagnostic tests, and prognostic tests for each of the above-mentioned conditions are known in the art. See, e.g., Harrison's Principles of Internal Medicine©,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models of are useful for establishing a range of therapeutically effective doses of FLT3 inhibitor compounds for treating any of the foregoing diseases.

Animal models for treatment of thromboembolic disorders are also known.

The therapeutic efficacy of the provided compounds for one of the foregoing diseases can be optimized during a course of treatment. For example, a subject being treated can undergo a diagnostic evaluation to correlate the relief of disease symptoms or pathologies to inhibition of in vivo FLT3 activity achieved by administering a given dose of a FLT3 inhibitor.

In certain embodiments, the amount of the FLT3 inhibitor compound is therapeutically effective. In certain embodiments, the amount of the menin inhibitor is therapeutically effective. In certain embodiments, the amount of the FLT3 inhibitor compound is therapeutically effective, and the amount of the menin inhibitor is therapeutically effective. In certain embodiments, the amount of the FLT3 inhibitor compound is sub-therapeutic. In certain embodiments, the amount of the menin inhibitor is sub-therapeutic. In certain embodiments, the amount of the FLT3 inhibitor compound is sub-therapeutic, and the amount of the menin inhibitor is sub-therapeutic. In certain sub-therapeutic embodiments, the combination is therapeutic while one or more components are at sub-therapeutic doses.

In certain embodiments, the FLT3 inhibitor compound and the menin inhibitor are administered consecutively in either order. As used herein, the terms “consecutively,” “serially,” and “sequentially” refer to administration of a FLT3 inhibitor compound after a menin inhibitor, or administration of the menin inhibitor after the FLT3 inhibitor compound. For instance, consecutive administration may involve administration of the FLT3 inhibitor compound in the absence of the menin inhibitor during an induction phase (primary therapy), which is followed by a post-induction treatment phase comprising administration of the menin inhibitor. The methods may further comprise a maintenance phase comprising administration of the FLT3 inhibitor compound or the menin inhibitor, or both. Alternatively, consecutive administration may involve administration of the menin inhibitor in the absence of the FLT3 inhibitor compound during an induction phase (primary therapy), which is followed by a post-induction treatment phase comprising administration of the FLT3 inhibitor compound. The methods may further comprise a maintenance phase comprising administration of the FLT3 inhibitor compound or the menin inhibitor, or both.

In certain embodiments, the FLT3 inhibitor compound and the menin inhibitor are administered concurrently. As used herein, the terms “concurrently,” “simultaneously,” and “in parallel” refer to administration of a FLT3 inhibitor compound and a menin inhibitor during the same doctor visit or during the same phase of treatment. For instance, both the FLT3 inhibitor compound and the menin inhibitor may be administered during one or more of an induction phase, a treatment phase, and a maintenance phase. However, concurrent administration does not require that the FLT3 inhibitor compound and the menin inhibitor be present together in a single formulation or pharmaceutical composition, or that the FLT3 inhibitor compound and the menin inhibitor be administered at precisely the same time.

In certain embodiments, provided herein is a method of treating cancer responsive to inhibition of FLT3 activity, the method comprising administering an effective amount of a combination provided herein to an individual to treat the cancer responsive to inhibition of FLT3 activity. In certain embodiments, provided herein is a method of treating cancer responsive to menin inhibition, the method comprising administering an effective amount of a combination provided herein to an individual to treat the cancer responsive to menin inhibition.

In certain embodiments, provided herein is a method of treating cancer that is nonresponsive to FLT3 inhibition alone, the method comprising administering an effective amount of a combination provided herein to such an individual to treat the cancer nonresponsive to FLT3 inhibition. In certain embodiments, provided herein is a method of treating cancer that is nonresponsive to menin inhibition alone, the method comprising administering an effective amount of a combination provided herein to such an individual to treat the cancer nonresponsive to menin inhibition.

The FLT3 inhibitor compound or composition thereof is suitably administered to the individual at one time or over a series of treatments. In certain embodiments, the treatment includes multiple administrations of the FLT3 inhibitor compound or composition, wherein the interval between administrations may vary. For example, the interval between the first administration and the second administration is about one month, and the intervals between the subsequent administrations are about three months. In certain embodiments, a FLT3 inhibitor compound is administered at a flat dose. In certain embodiments, a FLT3 inhibitor compound described herein is administered to an individual at a fixed dose based on the individual's weight (e.g., mg/kg).

The menin inhibitor or composition thereof is suitably administered to the individual at one time or over a series of treatments. In certain embodiments, the treatment includes multiple administrations of the menin inhibitor or composition, wherein the interval between administrations may vary. For example, the interval between the first administration and the second administration is about one month, and the intervals between the subsequent administrations are about three months. In certain embodiments, a menin inhibitor is administered at a flat dose. In certain embodiments, a menin inhibitor is administered to an individual at a fixed dose based on the individual's weight (e.g., mg/kg).

In one aspect, described herein are pharmaceutical compositions comprising an inhibitor of FLT3 and an inhibitor of menin. In some embodiments, the inhibitors of FLT3 are irreversible inhibitors. In some embodiments, the inhibitors of FLT3 are reversible inhibitors. In some embodiments, the inhibitors of FLT3 are covalent inhibitors. In some embodiments, the inhibitors of menin are irreversible inhibitors. In some embodiments, the inhibitors of menin are reversible inhibitors. In some embodiments, the inhibitors of menin are covalent inhibitors.

In another aspect, described herein are methods for using such compositions of FLT3 and menin inhibitors in the treatment of diseases (including diseases wherein inhibition of FLT3 and/or menin provides therapeutic benefit to a patient having the disease). Further described are pharmaceutical compositions that comprise an inhibitor of FLT3, an inhibitor of menin and one or more pharmaceutically acceptable carriers, excipients, or diluents. In certain embodiments, the combinations and pharmaceutical compositions described herein are used for the treatment of hematological malignancies, including but not limited to, acute myeloid leukemia.

### FLT3 Inhibitors

In the following description of FLT3 inhibitor compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

FLT3 inhibitor compounds can be used for the manufacture of a medicament for treating any of the foregoing conditions (e.g., hematological malignancies).

In some embodiments, the FLT3 inhibitor compound used for the methods described herein inhibits FLT3 activity with an in vitro IC₅₀ of less than about 1 μM (e.g., less than about 1 μM, less than about 0.5 μM, less than about 0.4 μM, less than about 0.3 μM, less than about 0.1 μM, less than about 0.08 μM, less than about 0.06 μM, less than about 0.05 μM, less than about 0.04 μM, less than about 0.03 μM, less than about 0.02 μM, less than about 0.01 μM, less than about 0.008 μM, less than about 0.006 μM, less than about 0.005 μM, less than about 0.004 μM, less than about 0.003 μM, less than about 0.002 μM, less than about 0.001 μM, less than about 0.00099 μM, less than about 0.00098 μM, less than about 0.00097 μM, less than about 0.00096 μM, less than about 0.00095 μM, less than about 0.00094 μM, less than about 0.00093 μM, less than about 0.00092 μM, or less than about 0.00090 μM).

Also described herein are methods for synthesizing such irreversible inhibitors, methods for using such irreversible inhibitors in the treatment of diseases (including diseases wherein inhibition of FLT3 provides therapeutic benefit to a patient having the disease). Further described are pharmaceutical compositions that include an inhibitor of FLT3.

Specifically described herein are irreversible inhibitors of FLT3 that form a covalent bond with a cysteine residue on FLT3. Further described herein are irreversible inhibitors of FLT3 that form a covalent bond with a Cys12 residue on FLT3. Irreversible inhibitor compounds described herein include a Michael acceptor moiety. Also described are pharmaceutical formulations that include a irreversible inhibitor of FLT3.

Generally, a reversible or irreversible inhibitor compound of FLT3 used in the methods described herein is identified or characterized in an in vitro assay, e.g., an acellular biochemical assay or a cellular functional assay. Such assays are useful to determine an in vitro IC₅₀ for a reversible or irreversible FLT3 inhibitor compound.

Further, covalent complex formation between FLT3 and a candidate irreversible FLT3 inhibitor is a useful indicator of irreversible inhibition of FLT3 that can be readily determined by a number of methods known in the art (e.g., mass spectrometry). For example, some irreversible FLT3 inhibitor compounds can form a covalent bond with Cys 12 of FLT3 GC12 (e.g., via a Michael reaction). See S. Xu et al. Angewandte Chemie International Ed. 57(6), 1601-1605 (2017) (incorporated by reference in its entirety).

### Menin Inhibitors

The menin inhibitors used in the combination of the present invention are described in U.S. Pat. No. 11,084,825 (incorporated by reference in its entirety).

### Combination Therapy

In a particular aspect, described herein are methods of treating cancer using a combination comprising a compound according to formula (P-I):

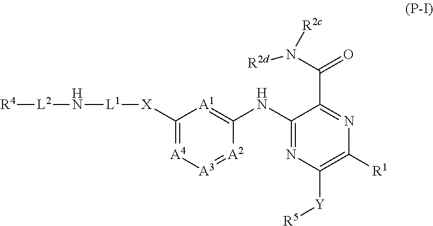

or a stereoisomer thereof, or a pharmaceutically acceptable salt thereof,
wherein: each A¹, A², A³, and A⁴ is independently —C(R⁷)═, or —N═; provided no more than two of A¹, A², A³, and A⁴ is N.
X is a single bond, —O—, or —NR^{2a};
Y is absent, —C(R^{2e}R^{2f}), —O—, or —NR^{2g}—;
L¹ is a single bond, substituted or unsubstituted C₁-C₄ alkylene, substituted or unsubstituted C₂-C₄ alkenylene; or substituted or unsubstituted C₂-C₄ alkynylene;
L² is a single bond, —C(O)-L³-NR^{2b}—, —S(O)-L³-NR^{2b}—, or —S(O)₂-L³-NR^{2b}—;
L³ is substituted or unsubstituted C₁-C₄ alkylene, substituted or unsubstituted C₂-C₄ alkenylene; or substituted or unsubstituted C₂-C₄ alkynylene;
Cy is substituted or unsubstituted cycloalkyl, or substituted or unsubstituted heterocycloalkyl;
R¹ is H, halo, CN, substituted or unsubstituted C₁₋₆ alkyl, substituted or unsubstituted alkoxy, substituted or unsubstituted alkenyl, substituted or unsubstituted alkynyl, substituted or unsubstituted cycloalkyl, or substituted or unsubstituted heterocycloalkyl;
each R^{2a}, R^{2b}, R^{2c}, R^{2d}, R^{2e}, R^{2f}, and R^{2g}, is independently H or C₁-C₄ alkyl; and wherein R^{2c} and R^{2d} may join together to form a 4-6 membered heterocycloalkyl;
R⁴ is i) —C(O)—C(R^{6a})═C(R^{6b})(R^{6c}), ii) —S(O)—C(R^{6a})═C(R^{6b})(R^{6c}), iii) —S(O)₂—C(R^{6a})═C(R^{6b})(R^{6c}), or iv) substituted or unsubstituted epoxide;
R⁵ is H, Cy, CN, halo, substituted or unsubstituted C₁₋₆ alkyl, substituted or unsubstituted haloalkyl, substituted or unsubstituted C₁₋₆ alkoxy, substituted or unsubstituted haloalkoxy, or substituted or unsubstituted alkylamino;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, wherein the C₁₋₆ alkyl is unsubstituted or substituted with one or more groups selected from substituted or unsubstituted amino, and substituted or unsubstituted heterocycloalkyl having 1-2 heteroatoms independently selected from nitrogen, oxygen, and sulfur; and
each R⁷ is independently H, halo, CN, substituted or unsubstituted C₁₋₆ alkyl, substituted or unsubstituted alkoxy, or substituted or unsubstituted heterocycloalkyl; and a menin inhibitor.

In a particular aspect, described herein are methods of treating cancer using a combination comprising a compound according to formula (P4-I) having the structure:

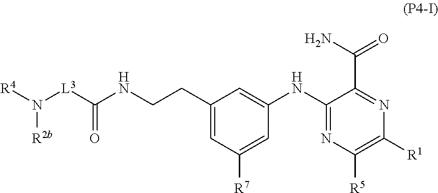

or a stereoisomer thereof, or a pharmaceutically acceptable salt thereof,
L³ is substituted or unsubstituted C₁-C₄ alkylene,

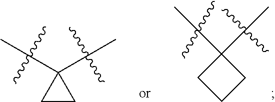 [SMILES: *C1(*)CC1 ; *C1(*)CCC1]

Cy is substituted or unsubstituted cycloalkyl, or substituted or unsubstituted heterocycloalkyl;
R¹ is substituted or unsubstituted C₁₋₆ alkyl, substituted or unsubstituted alkoxy, substituted or unsubstituted cycloalkyl, or substituted or unsubstituted heterocycloalkyl;
R^{2b} is H or C₁-C₄ alkyl;
R⁴ is i) —C(O)—C(R^{6a})═C(R^{6b})(R^{6c}), ii) —S(O)—C(R^{6a})═C(R^{6b})(R^{6c}), iii) —S(O)₂—C(R^{6a})═C(R^{6b})(R^{6c}), or iv) substituted or unsubstituted epoxide;
R⁵ is Cy, substituted or unsubstituted C₁₋₆ alkyl, substituted or unsubstituted haloalkyl, substituted or unsubstituted C₁₋₆ alkoxy, substituted or unsubstituted haloalkoxy, or substituted or unsubstituted alkylamino;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, wherein the C₁₋₆ alkyl is unsubstituted or substituted with one or more groups selected from substituted or unsubstituted amino, and substituted or unsubstituted heterocycloalkyl having 1-2 heteroatoms independently selected from nitrogen, oxygen, and sulfur;
each R⁷ is independently H, halo, CN, substituted or unsubstituted C₁₋₆ alkyl, substituted or unsubstituted alkoxy, or substituted or unsubstituted heterocycloalkyl; and an menin inhibitor.

In a particular aspect, described herein are methods of treating cancer using a combination comprising a compound according to Formula (P2-I) having the structure:

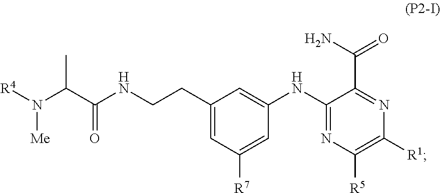

or a stereoisomer thereof, or a pharmaceutically acceptable salt thereof; and an menin inhibitor.

In certain embodiments, R^{2b} is Me.

In certain embodiments, L³ is —CH₂—, —C(Me)H—, —CH₂—CH₂—, or —CH₂—CH₂—CH₂—.

In certain embodiments, R⁷ is H, Me, Et, Cl, F, or OMe.

In certain embodiments, R⁷ is H or F.

In certain embodiments, R¹ is Me or Et.

In certain embodiments, R⁵ is cyclopropyl, Me, Et, N(Me)₂, or N(i-Pr)(Me).

In certain embodiments, the compound is according to Formula (XLIIIa), (XLIIIb), (XLIIIc) or (XLIIId):

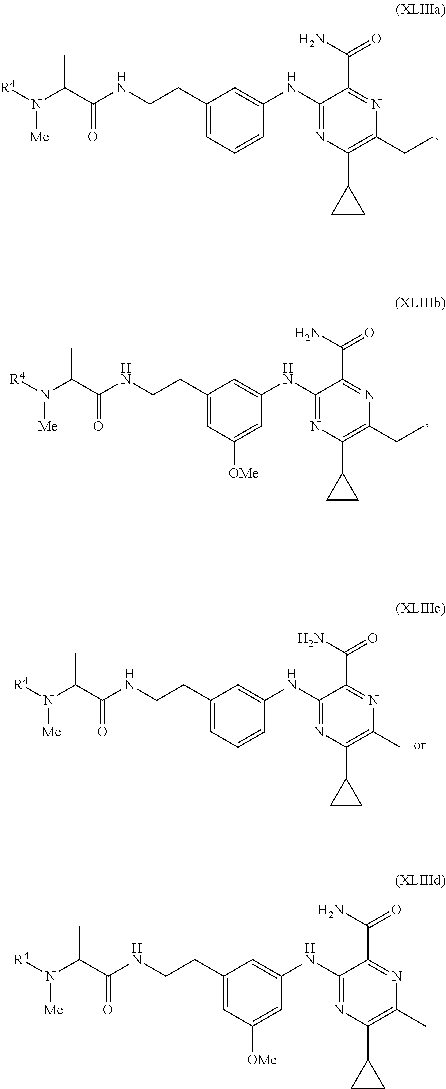

or a stereoisomer thereof, or a pharmaceutically acceptable salt thereof.

In certain embodiments, the compound is according to Formula (XLIVa), (XLIVb), (XLIVc), or (XLIVd):

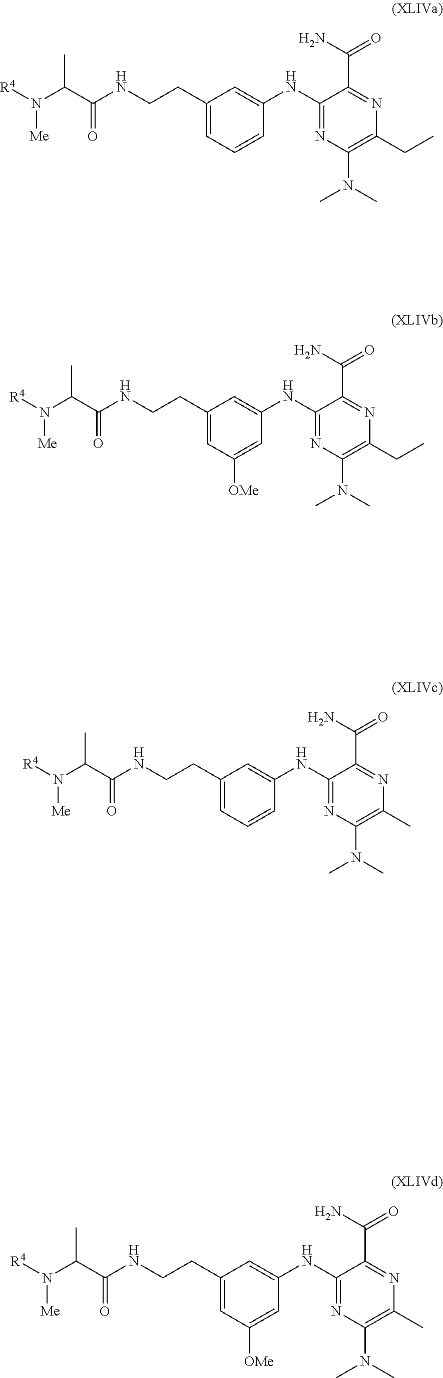

or a stereoisomer thereof, or a pharmaceutically acceptable salt thereof.

In certain embodiments, the compound is according to Formula (XLVIa), (XLVIb), (XLVIc), (XLVId), (XLVIe) or (XLVIf):

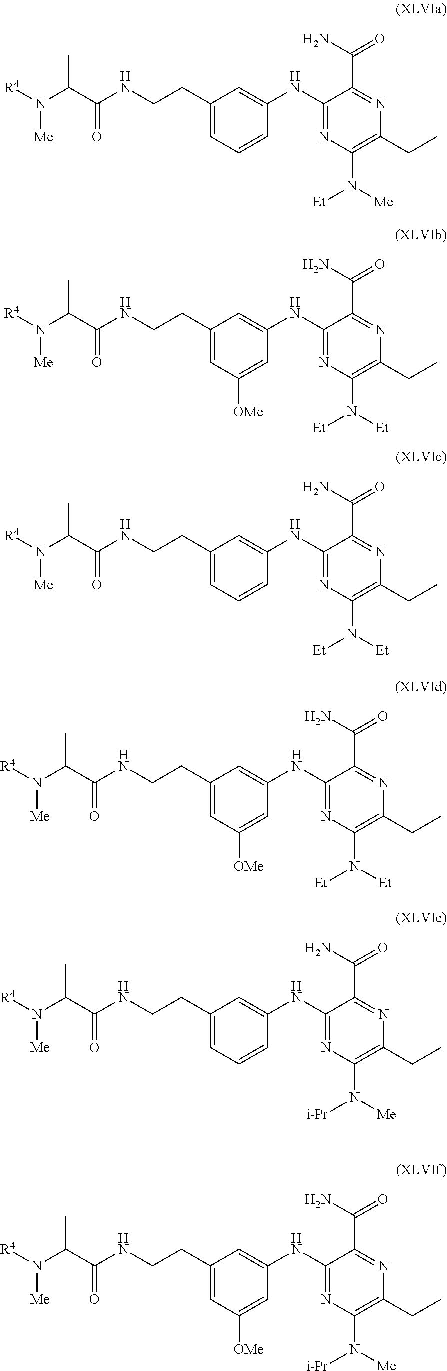

or a stereoisomer thereof, or a pharmaceutically acceptable salt thereof.

In certain embodiments, the compound is according to Formula (XLVIm), (XLVIn), (XLVIo), (XLVIp), (XLVIq) or (XLVIr):

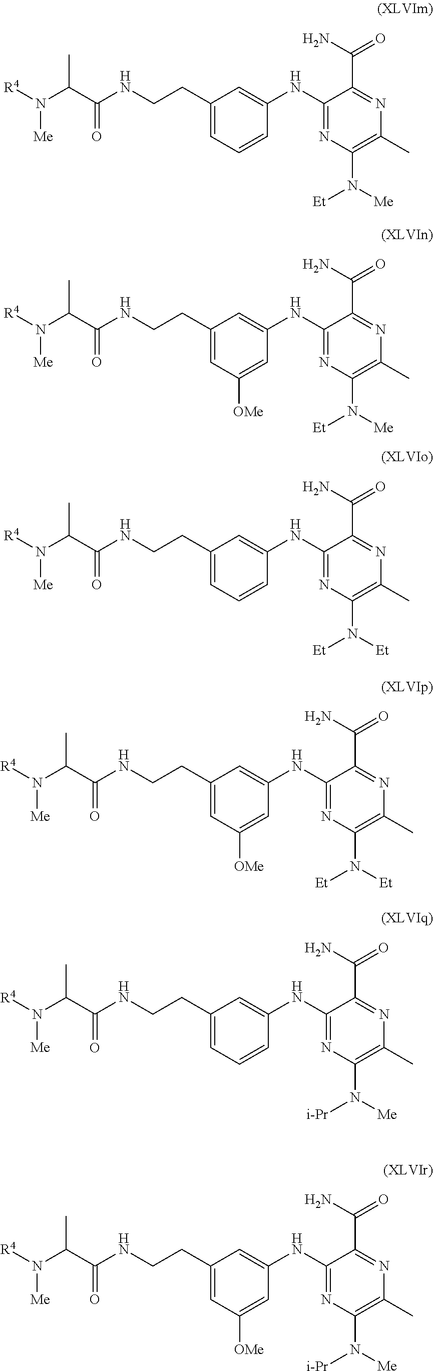

or a stereoisomer thereof, or a pharmaceutically acceptable salt thereof.

In certain embodiments, the compound is according to Formula (XLVIIIa), (XLVIIIc), (XLVIIIe), or (XLVIIIg):

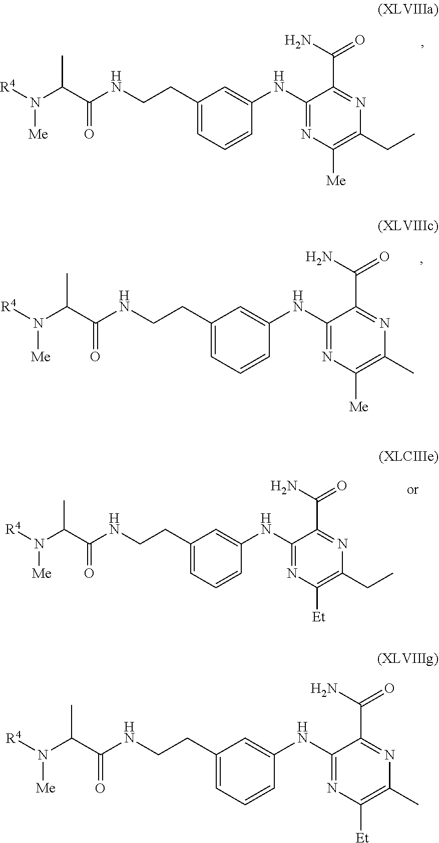

or a stereoisomer thereof, or a pharmaceutically acceptable salt thereof.

In certain embodiments, the compound is according to Formula (LXa), (LXb), (LXc), (LXd), (LXe) or (LXf):

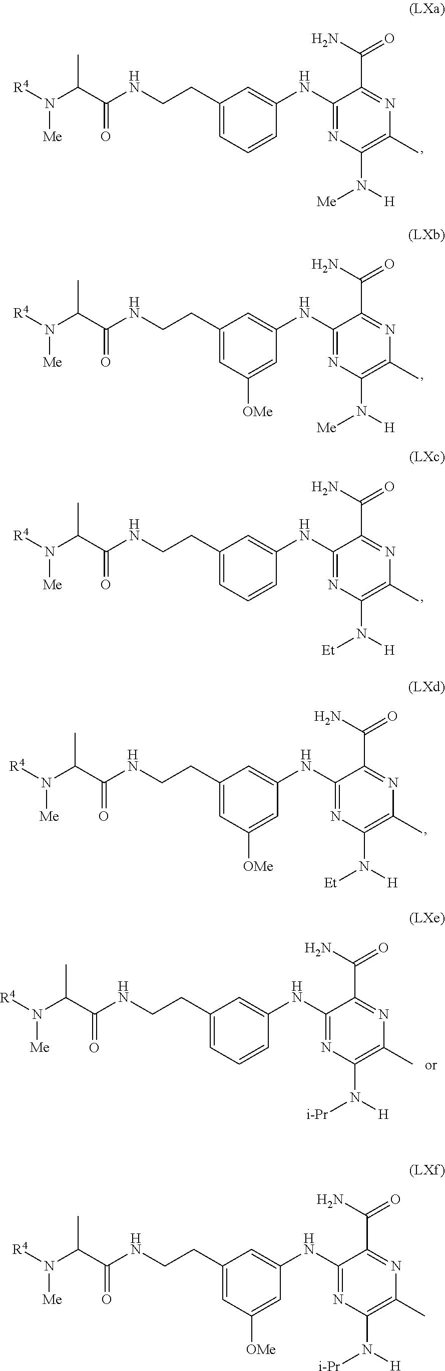

or a stereoisomer thereof, or a pharmaceutically acceptable salt thereof.

In certain embodiments, the compound is according to Formula (LXIa), (LXIb), (LXIc), or (LXId):

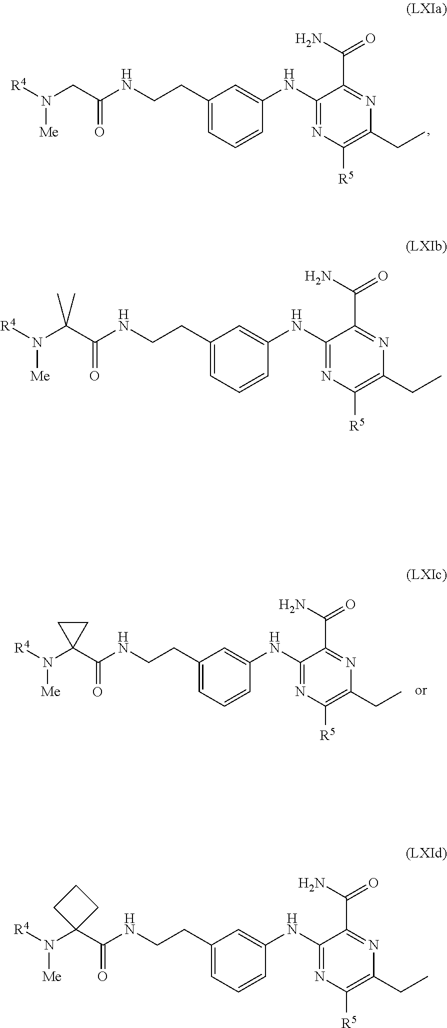

or a stereoisomer thereof, or a pharmaceutically acceptable salt thereof.

In certain embodiments, the compound is according to Formula (LXIa), (LXIIb), (LXIIc), or (LXIId):

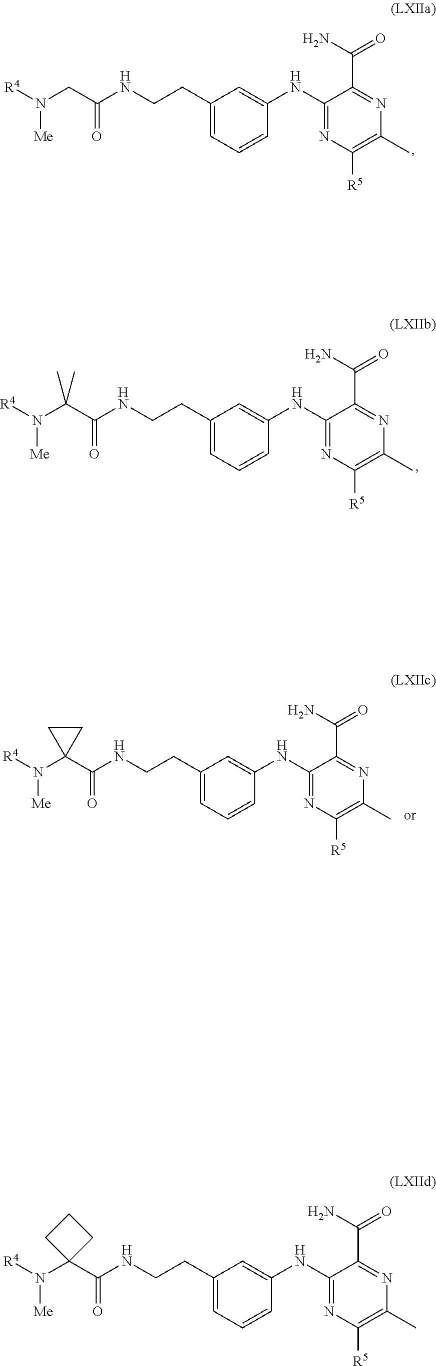

or a stereoisomer thereof, or a pharmaceutically acceptable salt thereof.

In certain embodiments, the compound is according to Formula (LXIIIa), or (LXIIIb):

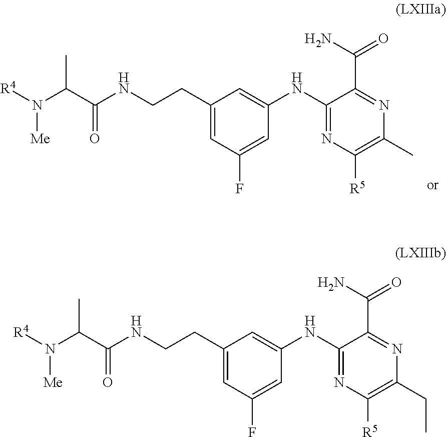

or a stereoisomer thereof, or a pharmaceutically acceptable salt thereof.

In certain embodiments, R⁵ is cyclopropyl, Me, Et, N(Me)₂, or N(i-Pr)(Me).

In certain embodiments, R⁴ is —C(O)—C(R^{6a})═C(R^{6b})(R^{6c}).

In certain embodiments, R⁴ is —C(O)—CH═CH₂.

In certain embodiments, R⁴ is —S(O)—C(R^{6a})═C(R^{6b})(R^{6c}) or —S(O)₂—C(R^{6a})═C(R^{6b})(R^{6c}).

In certain embodiments, R⁴ is —S(O)₂—CH═CH₂.

In certain embodiments, R⁷ is hydrogen.

In certain embodiments, each of R^{6a}, R^{6b}, and R^{6c} is H.

In certain embodiments, each of R^{6a} and R^{6b} is H or F; and R^{6c} is substituted or unsubstituted alkyl.

In certain embodiments, one of R^{6a} and R^{6b} is CN, the other is H; and R^{6c} is H or substituted or unsubstituted alkyl.

In certain embodiments, each of R^{6a} and R^{6b} is H; and R^{6c} is unsubstituted alkyl.

In certain embodiments, each of R^{6a} and R^{6b} is H; and R^{6c} is Me or Et.

In certain embodiments, each of R^{6a} and R^{6b} is H; and R^{6c} is alkyl substituted with amino, alkylamino or dialkylamino.

In certain embodiments, each of R^{6a} and R^{6b} is H; and R^{6c} is alkyl substituted with dimethylamino.

In certain embodiments, each of R^{6a} and R^{6b} is H; and R^{6c} is —CH₂NMe₂.

In certain embodiments, R^{6a} and R^{6b} form a bond (thereby forming a triple bond); and R^{6c} is H or substituted or unsubstituted alkyl.

In certain embodiments, R^{6a} and R^{6b} form a bond (thereby forming a triple bond); and R^{6c} is Me.

In certain embodiments, each of R^{6a} and R^{6b} is H; R^{6c} is —(CH₂)_{q}-heterocycloalkyl; and q is 1, 2, 3, or 4.

In certain embodiments, each of R^{6a} and R^{6b} is H; R^{6c} is —(CH₂)_{q}-heterocycloalkyl; and q is 1.

In certain embodiments, each of R^{6a} and R^{6b} is H; R^{6c} is —(CH₂)_{q}-heterocycloalkyl; and q is 2.

In certain embodiments, each of R^{6a} and R^{6b} is H; R^{6c} is —(CH₂)_{q}-heterocycloalkyl; and q is 3.

In certain embodiments, heterocycloalkyl is substituted or unsubstituted azetidinyl, pyrrolidinyl, piperidinyl, or azepinyl.

In certain embodiments, heterocycloalkyl is azetidin-1-yl, pyrrolidin-1-yl, piperidin-1-yl, or azepin-1-yl.

In certain embodiments, each of R^{6a} and R^{6b} is H or Me; and R^{6c} is —CH₂-azetidin-1-yl, —CH₂-pyrrolidin-1-yl, or —CH₂-piperidin-1-yl.

In certain embodiments, one of R^{6a} and R^{6b} is F; and R^{6c} is H or unsubstituted alkyl.

In certain embodiments, one of R^{6a} and R^{6b} is F; and R^{6c} is Me or Et.

In certain embodiments, one of R^{6a} and R^{6b} is F; and R^{6c} is alkyl substituted with amino, alkylamino or dialkylamino.

In certain embodiments, one of R^{6a} and R^{6b} is F; the other is H; and R^{6c} is H.

In certain embodiments, R⁴ is —C(O)—CH═CH₂, —C(O)—C(F)═CH₂, or —S(O)₂—CH═CH₂.

In certain embodiments, R^{1a} is Me, Et, or i-Pr.

In certain embodiments, R^{1a} is Me.

In certain embodiments, R⁴ is unsubstituted epoxide.

In certain embodiments, R⁴ is epoxide substituted with alkyl.

In certain embodiments, R⁴ is epoxide substituted with Me, Et, i-Pr, or n-Pr.

In certain embodiments, R^{6a} is H.

In certain embodiments, R^{6a} is F.

In certain embodiments, R^{6a} is CN.

In certain embodiments, R⁴ is —C(O)—CH═C(H)R^{6c}.

In certain embodiments, R⁴ is —C(O)—C≡CR^{6c}.

In certain embodiments, R^{6c} is H.

In certain embodiments, R^{6c} is substituted or unsubstituted alkyl.

In certain embodiments, R^{6c} is H or substituted or unsubstituted alkyl.

In certain embodiments, R^{6c} is unsubstituted alkyl.

In certain embodiments, R^{6c} is Me or Et.

In certain embodiments, R^{6c} is alkyl substituted with amino, alkylamino or dialkylamino.

In certain embodiments, R^{6c} is alkyl substituted with dimethylamino.

In certain embodiments, R^{6c} is —CH₂NMe₂.

In certain embodiments, R^{6c} is H or substituted or unsubstituted alkyl.

In certain embodiments, R^{6c} is Me.

In certain embodiments, R^{6c} is —(CH₂)_{q}-heterocycloalkyl; and q is 1, 2, 3, or 4.

In certain embodiments, R^{6c} is —(CH₂)_{q}-heterocycloalkyl; and q is 1.

In certain embodiments, R^{6c} is —(CH₂)_{q}-heterocycloalkyl; and q is 2.

In certain embodiments, R^{6c} is —(CH₂)_{q}-heterocycloalkyl; and q is 3.

In certain embodiments, the compound is according to Formula (XLIVa) or (XLIVc).

In certain embodiments, the compound is according to Formula (XLVIe) or (XLVIq).

In certain embodiments, the compound is according to Formula (XLVIIIa) or (XLVIIIc).

In certain embodiments, the compound is according to Formula (XLIIIa) or (XLIIIc).

In certain embodiments, the compound is according to Formula (XLIVa) or (XLIVc); and R⁴ is —C(O)—CH═CH₂. In particular embodiments, R⁴ is —C(O)—CH═CH—CH₂—NMe₂.

In certain embodiments, the compound is according to Formula (XLVIe) or (XLVIq); and R⁴ is —C(O)—CH═CH₂. In particular embodiments, R⁴ is —C(O)—CH═CH—CH₂—NMe₂.

In certain embodiments, the compound is according to Formula (XLVIIIa) or (XLVIIIc); and R⁴ is —C(O)—CH═CH₂. In particular embodiments, R⁴ is —C(O)—CH═CH—CH₂—NMe₂.

In certain embodiments, the compound is according to Formula (XLIIIa) or (XLIIIc); and R⁴ is —C(O)—CH═CH₂. In particular embodiments, R⁴ is —C(O)—CH═CH—CH₂—NMe₂.

In some particular embodiments, the compound is of Formula (P-1) is a compound of Table 1:

| Compd ID | Name |
|---|---|
| 137 | 5-(dimethylamino)-3-[3-[2-[[(2S)-2-[[(E)-4-(dimethylamino)but-2-enoyl]-methyl- |
| | amino]propanoyl]amino]ethyl]anilino]-6-ethyl-pyrazine-2-carboxamide; |
| 138 | 5-cyclopropyl-3-[3-[2-[[(2S)-2-[[(E)-4-(dimethylamino)but-2-enoyl]-methyl- |
| | amino]propanoyl]amino]ethyl]anilino]-6-ethyl-pyrazine-2-carboxamide; |
| 142A | 3-[3-[2-[[(2S)-2-[[(E)-4-(dimethylamino)but-2-enoyl]-methyl- |
| | amino]propanoyl]amino]ethyl]anilino]-6-ethyl-5-methyl-pyrazine-2-carboxamide; |
| 143 | 5-(dimethylamino)-3-[3-[2-[[(2S)-2-[[(E)-4-(dimethylamino)but-2-enoyl]-methyl- |
| | amino]propanoyl]amino]ethyl]-5-methoxy-anilino]-6-ethyl-pyrazine-2-carboxamide; |
| 144 | 5-cyclopropyl-3-[3-[2-[[(2S)-2-[[(E)-4-(dimethylamino)but-2-enoyl]-methyl- |
| | amino]propanoyl]amino]ethyl]-5-methoxy-anilino]-6-ethyl-pyrazine-2-carboxamide; |
| 156 | 3-[3-[2-[[(2S)-2-[[(E)-4-(azetidin-1-yl)but-2-enoyl]-methyl- |
| | amino]propanoyl]amino]ethyl]anilino]-5-(dimethylamino)-6-ethyl-pyrazine-2-carboxamide; |
| 158 | 5-(dimethylamino)-3-[3-[2-[[(2S)-2-[[(E)-4-(dimethylamino)but-2-enoyl]-methyl- |
| | amino]propanoyl]amino]ethyl]-5-fluoro-anilino]-6-ethyl-pyrazine-2-carboxamide; |
| 159 | 5-cyclopropyl-3-[3-[2-[[(2S)-2-[[(E)-4-(dimethylamino)but-2-enoyl]-methyl- |
| | amino]propanoyl]amino]ethyl]-5-fluoro-anilino]-6-ethyl-pyrazine-2-carboxamide; |
| 160 | 3-[3-[2-[[(2S)-2-[but-2-ynoyl(methyl)amino]propanoyl]amino]ethyl]anilino]-5-(dimethylamino)- |
| | 6-ethyl-pyrazine-2-carboxamide; |
| 162 | 5-(dimethylamino)-3-[3-[2-[2-[[(Z)-4-(dimethylamino)-2-fluoro-but-2-enoyl]-methyl- |
| | amino]propanoylamino]ethyl]anilino]-6-ethyl-pyrazine-2-carboxamide; |
| 163 | 3-[3-[2-[[(2S)-2-[[(E)-4-(dimethylamino)but-2-enoyl]-methyl-amino]propanoyl]amino]ethyl]-5- |
| | fluoro-anilino]-6-ethyl-5-methyl-pyrazine-2-carboxamide; |
| 164 | 3-[3-[2-[[(2S)-2-[[(E)-4-[bis(trideuteriomethyl)amino]but-2-enoyl]-methyl- |
| | amino]propanoyl]amino]ethyl]anilino]-6-ethyl-5-methyl-pyrazine-2-carboxamide; |
| 202 | 3-[3-[2-[[(2S)-2-[[(E)-4-(azetidin-1-yl)but-2-enoyl]-methyl- |
| | amino]propanoyl]amino]ethyl]anilino]-6-ethyl-5-methyl-pyrazine-2-carboxamide; |
| 203 | 5-(dimethylamino)-3-[3-[2-[[(2S)-2-[4-(dimethylamino)but-2-ynoyl-methyl- |
| | amino]propanoyl]amino]ethyl]anilino]-6-ethyl-pyrazine-2-carboxamide; |
| 204 | 3-[3-[2-[[(2S)-2-[[(E)-4-(dimethylamino)but-2-enoyl]-methyl-amino]propanoyl]- |
| | amino]ethyl]anilino]-6-ethyl-5-[isopropyl(methyl)amino]pyrazine-2-carboxamide; |
| 205 | 3-[3-[2-[[(2S)-2-[[(E)-4-(dimethylamino)but-2-enoyl]-methyl- |
| | amino]propanoyl]amino]ethyl]anilino]-6-ethyl-5-(isopropylamino)pyrazine-2-carboxamide; |
| 207 | 3-[3-[2-[[(2S)-2-[[(E)-4-(dimethylamino)but-2-enoyl]-methyl- |
| | amino]propanoyl]amino]ethyl]anilino]-5,6-dimethyl-pyrazine-2-carboxamide; |
| 208 | 5-cyclopropyl-6-ethyl-3-[3-[2-[[(2S)-2-[methyl(prop-2- |
| | enoyl)amino]propanoyl]amino]ethyl]anilino]pyrazine-2-carboxamide; |
| 209 | 3-[3-[2-[[(2S)-2-[but-2-ynoyl(methyl)amino]propanoyl]amino]ethyl]anilino]-5-cyclopropyl-6- |
| | ethyl-pyrazine-2-carboxamide; |
| 211 | 5-cyclopropyl-3-[3-[2-[2-[[(E)-4-(dimethylamino)but-2-enoyl]-methyl- |
| | amino]propanoylamino]ethyl]anilino]-6-methyl-pyrazine-2-carboxamide; |
| 213 | 5-(dimethylamino)-6-ethyl-3-[3-[2-[[(2S)-2-[methyl(prop-2- |
| | enoyl)amino]propanoyl]amino]ethyl]anilino]pyrazine-2-carboxamide; |
| 214 | 3-[3-[2-[[(2S)-2-[[(E)-4-(azetidin-1-yl)but-2-enoyl]-methyl- |
| | amino]propanoyl]amino]ethyl]anilino]-5,6-dimethyl-pyrazine-2-carboxamide; |
| 215 | 5-cyclopropyl-6-ethyl-3-[3-[2-[[(2S)-2-[2-fluoroprop-2- |
| | enoyl(methyl)amino]propanoyl]amino]ethyl]anilino]pyrazine-2-carboxamide; |
| 216 | 6-ethyl-5-methyl-3-[3-[2-[[(2S)-2-[methyl(prop-2- |
| | enoyl)amino]propanoyl]amino]ethyl]anilino]pyrazine-2-carboxamide; |
| 217 | 3-[3-[2-[[(2S)-2-[but-2-ynoyl(methyl)amino]propanoyl]amino]ethyl]anilino]-5-(dimethylamino)- |
| | 6-methyl-pyrazine-2-carboxamide; |
| 218 | 5-(dimethylamino)-6-methyl-3-[3-[2-[[(2S)-2-[methyl(prop-2- |
| | enoyl)amino]propanoyl]amino]ethyl]anilino]pyrazine-2-carboxamide; |
| 219 | 5-cyclopropyl-3-[3-[2-[[2-[[(E)-4-(dimethylamino)but-2-enoyl]-methyl- |
| | amino]acetyl]amino]ethyl]anilino]-6-ethyl-pyrazine-2-carboxamide; |
| 220 | 5-cyclopropyl-3-[3-[2-[[1-[[(E)-4-(dimethylamino)but-2-enoyl]-methyl- |
| | amino]cyclopropanecarbonyl]amino]ethyl]anilino]-6-ethyl-pyrazine-2-carboxamide; |
| 221 | 3-[3-[2-[[(2S)-2-[[(E)-4-(dimethylamino)but-2-enoyl]-methyl- |
| | amino]propanoyl]amino]ethyl]anilino]-6-ethyl-5-[ethyl(methyl)amino]pyrazine-2-carboxamide; |
| 222 | 5-cyclopropyl-6-methyl-3-[3-[2-[[(2S)-2-[methyl(prop-2- |
| | enoyl)amino]propanoyl]amino]ethyl]anilino]pyrazine-2-carboxamide; |
| 223 | 3-[3-[2-[[1-[[(E)-4-(dimethylamino)but-2-enoyl]-methyl- |
| | amino]cyclopropanecarbonyl]amino]ethyl]anilino]-6-ethyl-5-methyl-pyrazine-2-carboxamide; |
| 224 | 5-cyclopropyl-6-ethyl-3-[3-[2-[[(2S)-2-[methyl-[(E)-4-(methylamino)but-2- |
| | enoyl]amino]propanoyl]amino]ethyl]anilino]pyrazine-2-carboxamide; |
| 225 | 5-[ethyl(methyl)amino]-6-methyl-3-[3-[2-[[(2S)-2-[methyl(prop-2- |
| | enoyl)amino]propanoyl]amino]ethyl]anilino]pyrazine-2-carboxamide; |
| 226 | 3-[3-[2-[[2-[[(E)-4-(dimethylamino)but-2-enoyl]-methyl-amino]acetyl]amino]ethyl]anilino]-6- |
| | ethyl-5-methyl-pyrazine-2-carboxamide; |
| 227 | 6-ethyl-5-methyl-3-[3-[2-[[(2S)-2-[methyl-[(E)-4-(methylamino)but-2- |
| | enoyl]amino]propanoyl]amino]ethyl]anilino]pyrazine-2-carboxamide; |
| 228 | 5-cyclopropyl-3-[3-[2-[[(2S)-2-[[(E)-4-(dimethylamino)but-2-enoyl]-methyl- |
| | amino]propanoyl]amino]ethyl]anilino]-6-methyl-pyrazine-2-carboxamide; |
| 230 | 3-[3-[2-[[(2S)-2-[[(E)-4-(azetidin-1-yl)but-2-enoyl]-methyl- |
| | amino]propanoyl]amino]ethyl]anilino]-5-(dimethylamino)-6-methyl-pyrazine-2-carboxamide; |
| 231 | 5-[isopropyl(methyl)amino]-6-methyl-3-[3-[2-[[(2S)-2-[methyl(prop-2- |
| | enoyl)amino]propanoyl]amino]ethyl]anilino]pyrazine-2-carboxamide; |
| 232 | 3-[3-[2-[[(2S)-2-[[(E)-4-(dimethylamino)but-2-enoyl]-methyl- |
| | amino]propanoyl]amino]ethyl]anilino]-5-[isopropyl(methyl)amino]-6-methyl-pyrazine-2- |
| | carboxamide: |
| 233 | 5-(dimethylamino)-3-[3-[2-[[(2S)-2-[[(E)-4-(dimethylamino)but-2-enoyl]-methyl- |
| | amino]propanoyl]amino]ethyl]anilino]-6-methyl-pyrazine-2-carboxamide; |
| 234 | 3-[3-[2-[[(2R)-2-[[(E)-4-(dimethylamino)but-2-enoyl]-methyl- |
| | amino]propanoyl]amino]ethyl]anilino]-6-ethyl-5-methyl-pyrazine-2-carboxamide; |
| 236 | 5-cyclopropyl-6-ethyl-3-[3-[2-[[(2R)-2-[methyl(prop-2- |
| | enoyl)amino]propanoyl]amino]ethyl]anilino]pyrazine-2-carboxamide; |
| 237 | 5-cyclopropyl-6-ethyl-3-[3-[2-[[(2S)-2-[prop-2- |
| | enoyl(trideuteriomethyl)amino]propanoyl]amino]ethyl]anilino]pyrazine-2-carboxamide; |
| 238 | 5,6-dimethyl-3-[3-[2-[[(2S)-2-[methyl(prop-2- |
| | enoyl)amino]propanoyl]amino]ethyl]anilino]pyrazine-2-carboxamide; |
| 239 | 5-(dimethylamino)-3-[3-[2-[[(2S)-2-[[(E)-4-(dimethylamino)but-2-enoyl]-methyl- |
| | amino]propanoyl]amino]ethyl]-5-fluoro-anilino]-6-methyl-pyrazine-2-carboxamide; |
| 240 | 6-ethyl-3-[3-[2-[[(2S)-2-[[(E)-4-(3-fluoroazetidin-1-yl)but-2-enoyl]-methyl- |
| | amino]propanoyl]amino]ethyl]anilino]-5-methyl-pyrazine-2-carboxamide; |
| 241 | 5-cyclopropyl-3-[3-[2-[[2-[[(E)-4-(dimethylamino)but-2-enoyl]-methyl- |
| | amino]acetyl]amino]ethyl]anilino]-6-methyl-pyrazine-2-carboxamide; |
| 242 | 3-[3-[2-[[(2S)-2-[[(E)-4-(azetidin-1-yl)but-2-enoyl]-methyl- |
| | amino]propanoyl]amino]ethyl]anilino]-5-cyclopropyl-6-methyl-pyrazine-2-carboxamide; |
| 243 | 3-[3-[2-[[(2S)-2-[[(E)-4-(dimethylamino)but-2-enoyl]-methyl- |
| | amino]propanoyl]amino]ethyl]anilino]-5,6-diethyl-pyrazine-2-carboxamide; |
| 244 | 3-[3-[2-[[(2S)-2-[4-(dimethylamino)but-2-ynoyl-methyl-amino]propanoyl]amino]ethyl]anilino]-6- |
| | ethyl-5-methyl-pyrazine-2-carboxamide; |
| 245 | 3-[3-[2-[[(2S)-2-[[(E)-4-(dimethylamino)but-2-enoyl]-methyl-amino]propanoyl]amino]ethyl]-5- |
| | fluoro-anilino]-5,6-dimethyl-pyrazine-2-carboxamide; |
| 246 | 3-[3-[2-[[(2S)-2-[[(E)-4-[bis(trideuteriomethyl)amino]but-2-enoyl]-methyl- |
| | amino]propanoyl]amino]ethyl]anilino]-6-ethyl-5-[ethyl(methyl)amino]pyrazine-2-carboxamide; |
| 247 | 3-[3-[2-[[(2S)-2-[[(E)-4-[bis(trideuteriomethyl)amino]but-2-enoyl]-methyl- |
| | amino]propanoyl]amino]ethyl]anilino]-5-[isopropyl(methyl)amino]-6-methyl-pyrazine-2- |
| | carboxamide: |
| 248 | 3-[3-[2-[[(2S)-2-[[(E)-4-[bis(trideuteriomethyl)amino]but-2-enoyl]-methyl- |
| | amino]propanoyl]amino]ethyl]anilino]-5-cyclopropyl-6-methyl-pyrazine-2-carboxamide; |
| 249 | 3-[3-[2-[[(2S)-2-[[(E)-4-[bis(trideuteriomethyl)amino]but-2-enoyl]-methyl- |
| | amino]propanoyl]amino]ethyl]anilino]-6-ethyl-5-[isopropyl(methyl)amino]pyrazine-2- |
| | carboxamide: |
| 250 | 3-[3-[2-[[2-[[(E)-4-(dimethylamino)but-2-enoyl]-methyl-amino]acetyl]amino]ethyl]anilino]-5- |
| | [isopropyl(methyl)amino]-6-methyl-pyrazine-2-carboxamide; |
| 251 | 3-[3-[2-[[2-[[(E)-4-(dimethylamino)but-2-enoyl]-methyl-amino]acetyl]amino]ethyl]-5-fluoro- |
| | anilino]-5,6-dimethyl-pyrazine-2-carboxamide; |
| 252 | 3-[3-[2-[[2-[[(E)-4-(dimethylamino)but-2-enoyl]-methyl-amino]acetyl]amino]ethyl]anilino]-6- |
| | ethyl-5-[ethyl(methyl)amino]pyrazine-2-carboxamide; |
| 253 | 3-[3-[2-[[(2S)-2-[[(E)-4-(dimethylamino)but-2-enoyl]-methyl- |
| | amino]propanoyl]amino]ethyl]anilino]-5-[ethyl(methyl)amino]-6-methyl-pyrazine-2-carboxamide; |
| 254 | 5-cyclopropyl-3-[3-[2-[[(2S)-2-[[(E)-4-(dimethylamino)but-2-enoyl]-methyl- |
| | amino]propanoyl]amino]ethyl]-5-fluoro-anilino]-6-methyl-pyrazine-2-carboxamide; or |
| 255 | 3-[3-[2-[[(2S)-2-[[(E)-4-(dimethylamino)but-2-enoyl]-methyl- |
| | amino]propanoyl]amino]ethyl]anilino]-5-ethyl-6-methyl-pyrazine-2-carboxamide; |
| 301 | 3-[3-[2-[[2-[[(E)-4-(dimethylamino)but-2-enoyl]-methyl-amino]acetyl]amino]ethyl]anilino]-5,6- |
| | dimethyl-pyrazine-2-carboxamide |
| 302 | 3-[3-[2-[[(2R)-2-[[(E)-4-(dimethylamino)but-2-enoyl]-methyl- |
| | amino]propanoyl]amino]ethyl]anilino]-6-ethyl-5-[isopropyl(methyl)amino]pyrazine-2- |
| | carboxamide |
| 303 | 6-ethyl-5-(isopropylamino)-3-[3-[2-[[(2S)-2-[methyl-[(E)-4-(methylamino)but-2- |
| | enoyl]amino]propanoyl]amino]ethyl]anilino]pyrazine-2-carboxamide |
| 304 | 5-cyclopropyl-3-[[4-[2-[[(2S)-2-[[(E)-4-(dimethylamino)but-2-enoyl]-methyl- |
| | amino]propanoyl]amino]ethyl]-2-pyridyl]amino]-6-ethyl-pyrazine-2-carboxamide |
| 305 | 3-[3-[2-[[(2S)-2-[[(E)-4-(dimethylamino)but-2-enoyl]-methyl- |
| | amino]propanoyl]amino]ethyl]anilino]-5-isopropyl-6-methyl-pyrazine-2-carboxamide |
| 306 | 5-cyclopropyl-3-[[5-[2-[[(2S)-2-[[(E)-4-(dimethylamino)but-2-enoyl]-methyl- |
| | amino]propanoyl]amino]ethyl]-3-pyridyl]amino]-6-ethyl-pyrazine-2-carboxamide |
| 307 | 6-ethyl-5-[isopropyl(methyl)amino]-3-[3-[2-[[(2S)-2-[methyl-[(E)-4-(methylamino)but-2- |
| | enoyl]amino]propanoyl]amino]ethyl]anilino]pyrazine-2-carboxamide |
| 308 | 6-ethyl-5-[isopropyl(methyl)amino]-3-[3-[2-[[2-[methyl(prop-2- |
| | enoyl)amino]acetyl]amino]ethyl]anilino]pyrazine-2-carboxamide |
| 309 | 5-[isopropyl(methyl)amino]-6-methyl-3-[3-[2-[[2-[methyl(prop-2- |
| | enoyl)amino]acetyl]amino]ethyl]anilino]pyrazine-2-carboxamide |
| 310 | 5-cyclopropyl-3-[2-[[2-[[(2S)-2-[[(E)-4-(dimethylamino)but-2-enoyl]-methyl- |
| | amino]propanoyl]amino]ethyl]-4-pyridyl]amino]-6-ethyl-pyrazine-2-carboxamide |
| 311 | 3-[3-[(2R)-2-[[2-[[(E)-4-(dimethylamino)but-2-enoyl]-methyl- |
| | amino]acetyl]amino]propyl]anilino]-6-ethyl-5-methyl-pyrazine-2-carboxamide |
| 312 | 3-[3-[(2S)-2-[[2-[[(E)-4-(dimethylamino)but-2-enoyl]-methyl- |
| | amino]acetyl]amino]propyl]anilino]-6-ethyl-5-methyl-pyrazine-2-carboxamide |
| 313 | 5-cyclopropyl-3-[[6-[2-[[(2S)-2-[[(E)-4-(dimethylamino)but-2-enoyl]-methyl- |
| | amino]propanoyl]amino]ethyl]-2-pyridyl]amino]-6-ethyl-pyrazine-2-carboxamide |
| 314 | 3-[3-[(2R)-2-[[(2S)-2-[[(E)-4-(dimethylamino)but-2-enoyl]-methyl- |
| | amino]propanoyl]amino]propyl]anilino]-6-ethyl-5-methyl-pyrazine-2-carboxamide |
| 315 | 6-ethyl-5-[isopropyl(methyl)amino]-3-[3-[2-[[(2S)-2-[methyl(prop-2- |
| | enoyl)amino]propanoyl]amino]ethyl]anilino]pyrazine-2-carboxamide |
| 316 | 6-cyclopropyl-2-[3-[2-[[(2S)-2-[[(E)-4-(dimethylamino)but-2-enoyl]-methyl- |
| | amino]propanoyl]amino]ethyl]anilino]-5-ethyl-pyridine-3-carboxamide |
| 317 | 3-[3-[(2S)-2-[[(2S)-2-[[(E)-4-(dimethylamino)but-2-enoyl]-methyl- |
| | amino]propanoyl]amino]propyl]anilino]-6-ethyl-5-methyl-pyrazine-2-carboxamide |
| 318 | 3-[3-[2-[[(2S)-2-[4-(dimethylamino)butanoyl-methyl-amino]propanoyl]amino]ethyl]anilino]-6- |
| | ethyl-5-[isopropyl(methyl)amino]pyrazine-2-carboxamide |
| 319 | 6-cyclopropyl-2-[3-[2-[[(2S)-2-[[(E)-4-(dimethylamino)but-2-enoyl]-methyl- |
| | amino]propanoyl]amino]ethyl]anilino]-5-methyl-pyridine-3-carboxamide |
| 320 | 6-cyclopropyl-2-[[5-[2-[[(2S)-2-[[(E)-4-(dimethylamino)but-2-enoyl]-methyl- |
| | amino]propanoyl]amino]ethyl]-3-pyridyl]amino]-5-ethyl-pyridine-3-carboxamide |
| 321 | 3-[3-[(1R)-2-[[2-[[(E)-4-(dimethylamino)but-2-enoyl]-methyl-amino]acetyl]amino]-1-methyl- |
| | ethyl]anilino]-6-ethyl-5-methyl-pyrazine-2-carboxamide |
| 322 | 6-(dimethylamino)-2-[3-[2-[[(2S)-2-[[(E)-4-(dimethylamino)but-2-enoyl]-methyl- |
| | amino]propanoyl]amino]ethyl]anilino]-5-ethyl-pyridine-3-carboxamide |
| 323 | 6-cyclopropyl-2-[[5-[2-[[(2S)-2-[[(E)-4-(dimethylamino)but-2-enoyl]-methyl- |
| | amino]propanoyl]amino]ethyl]-3-pyridyl]amino]-5-methyl-pyridine-3-carboxamide |
| 324 | 3-[3-[(1S)-2-[[2-[[(E)-4-(dimethylamino)but-2-enoyl]-methyl-amino]acetyl]amino]-1-methyl- |
| | ethyl]anilino]-6-ethyl-5-methyl-pyrazine-2-carboxamide |
| 325 | 3-[3-[(1S)-2-[[(2S)-2-[[(E)-4-(dimethylamino)but-2-enoyl]-methyl-amino]propanoyl]amino]-1- |
| | methyl-ethyl]anilino]-6-ethyl-5-methyl-pyrazine-2-carboxamide |
| 326 | 3-[3-[(1R)-2-[[(2S)-2-[[(E)-4-(dimethylamino)but-2-enoyl]-methyl-amino]propanoyl]amino]-1- |
| | methyl-ethyl]anilino]-6-ethyl-5-methyl-pyrazine-2-carboxamide |
| 327 | 3-[3-[2-[[2-[[(E)-4-(dimethylamino)but-2-enoyl]-methyl-amino]acetyl]amino]ethyl]anilino]-6- |
| | ethynyl-5-[isopropyl(methyl)amino]pyrazine-2-carboxamide |
| 328 | 3-[3-[2-[[2-[[(E)-4-(dimethylamino)but-2-enoyl]-methyl-amino]acetyl]amino]ethyl]anilino]-5- |
| | [isopropyl(methyl)amino]-6-vinyl-pyrazine-2-carboxamide |

In some particular embodiments, the compound is any compound selected from Compound #163, 164, 202, 203, 213, 224, 226, 227, 228, 230, 232, 233, 241, and 247, or a pharmaceutically acceptable salt or solvate or stereoisomer thereof.

In some particular embodiments, the compound is any compound selected from Compound #156, 158, 159, and 160, or a pharmaceutically acceptable salt or solvate or stereoisomer thereof.

In some particular embodiments, the compound is any compound selected from Compound #142A, 204, 219, 221, 228, 232, and 246 or a pharmaceutically acceptable salt or solvate or stereoisomer thereof. In some particular embodiments, the compound is any compound selected from Compound #142A, 204, 228, and 232 or a pharmaceutically acceptable salt or solvate or stereoisomer thereof.

In some particular embodiments, the compound is Compound #142A, or a pharmaceutically acceptable salt or solvate or stereoisomer thereof.

In some particular embodiments, the compound is Compound #228, or a pharmaceutically acceptable salt or solvate or stereoisomer thereof.

In some particular embodiments, the compound is Compound #204, or a pharmaceutically acceptable salt or solvate or stereoisomer thereof.

In some particular embodiments, the compound is Compound #232, or a pharmaceutically acceptable salt or solvate or stereoisomer thereof.

In some particular embodiments, the compound is any one compound selected from:

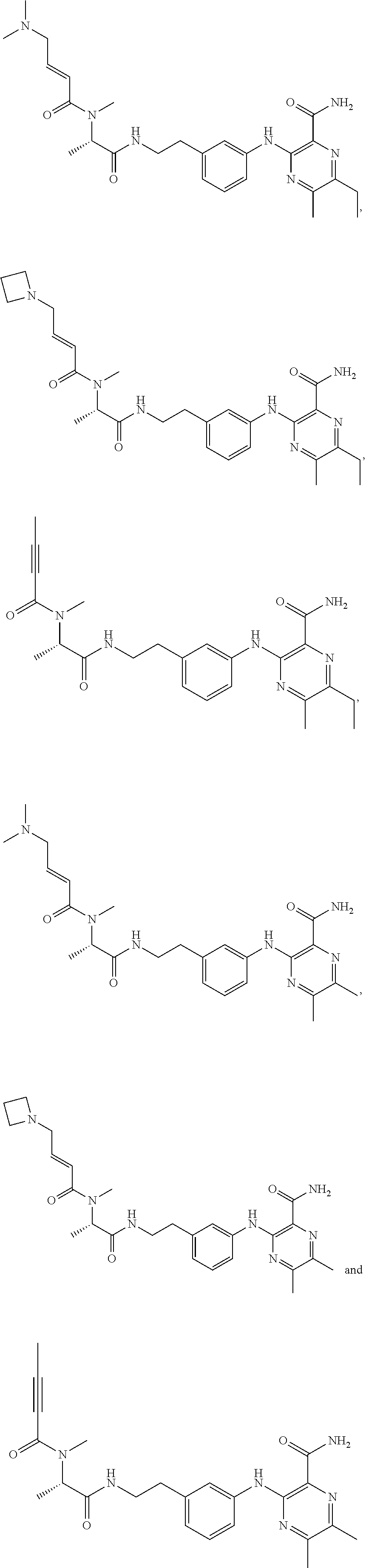

or a pharmaceutically acceptable salt or solvate or stereoisomer thereof.

In some particular embodiments, the compound is any one compound selected from:

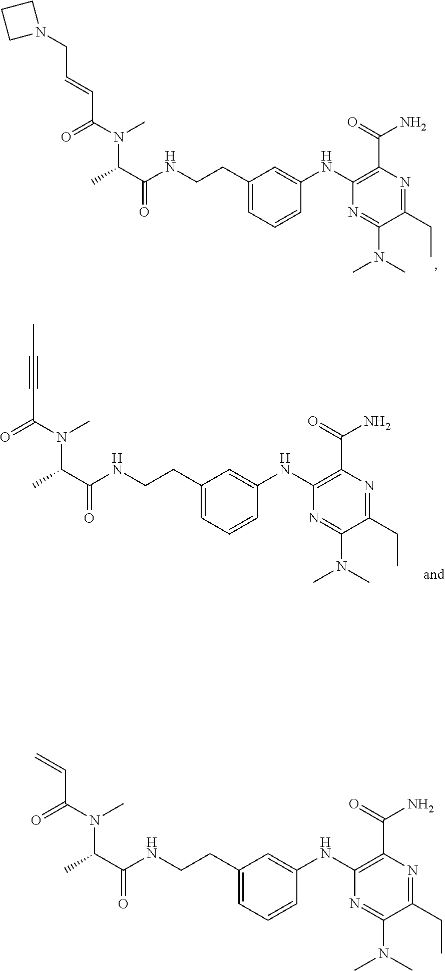 [SMILES: CCc1nc(C(N)=O)c(Nc2cccc(CCNC(=O)[C@H](C)N(C)C(=O)/C=C/CN3CCC3)c2)nc1N(C)C ; CC#CC(=O)N(C)[C@@H](C)C(=O)NCCc1cccc(Nc2nc(N(C)C)c(CC)nc2C(N)=O)c1 ; C=CC(=O)N(C)[C@@H](C)C(=O)NCCc1cccc(Nc2nc(N(C)C)c(CC)nc2C(N)=O)c1]

or a pharmaceutically acceptable salt or solvate or stereoisomer thereof.

In some particular embodiments, the compound is:

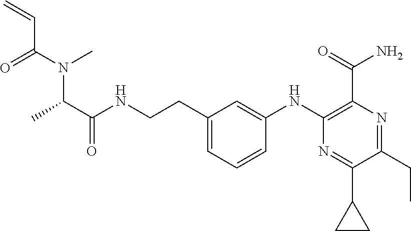 [SMILES: C=CC(=O)N(C)[C@@H](C)C(=O)NCCc1cccc(Nc2nc(C3CC3)c(CC)nc2C(N)=O)c1]

or a pharmaceutically acceptable salt or solvate or stereoisomer thereof.

In some particular embodiments, the compound is:

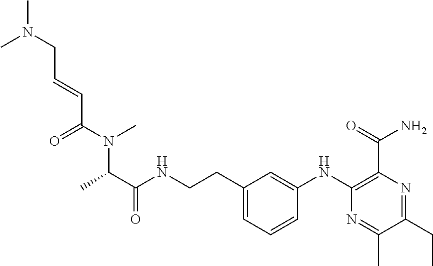 [SMILES: CCc1nc(C(N)=O)c(Nc2cccc(CCNC(=O)[C@H](C)N(C)C(=O)/C=C/CN(C)C)c2)nc1C]

or a pharmaceutically acceptable salt or solvate or stereoisomer thereof.

In some particular embodiments, the compound is:

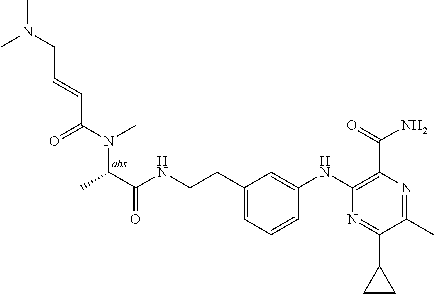

or a pharmaceutically acceptable salt or solvate or stereoisomer thereof.

In some particular embodiments, the compound is:

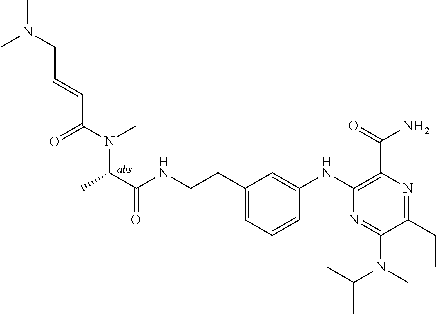 [SMILES: CCc1nc(C(N)=O)c(Nc2cccc(CCNC(=O)[C@H](C)N(C)C(=O)/C=C/CN(C)C)c2)nc1N(C)C(C)C]

or a pharmaceutically acceptable salt or solvate or stereoisomer thereof.

In some particular embodiments, the compound is:

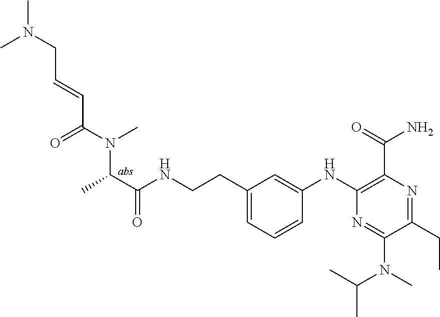 [SMILES: CCc1nc(C(N)=O)c(Nc2cccc(CCNC(=O)[C@H](C)N(C)C(=O)/C=C/CN(C)C)c2)nc1N(C)C(C)C]

or a pharmaceutically acceptable salt or solvate or stereoisomer thereof.

Embodiments of the compounds of Formula (P4-I) display improved potency against FLT3 with IC₅₀ values of as low as less than 1 nM or less than 0.1 nM, and/or high occupancy of active site of FLT3 (e.g., more than 50%, 70% or 90% occupancy) at low dosages of below 5 mg/kg (e.g., at or below 3 mg/kg) when administered in vivo (e.g., in rats).

### Menin Inhibitors

In one embodiment, the menin inhibitor is a compound according to formula (I):

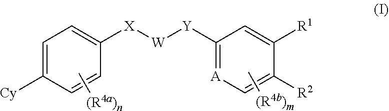

or a pharmaceutically acceptable salt thereof,
wherein:
A is C or N;
Cy is substituted or unsubstituted

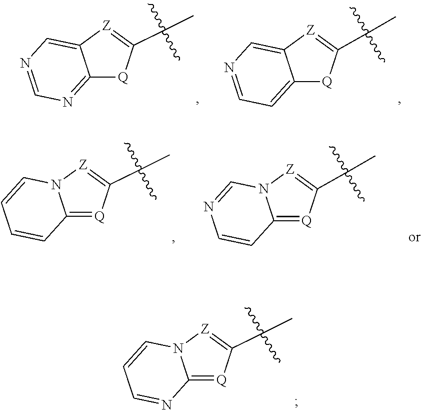

Q is N, —N(H)—, —O—, or —S—;
Z is —CR^{5a}═ or —N═;
X is —NR^{3a}—, —C(R^{3b})₂—, or —O—;
Y is a single bond, —NR^{3a}—, —C(R^{3b})₂—, or —O—;
W is —C(O)—, —S(O)—, or —S(O)₂—;
one of R¹ and R² is Cy²-N(H)C(O)—C(R^{6a})═C(R^{6b})(R^{6c}), or CH₂—Cy²-N(H)C(O)—C(R^{6a})═C(R^{6b})(R^{6c}); and the other is H, C₁₋₆ alkyl, C₁₋₆ haloalkyl, halo, or CN;
Cy² is an optionally substituted group selected from phenyl, pyridyl, or a 4-7 membered heterocycloalkyl ring having 1-2 heteroatoms independently selected from nitrogen, oxygen, or sulfur;
each R^{3a}, and R^{3b} is independently H or C₁₋₆ alkyl;
each R^{4a} and R^{4b} is independently H, halo, CN, OR, —N(R)₂, —C(O)N(R)₂, —NRC(O)R, —SO₂R, —C(O)R, —CO₂R, or an optionally substituted group selected from C₁₋₆ alkyl, C₃₋₇ cycloalkyl, a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur;
each R is independently H, or an optionally substituted group selected from C₁₋₆ aliphatic, phenyl, an 8-10 membered bicyclic aryl ring, a 4-7 membered saturated or partially unsaturated heterocyclic ring having 1-2 heteroatoms independently selected from nitrogen, oxygen, or sulfur, and a 5-6 membered heteroaryl ring having 1-4 heteroatoms independently selected from nitrogen, oxygen, or sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, or sulfur;
R^{5a} is H, C₁₋₆ alkyl, C₁₋₆ haloalkyl, halo, or CN;
each R^{6a} and R^{6b} is independently H or C₁₋₆ alkyl; or R^{6a} and R^{6b} are joined together to form a bond; R^{6c} is H or substituted or unsubstituted C₁₋₆ alkyl;
m is 1, 2, or 3; and
n is 1, 2, 3, or 4.

In certain embodiments, the menin inhibitor is a compound according to formula (XXI):

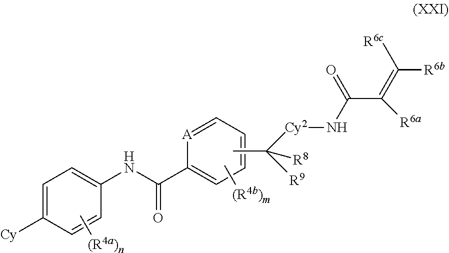

or a pharmaceutically acceptable salt thereof,
wherein each R⁸ and R⁹ is independently H, C₁₋₆ alkyl, C₁₋₆ haloalkyl, halo, or CN; and Cy, Cy², R^{4a}, R^{4b}, R^{6a}, R^{6b}, R^{6c}, m and n are as defined in Formula (I).

In certain embodiments, the menin inhibitor is according to formula (XIVa), (XIVb), or (XIVc):

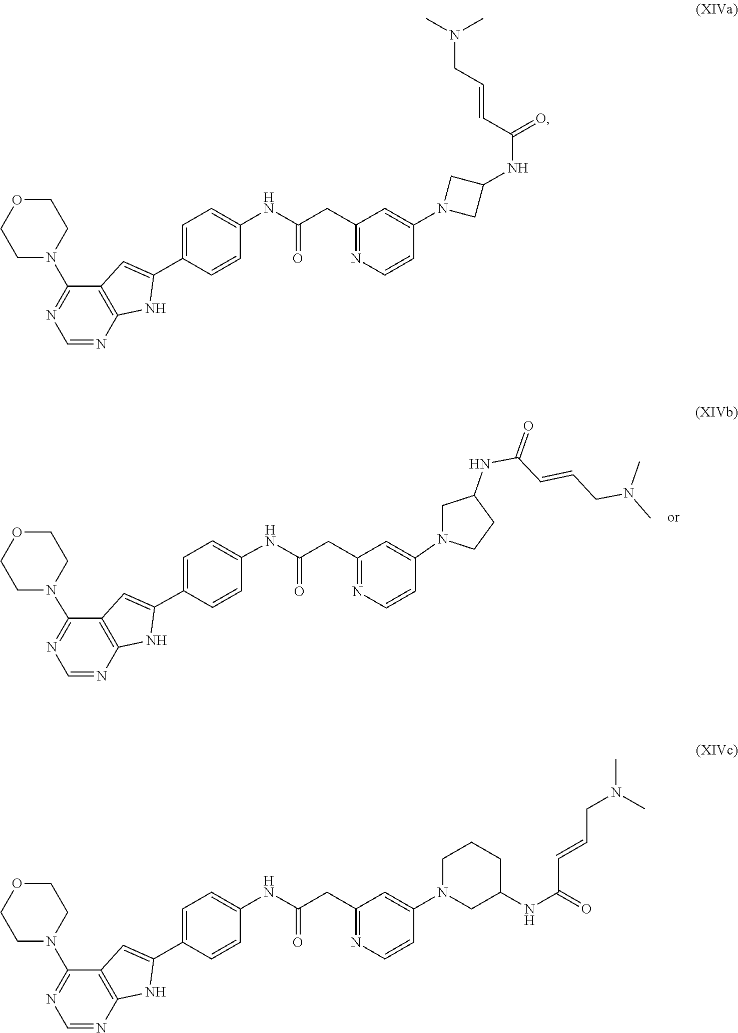 [SMILES: CN(C)C/C=C/C(=O)NC1CN(c2ccnc(CC(=O)Nc3ccc(-c4cc5c(N6CCOCC6)ncnc5[nH]4)cc3)c2)C1 ; CN(C)C/C=C/C(=O)NC1CCN(c2ccnc(CC(=O)Nc3ccc(-c4cc5c(N6CCOCC6)ncnc5[nH]4)cc3)c2)C1 ; CN(C)C/C=C/C(=O)NC1CCCN(c2ccnc(CC(=O)Nc3ccc(-c4cc5c(N6CCOCC6)ncnc5[nH]4)cc3)c2)C1]

or a pharmaceutically acceptable salt thereof.

In certain embodiments, the menin inhibitor is according to formula (XV):

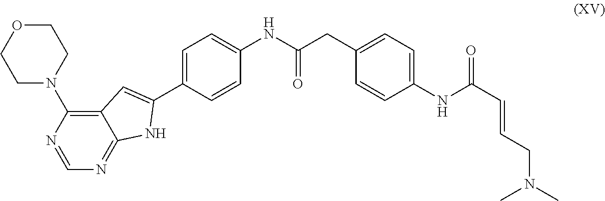 [SMILES: CN(C)C/C=C/C(=O)Nc1ccc(CC(=O)Nc2ccc(-c3cc4c(N5CCOCC5)ncnc4[nH]3)cc2)cc1]

or a pharmaceutically acceptable salt thereof.

In certain embodiments, the menin inhibitor is according to formula (XVI):

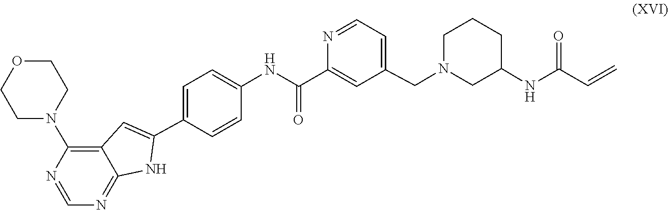 [SMILES: C=CC(=O)NC1CCCN(Cc2ccnc(C(=O)Nc3ccc(-c4cc5c(N6CCOCC6)ncnc5[nH]4)cc3)c2)C1]

or a pharmaceutically acceptable salt thereof.

In certain embodiments, the menin inhibitor is according to formula (XVII):

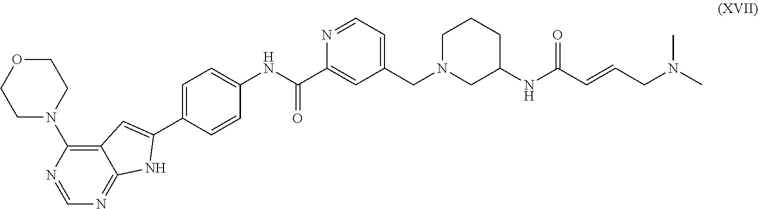 [SMILES: CN(C)C/C=C/C(=O)NC1CCCN(Cc2ccnc(C(=O)Nc3ccc(-c4cc5c(N6CCOCC6)ncnc5[nH]4)cc3)c2)C1]

or a pharmaceutically acceptable salt thereof.

In certain embodiments, the menin inhibitor is according to formula (XXVIIa), (XXVIIb), or (XXVIIc):

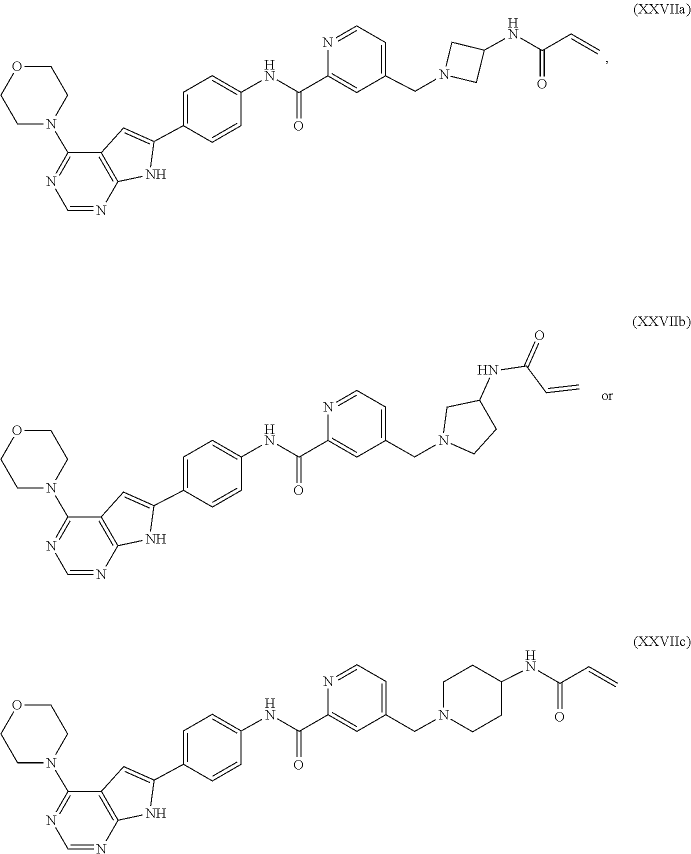 [SMILES: C=CC(=O)NC1CN(Cc2ccnc(C(=O)Nc3ccc(-c4cc5c(N6CCOCC6)ncnc5[nH]4)cc3)c2)C1 ; C=CC(=O)NC1CCN(Cc2ccnc(C(=O)Nc3ccc(-c4cc5c(N6CCOCC6)ncnc5[nH]4)cc3)c2)C1 ; C=CC(=O)NC1CCN(Cc2ccnc(C(=O)Nc3ccc(-c4cc5c(N6CCOCC6)ncnc5[nH]4)cc3)c2)CC1]

or a pharmaceutically acceptable salt thereof.

In certain embodiments, the menin inhibitor is according to formula (XXVIIIa), (XXVIIIb), or (XXVIIIc):

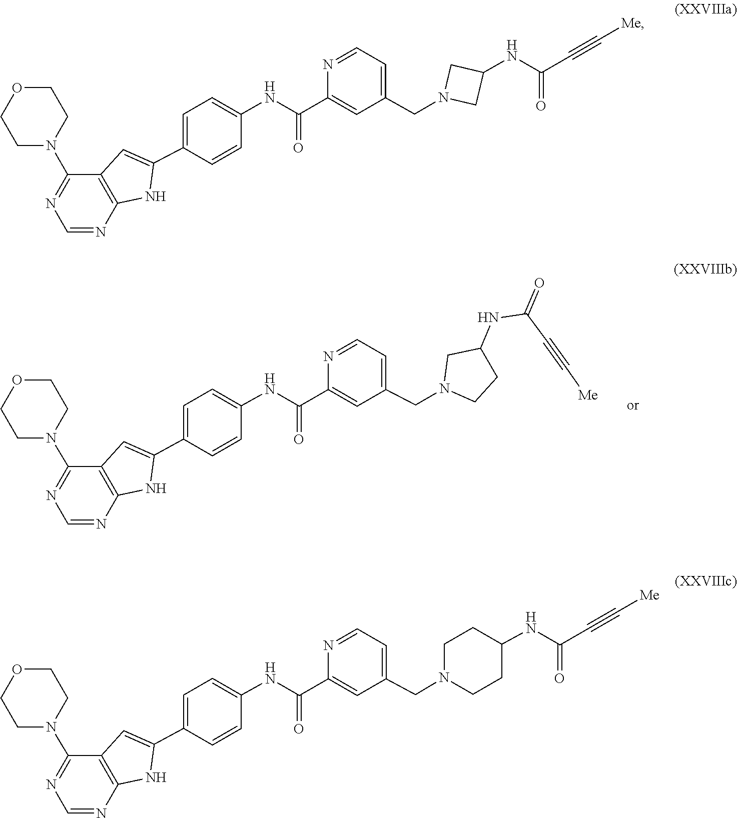 [SMILES: CC#CC(=O)NC1CN(Cc2ccnc(C(=O)Nc3ccc(-c4cc5c(N6CCOCC6)ncnc5[nH]4)cc3)c2)C1 ; CC#CC(=O)NC1CCN(Cc2ccnc(C(=O)Nc3ccc(-c4cc5c(N6CCOCC6)ncnc5[nH]4)cc3)c2)C1 ; CC#CC(=O)NC1CCN(Cc2ccnc(C(=O)Nc3ccc(-c4cc5c(N6CCOCC6)ncnc5[nH]4)cc3)c2)CC1]

or a pharmaceutically acceptable salt thereof.

In certain embodiments, the menin inhibitor is according to formula (XXIXa), (XXIXb), or (XXIXc):

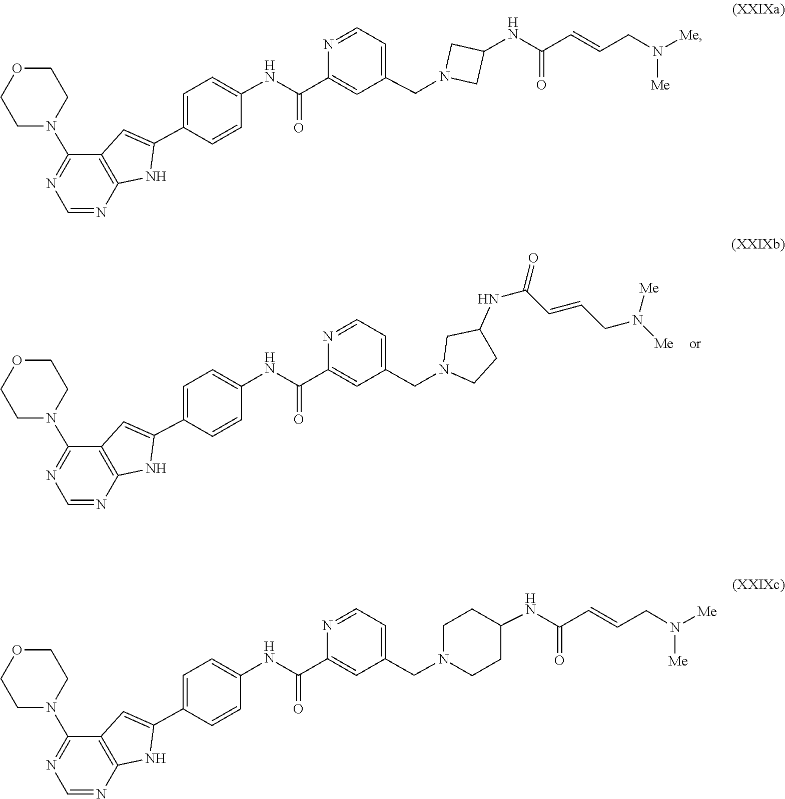 [SMILES: CN(C)C/C=C/C(=O)NC1CN(Cc2ccnc(C(=O)Nc3ccc(-c4cc5c(N6CCOCC6)ncnc5[nH]4)cc3)c2)C1 ; CN(C)C/C=C/C(=O)NC1CCN(Cc2ccnc(C(=O)Nc3ccc(-c4cc5c(N6CCOCC6)ncnc5[nH]4)cc3)c2)C1 ; CN(C)C/C=C/C(=O)NC1CCN(Cc2ccnc(C(=O)Nc3ccc(-c4cc5c(N6CCOCC6)ncnc5[nH]4)cc3)c2)CC1]

or a pharmaceutically acceptable salt thereof.

In certain embodiments, the menin inhibitor is according to formula (XLa), (XLb), or (XLc):

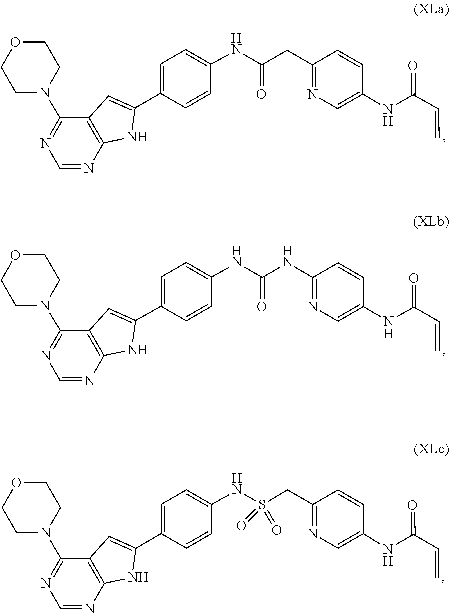 [SMILES: C=CC(=O)Nc1ccc(CC(=O)Nc2ccc(-c3cc4c(N5CCOCC5)ncnc4[nH]3)cc2)nc1 ; C=CC(=O)Nc1ccc(NC(=O)Nc2ccc(-c3cc4c(N5CCOCC5)ncnc4[nH]3)cc2)nc1 ; C=CC(=O)Nc1ccc(CS(=O)(=O)Nc2ccc(-c3cc4c(N5CCOCC5)ncnc4[nH]3)cc2)nc1]

or a pharmaceutically acceptable salt thereof.

In certain embodiments, the menin inhibitor is according to formula (XLIa), (XLIb), or (XLIc):

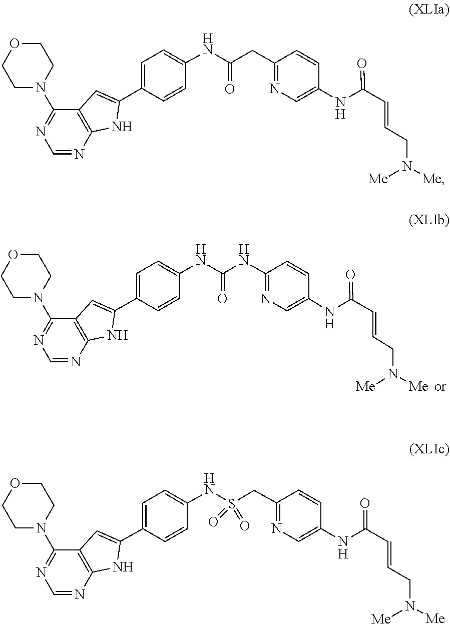 [SMILES: CN(C)C/C=C/C(=O)Nc1ccc(CC(=O)Nc2ccc(-c3cc4c(N5CCOCC5)ncnc4[nH]3)cc2)nc1 ; CN(C)C/C=C/C(=O)Nc1ccc(NC(=O)Nc2ccc(-c3cc4c(N5CCOCC5)ncnc4[nH]3)cc2)nc1 ; CN(C)C/C=C/C(=O)Nc1ccc(CS(=O)(=O)Nc2ccc(-c3cc4c(N5CCOCC5)ncnc4[nH]3)cc2)nc1]

or a pharmaceutically acceptable salt thereof.

In certain embodiments, the menin inhibitor is according to formula (XLIIa), (XLIIb), or (XLIIc):

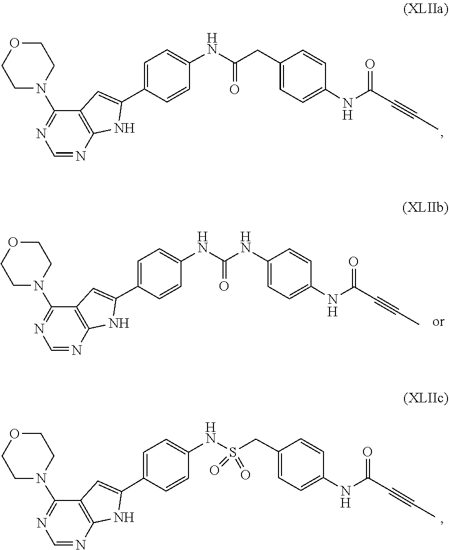 [SMILES: CC#CC(=O)Nc1ccc(CC(=O)Nc2ccc(-c3cc4c(N5CCOCC5)ncnc4[nH]3)cc2)cc1 ; CC#CC(=O)Nc1ccc(NC(=O)Nc2ccc(-c3cc4c(N5CCOCC5)ncnc4[nH]3)cc2)cc1 ; CC#CC(=O)Nc1ccc(CS(=O)(=O)Nc2ccc(-c3cc4c(N5CCOCC5)ncnc4[nH]3)cc2)cc1]

or a pharmaceutically acceptable salt thereof.

In certain embodiments, the menin inhibitor is according to formula (XLIIIa), (XLIIIb), or (XLIIIc):

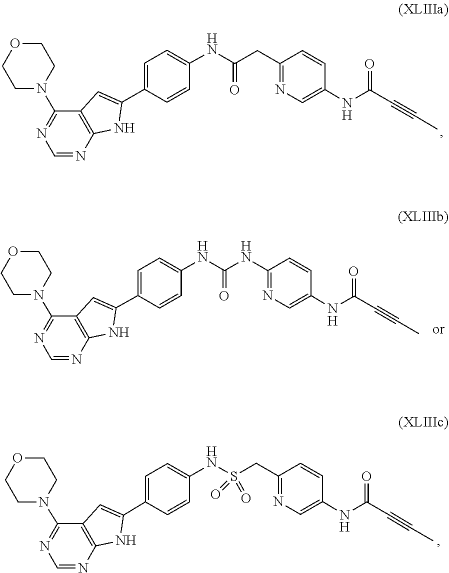 [SMILES: CC#CC(=O)Nc1ccc(CC(=O)Nc2ccc(-c3cc4c(N5CCOCC5)ncnc4[nH]3)cc2)nc1 ; CC#CC(=O)Nc1ccc(NC(=O)Nc2ccc(-c3cc4c(N5CCOCC5)ncnc4[nH]3)cc2)nc1 ; CC#CC(=O)Nc1ccc(CS(=O)(=O)Nc2ccc(-c3cc4c(N5CCOCC5)ncnc4[nH]3)cc2)nc1]

or a pharmaceutically acceptable salt thereof.

In certain embodiments, the menin inhibitor is selected from Compound 3, Compound 6, Compound 10, and Compound 13:

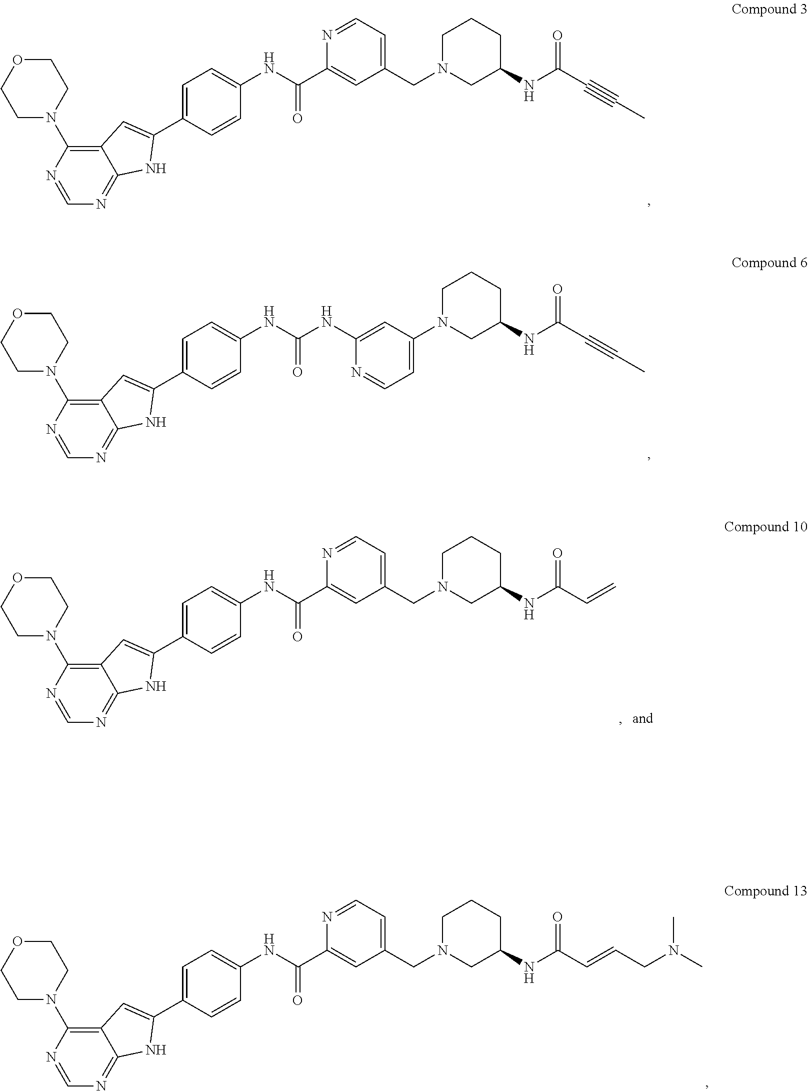 [SMILES: CCCC(=O)N[C@@H]1CCCN(Cc2ccnc(C(=O)Nc3ccc(-c4cc5c(N6CCOCC6)ncnc5[nH]4)cc3)c2)C1 ; CC#CC(=O)N[C@@H]1CCCN(c2ccnc(NC(=O)Nc3ccc(-c4cc5c(N6CCOCC6)ncnc5[nH]4)cc3)c2)C1 ; C=CC(=O)N[C@@H]1CCCN(Cc2ccnc(C(=O)Nc3ccc(-c4cc5c(N6CCOCC6)ncnc5[nH]4)cc3)c2)C1 ; CN(C)C/C=C/C(=O)N[C@@H]1CCCN(Cc2ccnc(C(=O)Nc3ccc(-c4cc5c(N6CCOCC6)ncnc5[nH]4)cc3)c2)C1]

or a salt thereof.

In certain embodiments, the menin inhibitor is

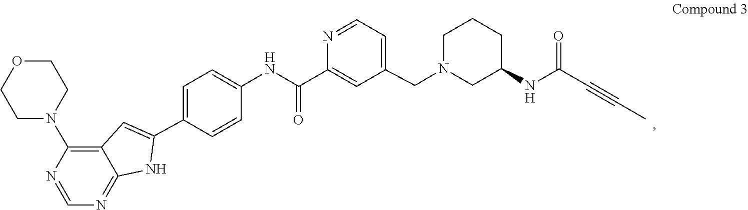 [SMILES: CC#CC(=O)N[C@@H]1CCCN(Cc2ccnc(C(=O)Nc3ccc(-c4cc5c(N6CCOCC6)ncnc5[nH]4)cc3)c2)C1]

or a salt thereof.

In certain embodiments, the menin inhibitor is

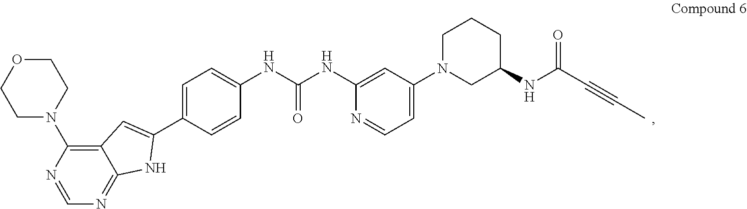 [SMILES: CC#CC(=O)N[C@@H]1CCCN(c2ccnc(NC(=O)Nc3ccc(-c4cc5c(N6CCOCC6)ncnc5[nH]4)cc3)c2)C1]

or a salt thereof.

In certain embodiments, the menin inhibitor is

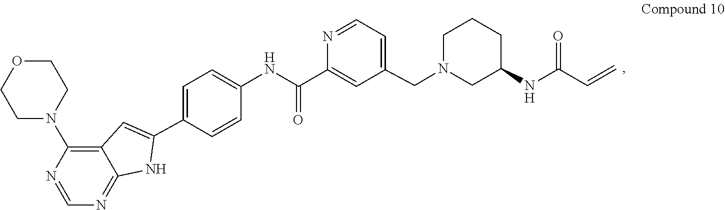 [SMILES: C=CC(=O)N[C@@H]1CCCN(Cc2ccnc(C(=O)Nc3ccc(-c4cc5c(N6CCOCC6)ncnc5[nH]4)cc3)c2)C1]

or a salt thereof.

In certain embodiments, the menin inhibitor is

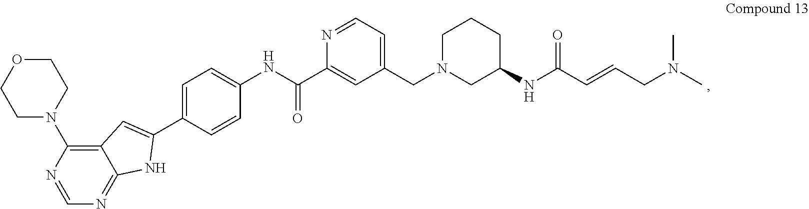 [SMILES: CN(C)C/C=C/C(=O)N[C@@H]1CCCN(Cc2ccnc(C(=O)Nc3ccc(-c4cc5c(N6CCOCC6)ncnc5[nH]4)cc3)c2)C1]

or a salt thereof.

In some embodiments, the combination comprises a compound or FLT3 inhibitor selected from any one of the compounds listed in Table 1 and the menin inhibitor Compound 3. In certain embodiments, the combination comprises a compound or FLT3 inhibitor selected from any one of the compounds listed in Table 1 and the menin inhibitor Compound 6. In certain embodiments, the combination comprises a compound or FLT3 inhibitor selected from any one of the compounds listed in Table 1 and the menin inhibitor Compound 10 In certain embodiments, the combination comprises a compound or FLT3 inhibitor selected from any one of the compounds listed in Table 1 and the menin inhibitor Compound 13. In certain embodiments, the combination comprises a compound or FLT3 inhibitor selected from any one of the compounds listed in Table 1 and a menin inhibitor according to formula (XLIIIa). In certain embodiments, the combination comprises a compound or FLT3 inhibitor selected from any one of the compounds listed in Table 1 and the menin inhibitor Compound 13. In certain embodiments, the combination comprises a compound or FLT3 inhibitor selected from any one of the compounds listed in Table 1 and a menin inhibitor according to formula (XLIIIb). In certain embodiments, the combination comprises a compound or FLT3 inhibitor selected from any one of the compounds listed in Table 1 and the menin inhibitor Compound 13. In certain embodiments, the combination comprises the compound or FLT3 inhibitor selected from any one of the compounds listed in Table 1 and a menin inhibitor according to formula (XLIIIc).

In some embodiments, the combination comprises the compound or FLT3 inhibitor according to compound ID 37 and the menin inhibitor Compound 3. In certain embodiments, the combination comprises the compound or FLT3 inhibitor according to compound ID 37 and the menin inhibitor Compound 6. In certain embodiments, the combination comprises the compound or FLT3 inhibitor according to compound ID 37 and the menin inhibitor Compound 10. In certain embodiments, the combination comprises the compound or FLT3 inhibitor according to compound ID 37 and the menin inhibitor Compound 13. In certain embodiments, the combination comprises the compound or FLT3 inhibitor according to compound ID 37 and a menin inhibitor according to formula (XLIIIa). In certain embodiments, the combination comprises the compound or FLT3 inhibitor according to compound ID 37 and the menin inhibitor Compound 13. In certain embodiments, the combination comprises the compound or FLT3 inhibitor according to compound ID 37 and a menin inhibitor according to formula (XLIIIb). In certain embodiments, the combination comprises the compound or FLT3 inhibitor according to compound ID 37 and the menin inhibitor Compound 13. In certain embodiments, the combination comprises the compound or FLT3 inhibitor according to compound ID 37 and a menin inhibitor according to formula (XLIIIc). In certain embodiments, the combination comprises the compound or FLT3 inhibitor according to compound ID 37 and the menin inhibitor KO-539 or Zifomenib. In certain embodiments, the combination comprises the compound or FLT3 inhibitor according to compound ID 37 and the menin inhibitor SNDX-5613 or Revumenib.

In some embodiments, the combination comprises the compound or FLT3 inhibitor according to compound ID 116 and the menin inhibitor Compound 3. In certain embodiments, the combination comprises the compound or FLT3 inhibitor according to compound ID 116 and the menin inhibitor Compound 6. In certain embodiments, the combination comprises the compound or FLT3 inhibitor according to compound ID 116 and the menin inhibitor Compound 10. In certain embodiments, the combination comprises the compound or FLT3 inhibitor according to compound ID 116 and the menin inhibitor Compound 13. In certain embodiments, the combination comprises the compound or FLT3 inhibitor according to compound ID 116 and a menin inhibitor according to formula (XLIIIa). In certain embodiments, the combination comprises the compound or FLT3 inhibitor according to compound ID 116 and the menin inhibitor Compound 13. In certain embodiments, the combination comprises the compound or FLT3 inhibitor according to compound ID 116 and a menin inhibitor is according to formula (XLIIIb). In certain embodiments, the combination comprises the compound or FLT3 inhibitor according to compound ID 116 and the menin inhibitor Compound 13. In certain embodiments, the combination comprises the compound or FLT3 inhibitor according to compound ID 116 and a menin inhibitor is according to formula (XLIIIc). In certain embodiments, the combination comprises the compound or FLT3 inhibitor according to compound ID 116 and the menin inhibitor KO-539 or Zifomenib. In certain embodiments, the combination comprises the compound or FLT3 inhibitor according to compound ID 116 and the menin inhibitor SNDX-5613 or Revumenib.

In some embodiments, the combination comprises the compound or FLT3 inhibitor according to compound ID 122 and the menin inhibitor Compound 3. In certain embodiments, the combination comprises the compound or FLT3 inhibitor according to compound ID 122 and the menin inhibitor Compound 6. In certain embodiments, the combination comprises the compound or FLT3 inhibitor according to compound ID 122 and the menin inhibitor Compound 10. In certain embodiments, the combination comprises the compound or FLT3 inhibitor according to compound ID 122 and the menin inhibitor Compound 13. In certain embodiments, the combination comprises the compound or FLT3 inhibitor according to compound ID 122 and a menin inhibitor according to formula (XLIIIa). In certain embodiments, the combination comprises the compound or FLT3 inhibitor according to compound ID 122 and the menin inhibitor Compound 13. In certain embodiments, the combination comprises the compound or FLT3 inhibitor according to compound ID 122 and a menin inhibitor according to formula (XLIIIb). In certain embodiments, the combination comprises the compound or FLT3 inhibitor according to compound ID 122 and the menin inhibitor Compound 13. In certain embodiments, the combination comprises the compound or FLT3 inhibitor according to compound ID 122 and a menin inhibitor according to formula (XLIIIc). In certain embodiments, the combination comprises the compound or FLT3 inhibitor according to compound ID 122 and the menin inhibitor KO-539 or Zifomenib. In certain embodiments, the combination comprises the compound or FLT3 inhibitor according to compound ID 122 and the menin inhibitor SNDX-5613 or Revumenib.

In some embodiments, the combination comprises the compound or FLT3 inhibitor according to compound ID 124 and the menin inhibitor Compound 3. In certain embodiments, the combination comprises the compound or FLT3 inhibitor according to compound ID 124 and the menin inhibitor Compound 6. In certain embodiments, the combination comprises the compound or FLT3 inhibitor according to compound ID 124 and the menin inhibitor Compound 10. In certain embodiments, the combination comprises the compound or FLT3 inhibitor according to compound ID 124 and the menin inhibitor Compound 13. In certain embodiments, the combination comprises the compound or FLT3 inhibitor according to compound ID 124 and a menin inhibitor according to formula (XLIIIa). In certain embodiments, the combination comprises the compound or FLT3 inhibitor according to compound ID 124 and the menin inhibitor Compound 13. In certain embodiments, the combination comprises the compound or FLT3 inhibitor according to compound ID 124 and a menin inhibitor according to formula (XLIIIb). In certain embodiments, the combination comprises the compound or FLT3 inhibitor according to compound ID 124 and the menin inhibitor Compound 13. In certain embodiments, the combination comprises the compound or FLT3 inhibitor according to compound ID 124 and a menin inhibitor according to formula (XLIIIc). In certain embodiments, the combination comprises the compound or FLT3 inhibitor according to compound ID 124 and the menin inhibitor KO-539 or Zifomenib. In certain embodiments, the combination comprises the compound or FLT3 inhibitor according to compound ID 124 and the menin inhibitor SNDX-5613 or Revumenib.

In some embodiments, the combination comprises the compound or FLT3 inhibitor according to compound ID 129 and the menin inhibitor Compound 3. In certain embodiments, the combination comprises the compound or FLT3 inhibitor according to compound ID 129 and the menin inhibitor Compound 6. In certain embodiments, the combination comprises the compound or FLT3 inhibitor according to compound ID 129 and the menin inhibitor Compound 10. In certain embodiments, the combination comprises the compound or FLT3 inhibitor according to compound ID 129 and the menin inhibitor Compound 13. In certain embodiments, the combination comprises the compound or FLT3 inhibitor according to compound ID 129 and a menin inhibitor according to formula (XLIIIa). In certain embodiments, the combination comprises the compound or FLT3 inhibitor according to compound ID 129 and the menin inhibitor Compound 13. In certain embodiments, the combination comprises the compound or FLT3 inhibitor according to compound ID 129 and a menin inhibitor according to formula (XLIIIb). In certain embodiments, the combination comprises the compound or FLT3 inhibitor according to compound ID 129 and the menin inhibitor Compound 13. In certain embodiments, the combination comprises the compound or FLT3 inhibitor according to compound ID 129 and a menin inhibitor according to formula (XLIIIc). In certain embodiments, the combination comprises the compound or FLT3 inhibitor according to compound ID 129 and the menin inhibitor KO-539 or Zifomenib. In certain embodiments, the combination comprises the compound or FLT3 inhibitor according to compound ID 129 and the menin inhibitor SNDX-5613 or Revumenib.

In some embodiments, the combination comprises the compound or FLT3 inhibitor according to compound ID 137 and the menin inhibitor Compound 3. In certain embodiments, the combination comprises the compound or FLT3 inhibitor according to compound ID 137 and the menin inhibitor Compound 6. In certain embodiments, the combination comprises the compound or FLT3 inhibitor according to compound ID 137 and the menin inhibitor Compound 10. In certain embodiments, the combination comprises the compound or FLT3 inhibitor according to compound ID 137 and the menin inhibitor Compound 13. In certain embodiments, the combination comprises the compound or FLT3 inhibitor according to compound ID 137 and a menin inhibitor according to formula (XLIIIa). In certain embodiments, the combination comprises the compound or FLT3 inhibitor according to compound ID 137 and the menin inhibitor Compound 13. In certain embodiments, the combination comprises the compound or FLT3 inhibitor according to compound ID 137 and a menin inhibitor according to formula (XLIIIb). In certain embodiments, the combination comprises the compound or FLT3 inhibitor according to compound ID 137 and the menin inhibitor Compound 13. In certain embodiments, the combination comprises the compound or FLT3 inhibitor according to compound ID 137 and a menin inhibitor according to formula (XLIIIc). In certain embodiments, the combination comprises the compound or FLT3 inhibitor according to compound ID 137 and the menin inhibitor KO-539 or Zifomenib. In certain embodiments, the combination comprises the compound or FLT3 inhibitor according to compound ID 137 and the menin inhibitor SNDX-5613 or Revumenib.

In some embodiments, the combination comprises the compound or FLT3 inhibitor according to compound ID 143 and the menin inhibitor Compound 3. In certain embodiments, the combination comprises the compound or FLT3 inhibitor according to compound ID 143 and the menin inhibitor Compound 6. In certain embodiments, the combination comprises the compound or FLT3 inhibitor according to compound ID 143 and the menin inhibitor Compound 10. In certain embodiments, the combination comprises the compound or FLT3 inhibitor according to compound ID 143 and the menin inhibitor Compound 13. In certain embodiments, the combination comprises the compound or FLT3 inhibitor according to compound ID 143 and a menin inhibitor according to formula (XLIIIa). In certain embodiments, the combination comprises the compound or FLT3 inhibitor according to compound ID 143 and the menin inhibitor Compound 13. In certain embodiments, the combination comprises the compound or FLT3 inhibitor according to compound ID 143 and a menin inhibitor according to formula (XLIIIb). In certain embodiments, the combination comprises the compound or FLT3 inhibitor according to compound ID 143 and the menin inhibitor Compound 13. In certain embodiments, the combination comprises the compound or FLT3 inhibitor according to compound ID 143 and a menin inhibitor according to formula (XLIIIc). In certain embodiments, the combination comprises the compound or FLT3 inhibitor according to compound ID 143 and the menin inhibitor KO-539 or Zifomenib. In certain embodiments, the combination comprises the compound or FLT3 inhibitor according to compound ID 143 and the menin inhibitor SNDX-5613 or Revumenib.

In some embodiments, the combination comprises the compound or FLT3 inhibitor according to compound ID 144 and the menin inhibitor Compound 3. In certain embodiments, the combination comprises the compound or FLT3 inhibitor according to compound ID 144 and the menin inhibitor Compound 6. In certain embodiments, the combination comprises the compound or FLT3 inhibitor according to compound ID 144 and the menin inhibitor Compound 10. In certain embodiments, the combination comprises the compound or FLT3 inhibitor according to compound ID 144 and the menin inhibitor Compound 13. In certain embodiments, the combination comprises the compound or FLT3 inhibitor according to compound ID 144 and a menin inhibitor according to formula (XLIIIa). In certain embodiments, the combination comprises the compound or FLT3 inhibitor according to compound ID 144 and the menin inhibitor Compound 13. In certain embodiments, the combination comprises the compound or FLT3 inhibitor according to compound ID 144 and a menin inhibitor according to formula (XLIIIb). In certain embodiments, the combination comprises the compound or FLT3 inhibitor according to compound ID 144 and the menin inhibitor Compound 13. In certain embodiments, the combination comprises the compound or FLT3 inhibitor according to compound ID 144 and a menin inhibitor according to formula (XLIIIc). In certain embodiments, the combination comprises the compound or FLT3 inhibitor according to compound ID 144 and the menin inhibitor KO-539 or Zifomenib. In certain embodiments, the combination comprises the compound or FLT3 inhibitor according to compound ID 144 and the menin inhibitor SNDX-5613 or Revumenib.

In some embodiments, the combination comprises the compound or FLT3 inhibitor according to compound ID 156 and the menin inhibitor Compound 3. In certain embodiments, the combination comprises the compound or FLT3 inhibitor according to compound ID 156 and the menin inhibitor Compound 6. In certain embodiments, the combination comprises the compound or FLT3 inhibitor according to compound ID 156 and the menin inhibitor Compound 10. In certain embodiments, the combination comprises the compound or FLT3 inhibitor according to compound ID 156 and the menin inhibitor Compound 13. In certain embodiments, the combination comprises the compound or FLT3 inhibitor according to compound ID 156 and a menin inhibitor according to formula (XLIIIa). In certain embodiments, the combination comprises the compound or FLT3 inhibitor according to compound ID 156 and the menin inhibitor Compound 13. In certain embodiments, the combination comprises the compound or FLT3 inhibitor according to compound ID 156 and a menin inhibitor according to formula (XLIIIb). In certain embodiments, the combination comprises the compound or FLT3 inhibitor according to compound ID 156 and the menin inhibitor Compound 13. In certain embodiments, the combination comprises the compound or FLT3 inhibitor according to compound ID 156 and a menin inhibitor according to formula (XLIIIc). In certain embodiments, the combination comprises the compound or FLT3 inhibitor according to compound ID 156 and the menin inhibitor KO-539 or Zifomenib. In certain embodiments, the combination comprises the compound or FLT3 inhibitor according to compound ID 156, and the menin inhibitor SNDX-5613 or Revumenib.

In some embodiments, the combination comprises the compound or FLT3 inhibitor according to compound ID 158 and the menin inhibitor Compound 3. In certain embodiments, the combination comprises the compound or FLT3 inhibitor according to compound ID 158 and the menin inhibitor Compound 6. In certain embodiments, the combination comprises the compound or FLT3 inhibitor according to compound ID 158 and the menin inhibitor Compound 10. In certain embodiments, the combination comprises the compound or FLT3 inhibitor according to compound ID 158 and the menin inhibitor Compound 13. In certain embodiments, the combination comprises the compound or FLT3 inhibitor according to compound ID 158 and a menin inhibitor according to formula (XLIIIa). In certain embodiments, the combination comprises the compound or FLT3 inhibitor according to compound ID 158 and the menin inhibitor Compound 13. In certain embodiments, the combination comprises the compound or FLT3 inhibitor according to compound ID 158 and a menin inhibitor according to formula (XLIIIb). In certain embodiments, the combination comprises the compound or FLT3 inhibitor according to compound ID 158 and the menin inhibitor Compound 13. In certain embodiments, the combination comprises the compound or FLT3 inhibitor according to compound ID 158 and a menin inhibitor according to formula (XLIIIc). In certain embodiments, the combination comprises the compound or FLT3 inhibitor according to compound ID 158 and the menin inhibitor KO-539 or Zifomenib. In certain embodiments, the combination comprises the compound or FLT3 inhibitor according to compound ID 158 and the menin inhibitor SNDX-5613 or Revumenib.

In some embodiments, the combination comprises the compound or FLT3 inhibitor according to compound ID 204 and the menin inhibitor Compound 3. In certain embodiments, the combination comprises the compound or FLT3 inhibitor according to compound ID 204 and the menin inhibitor Compound 6. In certain embodiments, the combination comprises the compound or FLT3 inhibitor according to compound ID 204 and the menin inhibitor Compound 10. In certain embodiments, the combination comprises the compound or FLT3 inhibitor according to compound ID 204 and the menin inhibitor Compound 13. In certain embodiments, the combination comprises the compound or FLT3 inhibitor according to compound ID 204 and a menin inhibitor according to formula (XLIIIa). In certain embodiments, the combination comprises the compound or FLT3 inhibitor according to compound ID 204 and the menin inhibitor Compound 13. In certain embodiments, the combination comprises the compound or FLT3 inhibitor according to compound ID 204 and a menin inhibitor according to formula (XLIIIb). In certain embodiments, the combination comprises the compound or FLT3 inhibitor according to compound ID 204 and the menin inhibitor Compound 13. In certain embodiments, the combination comprises the compound or FLT3 inhibitor according to compound ID 204 and a menin inhibitor according to formula (XLIIIc). In certain embodiments, the combination comprises the compound or FLT3 inhibitor according to compound ID 204 and the menin inhibitor KO-539 or Zifomenib. In certain embodiments, the combination comprises the compound or FLT3 inhibitor according to compound ID 204, and the menin inhibitor SNDX-5613 or Revumenib.

In some embodiments, the combination comprises the compound or FLT3 inhibitor according to compound ID 205 and the menin inhibitor Compound 3. In certain embodiments, the combination comprises the compound or FLT3 inhibitor according to compound ID 205 and the menin inhibitor Compound 6. In certain embodiments, the combination comprises the compound or FLT3 inhibitor according to compound ID 205 and the menin inhibitor Compound 10. In certain embodiments, the combination comprises the compound or FLT3 inhibitor according to compound ID 205 and the menin inhibitor Compound 13. In certain embodiments, the combination comprises the compound or FLT3 inhibitor according to compound ID 205 and a menin inhibitor according to formula (XLIIIa). In certain embodiments, the combination comprises the compound or FLT3 inhibitor according to compound ID 205 and the menin inhibitor Compound 13. In certain embodiments, the combination comprises the compound or FLT3 inhibitor according to compound ID 205 and a menin inhibitor according to formula (XLIIIb). In certain embodiments, the combination comprises the compound or FLT3 inhibitor according to compound ID 205 and the menin inhibitor Compound 13. In certain embodiments, the combination comprises the compound or FLT3 inhibitor according to compound ID 205 and a menin inhibitor according to formula (XLIIIc). In certain embodiments, the combination comprises the compound or FLT3 inhibitor according to compound ID 205 and the menin inhibitor KO-539 or Zifomenib. In certain embodiments, the combination comprises the compound or FLT3 inhibitor according to compound ID 205 and the menin inhibitor SNDX-5613 or Revumenib.

In some embodiments, the combination comprises the compound or FLT3 inhibitor according to compound ID 219 and the menin inhibitor Compound 3. In certain embodiments, the combination comprises the compound or FLT3 inhibitor according to compound ID 219 and the menin inhibitor Compound 6. In certain embodiments, the combination comprises the compound or FLT3 inhibitor according to compound ID 219 and the menin inhibitor Compound 10. In certain embodiments, the combination comprises the compound or FLT3 inhibitor according to compound ID 219 and the menin inhibitor Compound 13. In certain embodiments, the combination comprises the compound or FLT3 inhibitor according to compound ID 219 and a menin inhibitor according to formula (XLIIIa). In certain embodiments, the combination comprises the compound or FLT3 inhibitor according to compound ID 219 and the menin inhibitor Compound 13. In certain embodiments, the combination comprises the compound or FLT3 inhibitor according to compound ID 219 and a menin inhibitor according to formula (XLIIIb). In certain embodiments, the combination comprises the compound or FLT3 inhibitor according to compound ID 219 and the menin inhibitor Compound 13. In certain embodiments, the combination comprises the compound or FLT3 inhibitor according to compound ID 219 and a menin inhibitor according to formula (XLIIIc). In certain embodiments, the combination comprises the compound or FLT3 inhibitor according to compound ID 219 and the menin inhibitor KO-539 or Zifomenib. In certain embodiments, the combination comprises the compound or FLT3 inhibitor according to compound ID 219 and the menin inhibitor SNDX-5613 or Revumenib.

In some embodiments, the combination comprises the compound or FLT3 inhibitor according to compound ID 221 and the menin inhibitor Compound 3. In certain embodiments, the combination comprises the compound or FLT3 inhibitor according to compound ID 221 and the menin inhibitor Compound 6. In certain embodiments, the combination comprises the compound or FLT3 inhibitor according to compound ID 221 and the menin inhibitor Compound 10. In certain embodiments, the combination comprises the compound or FLT3 inhibitor according to compound ID 221 and the menin inhibitor Compound 13. In certain embodiments, the combination comprises the compound or FLT3 inhibitor according to compound ID 221 and a menin inhibitor according to formula (XLIIIa). In certain embodiments, the combination comprises the compound or FLT3 inhibitor according to compound ID 221 and the menin inhibitor Compound 13. In certain embodiments, the combination comprises the compound or FLT3 inhibitor according to compound ID 221 and a menin inhibitor according to formula (XLIIIb). In certain embodiments, the combination comprises the compound or FLT3 inhibitor according to compound ID 221 and the menin inhibitor Compound 13. In certain embodiments, the combination comprises the compound or FLT3 inhibitor according to compound ID 221 and a menin inhibitor according to formula (XLIIIc). In certain embodiments, the combination comprises the compound or FLT3 inhibitor according to compound ID 221 and the menin inhibitor KO-539 or Zifomenib. In certain embodiments, the combination comprises the compound or FLT3 inhibitor according to compound ID 221 and the menin inhibitor SNDX-5613 or Revumenib.

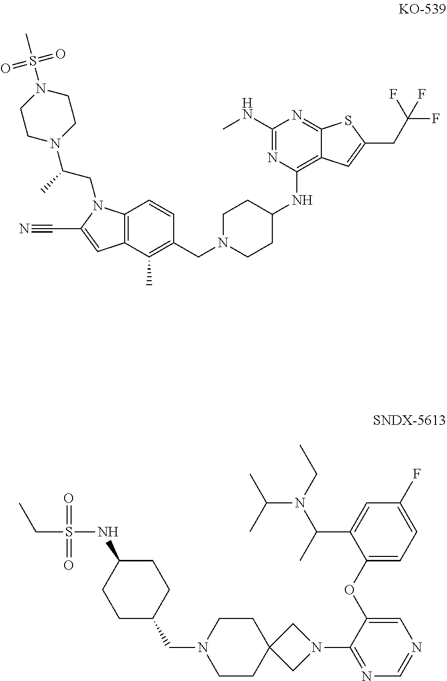

In some embodiments, provided herein is a pharmaceutical composition comprising a FLT3 inhibitor or a pharmaceutically acceptable salt thereof and a menin inhibitor or a pharmaceutically acceptable salt thereof, and a pharmaceutically acceptable excipient.

In some embodiments, provided herein is a pharmaceutical composition comprising a compound selected from Formula (P4-I) or a pharmaceutically acceptable salt thereof, an menin inhibitor or a pharmaceutically acceptable salt thereof, and a pharmaceutically acceptable excipient.

In some embodiments, provided herein is a pharmaceutical composition comprising a therapeutically effective amount of a compound of Formula (P4-I), an menin inhibitor, and a pharmaceutically acceptable excipient.

The pharmaceutical composition comprising the FLT3 inhibitor and the pharmaceutical composition comprising the menin inhibitor can be administered in separate or simultaneous compositions.

In some embodiments, provided herein is a pharmaceutical composition comprising a FLT3 inhibitor or a pharmaceutically acceptable salt thereof and a pharmaceutically acceptable excipient for administration in combination with a menin inhibitor or a pharmaceutically acceptable salt thereof, and a pharmaceutically acceptable excipient.

In some embodiments, the pharmaceutical compositions provided herein are formulated for a route of administration selected from oral administration, parenteral administration, buccal administration, nasal administration, topical administration, or rectal administration.

In some embodiments, provided herein are methods for treating an proliferative disease or condition comprising administering to a patient in need the pharmaceutical composition(s) provided herein.

In some embodiments, provided herein are methods for treating an proliferative disease or condition comprising administering to a patient in need a pharmaceutical composition comprising a FLT3 inhibitor provided herein in combination with administering a menin inhibitor provided herein.

In some embodiments, provided herein are methods for treating a autoimmune disease comprising administering to a patient in need the pharmaceutical composition(s) provided herein.

In some embodiments, provided herein are methods for treating a autoimmune disease comprising administering to a patient in need a pharmaceutical composition comprising a FLT3 inhibitor provided herein in combination with administering a menin inhibitor provided herein.

In some embodiments, the autoimmune disease is selected from hematological malignancies.

In some embodiments, provided herein are methods for treating a heteroimmune disease or condition comprising administering to a patient in need the pharmaceutical composition(s) provided herein.

In some embodiments, provided herein are methods for treating a heteroimmune disease or condition comprising administering to a patient in need a pharmaceutical composition comprising a FLT3 inhibitor provided herein in combination with administering a menin inhibitor provided herein.

In some embodiments, provided herein are methods for treating a cancer comprising administering to a patient in need the pharmaceutical composition(s) provided herein.

In some embodiments, provided herein are methods for treating a cancer comprising administering to a patient in need a pharmaceutical composition comprising a FLT3 inhibitor provided herein in combination with administering a menin inhibitor provided herein.

In some embodiments, the cancer is a hematological malignancy.

In some embodiments, the disease or condition is leukemia, lymphoma, or multiple myeloma. In certain embodiments, the disease or condition is acute lymphocytic leukemia (ALL), acute myeloid leukemia (AML), chronic lymphocytic leukemia (CLL), chronic myeloid leukemia (CML), prolymphocytic leukemia (PLL), large granular lymphocytic (LGL), hairy cell leukemia (HCL), mast-cell leukemia (MCL), or myelodysplastic syndrome (MDS).

In certain embodiments, the disease or condition is acute myeloid leukemia (AML). In certain embodiments, the AML is FLT3 mutation-positive. In certain embodiments, the AML is newly diagnosed. In certain embodiments, the AML is FLT3 mutation-positive and newly diagnosed. In certain embodiments, the AML is relapsed or refractory. In certain embodiments, the AML is relapsed or refractory and is FLT3 mutation-positive. In certain embodiments, the FLT3 mutation is an FLT3-ITD mutation. In certain embodiments, the FLT3 mutation is at D835.

In certain embodiments, the AML is resistant to chemotherapy. In certain embodiments, the AML is resistant to chemotherapy and has developed a FLT3-IND mutation, including, but not limited to at least one mutation selected from N676K, F691L, D835V, and Y842C.

In certain embodiments, the AML is resistant to a previously administered FLT3 inhibitor. In certain embodiments, the AML is resistant to gilteritinib. In certain embodiments, the AML is resistant to midostaurin.

In some embodiments, provided herein are methods for treating mastocytosis comprising administering to a patient in need the pharmaceutical composition(s) provided herein.

In some embodiments, provided herein are methods for treating mastocytosis comprising administering to a patient in need a pharmaceutical composition comprising a FLT3 inhibitor provided herein in combination with administering a menin inhibitor provided herein.

In some embodiments, provided herein are methods for treating osteoporosis or bone resorption disorders comprising administering to a patient in need the pharmaceutical composition(s) provided herein.

In some embodiments, provided herein are methods for treating osteoporosis or bone resorption disorders comprising administering to a patient in need a pharmaceutical composition comprising a FLT3 inhibitor provided herein in combination with administering a menin inhibitor provided herein.

In some embodiments, provided herein are methods for treating an inflammatory disease or condition comprising administering to a patient in need the pharmaceutical composition(s) provided herein.

In some embodiments, provided herein are methods for treating an inflammatory disease or condition comprising administering to a patient in need a pharmaceutical composition comprising a FLT3 inhibitor provided herein in combination with administering a menin inhibitor provided herein.

In some embodiments, provided herein are methods for treating lupus comprising administering to a subject in need thereof a composition containing a therapeutically effective amount of an FLT3 inhibitor or a pharmaceutically acceptable salt thereof provided herein in combination with administering a therapeutically effective amount of a menin inhibitor or a pharmaceutically acceptable salt thereof provided herein.

In some embodiments, provided herein are methods for treating lupus comprising administering to a subject in need thereof a composition containing a therapeutically effective amount of an FLT3 inhibitor of Formula (P4-I) or a pharmaceutically acceptable salt thereof in combination with administering a menin inhibitor or a pharmaceutically acceptable salt thereof provided herein.

In some embodiments, provided herein are methods for treating a heteroimmune disease or condition comprising administering to a subject in need thereof a composition containing a therapeutically effective amount of an FLT3 inhibitor or a pharmaceutically acceptable salt thereof in combination with administering a therapeutically effective amount of a menin inhibitor or a pharmaceutically acceptable salt thereof provided herein.

In some embodiments, provided herein are methods for treating a heteroimmune disease or condition comprising administering to a subject in need thereof a composition containing a therapeutically effective amount of an FLT3 inhibitor of Formula (P4-I) or a pharmaceutically acceptable salt thereof in combination with administering a therapeutically effective amount of a menin inhibitor or a pharmaceutically acceptable salt thereof provided herein.

In some embodiments provided herein are methods for treating diffuse large B cell lymphoma, follicular lymphoma, or chronic lymphocytic leukemia comprising administering to a subject in need thereof a composition containing a therapeutically effective amount of an FLT3 inhibitor or a pharmaceutically acceptable salt thereof provided herein in combination with administering a therapeutically effective amount of a menin inhibitor or a pharmaceutically acceptable salt thereof provided herein.

In some embodiments provided herein are methods for treating diffuse large B cell lymphoma, follicular lymphoma, or chronic lymphocytic leukemia comprising administering to a subject in need thereof a composition containing a therapeutically effective amount of a FLT3 inhibitor of Formula (P4-I) or a pharmaceutically acceptable salt thereof in combination with administering a therapeutically effective amount of a menin inhibitor or a pharmaceutically acceptable salt thereof provided herein.

In some embodiments, provided herein are methods for treating mastocytosis comprising administering to a subject in need thereof a composition containing a therapeutically effective amount of a FLT3 inhibitor of Formula (P4-I) or a pharmaceutically acceptable salt thereof in combination with administering a therapeutically effective amount of a menin inhibitor or a pharmaceutically acceptable salt thereof provided herein.

In some embodiments, provided herein are methods for treating osteoporosis or bone resorption disorders comprising administering to a subject in need thereof a composition containing a therapeutically effective amount of a FLT3 inhibitor of Formula (P4-I) or a pharmaceutically acceptable salt thereof in combination with administering a therapeutically effective amount of a menin inhibitor or a pharmaceutically acceptable salt thereof provided herein.

In some embodiments, provided herein are methods for treating an inflammatory disease or condition comprising administering to a subject in need thereof a composition containing a therapeutically effective amount of a FLT3 inhibitor of Formula (P4-I) or a pharmaceutically acceptable salt thereof in combination with administering a therapeutically effective amount of a menin inhibitor or a pharmaceutically acceptable salt thereof provided herein.

In some embodiments, provided herein is a pharmaceutical composition comprising a pharmaceutically acceptable carrier and a pharmaceutically effective amount of a compound according to any one of the formulas described herein. In some embodiments, the compound is according to any one of Formula (I), Formula (P-I), Formula (P2-I), and Formula (P4-I).

In some embodiments, provided herein is a pharmaceutical composition comprising a pharmaceutically acceptable carrier and a pharmaceutically effective amount of an FLT3 inhibitor according to any one of the formulas described herein for administration in combination with a pharmaceutical composition comprising a pharmaceutically acceptable carrier and a pharmaceutically effective amount of a menin inhibitor according to any of the other formulas described herein.

In some embodiments, provided herein is a pharmaceutical composition comprising a pharmaceutically acceptable carrier and a pharmaceutically effective amount of a compound of Formula (I) for administration in combination with and a pharmaceutical composition comprising a pharmaceutically acceptable carrier and a pharmaceutically effective amount of a compound of (P-1).

In some embodiments, provided herein is a pharmaceutical composition comprising a pharmaceutically acceptable carrier and a pharmaceutically effective amount of a compound of Formula (I) for administration in combination with and a pharmaceutical composition comprising a pharmaceutically acceptable carrier and a pharmaceutically effective amount of a compound of (P-4).

In some embodiments, provided herein is a pharmaceutical composition comprising a pharmaceutically acceptable carrier and a pharmaceutically effective amount of a compound of Formula (I) for administration in combination with and a pharmaceutical composition comprising a pharmaceutically acceptable carrier and a pharmaceutically effective amount of a compound of (P-2).

In some embodiments, a pharmaceutical composition described herein is formulated for a route of administration selected from oral administration, parenteral administration, buccal administration, nasal administration, topical administration, or rectal administration.

In some embodiments, the carrier is a parenteral carrier.

In some embodiments, the carrier is an oral carrier.

In some embodiments, the carrier is a topical carrier.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

Further representative embodiments of compounds of Formula (P-I), include compounds listed in Table 1, or a pharmaceutically acceptable salt, solvate, hydrate, or stereoisomer thereof.

Throughout the specification, groups and substituents thereof can be chosen by one skilled in the field to provide stable moieties and compounds.

In some embodiments, the pharmaceutical composition comprising a FLT3 inhibitor described herein is administered in combination with a menin inhibitor described herein inhibit FLT3. In some embodiments, the compounds of Formula (P4-I) inhibit FLT3. In some embodiments, the FLT3 inhibitors, for example, the compounds of Formula (P-1), (P2-I), or (P4-I), are administered in combination with a menin inhibitor are used to treat patients suffering from FLT3-dependent or FLT3 mediated conditions or diseases, including, but not limited to, proliferative diseases such as hematological malignancies. In some embodiments, the compounds of Formula (P4-I) are used to treat patients suffering from FLT3-dependent or FLT3 mediated conditions or diseases, including, but not limited to, proliferative diseases such as hematological malignancies.

### Preparation of Compounds

The FLT3 inhibitors described herein, including those of Formula (P4-I), may be synthesized using standard synthetic reactions known to those of skill in the art or using methods known in the art. The reactions can be employed in a linear sequence to provide the compounds or they may be used to synthesize fragments which are subsequently joined by the methods known in the art. Exemplary methods are provided in the Examples herein. Menin inhibitors may be prepared according to U.S. Pat. No. 11,084,825 B2 or 11,174,263 B2, the contents of which are incorporated by reference in their entireties.

Described herein are compounds that inhibit the activity of FLT3, and processes for their preparation. Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided.

The starting material used for the synthesis of the compounds described herein may be synthesized or can be obtained from commercial sources, such as, but not limited to, Aldrich Chemical Co. (Milwaukee, Wisconsin), Bachem (Torrance, California), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). (all of which are incorporated by reference in their entirety). Additional methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography, and the like. Such materials may be characterized using conventional means, including physical constants and spectral data.

Compounds described herein may be prepared as a single isomer or a mixture of isomers.

In some embodiments, representative compounds of Formula (P4-I) are prepared according to synthetic schemes depicted herein.

### Further Forms of Compounds

In some embodiments, the FLT3 inhibitors disclosed herein have a structure of Formula (P-I), (P2-I), or (P4-I). It is understood that when reference is made to compounds described herein, it is meant to include compounds of any of Formula (P-I), (P2-I), or (P4-I) as well as to all of the specific compounds that fall within the scope of these generic formulae, unless otherwise indicated.

Compounds described herein may possess one or more stereocenters and each center may exist in the R or S configuration. Compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

Diastereomeric mixtures can be separated into their individual diastereomers on the basis of their physical chemical differences by methods known, for example, by chromatography and/or fractional crystallization. In some embodiments, enantiomers can be separated by chiral chromatographic columns. In some embodiments, enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

Methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, compounds described herein can exist in unsolvated as well as solvated forms with pharmaceutically acceptable solvents such as water, ethanol, and the like. Solvated forms of compounds presented herein are also considered to be disclosed herein.

Compounds described herein in unoxidized form can be prepared from N-oxides of compounds described herein by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985).

Prodrug forms of the herein described compounds, wherein the prodrug is metabolized in vivo to produce a derivative as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Sites on the aromatic ring portion of the compounds described herein can be susceptible to various metabolic reactions, therefore incorporation of appropriate substituents on the aromatic ring structures, such as, by way of example only, halogens can reduce, minimize or eliminate this metabolic pathway.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

In additional or some embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs.

It should be understood that a reference to a salt includes the solvent addition forms or crystal forms thereof, particularly solvates or polymorphs.

Compounds described herein may be in various forms, including but not limited to, amorphous forms, milled forms and nano-particulate forms. In addition, compounds described herein include crystalline forms, also known as polymorphs.

The screening and characterization of the pharmaceutically acceptable salts, polymorphs, and/or solvates may be accomplished using a variety of techniques including, but not limited to, thermal analysis, x-ray diffraction, spectroscopy, vapor sorption, and microscopy.

Throughout the specification, groups and substituents thereof can be chosen by one skilled in the field to provide stable moieties and compounds.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

The term “pharmaceutical combination” as used herein, means a product that results from the mixing or combining of more than one active ingredient and includes both fixed and non-fixed combinations of the active ingredients.

The pharmaceutical compositions described herein can be administered to a subject by multiple administration routes, including but not limited to, oral, parenteral (e.g., intravenous, subcutaneous, intramuscular), intranasal, buccal, topical, rectal, or transdermal administration routes.

The pharmaceutical compositions will include at least one compound described herein, such as, for example, a compound of any of Formula (P4-I) as an active ingredient in free-acid or free-base form, or in a pharmaceutically acceptable salt form. Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

### Examples of Methods of Dosing and Treatment Regimens

The compounds described herein can be used in the preparation of medicaments for the inhibition of FLT3 or a homolog thereof, or for the treatment of diseases or conditions that would benefit, at least in part, from inhibition of FLT3 or a homolog thereof. In addition, a method for treating any of the diseases or conditions described herein in a subject in need of such treatment, involves administration of pharmaceutical compositions containing at least one FLT3 inhibitor described herein in combination with the administration of a menin inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments.

### Methods of Treatment.

In particular embodiments, provided herein are methods of treating, ameliorating, or preventing a disease or condition in a patient in need thereof comprising administering an amount of an FLT3 inhibitor compound described herein in combination with the administration of an amount of a menin inhibitor described herein to treat, ameliorate, or prevent the disease or condition. In particular embodiments, provided herein are methods of treating, ameliorating, or preventing a disease or condition in a patient in need thereof comprising administering an amount of a pharmaceutical composition comprising a FLT3 inhibitor described herein in combination with the administration of an amount of a menin inhibitor described herein to treat, ameliorate, or prevent the disease or condition. In certain embodiments, provided herein are any of the compounds described herein for use in therapy. In certain embodiments, provided herein are any of the pharmaceutical compositions described herein for use in therapy. In certain embodiments, provided herein are any of the compounds described herein for use in treating, ameliorating, or preventing a disease or condition in a patient in need thereof. In certain embodiments, provided herein are any of the pharmaceutical compositions described herein for use in treating, ameliorating, or preventing a disease or condition in a patient in need thereof. In certain embodiments, provided herein are any of the compounds described herein for use the manufacture of a medicament for therapy. In certain embodiments, provided herein are any of the pharmaceutical compositions described herein for therapy. In certain embodiments, provided herein are any of the compounds described herein for use the manufacture of a medicament for treating, ameliorating, or preventing a disease or condition in a patient in need thereof. In certain embodiments, provided herein are any of the pharmaceutical compositions described herein for the manufacture of a medicament for treating, ameliorating, or preventing a disease or condition in a patient in need thereof. Useful conditions and disorders are described herein.

In certain embodiments, the disease or condition is associated with FLT3 dysfunction. In certain embodiments, the disease or condition is associated with undesired FLT3 expression. In certain embodiments, the disease or condition is associated with excessive FLT3 expression. In certain embodiments, the disease or condition is associated with undesired FLT3 levels. In certain embodiments, the disease or condition is associated with excessive FLT3 levels. In certain embodiments, the disease or condition is associated with undesired FLT3 activity. In certain embodiments, the disease or condition is associated with excessive FLT3 activity. In certain embodiments, the disease or condition is associated with undesired FLT3-MLL interaction. In certain embodiments, the disease or condition is associated with excessive FLT3-MLL interaction. In certain embodiments, the disease or condition is amenable to treatment by inhibiting FLT3 expression. In certain embodiments, the disease or condition is amenable to treatment by inhibiting excessive FLT3 expression. In certain embodiments, the disease or condition is amenable to treatment by inhibiting FLT3 levels. In certain embodiments, the disease or condition is amenable to treatment by inhibiting excessive FLT3 levels. In certain embodiments, the disease or condition is amenable to treatment by inhibiting undesired FLT3 activity. In certain embodiments, the disease or condition is amenable to treatment by inhibiting excessive FLT3 activity. In certain embodiments, the disease or condition is amenable to treatment by inhibiting FLT3-MLL interaction. In certain embodiments, the disease or condition is amenable to treatment by inhibiting excessive FLT3-MLL interaction.

In certain embodiments, the disease or condition is associated with a mutation in the FLT3 gene. In certain embodiments, the mutation in the FLT3 gene is an internal tandem duplicate mutation (FLT3-IND). In certain embodiments, the FLT3-IND mutation is a D835 mutation. In one embodiment, the FLT3-IND mutation is D835V. In certain embodiments, the FLT3-IND mutation is D835Y. In certain embodiments, the mutation is a point mutation in the tyrosine kinase domain (FLT3-TKD).

In some embodiments, compounds provided herein are administered to a human.

In some embodiments, compounds provided herein are orally administered.

In some embodiments, the disease or condition is a hematologic malignancy, including, but not limited to leukemia, lymphoma, or multiple myeloma. In certain embodiments, the disease or condition is a leukemia, including, but not limited to, acute lymphocytic leukemia (ALL), acute myeloid leukemia (AML), chronic lymphocytic leukemia (CLL), chronic myeloid leukemia (CML), prolymphocytic leukemia (PLL), acute prolymphocytic leukemia (APL), large granular lymphocytic (LGL), hairy cell leukemia (HCL), chronic neutrophilic leukemia (CNL), acute undifferentiated leukemia (AUL), anaplastic large cell lymphoma (ALCL), prolymphocytic leukemia (PML), juvenile myelomonocytic leukemia (JMML), adult T cell ALL, or myeloproliferative disorder (MPD), mast-cell lymphoma (MCC), myelodysplastic syndromes (MDS). In certain embodiments, the disease or condition is acute myeloid leukemia (AML). In certain embodiments, the disease or condition is relapsed or refractory AML.

In certain embodiments, the disease or condition is a lymphoma, including, but not limited to, non-Hodgkin's lymphoma or Hodgkin's lymphoma. In certain embodiments, the disease or condition is non-Hodgkin's lymphoma, including, but not limited to, B-cell lymphoma, diffuse large B-cell lymphoma (DLBCL), T-cell lymphoma, Burkitt's lymphoma, follicular lymphoma (FL), mantle cell lymphoma, primary mediastinal B cell lymphoma, small lymphocytic lymphoma, or Waldenstrom macroglobulinemia (lymphoplasmacytic lymphoma). In certain embodiments, the disease or condition is Hodgkin's lymphoma, including but not limited to, lymphocyte-deleted Hodgkin's disease, lymphocyte-rich Hodgkin's disease, mixed cellularity Hodgkin's lymphoma, nonular lymphocyte-predominant Hodgkin's disease, or nondular sclerosis Hodgkin's lymphoma.

In certain embodiments, the disease or condition is multiple myeloma. In one embodiment, the multiple myeloma is hyperdiploid. In one embodiment, the multiple myeloma is hypodiploid.

In certain embodiments, the disease or condition is relapsed or refractory. In one embodiment, the disease or condition is relapsed or refractory acute myeloid leukemia (AML). In certain embodiments, the disease or condition is newly diagnosed. In one embodiment, the disease or condition is newly diagnosed AML.

embodiments, the disease or condition is cancer.

In certain embodiments, the cancer is a hematologic cancer.

In certain embodiments, the hematologic cancer is a leukemia, a lymphoma, a myeloma, a non-Hodgkin's lymphoma, a Hodgkin's lymphoma, or a B-cell malignancy.

In certain embodiments, the B-cell malignancy is diffuse large B-cell lymphoma (DLBCL).

In certain embodiments, DLBCL is activated B-cell diffuse large B-cell lymphoma (ABC-DLBCL).

In certain embodiments, the B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenstrom's macroglobulinemia, or a combination thereof.

In certain embodiments, the B-cell malignancy is a relapsed or refractory B-cell malignancy.

In certain embodiments, the relapsed or refractory B-cell malignancy is diffuse large B-cell lymphoma (DLBCL).

In certain embodiments, the relapsed or refractory DLBCL is activated B-cell diffuse large B-cell lymphoma (ABC-DLBCL).

In certain embodiments, the relapsed or refractory B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenstrom's macroglobulinemia, or a combination thereof.

In certain embodiments, the B-cell malignancy is a metastasized B-cell malignancy.

In certain embodiments, the metastasized B-cell malignancy is diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenstrom's macroglobulinemia, or a combination thereof.

In certain embodiments, the cancer is a sarcoma or carcinoma.

In certain embodiments, the cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

In certain embodiments, the cancer is a breast cancer.

In certain embodiments, the breast cancer is ductal carcinoma in situ, lobular carcinoma in situ, invasive or infiltrating ductal carcinoma, invasive or infiltrating lobular carcinoma, inflammatory breast cancer, triple-negative breast cancer, paget disease of the nipple, phyllodes tumor, angiosarcoma or invasive breast carcinoma.

In certain embodiments, the cancer is a colon cancer.

In certain embodiments, the colon cancer is adenocarcinoma, gastrointestinal carcinoid tumors, gastrointestinal stromal tumors, primary colorectal lymphoma, leiomyosarcoma, melanoma, squamous cell-carcinoma, mucinous adenocarcinoma, or Signet ring cell adenocarcinoma.

In certain embodiments, the cancer is a relapsed or refractory cancer.

In certain embodiments, the relapsed or refractory cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

In certain embodiments, the cancer is a metastasized cancer.

In certain embodiments, the metastasized cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

### Combination Treatments

The FLT3 inhibitor and menin inhibitor compositions described herein can also be used in combination with other well known therapeutic reagents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In certain instances, it may be appropriate to administer at least one FLT3 inhibitor compound described herein and at least one menin inhibitor described herein in combination with another therapeutic agent. By way of example only, if one of the side effects experienced by a patient upon receiving one of the FLT3 inhibitor compounds described herein is nausea, then it may be appropriate to administer an anti-nausea agent in combination with the initial therapeutic agents. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature Combination treatment further includes periodic treatments that start and stop at various times to assist with the clinical management of the patient.

Exemplary Therapeutic Agents for Use in Combination with a FLT3 Inhibitor Compound

Other anti-cancer agents that can be employed in combination with the FLT3 inhibitor compound and menin inhibitor compound include anticancer agents.

In certain embodiments, the combination is administered once a day, two times per day, three times per day, four times per day, or five times per day.

In certain embodiments, the menin inhibitor is administered at a dosage of about 40 mg/day to about 1000 mg/day.

In certain embodiments, the menin inhibitor is administered orally.

In certain embodiments, the FLT3 inhibitor and the menin inhibitor are administered in separate compositions. In certain embodiments, the FLT3 inhibitor and the menin inhibitor are administered simultaneously, sequentially or intermittently.

In certain embodiments, the compound of formula (P4-I) and the menin inhibitor are administered in separate compositions. In certain embodiments, the compound of formula (P4-I) and the menin inhibitor are administered simultaneously, sequentially or intermittently.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

The examples below as well as throughout the application, the following abbreviations have the following meanings. If not defined, the terms have their generally accepted meanings.

aq = aqueous
Boc = tert-butyloxycarbonyl
t-BuOH = tertiary butanol
DCE = 1,2-dichloroethane
DCM = dichloromethane
DIAD = diisopropyl azodicarboxylate
DIEA or DIPEA = N,N-diisopropylethylamine
DMAP = dimethylaminopyridine
DMF = dimethylformamide
DMSO = dimethylsulfoxide
ESI = electron spray ionization
EA = ethyl acetate
g = gram
HCl = hydrogen chloride
HPLC = high performance liquid chromatography
hr = hour
¹H NMR = proton nuclear magnetic resonance
IPA = isopropyl alcohol
KOAc = potassium acetate
LC-MS = liquid chromatography mass spectroscopy
M = molar
MeCN = acetonitrile
MeOH = methanol
mg = milligram
min = minute
ml = milliliter
mM = millimolar
mmol = millimole
m.p. = melting point
MS = mass spectrometry
m/z = mass-to-charge ratio
N = normal
NIS = N-iodosuccinimide
nM = nanomolar
nm = nanometer
Pd(dppf)Cl₂ = [1,1′-
Bis(diphenylphosphino)ferrocene]dichloropalladium(II)
PE = petroleum ether
PyBOP = benzotriazol-1-yl-oxytripyrrolidinophosphonium
hexafluorophosphate
quant. = quantitative
RP = reverse phase
rt or r.t. = room temperature
Sat. = saturated
TEA = triethylamine
TFA = trifluoroacetic acid
μL = microliter
μM = Micromolar

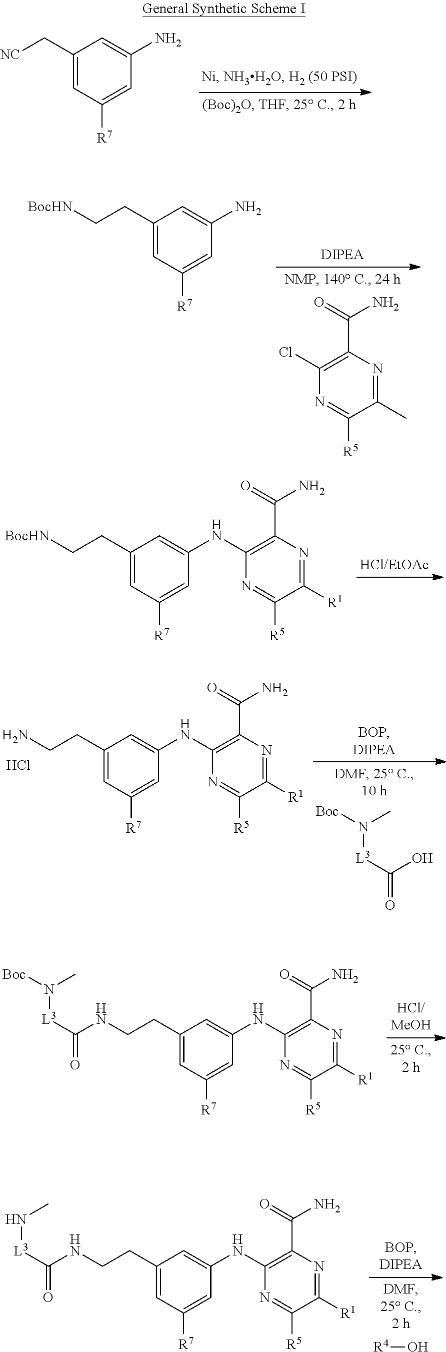

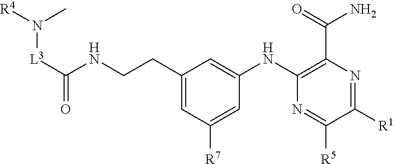

wherein L³, R¹, R⁴, R⁵, and R⁷ are as described herein.

### Synthesis of Intermediates

### Intermediate 6

### tert-butyl (S)-(1-((3-((3-carbamoyl-6-chloro-5-ethylpyrazin-2-yl)amino)-5-methoxyphenethyl)amino)-1-oxopropan-2-yl)(methyl)carbamate

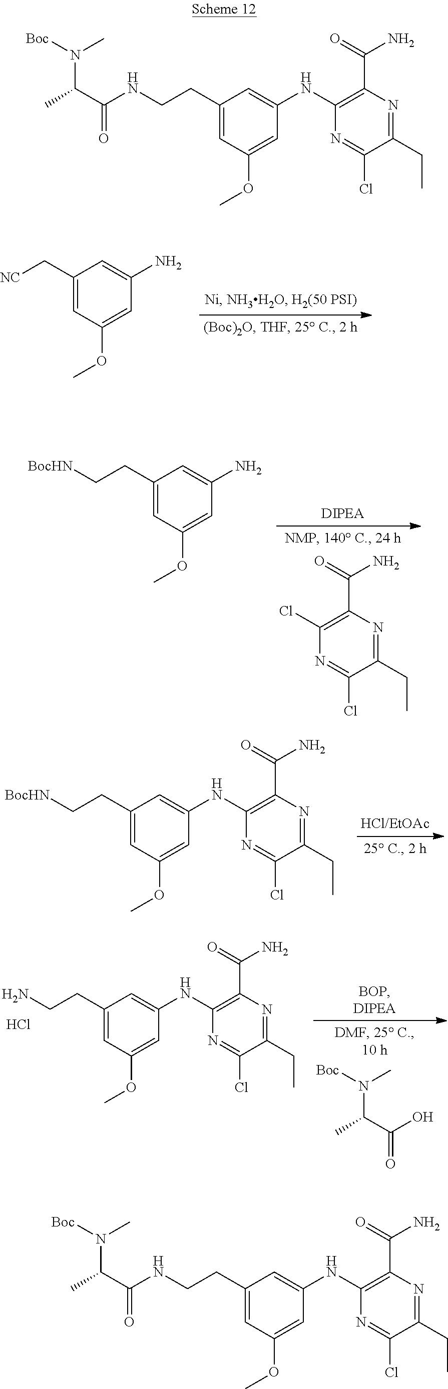

### Step 1: tert-butyl (3-amino-5-methoxyphenethyl)carbamate

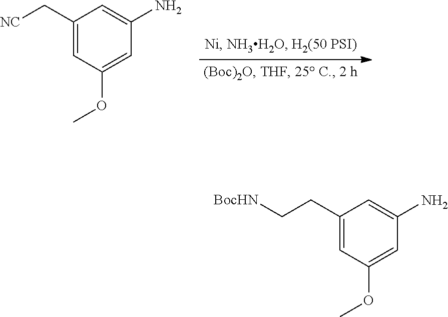 [SMILES: COc1cc(N)cc(CC#N)c1 ; COc1cc(N)cc(CCNC(=O)OC(C)(C)C)c1]

To a solution of Ni (723.77 mg, 12.33 mmol, 1 eq) in THF (50 mL) at 25° C., 2-(3-amino-5-methoxyphenyl)acetonitrile (2 g, 12.33 mmol, 1 eq) (Boc)₂O (2.69 g, 12.33 mmol, 2.83 mL, 1 eq) DIPEA (3.19 g, 24.66 mmol, 4.30 mL, 2 eq) was added. The mixture was stirred at 25° C. under 50 psi of H₂ for 2 hrs. TLC indicated one major new spot with larger polarity was detected. The crude mixture was concentrated under reduced pressure. The crude product was purified by chromatography on silica thiol gel (petroleum ether/EtOAc=3/1). To afford tert-butyl (3-amino-5-methoxyphenethyl)carbamate (1.3 g, 4.88 mmol, 39.58% yield) As white solid. ¹H NMR (400 MHz, DMSO-d₆) δ=6.81 (br t, J=5.4 Hz, 1H), 6.00-5.96 (m, 2H), 5.92 (s, 1H), 4.98 (s, 2H), 3.63 (s, 3H), 3.11-3.01 (m, 2H), 2.49-2.45 (m, 2H), 1.38 (s, 9H).

### Step 2: tert-butyl (3-((3-carbamoyl-6-chloro-5-ethylpyrazin-2-yl)amino)-5-methoxyphenethyl)carbamate

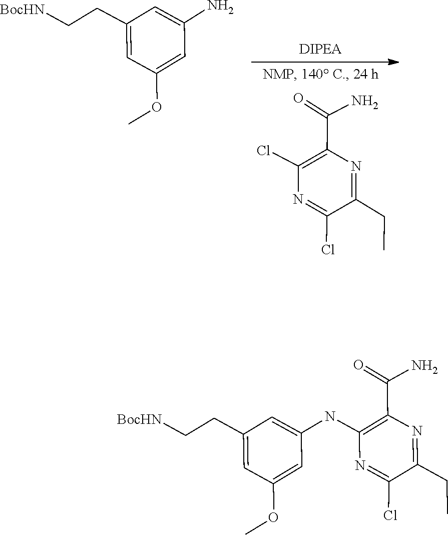

To a solution of tert-butyl (3-amino-5-methoxyphenethyl)carbamate (1.65 g, 7.51 mmol, 1 eq), 3,5-dichloro-6-ethylpyrazine-2-carboxamide (2 g, 7.51 mmol, 1 eq) in 872-50-4 (5 mL) was added DIPEA (19.41 g, 150.19 mmol, 26.16 mL, 20 eq). The mixture was stirred at 140° C. for 24 h under N₂. LCMS showed the reaction was completed. The reaction was poured into water (30 mL) and extracted with EtOAc (20 mL*3). The organic layers were combined, washed with water (50 mL*2), sat. brine (100 mL), dried with anhydrous Na₂SO₄, filtered and concentrated to give crude product. The crude product was purified by chromatography on silica thiol gel (Petroleum ether/Ethyl acetate/Dichloromethane=2/1/1). To afford tert-butyl (3-((3-carbamoyl-6-chloro-5-ethylpyrazin-2-yl)amino)-5-methoxyphenethyl)carbamate (1.93 g, 4.01 mmol, 53.40% yield) as yellow solid. 1H NMR (400 MHz, DMSO-d6) δ=11.27 (s, 1H), 8.33 (s, 1H), 8.11 (s, 1H), 7.42 (s, 1H), 6.92 (br t, J=5.3 Hz, 1H), 6.86 (s, 1H), 6.54 (s, 1H), 3.81 (s, 3H), 3.21 (q, J=6.6 Hz, 2H), 3.30-3.26 (m, 2H), 2.81-2.79 (m, 2H) 2.50-2.48 (m, 2H), 1.42 (s, 9H), 1.32 (t, J=7.5 Hz, 4H). LC-MS (ES+, m/z): 351.4 [(M+H)⁺]; Rt=0.824 min.

### Step 3: 3-((3-(2-aminoethyl)-5-methoxyphenyl)amino)-5-chloro-6-ethylpyrazine-2-carboxamide

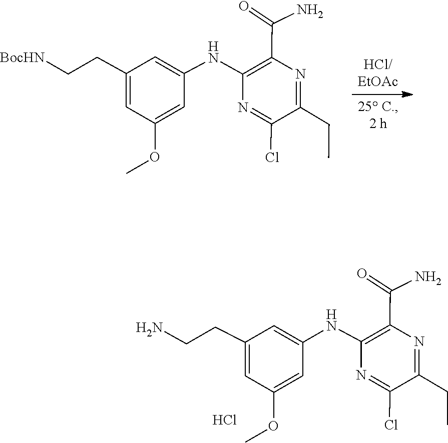 [SMILES: CCc1nc(C(N)=O)c(Nc2cc(CCNC(=O)OC(C)(C)C)cc(OC)c2)nc1Cl ; CCc1nc(C(N)=O)c(Nc2cc(CCN)cc(OC)c2)nc1Cl ; Cl]

To a solution of tert-butyl (3-((3-carbamoyl-6-chloro-5-ethylpyrazin-2-yl)amino)-5-methoxyphenethyl)carbamate (1.93 g, 3.43 mmol, 80% purity, 1 eq) in HCl/EtOAc (100 mL). The mixture was stirred at 25° C. for 2 h. LCMS showed the reaction was completed. The crude mixture was worked up by filtration. The crude product was purified by re-crystallization from EtOAc (20 mL) at 25° C. to afford 3-((3-(2-aminoethyl)-5-methoxyphenyl)amino)-5-chloro-6-ethylpyrazine-2-carboxamide (1.3 g, 3.30 mmol, 96.11% yield, HCl) as yellow solid. LC-MS (ES+, m/z): 350.1 [(M+H)⁺]; Rt=0.698 min.

Note: HCl/EtOAc (4 M): HCl was bubbled into a solution EtOAc at 0° C. for 0.5 h. Then, the solution was weighed to obtained the HCl/EtOAc (4 M)

### Step 4: tert-butyl (S)-(1-((3-((3-carbamoyl-6-chloro-5-ethylpyrazin-2-yl)amino)-5-methoxyphenethyl)amino)-1-oxopropan-2-yl)(methyl)carbamate

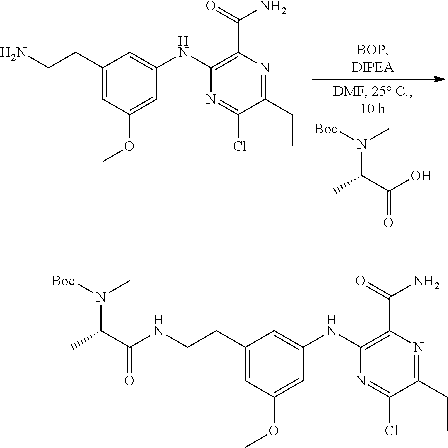

To a solution of 3-((3-(2-aminoethyl)-5-methoxyphenyl)amino)-5-chloro-6-ethylpyrazine-2-carboxamide (1.3 g, 3.65 mmol, 98.2% purity, 1 eq), N-(tert-butoxycarbonyl)-N-methyl-L-alanine (1.11 g, 5.47 mmol, 1.5 eq) in DMF (10 mL) was added BOP (1.54 g, 5.47 mmol, 1.5 eq), DIPEA (3.00 g, 36.49 mmol, 2.91 mL, 10 eq). The mixture was stirred at 25° C. for 10 hrs under N₂. LCMS showed the reaction was completed. The crude product was purified by chromatography on silica thiol gel (petroleum ether/EtOAc=1/2). To afford tert-butyl (S)-(1-((3-((3-carbamoyl-6-chloro-5-ethylpyrazin-2-yl)amino)-5-methoxyphenethyl)amino)-1-oxopropan-2-yl)(methyl) carbamate (1.84 g, 2.48 mmol, 67.85% yield) as yellow oil. 1H NMR (400 MHz, DMSO-d6) δ=11.22 (s, 1H), 8.28 (s, 1H), 8.06 (s, 1H), 7.81 (br s, 1H), 7.34 (s, 1H), 6.85 (s, 1H), 6.50 (s, 1H), 4.55-4.52 (m, 1H), 3.76 (s, 3H), 3.30-3.24 (m, 2H), 2.90-2.78 (m, 2H), 2.74-2.66 (m, 5H), 1.36 (br s, 9H), 1.28-1.11 (m, 6H). LC-MS (ES+, m/z): 435.2 [(M+H)⁺]; Rt=0.884 min; HRMS: 435.1911.

### Intermediate 7

### tert-butyl (S)-(1-((3-((3-carbamoyl-6-chloro-5-ethylpyrazin-2-yl)amino)phenethyl)amino)-1-oxopropan-2-yl)(methyl)carbamate

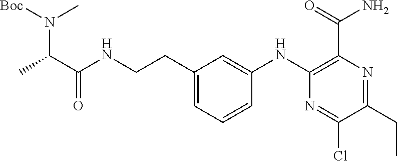 [SMILES: CCc1nc(C(N)=O)c(Nc2cccc(CCNC(=O)[C@H](C)N(C)C(=O)OC(C)(C)C)c2)nc1Cl]

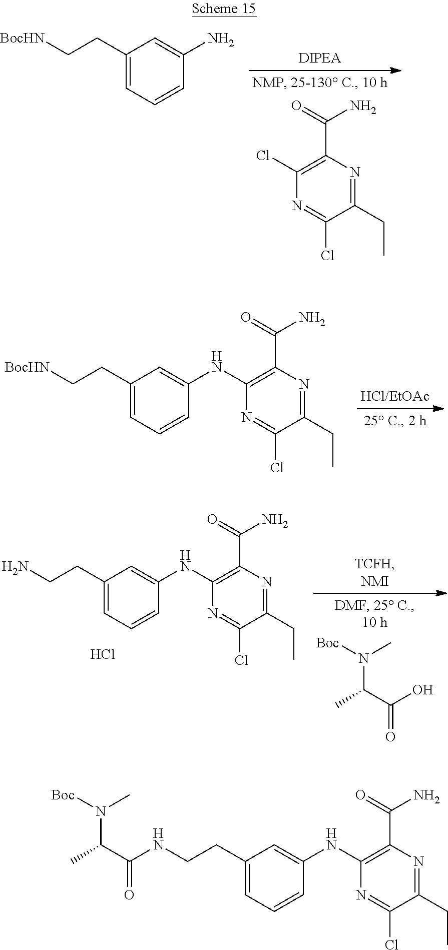

### Step 1: tert-butyl (3-((3-carbamoyl-6-chloro-5-ethylpyrazin-2-yl)amino)phenethyl)carbamate

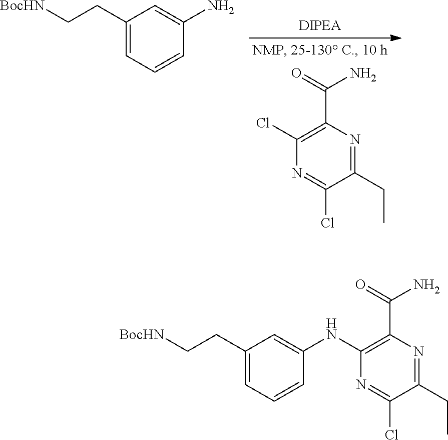 [SMILES: CC(C)(C)OC(=O)NCCc1cccc(N)c1 ; CCc1nc(C(N)=O)c(Cl)nc1Cl ; CCc1nc(C(N)=O)c(Nc2cccc(CCNC(=O)OC(C)(C)C)c2)nc1Cl]

To a solution of tert-butyl (3-aminophenethyl)carbamate (2.25 g, 9.52 mmol, 1 eq) 3,5-dichloro-6-ethylpyrazine-2-carboxamide (2.10 g, 9.52 mmol, 1 eq) in dioxane (20 mL) at 25° C., DIPEA (12.31 g, 95.21 mmol, 16.58 mL, 10 eq) was added. The mixture was stirred at 130° C. for 10 hrs. LCMS indicated the reaction was completed. The mixture was poured into water (40 mL) and extracted with EtOAc (20 mL*2). The organic layers was washed with water (40 mL*2), saturated brine (80 mL*2), dried over anhydrous Na₂SO₄, filtered, concentrated under reduced pressure to give a residue. The crude product was purified by chromatography on silica gel (petroleum ether/EtOAc=1:1) to afford tert-butyl (3-((3-carbamoyl-6-chloro-5-ethylpyrazin-2-yl)amino)phenethyl)carbamate (3 g, 4.79 mmol, 50.27% yield) as yellow oil. LC-MS (ES⁺, m/z): 420.2 [(M+H)⁺]; Rt=0.939 min.

### Step 2: 3-((3-(2-aminoethyl)phenyl)amino)-5-chloro-6-ethylpyrazine-2-carboxamide

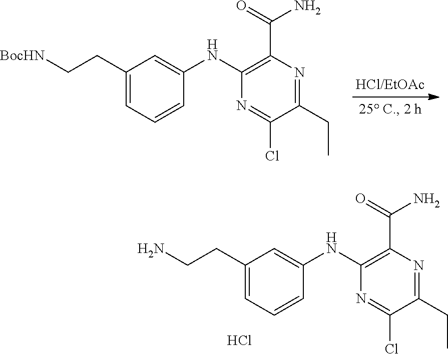 [SMILES: CCc1nc(C(N)=O)c(Nc2cccc(CCNC(=O)OC(C)(C)C)c2)nc1Cl ; CCc1nc(C(N)=O)c(Nc2cccc(CCN)c2)nc1Cl ; Cl]

The mixture tert-butyl N-[2-[3-[(3-carbamoyl-6-chloro-5-ethyl-pyrazin-2-yl)amino]phenyl]ethyl]carbamate (2 g, 4.76 mmol, 1 eq) and HCl/EtOAc (4 M, 50 mL, 41.99 eq) was stirred at 25° C. for 2 hrs. LCMS indicated the reaction was completed. The mixture was concentrated under reduced pressure to give a residue. The crude product was purified by chromatography on silica gel (petroleum ether/EtOAc=1:1) to afford 3-((3-(2-aminoethyl)phenyl)amino)-5-chloro-6-ethylpyrazine-2-carboxamide (1.38 g, 4.32 mmol, 90.60% yield) as yellow solid. LC-MS (ES⁺, m/z): 320.2 [(M+H)⁺]; Rt=0.671 min.

Note: HCl/EtOAc (4 M): HCl was bubbled into a solution EtOAc at 0° C. for 0.5 h. Then, the solution was weighed to obtained the HCl/EtOAc (4 M)

### Step 3: tert-butyl (S)-(1-((3-((3-carbamoyl-6-chloro-5-ethylpyrazin-2-yl)amino)phenethyl)amino)-1-oxopropan-2-yl)(methyl)carbamate

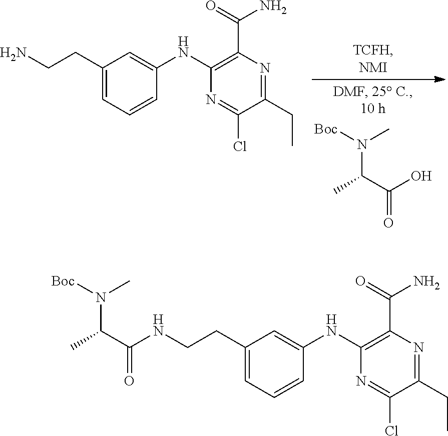

To a solution of 3-((3-(2-aminoethyl)phenyl)amino)-5-chloro-6-ethylpyrazine-2-carboxamide (1.38 g, 4.32 mmol, 1 eq), TCFH (1.82 g, 6.47 mmol, 1.5 eq) NMI (3.54 g, 43.15 mmol, 10 eq) in DMF (15 mL) at 25° C., N-(tert-butoxycarbonyl)-N-methyl-L-alanine (1.32 g, 6.47 mmol, 1.5 eq) was added. The mixture was stirred at 25° C. for 10 hrs. LCMS indicated the reaction was completed. The mixture was poured into water (30 mL) and extracted with EtOAc (15 mL*2). The organic layers was washed with water (30 mL*2), saturated brine (30 mL*2), dried over anhydrous Na₂SO₄, filtered, concentrated under reduced pressure to give a residue. The crude product was purified by chromatography on silica gel (petroleum ether/EtOAc=1:1) to afford tert-butyl N-[(1S)-2-[2-[3-[(3-carbamoyl-6-chloro-5-ethyl-pyrazin-2-yl)amino]phenyl]ethylamino]-1-methyl-2-oxo-ethyl]-N-methyl-carbamate (2.55 g, crude) as yellow oil. LC-MS (ES⁺, m/z): 505.2 [(M+H)⁺]; Rt=7.177 min.

### Intermediate 8

### tert-butyl (S)-(1-((3-((3-carbamoyl-6-chloro-5-ethylpyrazin-2-yl)amino)-5-fluorophenethyl)amino)-1-oxopropan-2-yl)(methyl)carbamate

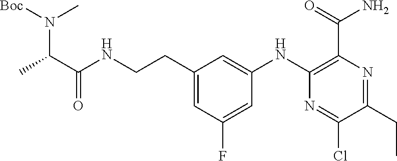 [SMILES: CCc1nc(C(N)=O)c(Nc2cc(F)cc(CCNC(=O)[C@H](C)N(C)C(=O)OC(C)(C)C)c2)nc1Cl]

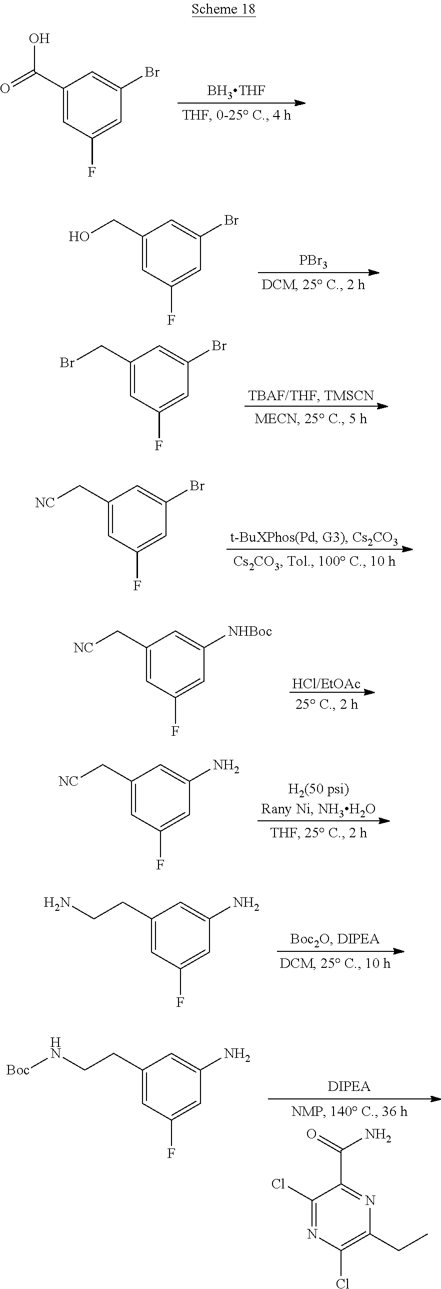

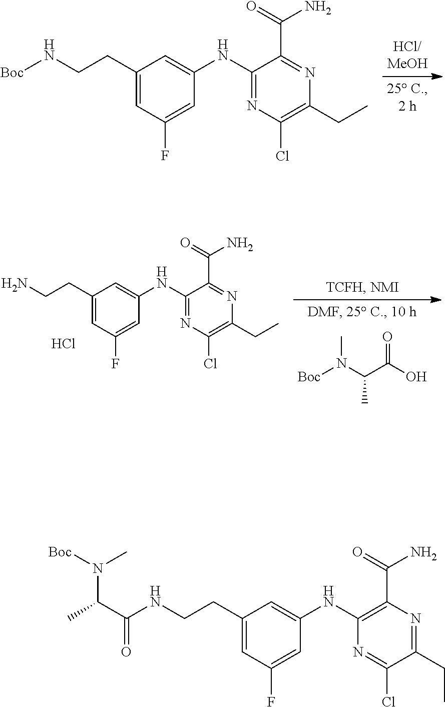

### Step 1: (3-bromo-5-fluorophenyl)methanol

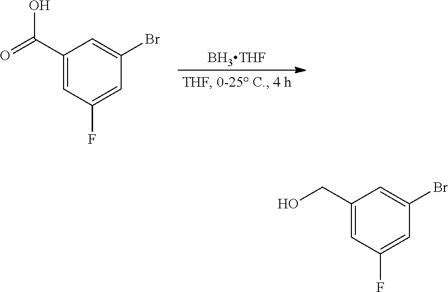 [SMILES: O=C(O)c1cc(F)cc(Br)c1 ; OCc1cc(F)cc(Br)c1 ; "BH3·THF"]

To a solution of 3-bromo-5-fluorobenzoic acid (50 g, 228.30 mmol, 1 eq) in THF (500 mL) at 0° C., BH₃·THF (1 M, 570.76 mL, 2.5 eq) was added. The mixture was allowed to warm to 25° C. for 4 hrs. LCMS indicated the reaction was completed. The reaction mixture was slowly added into MeOH (300 mL) at 0° C. Then the combined organic phase was stirred at 70° C. for 1 h, filtered and concentrated in vacuum. The residue was poured into EtOAc (500 mL) and H₂O (1 L). The aqueous phase was separated and extracted with ethyl acetate (600 mL*3). The combined organic phase was washed with saturated brine (600 mL*3), dried with anhydrous Na₂SO₄, filtered and concentrated to give (3-bromo-5-fluoro-phenyl)methanol (50 g, crude) as yellow oil.

¹H NMR (400 MHz, DMSO-d₆) δ=7.49-7.36 (m, 2H), 7.17 (dd, J=1.0, 9.7 Hz, 1H), 5.45 (t, J=5.8 Hz, 1H), 4.52 (d, J=5.8 Hz, 2H). LC-MS (ES⁺, m/z): 202.9 [(M−H)⁻]; Rt=1.493 min

### Step 2: 1-bromo-3-(bromomethyl)-5-fluorobenzene

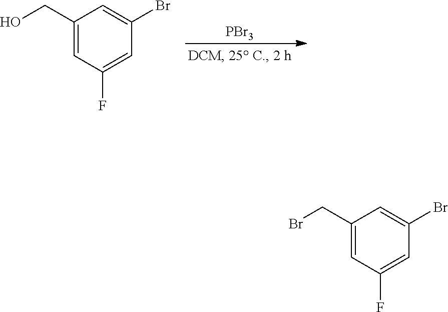

To a solution of (3-bromo-5-fluoro-phenyl)methanol (50 g, 243.87 mmol, 1 eq) in DCM (500 mL) was added PBr₃ (132.03 g, 487.75 mmol, 2 eq). The mixture was stirred at 25° C. for 2 hrs under N₂ atmosphere. TLC indicated the reaction was completed. The mixture was concentrated. The residue was diluted with saturated NaHCO₃ (800 mL) and extracted with EtOAc (800 mL*2). The organic layers were combined, washed with water (800 mL*2), saturated brine (800 mL), dried with anhydrous Na₂SO₄, filtered and concentrated to give 1-bromo-3-(bromomethyl)-5-fluorobenzene (60 g, crude) as white solid. ¹H NMR (400 MHz, DMSO-d₆) δ=7.47 (s, 1H), 7.46-7.41 (m, 1H), 7.32-7.25 (m, 1H), 4.62 (s, 2H)

### Step 3: 2-(3-bromo-5-fluorophenyl)acetonitrile

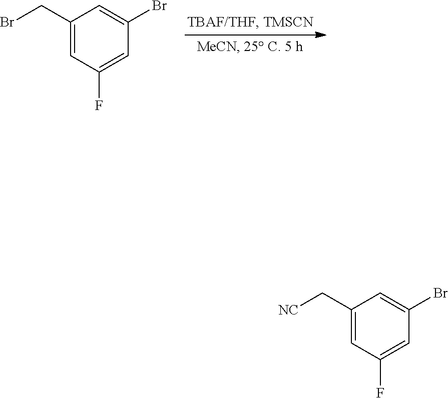 [SMILES: Fc1cc(Br)cc(CBr)c1 ; N#CCc1cc(F)cc(Br)c1]

To a solution of 1-bromo-3-(bromomethyl)-5-fluorobenzene (50 g, 186.62 mmol, 1 eq) and TMSCN (24.07 g, 242.61 mmol, 30.35 mL, 1.3 eq) in CH₃CN (600 mL) at 25° C., TBAF in THF (1 M, 242.61 mL, 1.3 eq) was added. The mixture was stirred at 25° C. for 5 hrs. TLC indicated the reaction was completed. The mixture was concentrated. The residue was diluted with H₂O (1000 mL) and extracted with EtOAc (800 mL*2). The organic layers were combined, washed with water (800 mL*2), saturated brine (800 mL), dried with anhydrous Na₂SO₄, filtered and concentrated to give crude product. The crude product was purified by chromatography on silica gel (petroleum ether/EtOAc=20/1) to give 2-(3-bromo-5-fluorophenyl)acetonitrile (29.1 g, 135.96 mmol, 72.85% yield) as yellow oil. ¹H NMR (400 MHz, DMSO-d₆) δ=7.54 (td, J=2.0, 8.4 Hz, 1H), 7.46 (s, 1H), 7.28 (br d, J=9.4 Hz, 1H), 4.10 (s, 2H).

### Step 4: tert-butyl (3-(cyanomethyl)-5-fluorophenyl)carbamate

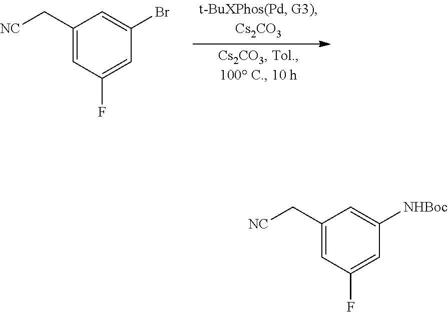 [SMILES: N#CCc1cc(F)cc(Br)c1 ; CC(C)(C)OC(=O)Nc1cc(F)cc(CC#N)c1]

A mixture of 2-(3-bromo-5-fluorophenyl)acetonitrile (10 g, 46.72 mmol, 1 eq), tert-butyl carbamate (8.21 g, 70.08 mmol, 1.5 eq), t-Bu Xphos (991.99 mg, 2.34 mmol, 0.05 eq), tBuXPhos Pd G3 (1.86 g, 2.34 mmol, 0.05 eq) and Cs₂CO₃ (30.45 g, 93.44 mmol, 2 eq) in toluene (100 mL) was degassed and purged with N₂ for 3 times, and then the mixture was stirred at 100° C. for 10 hrs under N₂ atmosphere. LC-MS showed the reaction was completed. The reaction mixture was filtered and concentrated under reduced pressure to give a residue. The residue was purified by column chromatography (SiO2, Petroleum ether/Ethyl acetate=10/1 to 1/1) to afford tert-butyl (3-(cyanomethyl)-5-fluorophenyl)carbamate (9 g, 35.96 mmol, 76.97% yield) as yellow solid. 1H NMR (400 MHz, DMSO-d6) δ=9.70 (s, 1H), 7.34 (s, 1H), 7.25 (br d, J=11.5 Hz, 1H), 6.77 (br d, J=8.8 Hz, 1H), 4.03 (s, 2H), 1.48 (s, 9H). LC-MS (ES⁺, m/z): 251.2 [(M+H)]; Rt=0.795 min.

### Step 5: 2-(3-amino-5-fluorophenyl)acetonitrile

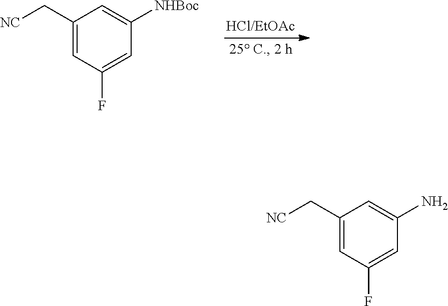 [SMILES: CC(C)(C)OC(=O)Nc1cc(F)cc(CC#N)c1 ; N#CCc1cc(N)cc(F)c1]

A mixture of tert-butyl N-[3-(cyanomethyl)-5-fluoro-phenyl]carbamate (9 g, 35.96 mmol, 1 eq) in HCl/EtOAc (4 M, 99.96 mL, 11.12 eq) was stirred at 25° C. for 2 hrs. LC-MS showed reaction was completed. The reaction mixture was poured into saturated Na₂CO₃ (50 mL) and extracted with EA 150 mL (50 mL*3). The combined organic layers were washed with saturated brine 60 mL (20 mL*3), dried over anhydrous Na₂SO₄, filtered and concentrated under reduced pressure to afford 2-(3-amino-5-fluorophenyl)acetonitrile (4.3 g, crude) as yellow oil. LC-MS (ES⁺, m/z): 151.2 [(M+H)⁺]; Rt=0.120 min.

Note: HCl/EtOAc (4 M): HCl was bubbled into a solution EtOAc at 0° C. for 0.5 h. Then, the solution was weighed to obtained the HCl/EtOAc (4 M)

### Step 6: 3-(2-aminoethyl)-5-fluoroaniline

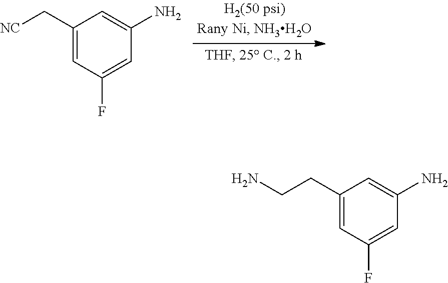 [SMILES: N#CCc1cc(N)cc(F)c1 ; NCCc1cc(N)cc(F)c1]

To a solution of 2-(3-amino-5-fluoro-phenyl)acetonitrile (4.3 g, 28.64 mmol, 1 eq) and NH3·H2O (4.55 g, 45.44 mmol, 5.00 mL, 35% purity, 1.59 eq) in THF (100 mL) was added Raney-Ni (15.00 g, 175.08 mmol, 6.11 eq) under N₂ atmosphere. The suspension was degassed and purged with H₂ for 3 times. The mixture was stirred under 50 Psi of H₂ (57.85 mg, 28.64 mmol, 1 eq) at 25° C. for 2 hrs. LC-MS showed reaction was completed. The reaction mixture was filtered and concentrated under reduced pressure to afford 3-(2-aminoethyl)-5-fluoroaniline (4.3 g, crude) as yellow oil. LC-MS (ES⁺, m/z): 155.1 [(M+H)⁺]; Rt=0.146 min.

### Step 7: tert-butyl (3-amino-5-fluorophenethyl)carbamate

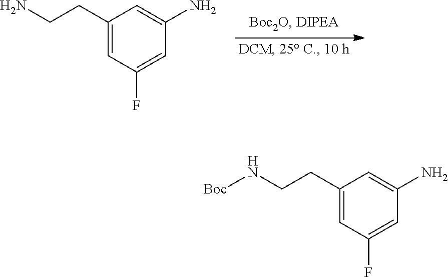 [SMILES: NCCc1cc(N)cc(F)c1 ; CC(C)(C)OC(=O)NCCc1cc(N)cc(F)c1]

A mixture of 3-(2-aminoethyl)-5-fluoroaniline (4.3 g, 27.89 mmol, 1 eq), (Boc)₂O (6.09 g, 27.89 mmol, 6.41 mL, 1 eq), DIPEA (7.21 g, 55.78 mmol, 9.72 mL, 2 eq) in DCM (50 mL) was degassed and purged with N₂ for 3 times, and then the mixture was stirred at 25° C. for 10 hrs under N₂ atmosphere. LC-MS showed the reaction was completed. The reaction mixture was concentrated under reduced pressure to remove DCM. The residue was diluted with H₂O 10 mL and extracted with EA (30 mL*3). The combined organic layers were washed with saturated brine (100 mL*1), dried over anhydrous Na₂SO₄, filtered and concentrated under reduced pressure to give a residue. The residue was purified by column chromatography (SiO2, Petroleum ether/Ethyl acetate=10/1 to 1/1) to afford tert-butyl N-[2-(3-amino-5-fluoro-phenyl)ethyl]carbamate (3.4 g, 13.37 mmol, 47.94% yield) as yellow oil. 1H NMR (400 MHz, DMSO-d6) δ=6.91-6.76 (m, 1H), 6.24-6.03 (m, 3H), 5.37-5.25 (m, 2H), 3.19-3.15 (d, 1H), 3.11-3.03 (m, 2H), 2.57-2.52 (m, 1H), 1.42-1.34 (m, 9H). LC-MS (ES⁺, m/z): 155.2 [(M+H-100)⁺], 199.2 [(M+H-56)⁺]; Rt=0.666 min.

### Step 8: tert-butyl (3-((3-carbamoyl-6-chloro-5-ethylpyrazin-2-yl)amino)-5-fluorophenethyl)carbamate

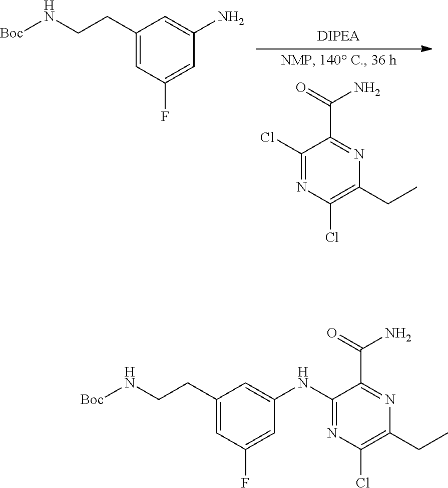 [SMILES: CC(C)(C)OC(=O)NCCc1cc(N)cc(F)c1 ; CCc1nc(C(N)=O)c(Cl)nc1Cl ; CCc1nc(C(N)=O)c(Nc2cc(F)cc(CCNC(=O)OC(C)(C)C)c2)nc1Cl]

To a solution of tert-butyl (3-amino-5-fluorophenethyl)carbamate (2 g, 7.86 mmol, 1 eq) and 3,5-dichloro-6-ethylpyrazine-2-carboxamide (1.73 g, 7.86 mmol, 1 eq) in NMP (4 mL) was added DIPEA (40.66 g, 314.59 mmol, 54.80 mL, 40 eq). The mixture was stirred at 140° C. for 36 hrs. LC-MS showed the reaction was completed. The reaction mixture was poured into H₂O (15 mL), filtered to give a residue. The residue was diluted with EA 15 mL and poured into H₂O 15 mL, then extracted with EA (15 mL*3). The organic phase was separated, washed with saturated brine (10 mL*3), dried over anhydrous Na₂SO₄, filtered and concentrated under reduced pressure to give a residue. The residue was purified by column chromatography (SiO₂, Petroleum ether/Ethyl acetate=5/1 to 1/1) to afford tert-butyl (3-((3-carbamoyl-6-chloro-5-ethylpyrazin-2-yl)amino)-5-fluorophenethyl)carbamate (1.78 g, 3.13 mmol, 39.86% yield, 77.12% purity) as yellow oil. ¹H NMR (400 MHz, DMSO-d6) δ=11.36 (s, 1H), 8.30 (br s, 1H), 8.09 (br s, 1H), 7.65 (br d, J=11.5 Hz, 1H), 6.98 (s, 1H), 6.87 (br t, J=5.0 Hz, 1H), 6.71 (br d, J=9.4 Hz, 1H), 3.19-3.13 (m, 2H), 2.83 (q, J=7.4 Hz, 2H), 2.69 (br t, J=6.9 Hz, 2H), 1.34 (s, 9H), 1.26 (t, J=7.4 Hz, 3H). LC-MS (ES⁺, m/z): 438.2 [(M+H)⁺]; Rt=0.914 min; 77.12% purity.

### Step 9: 3-((3-(2-aminoethyl)-5-fluorophenyl)amino)-5-chloro-6-ethylpyrazine-2-carboxamide

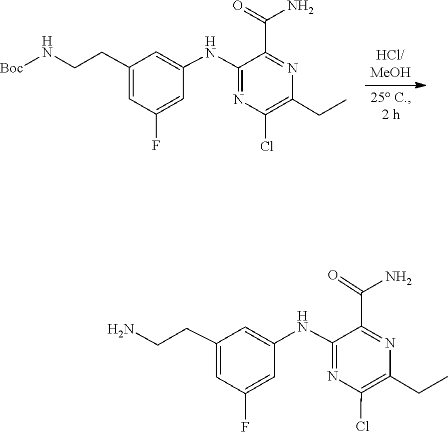 [SMILES: CCc1nc(C(N)=O)c(Nc2cc(F)cc(CCNC(=O)OC(C)(C)C)c2)nc1Cl ; CCc1nc(C(N)=O)c(Nc2cc(F)cc(CCN)c2)nc1Cl]

To a solution of tert-butyl (3-((3-carbamoyl-6-chloro-5-ethylpyrazin-2-yl)amino)-5-fluorophenethyl)carbamate (1.3 g, 2.97 mmol, 1 eq) in HCl/MeOH (4 M, 50 mL, 67.37 eq) was stirred at 25° C. for 2 hrs. LC-MS showed the reaction was completed. The reaction mixture was filtered and concentrated under reduced pressure to give a residue to afford 3-((3-(2-aminoethyl)-5-fluorophenyl)amino)-5-chloro-6-ethylpyrazine-2-carboxamide (1 g, crude, HCl) as yellow solid. LC-MS (ES⁺, m/z): 338.2 [(M+H)⁺]; Rt=0.710 min.

Note: HCl/MeOH (4 M): HCl was bubbled into a solution MeOH at 0° C. for 0.5 h.

### Step 10: tert-butyl (S)-(1-((3-((3-carbamoyl-6-chloro-5-ethylpyrazin-2-yl)amino)-5-fluorophenethyl)amino)-1-oxopropan-2-yl)(methyl)carbamate

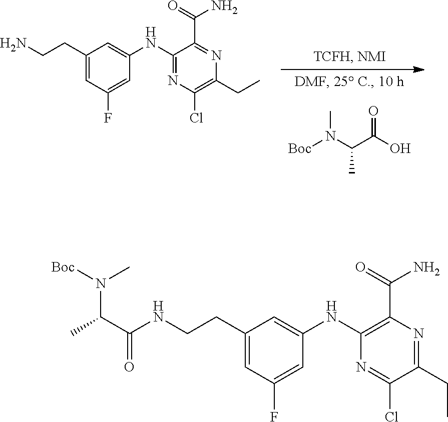 [SMILES: CCc1nc(C(N)=O)c(Nc2cc(F)cc(CCN)c2)nc1Cl ; C[C@@H](C(=O)O)N(C)C(=O)OC(C)(C)C ; CCc1nc(C(N)=O)c(Nc2cc(F)cc(CCNC(=O)[C@H](C)N(C)C(=O)OC(C)(C)C)c2)nc1Cl]

To a solution of 3-((3-(2-aminoethyl)-5-fluorophenyl)amino)-5-chloro-6-ethylpyrazine-2-carboxamide (1 g, 2.96 mmol, 1 eq) and N-(tert-butoxycarbonyl)-N-methyl-L-alanine (902.52 mg, 4.44 mmol, 1.5 eq) in DMF (15 mL) was added 1-methyl-1H-imidazole (2.43 g, 29.61 mmol, 2.36 mL, 10 eq) and chloro-N,N,N′,N′-tetramethylformamidinium hexafluorophosphate (1.25 g, 4.44 mmol, 1.5 eq). The mixture was stirred at 25° C. for 10 hrs. LC-MS showed the reaction was completed. The reaction mixture was poured into H₂O (15 mL) and extracted with EA (10 mL*3). The combined organic layers were washed with saturated brine (10 mL*3), dried over anhydrous Na₂SO₄, filtered and concentrated under reduced pressure to give a residue. The residue was purified by column chromatography (SiO2, Petroleum ether/Ethyl acetate=5/1 to 1/5) to afford tert-butyl (S)-(1-((3-((3-carbamoyl-6-chloro-5-ethylpyrazin-2-yl)amino)-5-fluorophenethyl)amino)-1-oxopropan-2-yl)(methyl)carbamate (1.5 g, 2.87 mmol, 96.88% yield) was obtained as a yellow oil. ¹H NMR (400 MHz, DMSO-d₆) δ=11.42-11.35 (m, 1H), 8.38-8.05 (m, 2H), 7.96 (s, 1H), 7.82 (br t, J=5.1 Hz, 1H), 7.70-7.61 (m, 1H), 7.08-7.00 (m, 1H), 6.76-6.70 (m, 1H), 3.32 (s, 3H), 2.89 (s, 2H), 2.87 (s, 2H), 2.73 (s, 2H), 1.35 (br s, 9H), 1.27 (t, J=7.5 Hz, 3H), 1.22-1.16 (m, 3H). LC-MS (ES⁺, m/z): 415.1 [(M+H)⁺]; Rt=1.901 min, 99.09% purity; HRMS: 415.2488.

### Intermediate 9

### 3,5-dichloro-6-ethyl-N-methylpyrazine-2-carboxamide

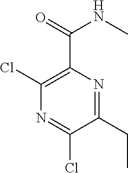

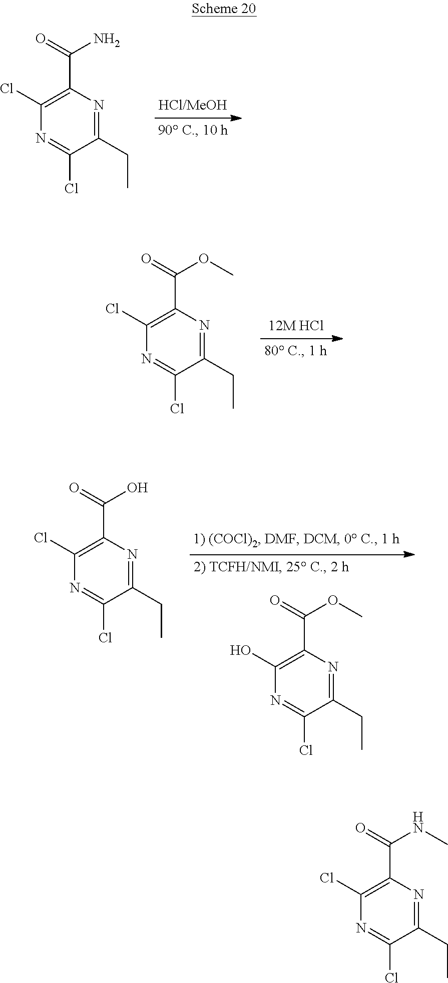

### Step 1: methyl 3,5-dichloro-6-ethylpyrazine-2-carboxylate

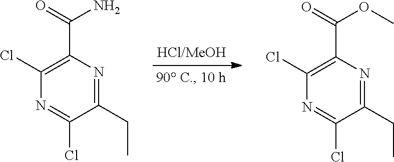

A mixture of 3,5-dichloro-6-ethyl-pyrazine-2-carboxamide (15 g, 68.16 mmol, 1.0 eq), HCl/MeOH (4 M, 350.03 mL, 20.54 eq) was stirred at 90° C. for 10 hrs. LCMS showed the reaction was completed. The mixture was concentrated. The residue was diluted with saturated NaHCO₃ (500 mL) and extracted with EtOAc (400 mL*2). The organic layers were combined, washed with water (300 mL*2), sat. saturated brine (300 mL), dried with anhydrous Na2CO3, filtered and concentrated to give crude product. The crude product was purified by chromatography on silica gel (petroleum ether/EtOAc=100/1) TLC (plate1) Rf=0.67) to afford methyl 3,5-dichloro-6-ethyl-pyrazine-2-carboxylate (13 g, 55.30 mmol, 81.13% yield) as a yellow oil. LC-MS (ES⁺, m/z): 235.1 [(M+H)⁺]; Rt=0.830 min.

Note: HCl/MeOH (4 M): HCl was bubbled into a solution MeOH at 0° C. for 0.5 h.

### Step 2: 3,5-dichloro-6-ethylpyrazine-2-carboxylic acid

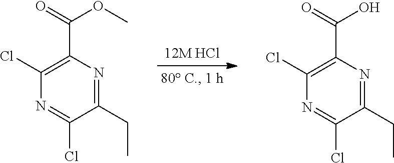

The mixture of methyl 3,5-dichloro-6-ethyl-pyrazine-2-carboxylate (4.8 g, 20.42 mmol, 1.0 eq) in HCl (12 M, 671.40 mL, 131.52 eq). LCMS showed the reaction was completed. The reaction was poured into H₂O (100 mL). The aqueous phase was extracted with ethyl acetate (50 mL*3). The combined organic phase was washed with saturated brine (100 mL*1), dried with anhydrous Na₂SO₄. Filtered and concentrated in vacuum to afford 3,5-dichloro-6-ethyl-pyrazine-2-carboxylic acid (3 g, 13.57 mmol, 66.47% yield) as a yellow solid. LC-MS (ES⁺, m/z): 221.1 [(M+H)⁺]; Rt=0.688 min.

### Step 3: 3,5-dichloro-6-ethylpyrazine-2-carbonyl chloride

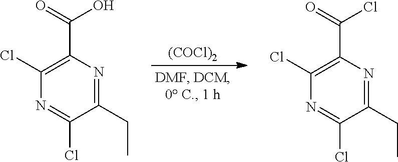

To a mixture of 3,5-dichloro-6-ethyl-pyrazine-2-carboxylic acid (1 g, 4.52 mmol, 1.0 eq) in DCM (10 mL) was added DMF (16.53 mg, 226.20 umol, 0.05 eq) finally added (COCl)₂ (1.15 g, 9.05 mmol, 2.0 eq) in one portion at 0° C. under N₂ for 1 hr. TLC showed the reaction was completed. The residue was concentrated in vacuum to afford 3,5-dichloro-6-ethyl-pyrazine-2-carbonyl chloride (0.8 g, 3.34 mmol, 73.84% yield) as a yellow solid.

### Step 4: 3,5-dichloro-6-ethyl-N-methylpyrazine-2-carboxamide

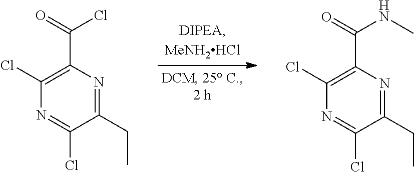

A mixture of 3,5-dichloro-6-ethyl-pyrazine-2-carbonyl chloride (0.8 g, 3.34 mmol, 1.0 eq), methanamine;hydrochloride (451.08 mg, 6.68 mmol, 2.0 eq), and DIPEA (2.16 g, 16.70 mmol, 5.0 eq) in DCM (10 mL). The mixture was stirred at 25° C. for 2 hours. LCMS showed the reaction was completed. The reaction was poured into H₂O (50 mL). The aqueous phase was extracted with ethyl acetate (10 mL*3). The combined organic phase was washed with saturated brine (50 mL*1), dried with anhydrous Na₂SO₄. Filtered and concentrated in vacuum. The residue was purified by silica gel chromatography (column height: 250 mm, diameter: 100 mm, 100-200 mesh silica gel, Petroleum ether/Ethyl acetate=1/1, 10/1) to afford 3,5-dichloro-6-ethyl-N-methyl-pyrazine-2-carboxamide (500 mg, 2.14 mmol, 63.94% yield) as a brown solid. LC-MS (ES⁺, m/z): 234.1 [(M+H)⁺]; Rt=0.705 min.

### Intermediate 10

### (E)-N-(4-(dimethylamino)but-2-enoyl)-N-methyl-L-alanine

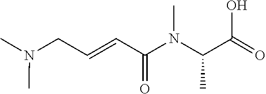

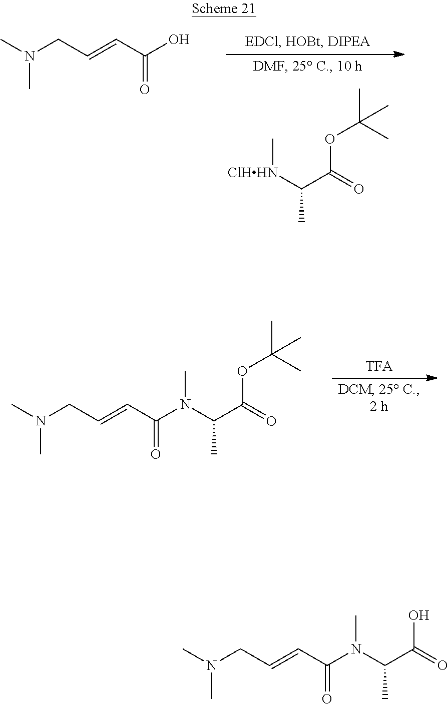

### Step 1: tert-butyl (E)-N-(4-(dimethylamino)but-2-enoyl)-N-methyl-L-alaninate

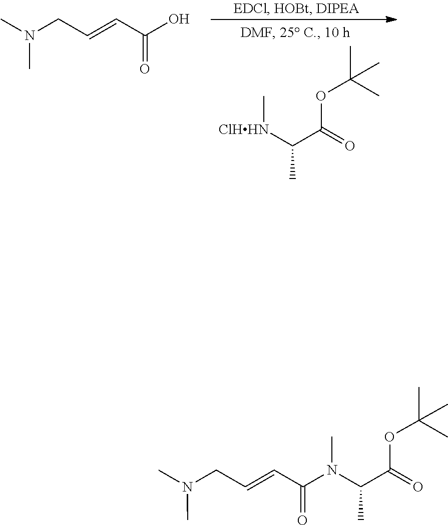

To a solution of tert-butyl methyl-L-alaninate hydrochloride (3 g, 15.33 mmol, 1 eq) (E)-4-(dimethylamino)but-2-enoic acid (2.79 g, 16.86 mmol, 1.1 eq) EDCI (4.41 g, 23.00 mmol, 1.5 eq) HOBt (2.07 g, 15.33 mmol, 1 eq) in DMF (30 mL) at 25° C., DIPEA (19.81 g, 153.31 mmol, 26.70 mL, 10 eq) was added. The mixture was stirred at 25° C. for 10 hrs. LCMS indicated the reaction was completed. The mixture was poured into water (100 mL) and extracted with EtOAc (60 mL*2). The organic layers was washed with water (60 mL*2), saturated brine (60 mL*2), dried over anhydrous Na2SO4, filtered, concentrated under reduced pressure to give a residue. The crude product was purified by chromatography on silica gel (Dichloromethane:Methanol=10:1) to afford tert-butyl (E)-N-(4-(dimethylamino)but-2-enoyl)-N-methyl-L-alaninate (2.75 g, 9.56 mmol, 62.37% yield, 94% purity) as black brown oil. LC-MS (ES⁺, m/z): 217.3 [(M+H)+]; Rt=0.556 min.

### Step 2: (E)-N-(4-(dimethylamino)but-2-enoyl)-N-methyl-L-alanine

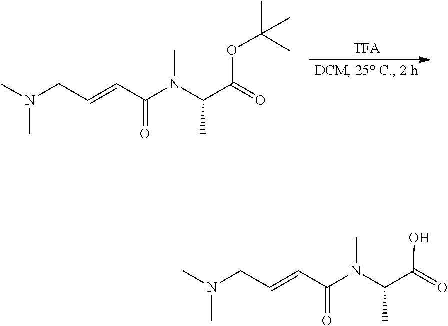

To a solution of tert-butyl (E)-N-(4-(dimethylamino)but-2-enoyl)-N-methyl-L-alaninate (1 g, 3.70 mmol, 1 eq) TFA (7.70 g, 67.53 mmol, 5 mL, 18.26 eq) in DCM (10 mL) at 25° C. The mixture was stirred at 25° C. for 2 hrs. LCMS indicated the reaction was completed. The mixture was a concentrated under reduced pressure to give (E)-N-(4-(dimethylamino)but-2-enoyl)-N-methyl-L-alanine (760 mg, crude) as black brown oil. LC-MS (ES⁺, m/z): 215.2 [(M+H)⁺]; Rt=0.339 min.

### Intermediate 11

### 3-((3-(2-aminoethyl)phenyl)amino)-5-(dimethylamino)-6-ethylpyrazine-2-carboxamide

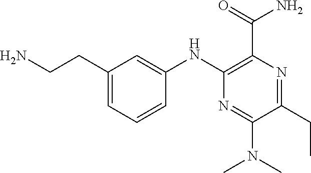 [SMILES: CCc1nc(C(N)=O)c(Nc2cccc(CCN)c2)nc1N(C)C]

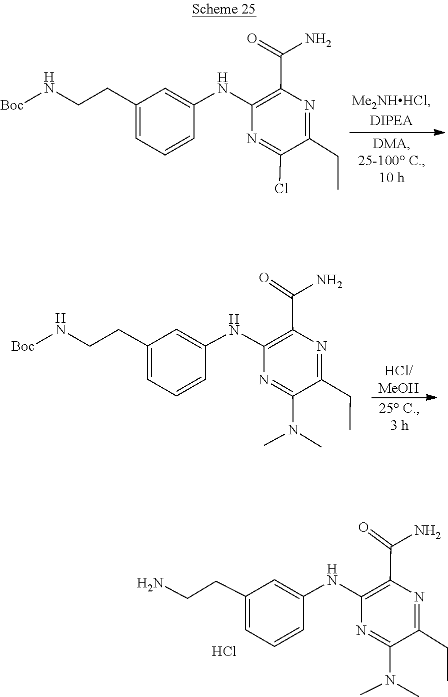 [SMILES: CCc1nc(C(N)=O)c(Nc2cccc(CCNC(=O)OC(C)(C)C)c2)nc1Cl ; CCc1nc(C(N)=O)c(Nc2cccc(CCNC(=O)OC(C)(C)C)c2)nc1N(C)C ; CCc1nc(C(N)=O)c(Nc2cccc(CCN)c2)nc1N(C)C ; Cl]

### Step 1: tert-butyl (3-((3-carbamoyl-6-(dimethylamino)-5-ethylpyrazin-2-yl)amino)phenethyl)carbamate

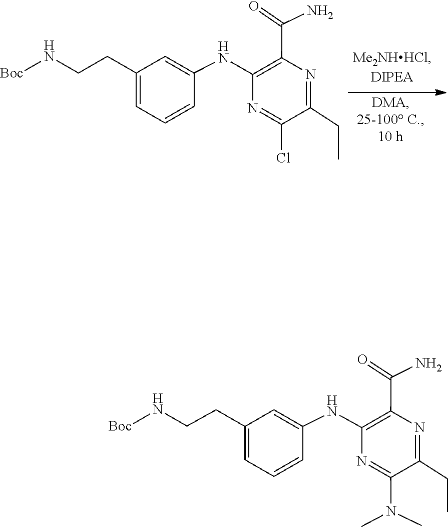 [SMILES: CCc1nc(C(N)=O)c(Nc2cccc(CCNC(=O)OC(C)(C)C)c2)nc1Cl ; CCc1nc(C(N)=O)c(Nc2cccc(CCNC(=O)OC(C)(C)C)c2)nc1N(C)C]

To a solution of tert-butyl (3-((3-carbamoyl-6-chloro-5-ethylpyrazin-2-yl)amino)phenethyl)carbamate (3 g, 7.14 mmol, 1 eq) DIEA (9.23 g, 71.44 mmol, 12.44 mL, 10 eq) N-methylmethanamine (3.22 g, 39.50 mmol, 3.62 mL, 5.53 eq, HCl) and DMA (20 mL) at 25° C., the mixture was stirred at 100° C. for 10 hrs. LCMS indicated the reaction was completed. The reaction was poured into water (50 mL) and extracted with EtOAc (30 mL*3). The organic layers were combined, washed with water (10 mL*2), sat. brine (10 mL), dried with anhydrous Na₂SO₄, filtered and concentrated to give crude product. The crude product was purified by chromatography on silica thiol gel (petroleum ether/EtOAc=1/1). To give tert-butyl (3-((3-carbamoyl-6-(dimethylamino)-5-ethylpyrazin-2-yl)amino)phenethyl)carbamate (1.34 g, 3.13 mmol, 43.77% yield) as yellow solid. LC-MS (ES⁺, m/z): 429.3 [(M+H)⁺]; Rt=0.929 min.

### Step 2: 3-((3-(2-aminoethyl)phenyl)amino)-5-(dimethylamino)-6-ethylpyrazine-2-carboxamide

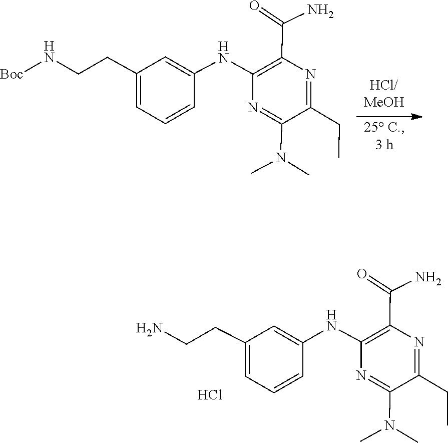 [SMILES: CCc1nc(C(N)=O)c(Nc2cccc(CCNC(=O)OC(C)(C)C)c2)nc1N(C)C ; CCc1nc(C(N)=O)c(Nc2cccc(CCN)c2)nc1N(C)C ; Cl]

A mixture of tert-butyl (3-((3-carbamoyl-6-(dimethylamino)-5-ethylpyrazin-2-yl)amino)phenethyl)carbamate (1.3 g, 3.03 mmol, 1 eq) HCl/EtOAc (4 M, 30 mL, 39.56 eq) was stirred at 25° C. for 3 hr. LCMS indicated the reaction was completed. The mixture was concentrated under reduced pressure to give 3-((3-(2-aminoethyl)phenyl)amino)-5-(dimethylamino)-6-ethylpyrazine-2-carboxamide (900 mg, crude, HCl) was yellow solid. LC-MS (ES⁺, m/z): 329.4 [(M+H)⁺]; Rt=0.641 min.

Note: HCl/MeOH (4 M): HCl gas was bubbled into a solution MeOH at 0° C. for 0.5 h. Then, the solution was weighed to obtained the HCl/MeOH (4 M)

### Intermediate 12

### (S)-6-ethyl-5-(isopropyl(methyl)amino)-3-((3-(2-(2-(methylamino)propanamido)ethyl)phenyl)amino)pyrazine-2-carboxamide

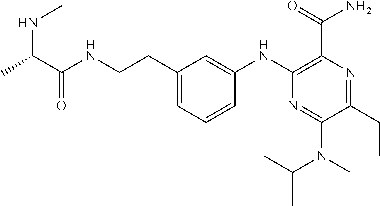 [SMILES: CCc1nc(C(N)=O)c(Nc2cccc(CCNC(=O)[C@H](C)NC)c2)nc1N(C)C(C)C]

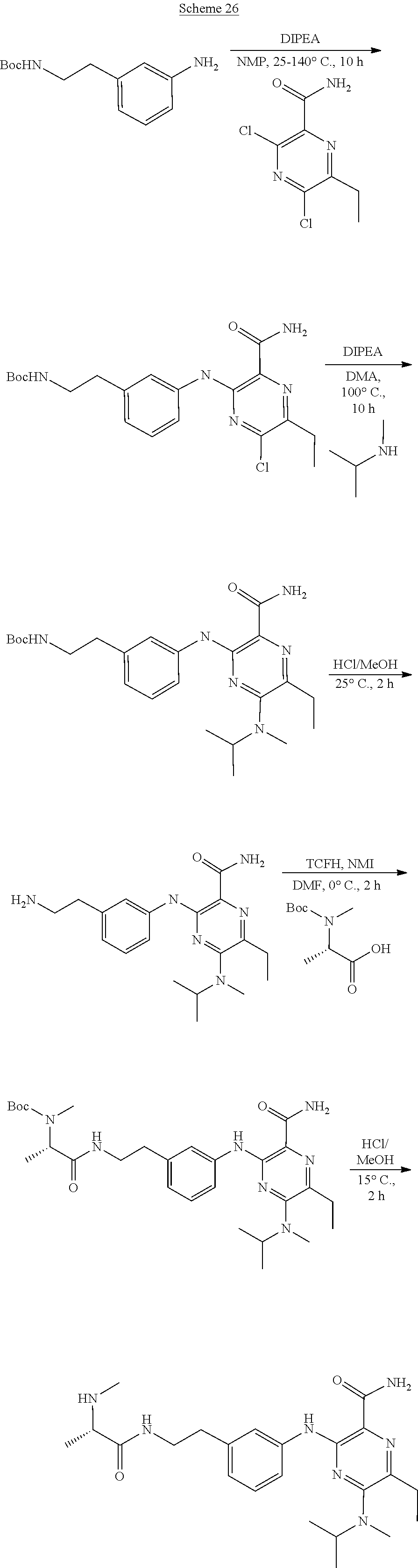

### Step 1: tert-butyl (3-((3-carbamoyl-6-chloro-5-ethylpyrazin-2-yl)amino)phenethyl)carbamate

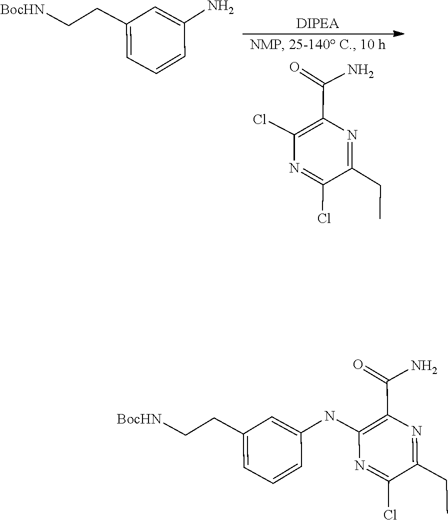

A mixture of tert-butyl (3-aminophenethyl)carbamate (2 g, 8.46 mmol, 1 eq), 3,5-dichloro-6-ethylpyrazine-2-carboxamide (1.86 g, 8.46 mmol, 1 eq) in NMP (5 mL), DIPEA (21.88 g, 169.27 mmol, 29.48 mL, 20 eq) was added at 25° C. The mixture was stirred at 140° C. for 10 hours. LCMS showed the reaction was completed. The reaction was poured into H₂O (50 mL). The aqueous phase was extracted with ethyl acetate (20 mL*3). The combined organic phase was washed with saturated brine (50 mL*1), dried with anhydrous Na₂SO₄. Filtered and concentrated in vacuum. The residue was purified by silica gel chromatography (column height: 250 mm, diameter: 100 mm, 100-200 mesh silica gel, Petroleum ether/Ethyl acetate=1/1, 10/1) to afford tert-butyl (3-((3-carbamoyl-6-chloro-5-ethylpyrazin-2-yl)amino)phenethyl)carbamate (3.5 g, 6.67 mmol, 78.79% yield) as a yellow solid. 1H NMR (400 MHz, DMSO-d6) δ=11.19 (s, 1H), 8.26 (s, 1H), 8.04 (br s, 1H), 7.59 (br d, J=7.9 Hz, 1H), 7.32-7.23 (m, 2H), 6.93-6.83 (m, 2H), 3.16 (q, J=6.5 Hz, 2H), 2.82 (q, J=7.5 Hz, 2H), 2.69 (t, J=7.3 Hz, 2H), 1.36 (s, 9H), 1.28-1.24 (m, 3H) LC-MS (ES+, m/z): 420.3 [(M+H)+]; Rt=0.941 min.

### Step 2: tert-butyl (3-((3-carbamoyl-5-ethyl-6-(isopropyl(methyl)amino)pyrazin-2-yl)amino)phenethyl)carbamate

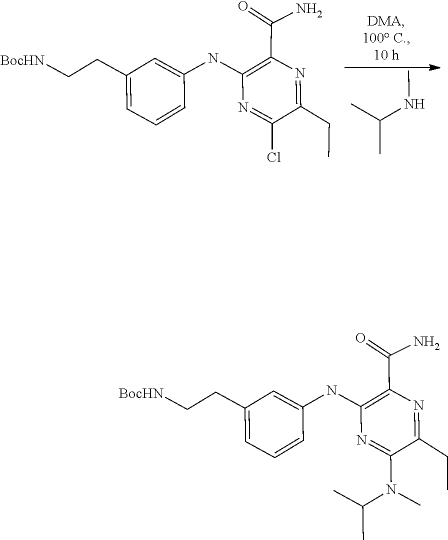

To a solution of tert-butyl (3-((3-carbamoyl-6-chloro-5-ethylpyrazin-2-yl)amino)phenethyl)carbamate (400 mg, 952.60 umol, 1 eq) and N-methylpropan-2-amine (696.70 mg, 9.53 mmol, 992.44 uL, 10 eq) in DMA (10 mL) was added DIPEA (123.12 mg, 952.60 umol, 165.93 uL, 1 eq) at 20° C. The mixture was stirred at 100° C. for 10 hrs in sealed tube. LCMS indicated the reaction was complete. The reaction was poured into water (30 mL) and extracted with EtOAc (30 mL*2). The organic layers were combined, washed with water (15 mL*2), saturated brine (10 mL), dried (Na2SO4), filtered and concentrated to give crude product. The crude product was purified by chromatography on silica thiol gel (petroleum ether/EtOAc=1/2) to give tert-butyl (3-((3-carbamoyl-5-ethyl-6-(isopropyl(methyl)amino)pyrazin-2-yl)amino)phenethyl)carbamate (350 mg, 766.57 umol, 80.47% yield) as yellow solid. LC-MS (ES⁺, m/z): 457.5 [(M+H)⁺]; Rt=0.992 min.

### Step 3: 3-((3-(2-aminoethyl)phenyl)amino)-6-ethyl-5-(isopropyl(methyl)amino)pyrazine-2-carboxamide

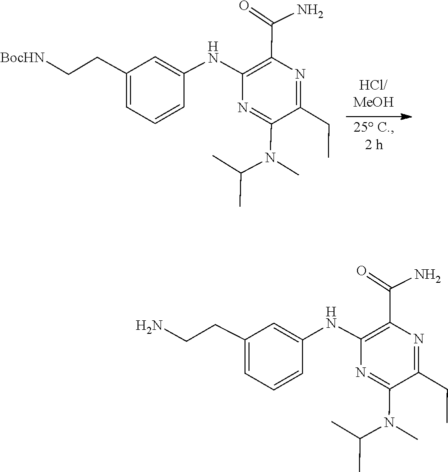 [SMILES: CCc1nc(C(N)=O)c(Nc2cccc(CCNC(=O)OC(C)(C)C)c2)nc1N(C)C(C)C ; CCc1nc(C(N)=O)c(Nc2cccc(CCN)c2)nc1N(C)C(C)C]

A solution of tert-butyl (3-((3-carbamoyl-5-ethyl-6-(isopropyl(methyl)amino)pyrazin-2-yl)amino)phenethyl)carbamate (350 mg, 766.57 umol, 1 eq) and HCl/MeOH (4 M, 10.94 mL, 57.07 eq) was stirred at 25° C. for 2 hrs. LCMS indicated the reaction was complete. The mixture was concentrated under reduced pressure to give 3-((3-(2-aminoethyl)phenyl)amino)-6-ethyl-5-(isopropyl(methyl)amino)pyrazine-2-carboxamide (260 mg, crude) as yellow solid. LC-MS (ES⁺, m/z): 357.3 [(M+H)⁺]; Rt=0.716 min.

Note: HCl/MeOH (4 M): HCl gas was bubbled into a solution MeOH at 0° C. for 0.5 h. Then, the solution was weighed to obtained the HCl/MeOH (4 M)

### Step 4: tert-butyl (S)-(1-((3-((3-carbamoyl-5-ethyl-6-(isopropyl(methyl)amino)pyrazin-2-yl)amino)phenethyl)amino)-1-oxopropan-2-yl)(methyl)carbamate

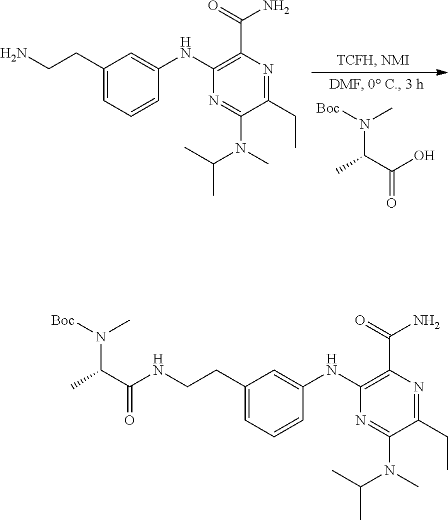

To a solution of 3-((3-(2-aminoethyl)phenyl)amino)-6-ethyl-5-(isopropyl(methyl)amino)pyrazine-2-carboxamide (260 mg, 729.39 umol, 1 eq), NMI (598.85 mg, 7.29 mmol, 581.41 uL, 10 eq), N-(tert-butoxycarbonyl)-N-methyl-L-alanine (222.36 mg, 1.09 mmol, 1.5 eq) in DMF (3 mL), was added TCFH (306.98 mg, 1.09 mmol, 1.5 eq). The mixture was stirred at 0° C. for 2 hrs. LCMS indicated the reaction was complete. The reaction was poured into water (15 mL) and extracted with EtOAc (10 mL*2). The organic layers were combined, washed with water (10 mL*2), saturated brine (5 mL), dried (Na₂SO₄), filtered and concentrated to give crude product. The crude product was purified by chromatography on silica thiol gel (petroleum ether/EtOAc=1/1) TLC (plate1 Rf=0.75) to give tert-butyl (S)-(1-((3-((3-carbamoyl-5-ethyl-6-(isopropyl(methyl)amino)pyrazin-2-yl)amino)phenethyl)amino)-1-oxopropan-2-yl)(methyl)carbamate (350 mg, 646.13 umol, 88.59% yield) as yellow solid. LC-MS (ES⁺, m/z): 542.4 [(M+H)⁺]; Rt=0.945 min.

### Step 5: (S)-6-ethyl-5-(isopropyl(methyl)amino)-3-((3-(2-(2-(methylamino)propanamido)ethyl)phenyl)amino)pyrazine-2-carboxamide

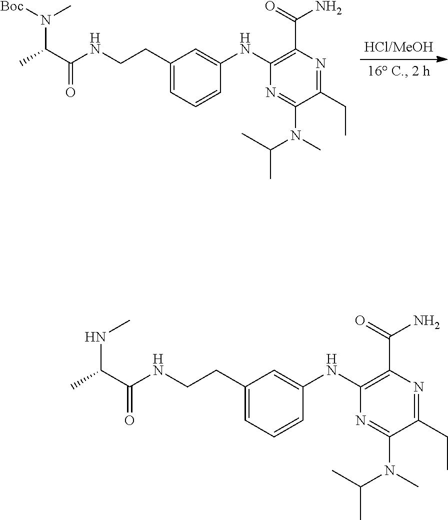

A mixture of tert-butyl (S)-(1-((3-((3-carbamoyl-5-ethyl-6-(isopropyl(methyl)amino)pyrazin-2-yl)amino)phenethyl)amino)-1-oxopropan-2-yl)(methyl)carbamate (350 mg, 646.13 umol, 1 eq) and HCl/MeOH (4 M, 10 mL, 61.91 eq) was stirred at 15° C. for 2 hrs. LCMS indicated the reaction was complete. The mixture was concentrated under reduced pressure to give (S)-6-ethyl-5-(isopropyl(methyl)amino)-3-((3-(2-(2-(methylamino)propanamido)ethyl)phenyl)amino)pyrazine-2-carboxamide (270 mg, crude) as yellow solid. LC-MS (ES⁺, m/z): 442.3 [(M+H)⁺]; Rt=0.726 min.

Note: HCl/MeOH (4 M): HCl gas was bubbled into a solution MeOH at 0° C. for 0.5 h. Then, the solution was weighed to obtained the HCl/MeOH (4 M)

### Intermediate 13

### tert-butyl (3-((3-carbamoyl-6-chloro-5-methylpyrazin-2-yl)amino)phenethyl)carbamate

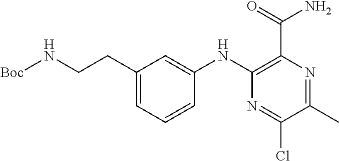 [SMILES: Cc1nc(C(N)=O)c(Nc2cccc(CCNC(=O)OC(C)(C)C)c2)nc1Cl]

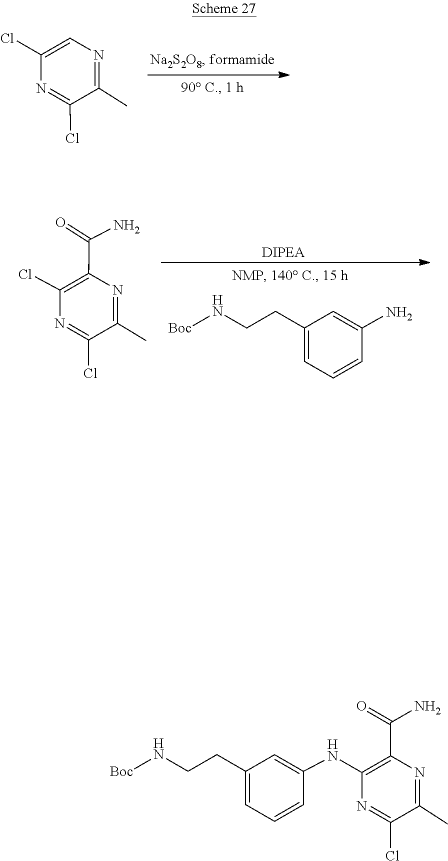

### Step 1: 3,5-dichloro-6-methylpyrazine-2-carboxamide

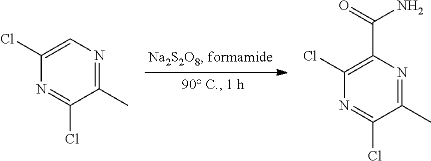

To a solution of 3,5-dichloro-2-methylpyrazine (8 g, 49.08 mmol, 1 eq) in formamide (53.05 g, 1.18 mol, 46.95 mL, 24 eq) at 25° C., Na₂S₂O₈ (18.70 g, 78.53 mmol, 17.00 mL, 1.6 eq) was added in batches at 90° C. The mixture was stirred at 90° C. for 1 h. LCMS indicated the reaction was complete. The mixture was poured into water (150 mL) and extracted with DCM (100 mL*2). The organic layers was washed with water (100 mL*2), saturated brine (100 mL*2), dried over Na₂SO₄, filtered, concentrated under reduced pressure to give a residue. The crude product was purified by chromatography on silica gel (petroleum ether/EtOAc=3:1) to give 3,5-dichloro-6-methylpyrazine-2-carboxamide (3 g, 14.56 mmol, 29.67% yield) as white solid. ¹H NMR (400 MHz, DMSO-d₆) δ=8.27-8.13 (m, 1H), 8.02 (br s, 1H), 2.67 (s, 3H); LC-MS (ES+, m/z): 206.2 [(M+H)⁺]. Rt=0.627 min.

### Step 2: tert-butyl (3-((3-carbamoyl-6-chloro-5-methylpyrazin-2-yl)amino)phenethyl)carbamate

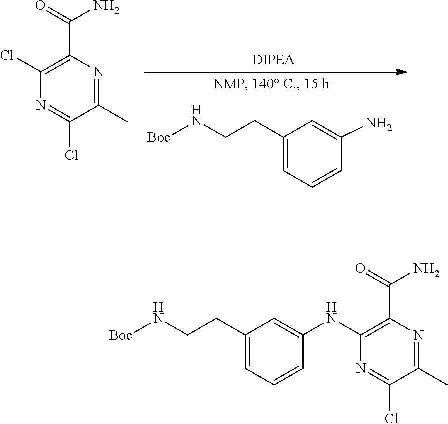

To a solution of 3,5-dichloro-6-methylpyrazine-2-carboxamide (5.3 g, 25.72 mmol, 1 eq), tert-butyl (3-aminophenethyl)carbamate (6.08 g, 25.72 mmol, 1 eq) in NMP (50 mL) at 16° C., DIPEA (66.49 g, 514.49 mmol, 89.61 mL, 20 eq) was added. The mixture was stirred at 140° C. for 10 hrs. LCMS indicated the reaction was complete. The mixture was poured into water (150 mL) and extracted with EtOAc (100 mL*2). The organic layers was washed with water (100 mL*2), saturated brine (100 mL*2), dried over Na₂SO₄, filtered, concentrated under reduced pressure to give a residue. The crude product was purified by chromatography on silica gel (petroleum ether/EtOAc=1:1) TLC (plate1) to give tert-butyl (3-((3-carbamoyl-6-chloro-5-methylpyrazin-2-yl)amino)phenethyl) carbamate (6 g, 14.78 mmol, 57.47% yield) as yellow solid. LC-MS (ES+, m/z): 406.2 [(M+H)⁺]. RT=0.880 min.

### Intermediate 14

### (S)-5-cyclopropyl-6-methyl-3-((3-(2-(2-(methylamino)propanamido)ethyl)phenyl)amino)pyrazine-2-carboxamide

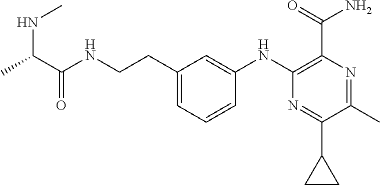 [SMILES: CN[C@@H](C)C(=O)NCCc1cccc(Nc2nc(C3CC3)c(C)nc2C(N)=O)c1]

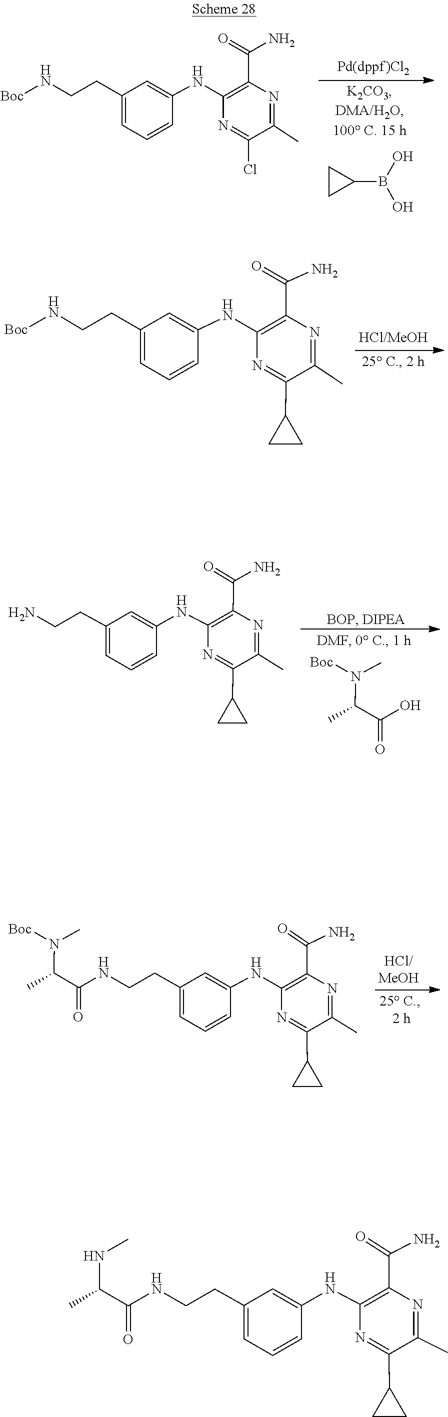

### Step 1: tert-butyl (3-((3-carbamoyl-6-cyclopropyl-5-methylpyrazin-2-yl)amino)phenethyl)carbamate

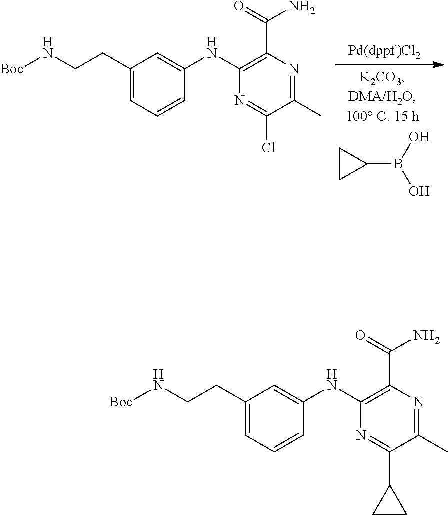

To a solution of tert-butyl (3-((3-carbamoyl-6-chloro-5-methylpyrazin-2-yl)amino)phenethyl)carbamate (3.47 g, 8.55 mmol, 1 eq), cyclopropylboronic acid (7.34 g, 85.49 mmol, 10 eq), K₂CO₃ (3.54 g, 25.65 mmol, 3 eq) in DMA (24 mL) and H₂O (12 mL) at 16° C., Pd(dppf)Cl₂ (625.57 mg, 854.94 umol, 0.1 eq) was added. The mixture was stirred at 100° C. for 15 hrs under N₂. LCMS indicated the reaction was complete. The residue was dissolved in DCM (40 mL), scavenger (Pd) was added and then stirred at 25° C. for 1 h, and then filtered. The mixture was poured into water (120 mL) and extracted with EtOAc (80 mL*2). The organic layers was washed with water (80 mL*2), saturated brine (80 mL*2), dried over Na₂SO₄, filtered, concentrated under reduced pressure to give a residue. The crude product was purified by chromatography on silica gel (petroleum ether/EtOAc=1:1) give tert-butyl (3-((3-carbamoyl-6-cyclopropyl-5-methylpyrazin-2-yl)amino)phenethyl) carbamate (1.9 g, 4.62 mmol, 54.01% yield) as yellow solid. LC-MS (ES+, m/z): 412.2 [(M+H)⁺]. RT=0.893 min.

### Step 2: 3-((3-(2-aminoethyl)phenyl)amino)-5-cyclopropyl-6-methylpyrazine-2-carboxamide

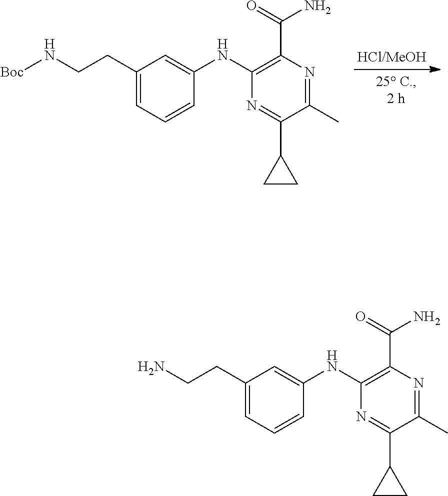 [SMILES: Cc1nc(C(N)=O)c(Nc2cccc(CCNC(=O)OC(C)(C)C)c2)nc1C1CC1 ; Cc1nc(C(N)=O)c(Nc2cccc(CCN)c2)nc1C1CC1]

The mixture tert-butyl (3-((3-carbamoyl-6-cyclopropyl-5-methylpyrazin-2-yl)amino) phenethyl) carbamate (1.9 g, 4.62 mmol, 1 eq) and HCl/MeOH (4 M, 50 mL, 43.32 eq) was stirred at 25° C. for 2 hrs. LCMS indicated the reaction was complete. The mixture was concentrated under reduced pressure to give 3-((3-(2-aminoethyl)phenyl)amino)-5-cyclopropyl-6-methylpyrazine-2-carboxamide (1.8 g, crude) as yellow solid. LC-MS (ES+, m/z): 312.2 [(M+H)⁺]; RT=0.643 min.

Note: HCl/MeOH (4 M): HCl was bubbled into a solution MeOH at 0° C. for 0.5 h.

### Step 3: tert-butyl (S)-(1-((3-((3-carbamoyl-6-cyclopropyl-5-methylpyrazin-2-yl)amino) phenethyl)amino)-1-oxopropan-2-yl)(methyl)carbamate

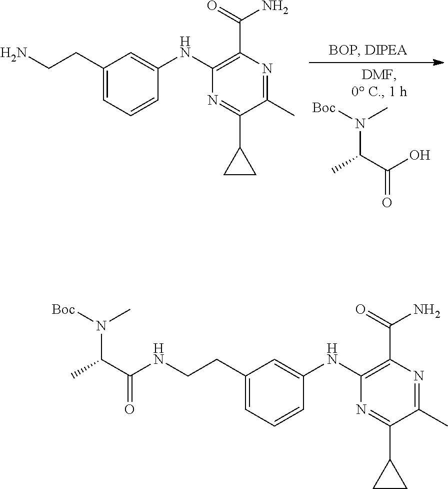

To a solution of, N-(tert-butoxycarbonyl)-N-methyl-L-alanine (1.16 g, 5.69 mmol, 1.1 eq) in DMF (15 mL), DIPEA (6.69 g, 51.75 mmol, 10 eq) 3-((3-(2-aminoethyl)phenyl)amino)-5-cyclopropyl-6-methylpyrazine-2-carboxamide (1.8 g, 5.17 mmol, 1 eq, HCl) was added at 0° C., and then BOP (3.43 g, 7.76 mmol, 1.5 eq) was added at 0° C. The mixture was stirred at 0° C. for 1 h. LCMS indicated the reaction was complete. The mixture was poured into water (80 mL) and extracted with EtOAc (50 mL*2). The organic layers was washed with water (50 mL*2), saturated brine (50 mL*2), dried over Na₂SO₄, filtered, concentrated under reduced pressure to give a residue. The crude product was purified by chromatography on silica gel (Petroleum ether:Ethyl acetate=2:1) to afford tert-butyl (S)-(1-((3-((3-carbamoyl-6-cyclopropyl-5-methylpyrazin-2-yl)amino)phenethyl)amino)-1-oxopropan-2-yl)(methyl)carbamate (2.42 g, 4.87 mmol, 94.17% yield) as yellow solid. LC-MS (ES+, m/z): 497.3 [(M+H)⁺]; RT=0.860 min.

### Step 4: (S)-5-cyclopropyl-6-methyl-3-((3-(2-(2-(methylamino)propanamido)ethyl)phenyl)amino)pyrazine-2-carboxamide

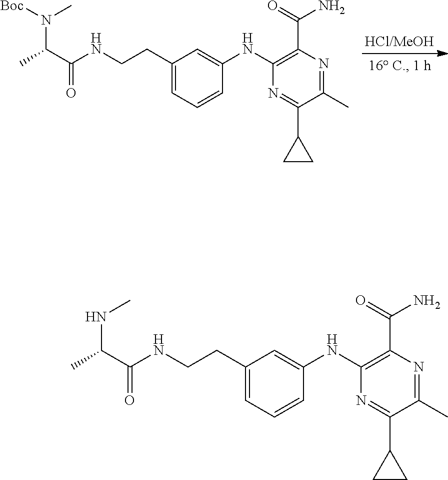

The mixture tert-butyl (S)-(1-((3-((3-carbamoyl-6-cyclopropyl-5-methylpyrazin-2-yl)amino)phenethyl)amino)-1-oxopropan-2-yl)(methyl)carbamate (1.5 g, 3.02 mmol, 1 eq) and HCl/MeOH (4 M, 50 mL, 66.21 eq) was stirred at 16° C. for 1 h. LCMS indicated the reaction was complete. The mixture was concentrated under reduced pressure to give (S)-5-cyclopropyl-6-methyl-3-((3-(2-(2-(methylamino)propanamido)ethyl)phenyl)amino)pyrazine-2-carboxamide (1.23 g, crude) as yellow solid. LC-MS (ES+, m/z): 397.3 [(M+H)⁺]; RT=0.680 min.

### Intermediate 15

### (S)-5-(isopropyl(methyl)amino)-6-methyl-3-((3-(2-(2-(methylamino)propanamido)ethyl)phenyl)amino)pyrazine-2-carboxamide

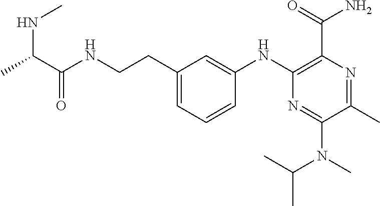 [SMILES: CN[C@@H](C)C(=O)NCCc1cccc(Nc2nc(N(C)C(C)C)c(C)nc2C(N)=O)c1]

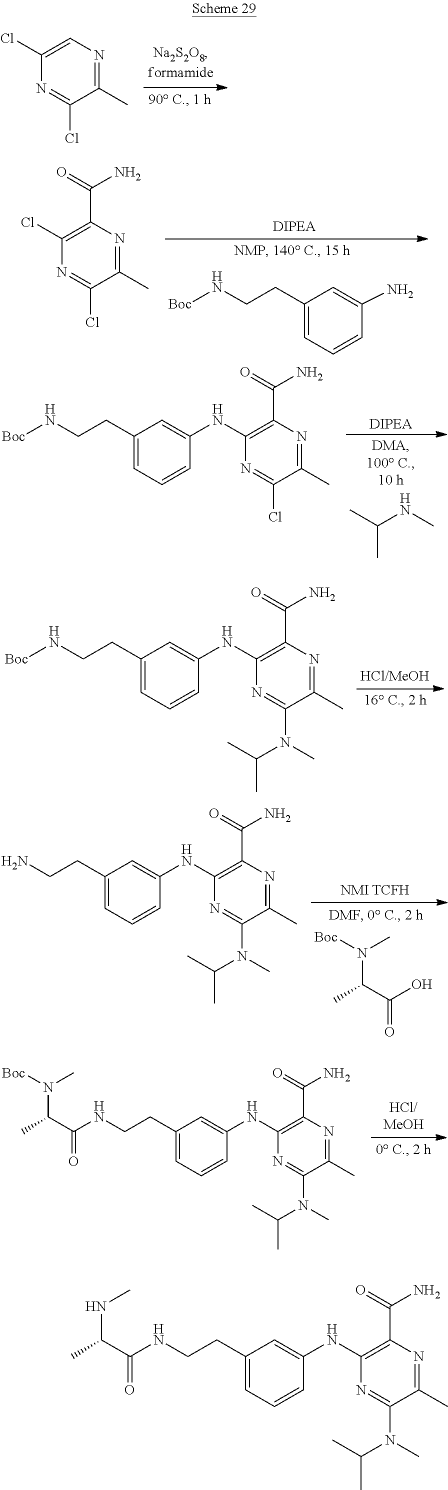

### Step 1: 3,5-dichloro-6-methylpyrazine-2-carboxamide

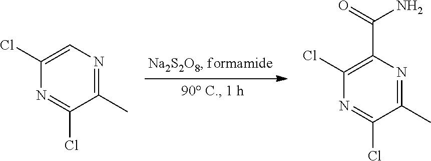

To a solution of 3,5-dichloro-2-methylpyrazine (8 g, 49.08 mmol, 1 eq) in formamide (53.05 g, 1.18 mol, 46.95 mL, 24 eq) at 25° C., Na₂S₂O₈ (18.70 g, 78.53 mmol, 17.00 mL, 1.6 eq) was added in batches at 90° C. The mixture was stirred at 90° C. for 1 h. LCMS indicated the reaction was complete. The mixture was poured into water (150 mL) and extracted with DCM (100 mL*2). The organic layers was washed with water (100 mL*2), saturated brine (100 mL*2), dried over Na₂SO₄, filtered, concentrated under reduced pressure to give a residue. The crude product was purified by chromatography on silica gel (petroleum ether/EtOAc=3:1) to give 3,5-dichloro-6-methylpyrazine-2-carboxamide (3 g, 14.56 mmol, 29.67% yield) as white solid. ¹H NMR (400 MHz, DMSO-d₆) δ=8.27-8.13 (m, 1H), 8.02 (br s, 1H), 2.67 (s, 3H); LC-MS (ES+, m/z): 206.2 [(M+H)+]. Rt=0.627 min. Step 2: tert-butyl (3-((3-carbamoyl-6-chloro-5-methylpyrazin-2-yl)amino)phenethyl)carbamate

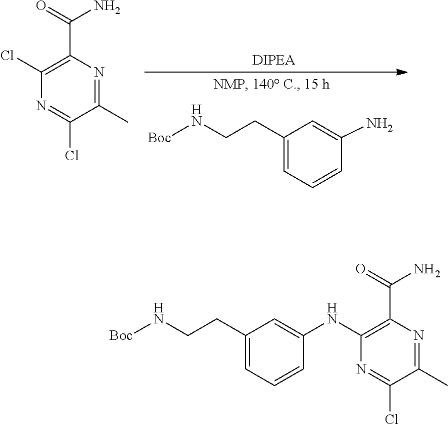

To a solution of 3,5-dichloro-6-methylpyrazine-2-carboxamide (1.7 g, 8.25 mmol, 1 eq) tert-butyl (3-aminophenethyl)carbamate (1.95 g, 8.25 mmol, 1 eq) in NMP (30 mL) at 25° C., DIPEA (31.99 g, 247.54 mmol, 43.12 mL, 30 eq) was added. The mixture was stirred at 140° C. for 15 hrs. LCMS indicated the reaction was complete. The mixture was poured into water (100 mL) and extracted with EtOAc (60 mL*2). The organic layers was washed with water (60 mL*2), saturated brine (60 mL*2), dried over Na₂SO₄, filtered, concentrated under reduced pressure to give a residue. The crude product was purified by chromatography on silica gel (petroleum ether/EtOAc=1:1) TLC to give tert-butyl (3-((3-carbamoyl-6-chloro-5-methylpyrazin-2-yl)amino)phenethyl)carbamate (2.52 g, 6.21 mmol, 75.25% yield) as yellow solid. LC-MS (ES+, m/z): 406.3 [(M+H)⁺]. Rt=0.893 min.

### Step 3: tert-butyl (3-((3-carbamoyl-6-(isopropyl(methyl)amino)-5-methylpyrazin-2-yl)amino)phenethyl)carbamate

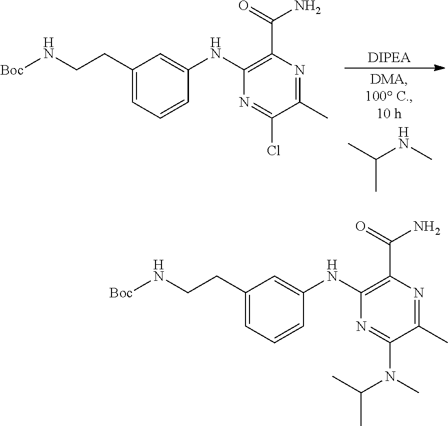

A solution of tert-butyl (3-((3-carbamoyl-6-chloro-5-methylpyrazin-2-yl)amino) phenethyl) carbamate (2.5 g, 6.16 mmol, 1 eq) in DMA (30 mL) saturated with N-methylpropan-2-amine (2.25 g, 30.80 mmol, 3.21 mL, 5 eq) DIEA (7.96 g, 61.59 mmol, 10.73 mL, 10 eq) was stirred at 100° C. for 10 hr in a 100 mL of autoclave. LCMS showed the reaction was completed. The reaction mixture was quenched by addition water (400 mL), and then extracted with EtOAc (300 mL*3). The combined organic layers were washed with saturated brine (350 mL*2), dried over Na₂SO₄, filtered and concentrated under reduced pressure to give a residue. The residue was purified by column chromatography (SiO₂, Petroleum ether/Ethyl acetate=10/1 to 1/1) to give tert-butyl (3-((3-carbamoyl-6-(isopropyl(methyl)amino)-5-methylpyrazin-2-yl)amino) phenethyl) carbamate as yellow solid (9 g, 18.30 mmol, 74.29% yield, 90% purity). ¹H NMR (400 MHz, DMSO-d₆) δ=11.10 (s, 1H), 7.77 (br d, J=2.1 Hz, 1H), 7.61-7.53 (m, 1H), 7.45-7.35 (m, 2H), 7.23-7.15 (m, 1H), 6.95-6.82 (m, 1H), 6.81-6.75 (m, 1H), 4.40-4.30 (m, 1H), 3.16-3.09 (m, 2H), 2.94-2.85 (m, 3H), 2.69-2.63 (m, 2H), 2.44 (s, 3H), 1.39-1.32 (m, 9H), 1.21 (d, J=6.6 Hz, 6H) LC-MS (ES⁺, m/z): 443.3 [(M+H)⁺]; Rt=0.947 min;

### Step 4: 3-((3-(2-aminoethyl)phenyl)amino)-5-(isopropyl(methyl)amino)-6-methylpyrazine-2-carboxamide

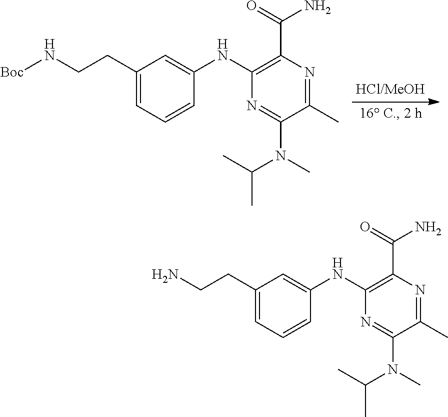 [SMILES: Cc1nc(C(N)=O)c(Nc2cccc(CCNC(=O)OC(C)(C)C)c2)nc1N(C)C(C)C ; Cc1nc(C(N)=O)c(Nc2cccc(CCN)c2)nc1N(C)C(C)C]

A mixture of tert-butyl (3-((3-carbamoyl-6-(isopropyl(methyl)amino)-5-methylpyrazin-2-yl)amino)phenethyl) carbamate (9 g, 20.34 mmol, 1 eq) was added HCl/MeOH (4 M, 100 mL) was stirred at 16° C. for 2 hr. LCMS showed the reaction was completed. The reaction mixture was concentrated under reduced pressure to give a residue. The crude product was triturated with EtOAc at 25° C. for 10 min and then filtered to give 3-((3-(2-aminoethyl)phenyl)amino)-5-(isopropyl(methyl)amino)-6-methylpyrazine-2-carboxamide (7.7 g, 18.29 mmol, 89.94% yield) as yellow solid. ¹H NMR (400 MHz, DMSO-d₆) δ=11.13 (s, 1H), 8.12-8.00 (m, 3H), 7.93-7.67 (m, 1H), 7.60-7.36 (m, 3H), 7.28-7.23 (m, 1H), 6.88-6.83 (m, 1H), 4.36-4.28 (m, 1H), 3.07-2.99 (m, 2H), 2.91-2.85 (m, 5H), 2.44 (s, 3H), 1.23-1.19 (m, 6H)(HCl salt). LC-MS (ES⁺, m/z): 343.2 [(M+H)+]; Rt=0.691 min.

Note: HCl/EtOAc (4 M): HCl was bubbled into a solution EtOAc at 0° C. for 0.5 h. Then, the solution was weighed to obtained the HCl/EtOAc (4 M).

### Step 5: tert-butyl (S)-(1-((3-((3-carbamoyl-6-(isopropyl(methyl)amino)-5-methylpyrazin-2-yl)amino)phenethyl)amino)-1-oxopropan-2-yl)(methyl)carbamate

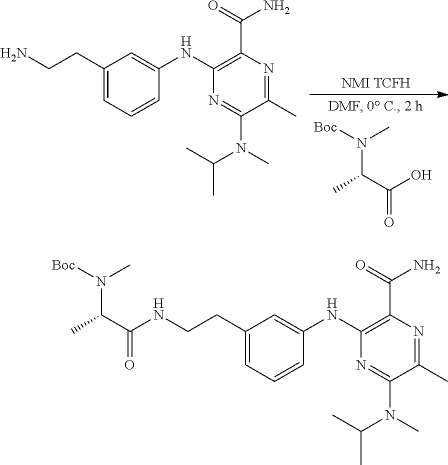

To a solution of N-(tert-butoxycarbonyl)-N-methyl-L-alanine (2.49 g, 12.27 mmol, 1.2 eq), 1-methylimidazole (8.39 g, 102.21 mmol, 8.15 mL, 10 eq) in DMF (35 mL) was added 3-((3-(2-aminoethyl)phenyl)amino)-5-(isopropyl(methyl)amino)-6-methylpyrazine-2-carboxamide (3.5 g, 10.22 mmol, 1 eq), and then added TCFH (3.44 g, 12.27 mmol, 1.2 eq) at 0° C. The mixture was stirred at 0° C. for 2 hr. LCMS showed the reaction was completed. The reaction mixture was quenched by addition water (120 mL) and then extracted with EtOAc (100 mL*3). The combined organic layers were washed with saturated brine (150 mL*2), dried over Na₂SO₄, filtered and concentrated under reduced pressure to give a residue. The residue was purified by column chromatography (SiO₂, Petroleum ether/Ethyl acetate=10/1 to 5/1) to give tert-butyl (S)-(1-((3-((3-carbamoyl-6-(isopropyl(methyl)amino)-5-methylpyrazin-2-yl)amino) phenethyl)amino)-1-oxopropan-2-yl)(methyl)carbamate (4.1 g, 7.38 mmol, 72.22% yield, 95% purity) as yellow solid. H NMR (400 MHz, DMSO-d₆) δ=11.11 (s, 1H), 7.84-7.73 (m, 2H), 7.56 (s, 1H), 7.44-7.37 (m, 2H), 7.24-7.14 (m, 1H), 6.83-6.75 (m, 1H), 4.41-4.31 (m, 1H), 4.04-4.01 (m, 1H), 3.31-3.22 (m, 2H), 2.93-2.86 (m, 3H), 2.72-2.65 (m, 5H), 2.46-2.41 (m, 3H), 1.42-1.32 (m, 9H), 1.17 (s, 9H). LC-MS (ES⁺, m/z): 528.4 [(M+H)+]; Rt=0.902 min.

### Step 6: (S)-5-(isopropyl(methyl)amino)-6-methyl-3-((3-(2-(2-(methylamino)propanamido)ethyl)phenyl)amino)pyrazine-2-carboxamide

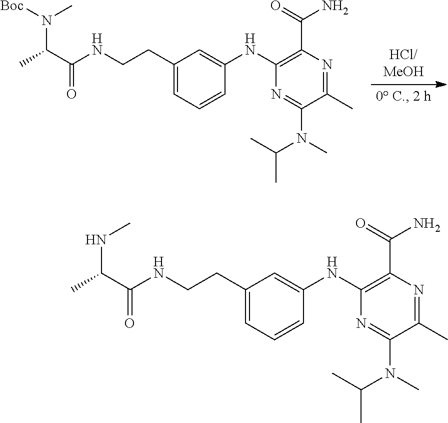

A mixture of tert-butyl (S)-(1-((3-((3-carbamoyl-6-(isopropyl(methyl)amino)-5-methylpyrazin-2-yl)amino)phenethyl)amino)-1-oxopropan-2-yl)(methyl)carbamate (4.1 g, 7.77 mmol, 1 eq) HCl/MeOH (4 M, 21.67 mL, 11.15 eq) was stirred at 0° C. for 2 hr. LCMS showed the reaction was completed. The reaction mixture was concentrated under reduced pressure to give a residue. The crude product was concentrated to give (S)-5-(isopropyl(methyl)amino)-6-methyl-3-((3-(2-(2-(methylamino)propanamido)ethyl)phenyl)amino)pyrazine-2-carboxamide (4.1 g, crude, HCl). ¹H NMR (400 MHz, DMSO-d₆) δ=11.14-11.05 (m, 1H), 9.49-9.38 (m, 1H), 8.90-8.78 (m, 1H), 8.76-8.70 (m, 1H), 7.52-7.47 (m, 2H), 7.23-7.18 (m, 1H), 6.82 (br d, J=7.5 Hz, 1H), 6.71-6.64 (m, 2H), 4.30 (s, 1H), 3.73-3.65 (m, 1H), 3.45-3.33 (m, 2H), 3.16-3.16 (m, 3H), 2.91-2.88 (m, 3H), 2.77-2.71 (m, 2H), 2.44 (s, 3H), 1.33-1.29 (m, 3H), 1.22-1.18 (m, 6H). LC-MS (ES⁺, m/z): 428.3 [(M+H)+]; Rt=0.693 min; 95.9% purity.

Note: HCl/EtOAc (4 M): HCl was bubbled into a solution EtOAc at 0° C. for 0.5 h. Then, the solution was weighed to obtained the HCl/EtOAc (4 M)

### Example 45

### (Compound 137)

### (S,E)-5-(dimethylamino)-3-((3-(2-(2-(4-(dimethylamino)-N-methylbut-2-enamido)propanamido)ethyl)phenyl)amino)-6-ethylpyrazine-2-carboxamide

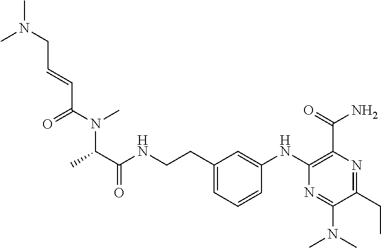 [SMILES: CCc1nc(C(N)=O)c(Nc2cccc(CCNC(=O)[C@H](C)N(C)C(=O)/C=C/CN(C)C)c2)nc1N(C)C]

### Step 1: tert-butyl (S)-(1-((3-((3-carbamoyl-6-(dimethylamino)-5-ethylpyrazin-2-yl)amino)phenethyl)amino)-1-oxopropan-2-yl)(methyl)carbamate

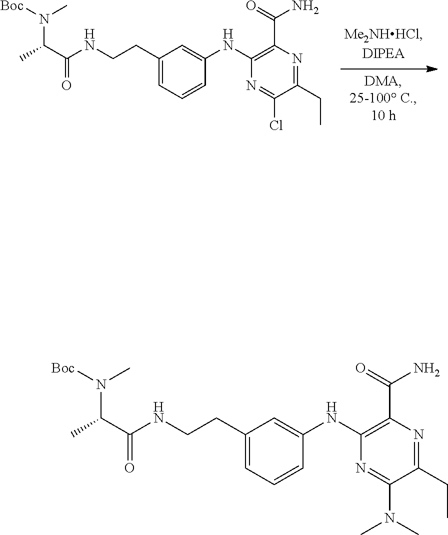

To a solution of tert-butyl (S)-(1-((3-((3-carbamoyl-6-chloro-5-ethylpyrazin-2-yl)amino)phenethyl)amino)-1-oxopropan-2-yl)(methyl)carbamate (600 mg, 1.19 mmol, 1 eq) Me₂NH (968.83 mg, 11.88 mmol, 10 eq, HCl) in DMA (6 mL) at 25° C., DIPEA (1.54 g, 11.88 mmol, 10 eq) was added. The mixture was stirred at 100° C. for 10 hrs. LCMS indicated the reaction was completed. The mixture was poured into water (20 mL) and extracted with EtOAc (15 mL*2). The organic layers was washed with water (15 mL*2), saturated brine (15 mL*2), dried over anhydrous Na₂SO₄, filtered, concentrated under reduced pressure to give a residue. The crude was purified by prep-HPLC column: C18-1 150*30 mm*5 um; mobile phase: [water(TFA)-ACN]; B %: 35%-80%, 8 min to afford tert-butyl N-[(1S)-2-[2-[3-[[3-carbamoyl-6-(dimethylamino)-5-ethyl-pyrazin-2-yl]amino]phenyl]ethylamino]-1-methyl-2-oxo-ethyl]-N-methyl-carbamate (251 mg, 449.58 umol, 37.84% yield) as brown solid. ¹H NMR (400 MHz, CDCl3-d) δ=10.67 (s, 1H), 7.65-7.39 (m, 4H), 7.17-7.12 (m, 2H), 6.74-6.71 (m, 1H), 4.17-3.94 (m, 1H), 3.51-3.39 (m, 2H), 3.03 (s, 6H), 2.72-2.68 (m, 4H), 2.60 (s, 3H), 1.34 (s, 9H), 1.24 (br d, J=7.1 Hz, 3H), 1.21-1.19 (m, 3H); LC-MS (ES+, m/z): 514.3 [(M+H)+]; Rt=0.872 min;

### Step 2: (S)-5-(dimethylamino)-6-ethyl-3-((3-(2-(2-(methylamino)propanamido)ethyl)phenyl)amino)pyrazine-2-carboxamide

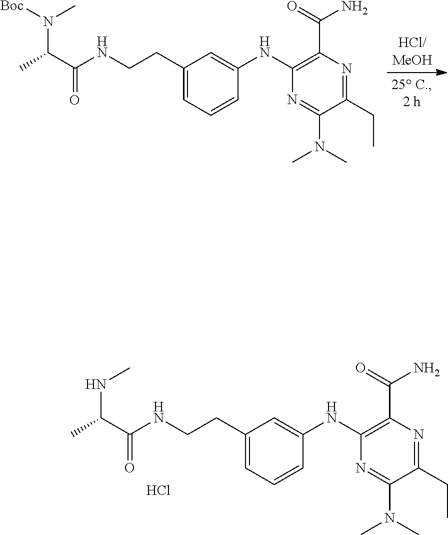

The mixture tert-butyl (S)-(1-((3-((3-carbamoyl-6-(dimethylamino)-5-ethylpyrazin-2-yl)amino)phenethyl)amino)-1-oxopropan-2-yl)(methyl)carbamate (251 mg, 488.68 umol, 1 eq) and HCl/MeOH (4 M, 50 mL, 409.27 eq) was stirred at 25° C. for 2 h. LCMS indicated the reaction was completed. The mixture was concentrated under reduced pressure to afford (S)-5-(dimethylamino)-6-ethyl-3-((3-(2-(2-(methylamino)propanamido)ethyl)phenyl)amino)pyrazine-2-carboxamide (230 mg, crude) as brown solid. LC-MS (ES⁺, m/z): 414.3 [(M+H)+]; Rt=0.684 min.

Note: HCl/MeOH (4 M): HCl gas was bubbled into a solution MeOH at 0° C. for 0.5 h. Then, the solution was weighed to obtained the HCl/MeOH (4 M)

### Step 3: (S,E)-5-(dimethylamino)-3-((3-(2-(2-(4-(dimethylamino)-N-methylbut-2-enamido)propanamido)ethyl)phenyl)amino)-6-ethylpyrazine-2-carboxamide

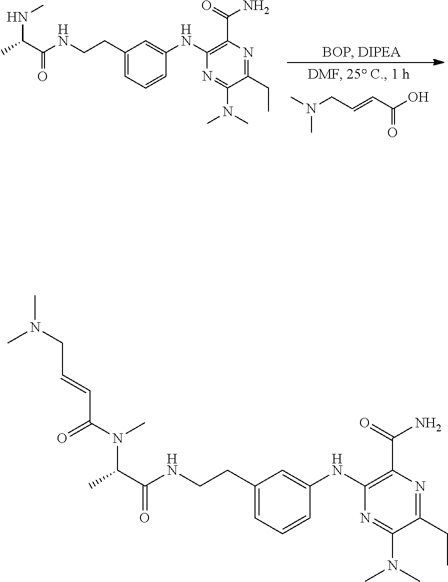

To a solution of (S)-5-(dimethylamino)-6-ethyl-3-((3-(2-(2-(methylamino)propanamido)ethyl)phenyl)amino)pyrazine-2-carboxamide (230 mg, 556.21 umol, 1 eq) (E)-4-(dimethylamino)but-2-enoic acid (138.18 mg, 834.31 umol, 1.5 eq), BOP (369.00 mg, 834.31 umol, 1.5 eq) in DMF (4 mL) at 25° C., DIPEA (718.86 mg, 5.56 mmol, 10 eq) was added. The mixture was stirred at 25° C. for 1 h. LCMS indicated the reaction was completed. The mixture was filtered to give a residue. The crude was purified by prep-HPLC column: C18-1 150*30 mm*5 um; mobile phase: [water(TFA)-ACN]; B %: 5%-50%, 8 min to afford (S,E)-5-(dimethylamino)-3-((3-(2-(2-(4-(dimethylamino)-N-methylbut-2-enamido)propanamido)ethyl)phenyl)amino)-6-ethylpyrazine-2-carboxamide (155.89 mg, 293.50 umol, 52.77% yield, 98.78% purity) as brown solid. ¹H NMR (400 MHz, DMSO-d₆, TFA) δ=11.15-11.07 (m, 1H), 9.76-9.63 (m, 1H), 8.10-7.85 (m, 1H), 7.76 (br d, J=2.5 Hz, 1H), 7.58-7.40 (m, 3H), 7.25-7.17 (m, 1H), 6.86-6.76 (m, 2H), 6.64-6.46 (m, 1H), 5.01-4.51 (m, 1H), 3.95-3.78 (m, 2H), 3.32-3.26 (m, 2H), 3.07 (s, 6H), 2.90 (s, 2H), 2.80-2.67 (m, 11H), 1.30-1.21 (m, 6H); LC-MS (ES⁺, m/z): 525.3 [(M+H)+]; Rt=2.099 min; 98.777% purity; HRMS (EI): m/z [M+H]+ found: 525.3279.

### Example 46

### (Compound 138)

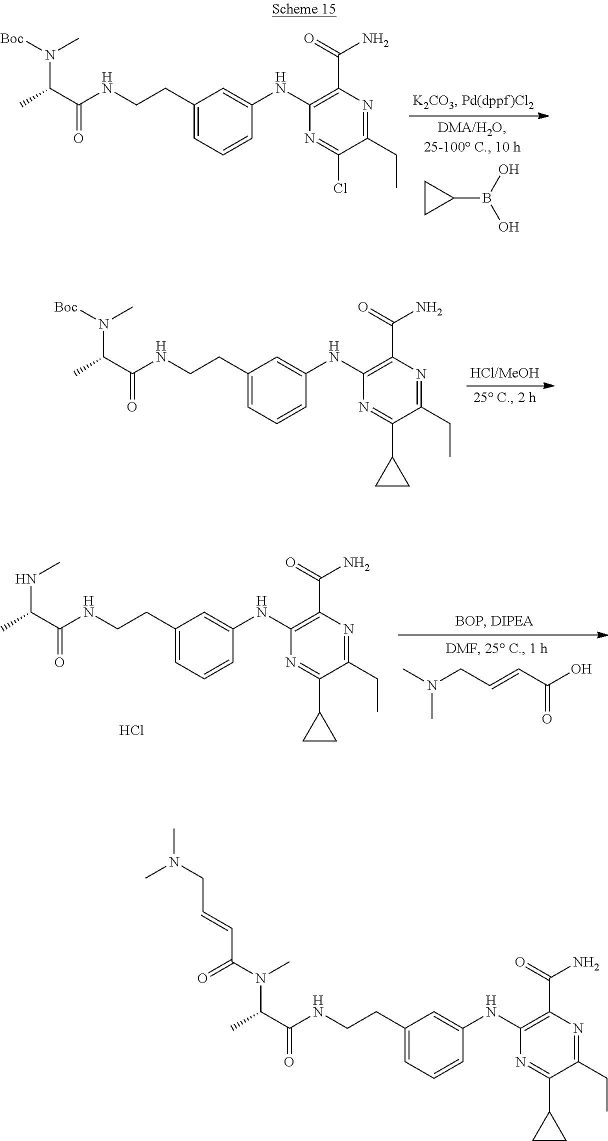

### (S,E)-5-cyclopropyl-3-((3-(2-(2-(4-(dimethylamino)-N-methylbut-2-enamido)propanamido)ethyl)phenyl)amino)-6-ethylpyrazine-2-carboxamide

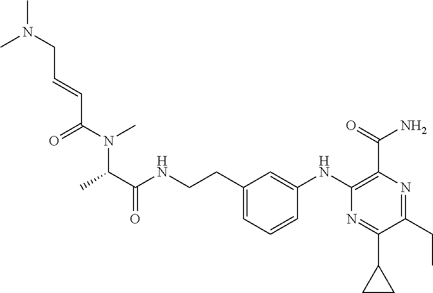 [SMILES: CCc1nc(C(N)=O)c(Nc2cccc(CCNC(=O)[C@H](C)N(C)C(=O)/C=C/CN(C)C)c2)nc1C1CC1]

### Step 1: tert-butyl (S)-(1-((3-((3-carbamoyl-6-cyclopropyl-5-ethylpyrazin-2-yl)amino)phenethyl)amino)-1-oxopropan-2-yl)(methyl)carbamate

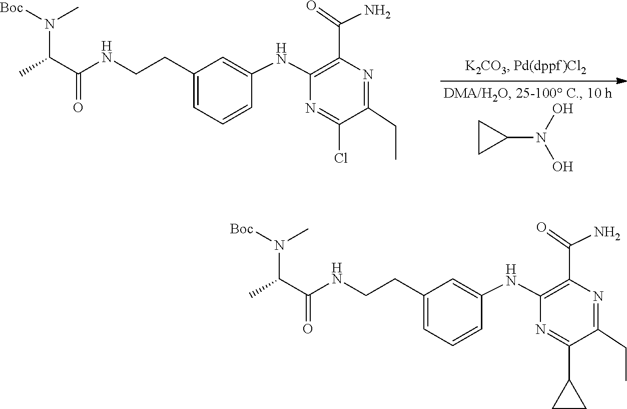

To a solution of tert-butyl (S)-(1-((3-((3-carbamoyl-6-chloro-5-ethylpyrazin-2-yl)amino)phenethyl)amino)-1-oxopropan-2-yl)(methyl)carbamate (600 mg, 1.19 mmol, 1 eq) cyclopropylboronic acid (1.02 g, 11.88 mmol, 10 eq) K₂CO₃ (492.61 mg, 3.56 mmol, 3 eq) in DMA (4 mL) and H₂O (2 mL) at 25° C., Pd(dppf)Cl₂ (86.93 mg, 118.81 umol, 0.1 eq) was added. The mixture was stirred at 100° C. for 10 hrs. LCMS indicated the reaction was completed. The mixture was poured into saturated EDTA (10 mL), EA (5 mL) and extracted with EtOAc (5 mL*2). The organic layers was washed with water (10 mL*2), saturated brine (10 mL*2), dried over anhydrous Na₂SO₄, filtered, concentrated under reduced pressure to give a residue. The crude was purified by prep-HPLC column: C18-1 150*30 mm*5 um; mobile phase: [water(TFA)-ACN]; B %: 35%-80%, 8 min to afford tert-butyl(S)-(1-((3-((3-carbamoyl-6-cyclopropyl-5-ethylpyrazin-2-yl)amino)phenethyl)amino)-1-oxopropan-2-yl)(methyl)carbamate (338 mg, 661.93 umol, 55.71% yield) as yellow solid. ¹H NMR (400 MHz, CDCl3-d) δ=10.61-10.54 (m, 1H), 7.86-7.75 (m, 1H), 7.48-7.40 (m, 2H), 7.19-7.13 (m, 1H), 6.76-6.73 (m, 1H), 6.18-5.88 (m, 11H), 5.58-5.41 (m, 11H), 4.10-4.00 (m, 11H), 3.58-3.41 (m, 2H), 2.84 (q, J=7.5 Hz, 2H), 2.77-2.69 (m, 2H), 2.65-2.57 (m, 3H), 2.12-2.06 (m, 1H), 1.36-1.32 (m, 9H), 1.25-1.17 (m, 6H), 1.17-1.11 (m, 2H), 1.06-1.00 (m, 2H); LC-MS (ES⁺, m/z): 511.3 [(M+H)⁺]; Rt=0.924 min.

### Step 2: (S)-5-cyclopropyl-6-ethyl-3-((3-(2-(2-(methylamino)propanamido)ethyl)phenyl)amino)pyrazine-2-carboxamide

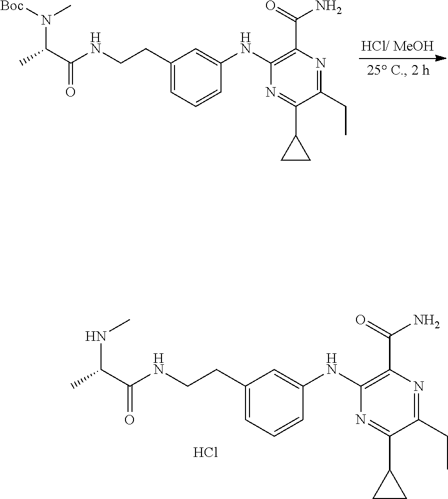

The mixture tert-butyl (S)-(1-((3-((3-carbamoyl-6-cyclopropyl-5-ethylpyrazin-2-yl)amino)phenethyl)amino)-1-oxopropan-2-yl)(methyl)carbamate (338 mg, 661.93 umol, 1 eq) and HCl/MeOH (4 M, 50 mL, 302.15 eq) was stirred at 25° C. for 2 hrs. LCMS indicated the reaction was completed. The mixture was concentrated under reduced pressure to afford (S)-5-cyclopropyl-6-ethyl-3-((3-(2-(2-(methylamino)propanamido)ethyl)phenyl)amino)pyrazine-2-carboxamide (300 mg, crude) as yellow solid. LC-MS (ES⁺, m/z): 411.3 [(M+H)⁺]; Rt=0.707 min.

Note: HCl/MeOH (4 M): HCl gas was bubbled into a solution MeOH at 0° C. for 0.5 h. Then, the solution was weighed to obtained the HCl/MeOH (4 M)

### Step 3: (S,E)-5-cyclopropyl-3-((3-(2-(2-(4-(dimethylamino)-N-methylbut-2-enamido)propanamido)ethyl)phenyl)amino)-6-ethylpyrazine-2-carboxamide

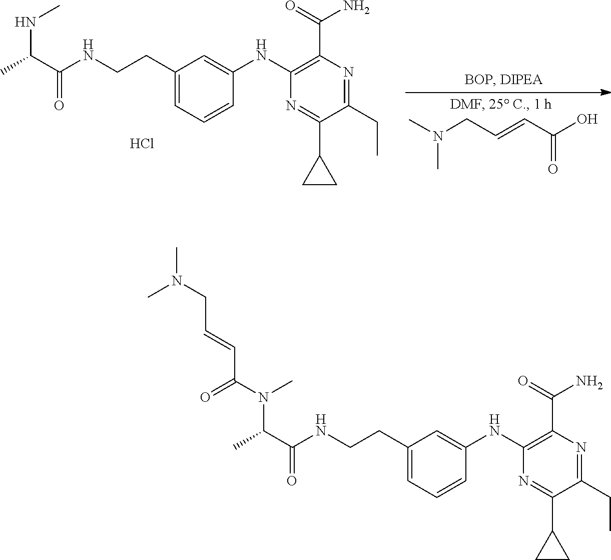

To a solution of (S)-5-cyclopropyl-6-ethyl-3-((3-(2-(2-(methylamino)propanamido)ethyl)phenyl)amino)pyrazine-2-carboxamide (300 mg, 730.80 umol, 1 eq) (E)-4-(dimethylamino)but-2-enoic acid (181.55 mg, 1.10 mmol, 1.5 eq) BOP (484.83 mg, 1.10 mmol, 1.5 eq) in DMF (4 mL) at 25° C., DIPEA (944.50 mg, 7.31 mmol, 10 eq) was added. The mixture was stirred at 25° C. for 1 h. LCMS indicated the reaction was completed. The mixture was filtered to give a residue. The crude was purified by prep-HPLC column: C18-1 150*30 mm*5 um; mobile phase: [water(TFA)-ACN]; B %: 5%-50%, 8 min to afford (S,E)-5-cyclopropyl-3-((3-(2-(2-(4-(dimethylamino)-N-methylbut-2-enamido)propanamido)ethyl)phenyl)amino)-6-ethylpyrazine-2-carboxamide (207.25 mg, 395.71 umol, 54.15% yield, 99.60% purity) as yellow solid. ¹H NMR (400 MHz, D2O) δ=7.39-7.30 (m, 1H), 7.12-7.05 (m, 2H), 6.78-6.72 (m, 1H), 6.66-6.56 (m, 1H), 6.51-6.40 (m, 1H), 4.86-4.75 (m, 1H), 3.74-3.62 (m, 2H), 3.49-3.29 (m, 2H), 2.79-2.60 (m, 13H), 2.14-2.06 (m, 1H), 1.28-1.13 (m, 6H), 1.03-0.97 (m, 2H), 0.90 (br s, 2H); ¹H NMR (400 MHz, DMSO-d₆) δ=11.10-11.00 (m, 1H), 9.74-9.59 (m, 1H), 8.21-8.12 (m, 1H), 8.07 (br s, 1H), 7.87-7.79 (m, 1H), 7.50-7.40 (m, 2H), 7.27-7.19 (m, 1H), 6.88-6.77 (m, 2H), 6.65-6.45 (m, 1H), 5.01-4.53 (m, 1H), 3.92-3.81 (m, 2H), 3.32-3.27 (m, 2H), 2.95-2.87 (m, 5H), 2.80-2.76 (m, 6H), 2.70 (br s, 2H), 2.35-2.31 (m, 1H), 1.31-1.20 (m, 6H), 1.14-1.05 (m, 4H); LC-MS (ES⁺, m/z): 522.3 [(M+H)⁺]; Rt=2.171 min; HRMS (EI): m/z [M+H]+ found: 522.3166.

### Example 49

### (Compound 141)

### (S,E)-5-(diethylamino)-3-((3-(3-(2-(4-(dimethylamino)-N-methylbut-2-enamido)propanamido)propoxy)phenyl)amino)-6-ethylpyrazine-2-carboxamide

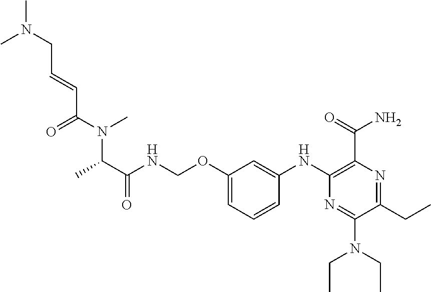 [SMILES: CCc1nc(C(N)=O)c(Nc2cccc(OCNC(=O)[C@H](C)N(C)C(=O)/C=C/CN(C)C)c2)nc1N(CC)CC]

### Step 1: tert-butyl (S)-(1-((3-(3-((3-carbamoyl-6-(diethylamino)-5-ethylpyrazin-2-yl)amino)phenoxy)propyl)amino)-1-oxopropan-2-yl)(methyl)carbamate

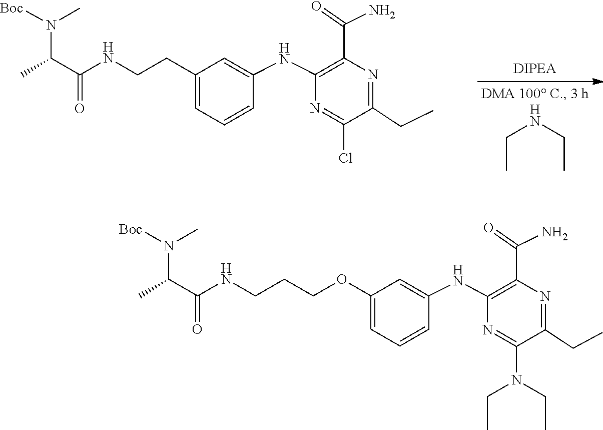

To a solution of tert-butyl (S)-(1-((3-(3-((3-carbamoyl-6-chloro-5-ethylpyrazin-2-yl)amino)phenoxy)propyl)amino)-1-oxopropan-2-yl)(methyl)carbamate (500 mg, 934.52 umol, 1 eq) in DMA (8 mL) was added DIPEA (1.21 g, 9.35 mmol, 1.63 mL, 10 eq) and N-ethylethanamine (683.47 mg, 9.35 mmol, 962.64 uL, 10 eq). The mixture was stirred at 100° C. for 3 hrs. LCMS showed the reaction was completed. The reaction mixture was quenched by addition water (50 mL) at 25° C., and then extracted with EtOAc (30 mL*3). The combined organic layers were washed with Saturated NaHCO₃ (20 mL*2) and saturated brine (20 mL*2), dried over Na₂SO₄, filtered and concentrated under reduced pressure to give a residue. The residue was purified by column chromatography (SiO₂, Petroleum ether/Ethyl acetate=10/1 to 1/1) to give tert-butyl (S)-(1-((3-(3-((3-carbamoyl-6-(diethylamino)-5-ethylpyrazin-2-yl)amino)phenoxy)propyl)amino)-1-oxopropan-2-yl)(methyl)carbamate (380 mg, 584.58 umol, 62.55% yield, 87.95% purity) as yellow solid. ¹H NMR (400 MHz, DMSO-d₆) δ=11.17-11.14 (m, 1H), 7.88-7.84 (m, 1H), 7.74 (br s, 1H), 7.46 (br d, J=2.1 Hz, 1H), 7.40-7.37 (m, 1H), 7.20-7.15 (m, 1H), 7.05-7.01 (m, 1H), 6.53 (dd, J=1.9, 7.9 Hz, 1H), 4.55-4.23 (m, 1H), 3.97 (br t, J=6.1 Hz, 2H), 3.49-3.43 (m, 4H), 2.95 (s, 3H), 2.79 (s, 4H), 2.73 (s, 4H), 1.96 (s, 2H), 1.36 (br s, 9H), 1.25 (br s, 3H), 1.21 (br s, 3H), 1.19-1.15 (m, 6H). LC-MS (ES⁺, m/z): 572.5 [(M+H)⁺]. Rt=0.893 min.

### Step 2: (S)-5-(diethylamino)-6-ethyl-3-((3-(3-(2-(methylamino)propanamido)propoxy)phenyl)amino)pyrazine-2-carboxamide

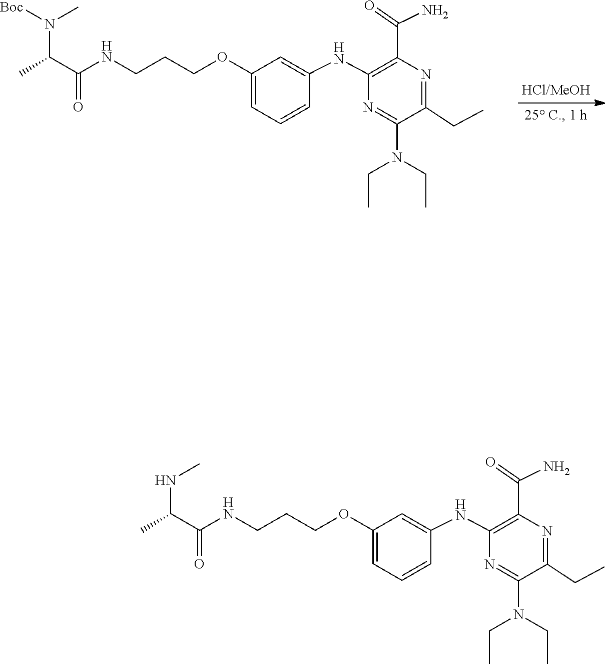

To (S)-tert-butyl (1-((3-(3-((3-carbamoyl-6-(diethylamino)-5-ethylpyrazin-2-yl)amino)phenoxy)propyl)amino)-1-oxopropan-2-yl)(methyl)carbamate (300 mg, 524.74 umol, 1 eq) was added HCl/MeOH (30 mL, 4 M). The mixture was stirred at 25° C. for 1 h. LCMS showed the reaction was completed. The precipitate was collected by filtration to give the crude product. The crude product was triturated with EtOAc (50 mL) at 25° C. for 10 min to give (S)-5-(diethylamino)-6-ethyl-3-((3-(3-(2-(methylamino)propanamido)propoxy)phenyl)amino)pyrazine-2-carboxamide (270 mg, 465.86 umol, 88.78% yield, 87.66% purity, HCl) as yellow solid. ¹H NMR (400 MHz, DMSO-d₆) δ=11.18-11.12 (m, 1H), 9.21-9.09 (m, 1H), 8.89-8.78 (m, 1H), 8.70-8.64 (m, 1H), 7.38-7.34 (m, 1H), 7.21-7.15 (m, 1H), 7.11-7.06 (m, 1H), 6.56-6.52 (m, 1H), 3.73 (br d, J=5.5 Hz, 1H), 3.46 (q, J=6.9 Hz, 4H), 3.33-3.28 (m, 2H), 3.17 (s, 2H), 2.95-2.78 (m, 1H), 2.73-2.66 (m, 3H), 1.94-1.87 (m, 2H), 1.38 (s, 2H), 1.36-1.13 (m, 12H). LC-MS (ES⁺, m/z): 472.5 [(M+H)+]. Rt=0.744 min

Note: HCl/MeOH (4 M): HCl gas was bubbled into a solution MeOH at 0° C. for 0.5 h. Then, the solution was weighed to obtained the HCl/MeOH (4 M)

### Step 3: (S,E)-5-(diethylamino)-3-((3-(3-(2-(4-(dimethylamino)-N-methylbut-2-enamido)propanamido)propoxy)phenyl)amino)-6-ethylpyrazine-2-carboxamide

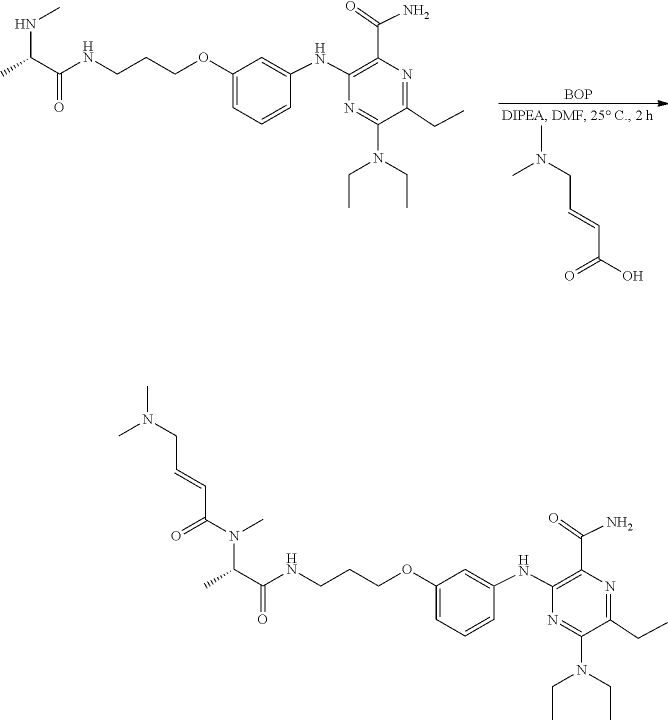

To a solution of (S)-5-(diethylamino)-6-ethyl-3-((3-(3-(2-(methylamino)propanamido)propoxy)phenyl)amino)pyrazine-2-carboxamide (270 mg, 572.53 umol, 1 eq) in DMF (3 mL) was added DIPEA (739.93 mg, 5.73 mmol, 997.21 uL, 10 eq) and BOP (379.83 mg, 858.79 umol, 1.5 eq) and (E)-4-(dimethylamino)but-2-enoic acid (110.92 mg, 858.79 umol, 1.5 eq). The mixture was stirred at 25° C. for 2 hrs. LCMS showed the reaction was completed. The reaction mixture was quenched by addition water (50 mL) at 25° C., and then extracted with EtOAc (30 mL*3). The combined organic layers were washed with saturated NaHCO₃ (20 mL*2) and saturated brine (20 mL*2), dried over Na₂SO₄, filtered and concentrated under reduced pressure to give a residue. The residue was purified by prep-HPLC (column: Phenomenex Luna C18 150*30 mm*5 um; mobile phase: [water (TFA)-ACN]; B %: 15%-45%, 8 min) to give (S,E)-5-(diethylamino)-3-((3-(3-(2-(4-(dimethylamino)-N-methylbut-2-enamido) propanamido)propoxy)phenyl)amino)-6-ethylpyrazine-2-carboxamide (73.45 mg, 125.59 umol, 21.94% yield, 99.64% purity) as yellow solid. ¹H NMR (400 MHz, DMSO-d₆) δ=11.20-11.11 (m, 1H), 9.96 (br s, 1H), 8.18-7.91 (m, 1H), 7.75 (br s, 1H), 7.47 (br s, 1H), 7.39-7.31 (m, 1H), 7.20-7.03 (m, 2H), 6.84 (d, J=15.1 Hz, 1H), 6.63-6.49 (m, 2H), 5.03-4.77 (m, 1H), 3.99-3.43 (m, 9H), 3.27-3.18 (m, 2H), 2.97-2.75 (m, 8H), 2.69 (d, J=7.4 Hz, 2H), 1.89-1.80 (m, 2H), 1.31-1.20 (m, 6H), 1.16 (t, J=7.0 Hz, 6H); LC-MS (ES⁺, m/z): 292.3 [(M/2+H)*]; Rt=2.285 min; HRMS (EI): m/z [M+H]⁺: 583.3702.

### Example 50

### (Compound 142)

### (S,E)-3-((3-(3-(2-(4-(dimethylamino)-N-methylbut-2-enamido)propanamido) propoxy)phenyl)amino)-6-ethyl-5-(pyrrolidin-1-yl)pyrazine-2-carboxamide

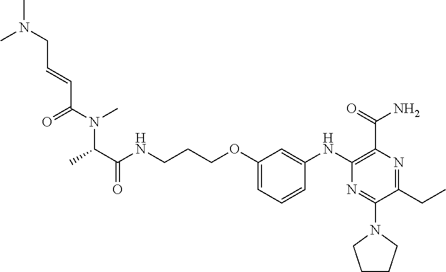 [SMILES: CCc1nc(C(N)=O)c(Nc2cccc(OCCCNC(=O)[C@H](C)N(C)C(=O)/C=C/CN(C)C)c2)nc1N1CCCC1]

### Step 1: tert-butyl (S)-(1-((3-(3-((3-carbamoyl-5-ethyl-6-(pyrrolidin-1-yl)pyrazin-2-yl)amino)phenoxy)propyl)amino)-1-oxopropan-2-yl)(methyl)carbamate

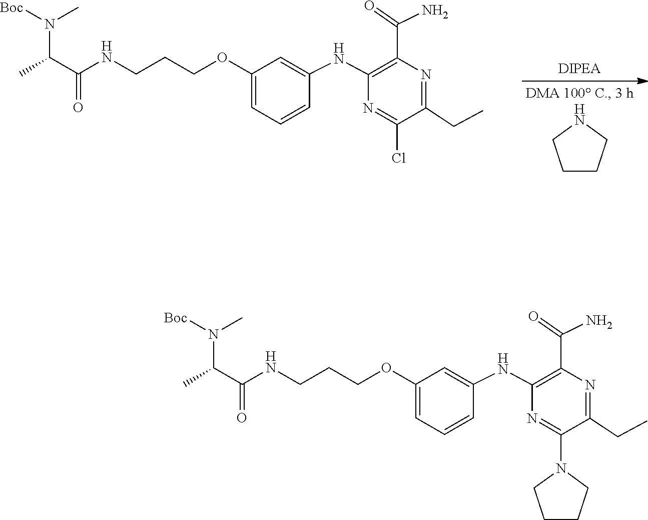

To a solution of tert-butyl (S)-(1-((3-(3-((3-carbamoyl-6-chloro-5-ethylpyrazin-2-yl)amino)phenoxy)propyl)amino)-1-oxopropan-2-yl)(methyl)carbamate (450 mg, 841.07 umol, 1 eq) in DMA (5 mL) was added DIPEA (1.09 g, 8.41 mmol, 1.46 mL, 10 eq) and pyrrolidine (598.17 mg, 8.41 mmol, 702.08 uL, 10 eq). The mixture was stirred at 100° C. for 3 hrs. LC-MS showed the reaction was completed. The reaction mixture was quenched by addition water (50 mL) at 25° C., and then extracted with EtOAc (30 mL*3). The combined organic layers were washed with Saturated NaHCO₃ (20 mL*2) and saturated brine (20 mL*2), dried over Na₂SO₄, filtered and concentrated under reduced pressure to give a residue. The residue was purified by column chromatography (SiO₂, Petroleum ether/Ethyl acetate=10/1 to 1/1) to give tert-butyl (S)-(1-((3-(3-((3-carbamoyl-5-ethyl-6-(pyrrolidin-1-yl)pyrazin-2-yl)amino)phenoxy)propyl)amino)-1-oxopropan-2-yl)(methyl)carbamate (360 mg, 584.02 umol, 69.44% yield, 92.42% purity) as white solid. LC-MS (ES⁺, m/z): 570.6 [(M+H)⁺]. Rt=0.877 min.

### Step 2: (S)-6-ethyl-3-((3-(3-(2-(methylamino)propanamido)propoxy)phenyl)amino)-5-(pyrrolidin-1-yl)pyrazine-2-carboxamide

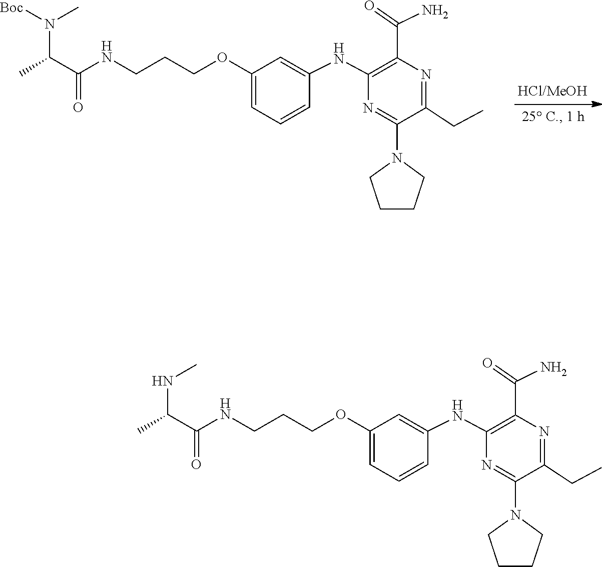

To tert-butyl (S)-(1-((3-(3-((3-carbamoyl-5-ethyl-6-(pyrrolidin-1-yl)pyrazin-2-yl)amino)phenoxy)propyl)amino)-1-oxopropan-2-yl)(methyl)carbamate (260 mg, 456.39 umol, 1 eq) was added HCl/MeOH (4 M, 50 mL). The mixture was stirred at 25° C. for 1 h. LCMS showed the reaction was completed. The precipitate was collected by filtration to give the crude product. The crude product was triturated with EtOAc (50 mL) at 25° C. for 10 min to give (S)-6-ethyl-3-((3-(3-(2-(methylamino)propanamido) propoxy)phenyl)amino)-5-(pyrrolidin-1-yl)pyrazine-2-carboxamide (210 mg, 430.75 umol, 94.38% yield, 96.32% purity) as yellow solid. LC-MS (ES⁺, m/z): 470.5 [(M+H)⁺]. Rt=0.727 min

Note: HCl/MeOH (4 M): HCl gas was bubbled into a solution MeOH at 0° C. for 0.5 h. Then, the solution was weighed to obtained the HCl/MeOH (4 M)

### Step 3: (S,E)-3-((3-(3-(2-(4-(dimethylamino)-N-methylbut-2-enamido) propanamido)propoxy)phenyl)amino)-6-ethyl-5-(pyrrolidin-1-yl)pyrazine-2-carboxamide

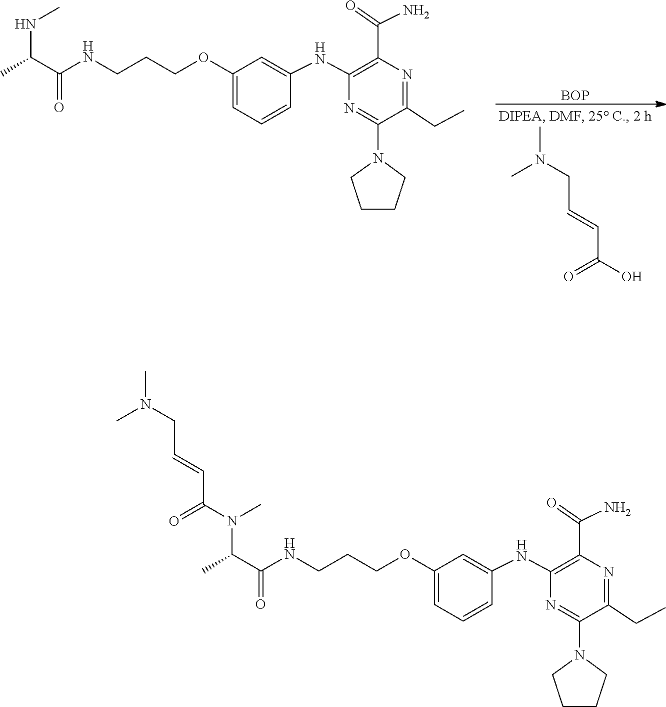

To a solution of (S)-6-ethyl-3-((3-(3-(2-(methylamino)propanamido)propoxy)phenyl)amino)-5-(pyrrolidin-1-yl)pyrazine-2-carboxamide (200 mg, 425.91 umol, 1 eq) in DMF (8 mL) was added DIPEA (550.46 mg, 4.26 mmol, 741.87 uL, 10 eq) and BOP (282.56 mg, 638.87 umol, 1.5 eq) and (E)-4-(dimethylamino)but-2-enoic acid (82.51 mg, 638.87 umol, 1.5 eq). The mixture was stirred at 25° C. for 2 hrs. LCMS showed the reaction was completed. The reaction mixture was quenched by addition water (50 mL) at 25° C., and then extracted with EtOAc (30 mL*3). The combined organic layers were washed with saturated NaHCO₃ (20 mL*2) and saturated brine (20 mL*2), dried over Na₂SO₄, filtered and concentrated under reduced pressure to give a residue. The residue was purified by prep-HPLC (column: C18-1 150*30 mm*5 um; mobile phase: [water (TFA)-ACN]; B %: 10%-55%, 8 min) to give (S,E)-3-((3-(3-(2-(4-(dimethylamino)-N-methylbut-2-enamido)propanamido) propoxy)phenyl)amino)-6-ethyl-5-(pyrrolidin-1-yl)pyrazine-2-carboxamide (61.65 mg, 88.74 umol, 20.83% yield, 100% purity, TFA) as light white solid. ¹H NMR (400 MHz, DMSO-d₆) δ=11.20 (s, 1H), 9.72 (br s, 1H), 8.15-7.91 (m, 1H), 7.64 (br s, 1H), 7.48-7.31 (m, 2H), 7.21-7.05 (m, 2H), 6.83 (br d, J=15.1 Hz, 1H), 6.61-6.46 (m, 2H), 4.99-4.57 (m, 1H), 3.99-3.94 (m, 2H), 3.91-3.81 (m, 2H), 3.71-3.65 (m, 4H), 3.25-3.19 (m, 2H), 2.96 (s, 2H), 2.89-2.83 (m, 2H), 2.80-2.73 (m, 7H), 1.95-1.82 (m, 6H), 1.32-1.17 (m, 6H) LC-MS (ES⁺, m/z): 291.3 [(M/2+H)*]. Rt=2.240 min. HRMS (EI): m/z [M+H]⁺: 581.3556.

### Example 50A

### (Compound 142A)

### (S,E)-3-((3-(2-(2-(4-(dimethylamino)-N-methylbut-2-enamido)propanamido)ethyl)phenyl)amino)-6-ethyl-5-methylpyrazine-2-carboxamide

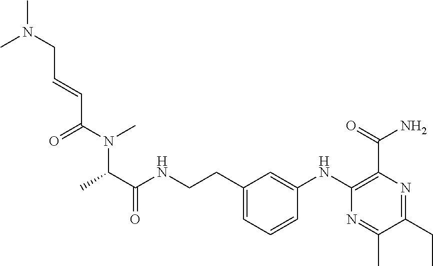 [SMILES: CCc1nc(C(N)=O)c(Nc2cccc(CCNC(=O)[C@H](C)N(C)C(=O)/C=C/CN(C)C)c2)nc1C]

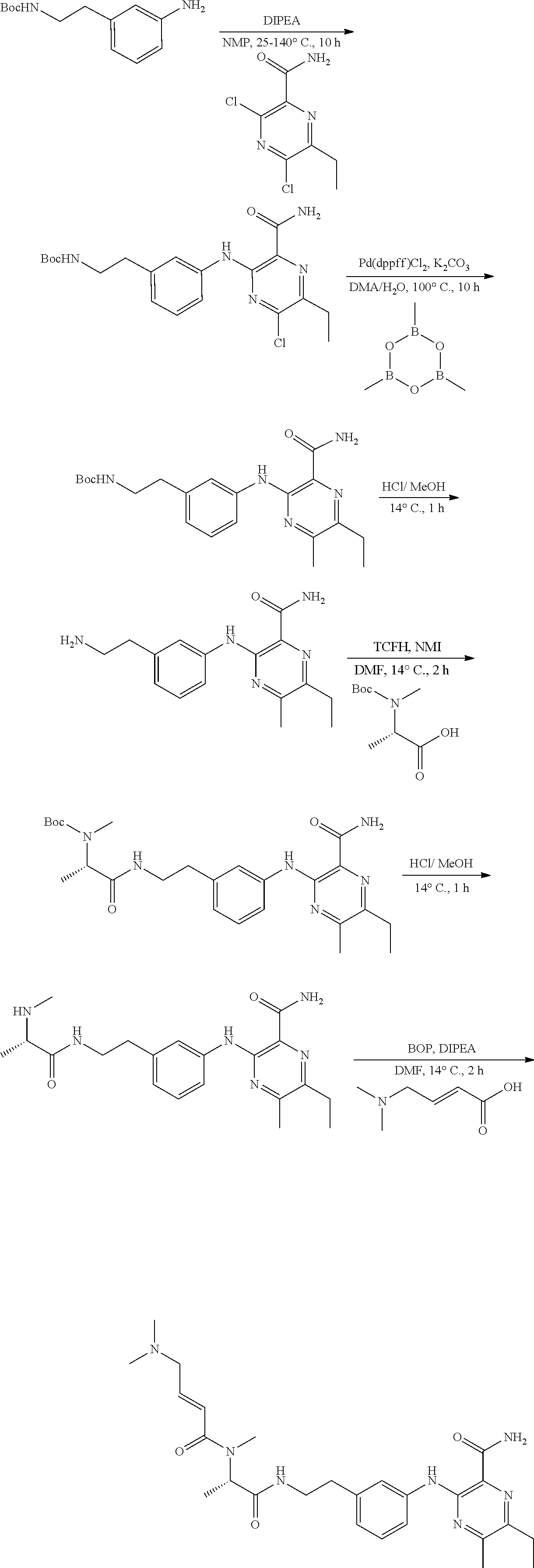

### Step 1: tert-butyl (3-((3-carbamoyl-6-chloro-5-ethylpyrazin-2-yl)amino)phenethyl)carbamate

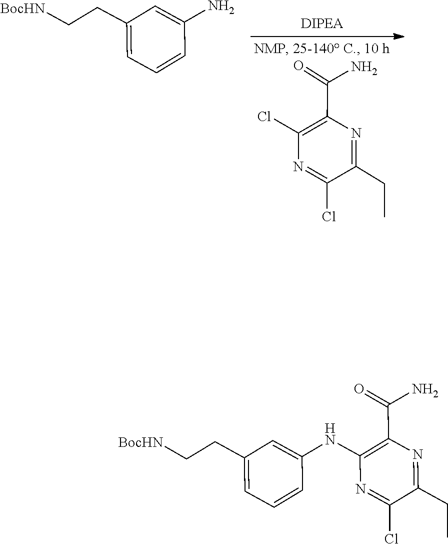 [SMILES: CC(C)(C)OC(=O)NCCc1cccc(N)c1 ; CCc1nc(C(N)=O)c(Cl)nc1Cl ; CCc1nc(C(N)=O)c(Nc2cccc(CCNC(=O)OC(C)(C)C)c2)nc1Cl]

A mixture of tert-butyl (3-aminophenethyl)carbamate (2 g, 8.46 mmol, 1 eq), 3,5-dichloro-6-ethylpyrazine-2-carboxamide (1.86 g, 8.46 mmol, 1 eq) in NMP (5 mL), DIPEA (21.88 g, 169.27 mmol, 29.48 mL, 20 eq) was added at 25° C. The mixture was stirred at 140° C. for 10 hours. LCMS showed the reaction was completed. The reaction was poured into H₂O (50 mL). The aqueous phase was extracted with ethyl acetate (20 mL*3). The combined organic phase was washed with saturated brine (50 mL*1), dried with anhydrous Na₂SO₄. Filtered and concentrated in vacuum. The residue was purified by silica gel chromatography (column height: 250 mm, diameter: 100 mm, 100-200 mesh silica gel, Petroleum ether/Ethyl acetate=1/1, 10/1) to afford tert-butyl (3-((3-carbamoyl-6-chloro-5-ethylpyrazin-2-yl)amino)phenethyl)carbamate (3.5 g, 6.67 mmol, 78.79% yield) as a yellow solid. 1H NMR (400 MHz, DMSO-d6) δ=11.19 (s, 1H), 8.26 (s, 1H), 8.04 (br s, 1H), 7.59 (br d, J=7.9 Hz, 1H), 7.32-7.23 (m, 2H), 6.93-6.83 (m, 2H), 3.16 (q, J=6.5 Hz, 2H), 2.82 (q, J=7.5 Hz, 2H), 2.69 (t, J=7.3 Hz, 2H), 1.36 (s, 9H), 1.28-1.24 (m, 3H) LC-MS (ES+, m/z): 420.3 [(M+H)⁺]; Rt=0.941 min.

### Step 2: tert-butyl (3-((3-carbamoyl-5-ethyl-6-methylpyrazin-2-yl)amino)phenethyl)carbamate

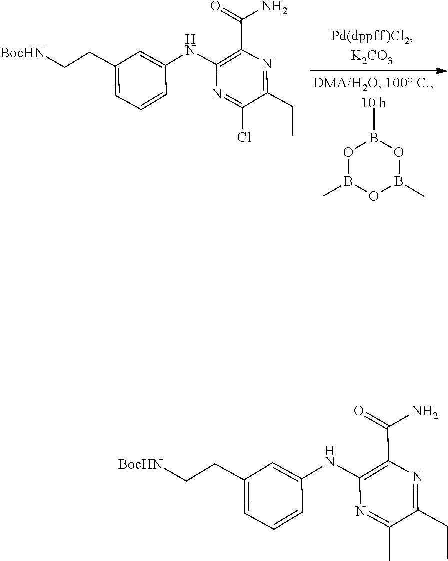

To a solution of tert-butyl (3-((3-carbamoyl-6-chloro-5-ethylpyrazin-2-yl)amino)phenethyl)carbamate (1 g, 2.38 mmol, 1 eq), 2,4,6-trimethyl-1,3,5,2,4,6-trioxatriborinane (1.49 g, 11.91 mmol, 5 eq) in DMA (12 mL) and H₂O (3 mL), K₂CO₃ (987.41 mg, 7.14 mmol, 3 eq) and Pd(dppf)Cl₂ (174.26 mg, 238.15 umol, 0.1 eq) was added, the mixture was stirred at 100° C. for 10 hours. LCMS showed the reaction was completed. The residue was poured into saturated EDTA (100 mL) and EtOAc (30 mL) stirred for 60 min. The mixture was concentrated. The residue was diluted with NaHCO₃ (50 mL) and extracted with EtOAc (50 mL*4). The organic layers were combined, washed with water (50 ml*2), saturated brine (50 ml), dried with anhydrous Na₂SO₄, Filtered and concentrated to give crude product. The residue was purified by silica gel chromatography (column height: 250 mm, diameter: 100 mm, 100-200 mesh silica gel, Petroleum ether/Ethyl acetate=1/1, 10/1). To afford tert-butyl (3-((3-carbamoyl-5-ethyl-6-methylpyrazin-2-yl)amino)phenethyl)carbamate (650 mg, 1.63 mmol, 68.32% yield) as a yellow oil. 1H NMR (400 MHz, DMSO-d6) δ=11.05 (s, 1H), 8.14 (br d, J=1.6 Hz, 1H), 7.86 (br d, J=1.6 Hz, 1H), 7.65 (br d, J=8.1 Hz, 1H), 7.44-7.42 (m, 1H), 7.22 (t, J=7.8 Hz, 1H), 6.88-6.80 (m, 2H), 3.18-3.13 (m, 2H), 2.78-2.72 (m, 2H), 2.71-2.67 (m, 2H), 1.36 (s, 9H), 1.26-1.22 (m, 3H), 1.20-1.10 (m, 3H). LC-MS (ES+, m/z): 400.3 [(M+H)⁺]; Rt=0.912 min.

### Step 3: 3-((3-(2-aminoethyl)phenyl)amino)-6-ethyl-5-methylpyrazine-2-carboxamide

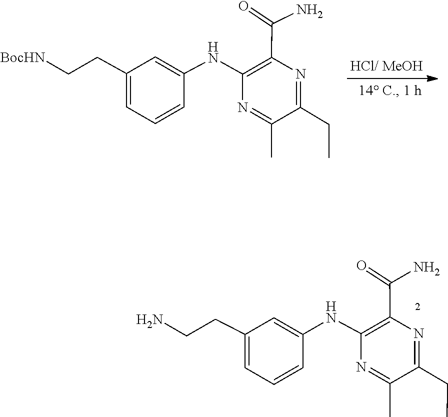 [SMILES: CCc1nc(C(N)=O)c(Nc2cccc(CCNC(=O)OC(C)(C)C)c2)nc1C ; CCc1nc(C(N)=O)c(Nc2cccc(CCN)c2)nc1C]

To a mixture of tert-butyl (3-((3-carbamoyl-5-ethyl-6-methylpyrazin-2-yl)amino)phenethyl)carbamate (650 mg, 1.63 mmol, 1 eq) in HCl/MeOH (4 M, 20 mL, 49.17 eq) at 14° C. for 1 hour. LCMS showed the reaction was completed. Filtered and concentrated in vacuum to afford 3-((3-(2-aminoethyl)phenyl)amino)-6-ethyl-5-methylpyrazine-2-carboxamide (450 mg, 1.50 mmol, 92.38% yield) as a yellow solid. 1H NMR (400 MHz, DMSO-d6) δ=11.08 (s, 1H), 8.15 (br s, 1H), 7.87 (br s, 1H), 7.71 (dd, J=1.3, 8.1 Hz, 1H), 7.49 (s, 1H), 7.31-7.19 (m, 2H), 7.18-7.12 (m, 1H), 6.88 (d, J=7.6 Hz, 1H), 3.07-3.01 (m, 2H), 2.91-2.86 (m, 2H), 2.74 (q, J=7.5 Hz, 2H), 1.33-1.11 (m, 5H), 1.10-0.76 (m, 1H) LC-MS (ES+, m/z): 300.3 [(M+H)+]; Rt=0.647 min. HCl/MeOH (4 M): HCl was bubbled into a solution MeOH at 0° C. for 0.5 h.

### Step 4 tert-butyl (S)-(1-((3-((3-carbamoyl-5-ethyl-6-methylpyrazin-2-yl)amino)phenethyl)amino)-1-oxopropan-2-yl)(methyl)carbamate

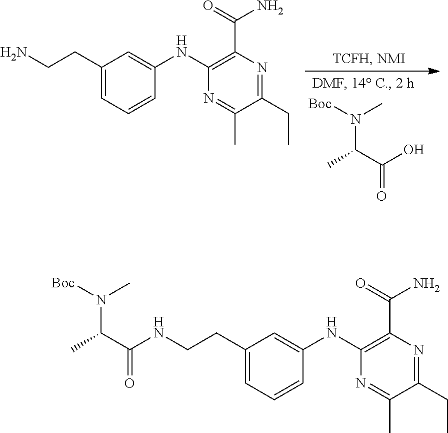

To a solution of 3-((3-(2-aminoethyl)phenyl)amino)-6-ethyl-5-methylpyrazine-2-carboxamide (450 mg, 1.50 mmol, 1 eq), N-(tert-butoxycarbonyl)-N-methyl-L-alanine (366.59 mg, 1.80 mmol, 1.2 eq) in DMF (8 mL), TCFH (632.63 mg, 2.25 mmol, 1.5 eq) and 1-methylimidazole (1.23 g, 15.03 mmol, 1.20 mL, 10 eq) was added, the mixture was stirred at 14° C. for 2 hours. LCMS showed the reaction was completed. The reaction was poured into H₂O (30 mL). The aqueous phase was extracted with ethyl acetate (10 mL*6). The combined organic phase was washed with saturated brine (30 mL*1), dried with anhydrous Na₂SO₄. Filtered and concentrated in vacuum. The residue was purified by silica gel chromatography (column height: 250 mm, diameter: 100 mm, 100-200 mesh silica gel, Petroleum ether/Ethyl acetate=1/1, 10/1) to afford tert-butyl (S)-(1-((3-((3-carbamoyl-5-ethyl-6-methylpyrazin-2-yl)amino)phenethyl)amino)-1-oxopropan-2-yl)(methyl)carbamate (1 g, 1.28 mmol, 85.12% yield) as a yellow solid. 1H NMR (400 MHz, DMSO-d6) δ=11.06 (s, 1H), 8.14 (br d, J=2.0 Hz, 1H), 7.85 (br d, J=2.1 Hz, 1H), 7.83-7.76 (m, 1H), 7.67-7.60 (m, 1H), 7.45 (br s, 1H), 7.22 (t, J=7.8 Hz, 1H), 6.82 (d, J=7.7 Hz, 1H), 4.61-4.16 (d, 1H), 3.30 (br d, J=6.8 Hz, 2H), 2.94-2.89 (m, 2H), 2.78-2.73 (m, 2H), 2.69 (br d, J=8.3 Hz, 3H), 1.36 (br s, 9H), 1.25-1.21 (m, 3H), 1.19-1.15 (m, 3H). LC-MS (ES+, m/z): 485.4 [(M+H)⁺]; Rt=0.877 min.

### Step 5: (S)-6-ethyl-5-methyl-3-((3-(2-(2-(methylamino)propanamido)ethyl)phenyl)amino)pyrazine-2-carboxamide

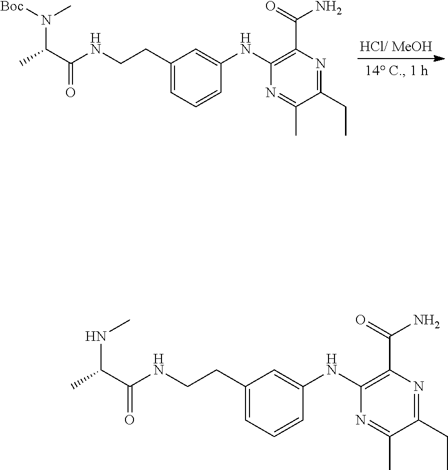

To a mixture of tert-butyl (S)-(1-((3-((3-carbamoyl-5-ethyl-6-methylpyrazin-2-yl)amino)phenethyl)amino)-1-oxopropan-2-yl)(methyl)carbamate (1 g, 1.28 mmol, 1 eq) in HCl/MeOH (4 M, 20 mL, 62.53 eq) at 14° C. for 1 hour. LCMS showed the reaction was completed. Filtered and concentrated in vacuum to afford (S)-6-ethyl-5-methyl-3-((3-(2-(2-(methylamino)propanamido)ethyl)phenyl)amino)pyrazine-2-carboxamide (430 mg, 1.12 mmol, 87.41% yield) as a yellow solid. LC-MS (ES+, m/z): 385.3 [(M+H)+]; Rt=0.671 min.

Note: HCl was bubbled into a solution MeOH at 0° C. for 0.5 h.

### Step 6: (S,E)-3-((3-(2-(2-(4-(dimethylamino)-N-methylbut-2-enamido)propanamido)ethyl)phenyl)amino)-6-ethyl-5-methylpyrazine-2-carboxamide

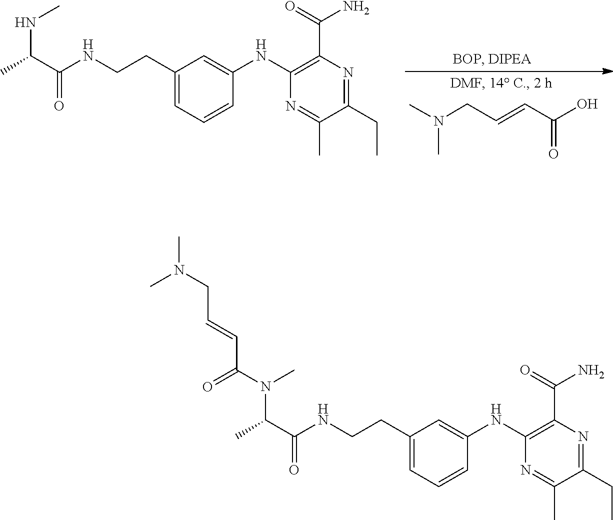

To a solution of (S)-6-ethyl-5-methyl-3-((3-(2-(2-(methylamino)propanamido)ethyl)phenyl)amino)pyrazine-2-carboxamide (400 mg, 1.04 mmol, 1 eq), (E)-4-(dimethylamino)but-2-enoic acid (161.25 mg, 1.25 mmol, 1.2 eq) in DMF (5 mL), BOP (690.21 mg, 1.56 mmol, 1.5 eq) and DIPEA (1.34 g, 10.40 mmol, 1.81 mL, 10 eq) was added at 14° C., the mixture was stirred at 14° C. for 2 hours. LCMS showed the reaction was completed. The residue was purified by prep-HPLC (column: Phenomenex luna C18 250*50 mm*10 um; mobile phase: [water(TFA)-ACN]; B %: 20%-50%, 10 min) to afford (S,E)-3-((3-(2-(2-(4-(dimethylamino)-N-methylbut-2-enamido)propanamido)ethyl)phenyl)amino)-6-ethyl-5-methylpyrazine-2-carboxamide (100.3 mg, 202.37 umol, 19.45% yield) as a yellow solid. 1H NMR (400 MHz, DMSO-d6) δ=11.09-11.03 (m, 1H), 9.75 (s, 1H) 8.16 (br s, 1H), 7.92 (br t, J=5.5 Hz, 1H), 7.86 (br s, 1H), 7.66 (br d, J=7.7 Hz, 1H), 7.43 (s, 1H), 7.28-7.17 (m, 1H), 6.88-6.73 (m, 2H), 6.66-6.44 (m, 1H), 5.03-4.51 (m, 1H), 3.94-3.78 (m, 2H), 3.34-3.26 (m, 2H), 2.90 (s, 2H), 2.78-2.70 (m, 11H), 2.50 (m, 3H), 1.29 (d, J=6.9 Hz, 1H), 1.27-1.19 (m, 5H). LC-MS (ES⁺, m/z): 496.4 [(M+H)⁺]; Rt=0.682 min; HRMS: 496.3003.

### Example 51

### (Compound 143)

### (S,E)-5-(dimethylamino)-6-ethyl-3-((3-methoxy-5-(2-(2-(N-methyl-4-(methylamino)but-2-enamido)propanamido)ethyl)phenyl)amino)pyrazine-2-carboxamide

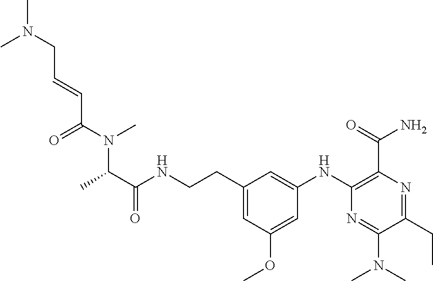 [SMILES: CCc1nc(C(N)=O)c(Nc2cc(CCNC(=O)[C@H](C)N(C)C(=O)/C=C/CN(C)C)cc(OC)c2)nc1N(C)C]

### Step 1: tert-butyl (S)-(1-((3-((3-carbamoyl-6-chloro-5-ethylpyrazin-2-yl)amino)-5-methoxyphenethyl)amino)-1-oxopropan-2-yl)(methyl)carbamate

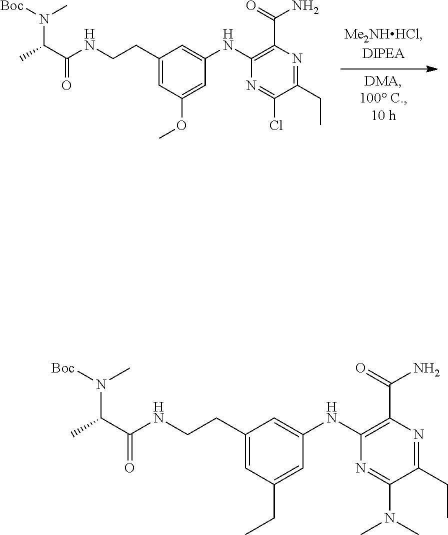

To a solution of tert-butyl (S)-(1-((3-((3-carbamoyl-6-chloro-5-ethylpyrazin-2-yl)amino)-5-methoxyphenethyl)amino)-1-oxopropan-2-yl)(methyl)carbamate (500 mg, 934.52 umol, 1 eq), N-methylmethanamine; hydrochloride (762.05 mg, 9.35 mmol, 10 eq) in DMA (5 mL) was added DIEA (1.21 g, 9.35 mmol, 1.63 mL, 10 eq). The mixture was stirred at 100° C. for 10 hrs under N₂. LCMS indicated the reaction was completed. The reaction was poured into water (10 mL) and extracted with EtOAc (5 mL*2). The organic layers were combined, washed with water (20 mL*2), sat. brine (20 mL), dried with anhydrous Na₂SO₄, filtered and concentrated to give crude product. The crude product was purified by chromatography on silica thiol gel (petroleum ether/EtOAc=1/2). To afford tert-butyl (S)-(1-((3-((3-carbamoyl-6-(dimethylamino)-5-ethylpyrazin-2-yl)amino)-5-methoxyphenethyl)amino)-1-oxopropan-2-yl)(methyl)carbamate (300 mg, 551.82 umol) as yellow solid. 1H NMR (400 MHz, CDCl3-d) δ=10.73 (s, 1H), 7.51 (br s, 1H), 7.22 (s, 1H), 7.06 (s, 1H), 6.31 (s, 1H), 5.27-5.22 (m, 1H), 4.70-4.42 (m, 1H), 3.72 (s, 3H), 3.52-3.41 (m, 3H), 3.04 (s, 6H), 2.72-2.66 (m, 4H), 2.62 (s, 3H), 1.33 (s, 9H), 1.27-1.19 (m, 6H). LC-MS (ES+, m/z): 544.4 [(M+H)⁺]; Rt=0.870 min.

### Step 2: (S)-5-(dimethylamino)-6-ethyl-3-((3-methoxy-5-(2-(2-(methylamino) propanamido)ethyl)phenyl)amino)pyrazine-2-carboxamide

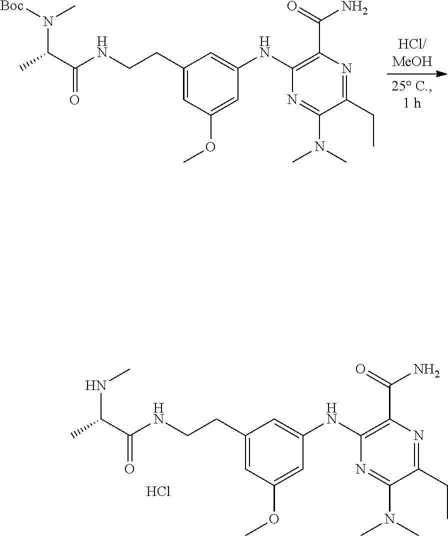

To a solution of tert-butyl (S)-(1-((3-((3-carbamoyl-6-(dimethylamino)-5-ethylpyrazin-2-yl)amino)-5-methoxyphenethyl)amino)-1-oxopropan-2-yl)(methyl) carbamate (300 mg, 551.82 umol, 1 eq) in HCl/MeOH (50 mL). The mixture was stirred at 25° C. for 1 h. LCMS indicated the reaction was completed. The crude mixture was concentrated under reduced pressure. To afford the crude mixture of (S)-5-(dimethylamino)-6-ethyl-3-((3-methoxy-5-(2-(2-(methylamino)propanamido)ethyl)phenyl)amino)pyrazine-2-carboxamide (230 mg, 518.55 umol, 93.97% yield) As yellow solid. LC-MS (ES+, m/z): 444.3 [(M+H)⁺]; Rt=0.734 min.

Note: HCl/MeOH (4 M): HCl was bubbled into a solution MeOH at 0° C. for 0.5 h. Then, the solution was weighed to obtained the HCl/MeOH (4 M)

### Step 3: (S,E)-5-(dimethylamino)-6-ethyl-3-((3-methoxy-5-(2-(2-(N-methyl-4-(methylamino)but-2-enamido)propanamido)ethyl)phenyl)amino)pyrazine-2-carboxamide

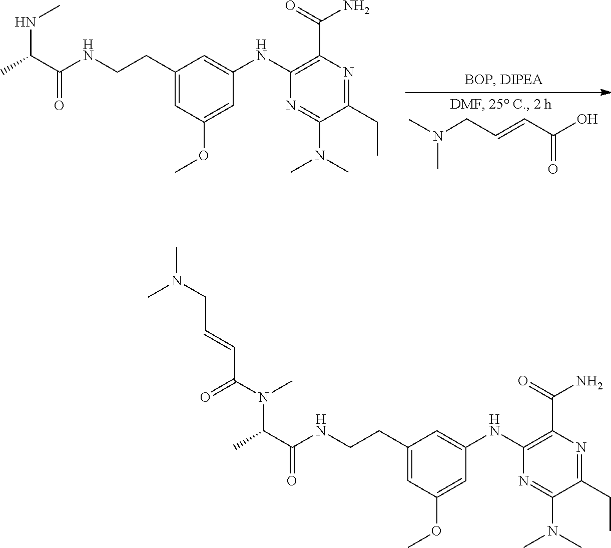

To a solution of (S)-5-(dimethylamino)-6-ethyl-3-((3-methoxy-5-(2-(2-(methylamino)propanamido)ethyl)phenyl)amino)pyrazine-2-carboxamide (230 mg, 518.55 umol, 1 eq), (E)-4-(dimethylamino)but-2-enoic acid (171.76 mg, 1.04 mmol, 2 eq, HCl) in DMF (4 mL) was added DIEA (670.19 mg, 5.19 mmol, 903.23 uL, 10 eq), BOP (344.02 mg, 777.83 umol, 1.5 eq). The mixture was stirred at 25° C. for 2 h under N₂. LCMS indicated the reaction has completed. The mixture was purified by prep-HPLC column: Phenomenex Luna 80*30 mm*3 um; mobile phase: [water(TFA)-ACN]; B %: 20%-50%, 8 min. to afford (S,E)-5-(dimethylamino)-6-ethyl-3-((3-methoxy-5-(2-(2-(N-methyl-4-(methylamino)but-2-enamido)propanamido)ethyl)phenyl)amino)pyrazine-2-carboxamide (60 mg, 108.17 umol, 20.86% yield) as yellow solid. 1H NMR (400 MHz, DMSO-d6, TFA) δ=11.24-11.00 (m, 1H), 9.74 (br d, J=2.7 Hz, 1H), 8.12-7.86 (m, 1H), 7.77 (br s, 1H), 7.47 (br s, 1H), 7.41-7.35 (m, 1H), 6.90 (s, 1H), 6.85-6.77 (m, 1H), 6.64-6.48 (m, 1H), 6.38 (s, 1H), 5.05-4.53 (m, 1H), 3.90-3.80 (m, 2H), 3.75 (s, 3H), 3.34-3.24 (m, 2H), 3.08 (s, 6H), 2.91 (s, 2H), 2.81-2.63 (m, 11H), 1.32-1.20 (m, 6H). LC-MS (ES+, m/z): 555.3 [(M+H)⁺]; Rt=2.117 min; HRMS: 555.3435.

### Example 52

### (Compound 144)

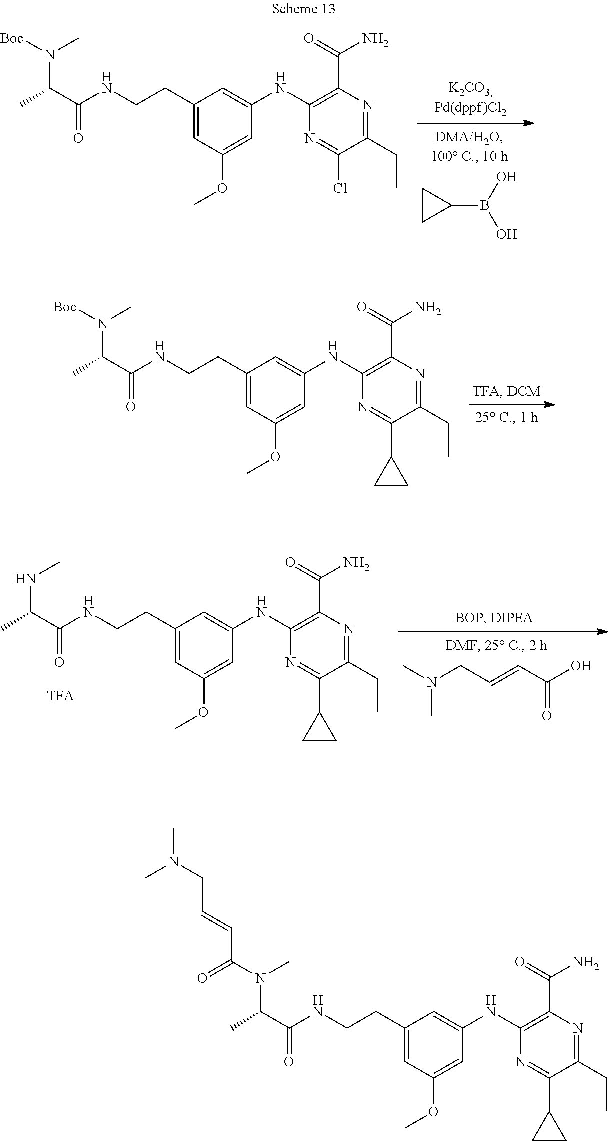

### (S,E)-5-cyclopropyl-3-((3-(2-(2-(4-(dimethylamino)-N-methylbut-2-enamido)propanamido)ethyl)-5-methoxyphenyl)amino)-6-ethylpyrazine-2-carboxamide

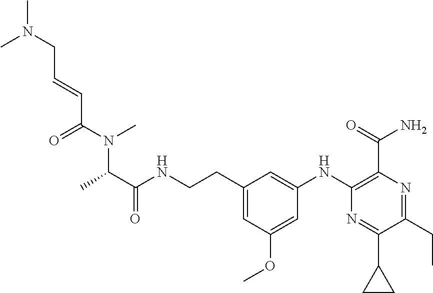 [SMILES: CCc1nc(C(N)=O)c(Nc2cc(CCNC(=O)[C@H](C)N(C)C(=O)/C=C/CN(C)C)cc(OC)c2)nc1C1CC1]

### Step 1: tert-butyl (S)-(1-((3-((3-carbamoyl-6-cyclopropyl-5-ethylpyrazin-2-yl)amino)-5-methoxyphenethyl)amino)-1-oxopropan-2-yl)(methyl)carbamate

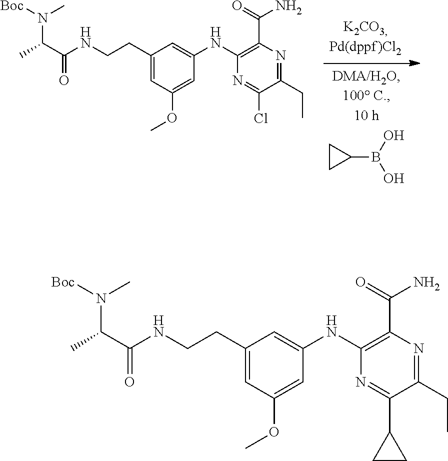

To a solution of tert-butyl (S)-(1-((3-((3-carbamoyl-6-chloro-5-ethylpyrazin-2-yl)amino)-5-methoxyphenethyl)amino)-1-oxopropan-2-yl)(methyl)carbamate (500 mg, 934.52 umol, 1 eq), cyclopropylboronic acid (802.73 mg, 9.35 mmol, 10 eq) in DMA (4 mL), H₂O (2 mL). The mixture was stirred at 100° C. for 10 hrs under N₂. LCMS indicated the reaction has completed. The reaction was poured into saturated EDTA (10 mL), EtOAc (5 mL) and stirred 60 min, and extracted with EtOAc (5 mL*3). The organic layers were combined, washed with water (20 mL*2), sat. brine (20 mL), dried with anhydrous Na₂SO₃, filtered and concentrated to give crude product. The crude product was purified by chromatography on silica thiol gel (petroleum ether/EtOAc=1/1). To afford tert-butyl (S)-(1-((3-((3-carbamoyl-6-cyclopropyl-5-ethylpyrazin-2-yl)amino)-5-methoxyphenethyl)amino)-1-oxopropan-2-yl)(methyl)carbamate (330 mg, 610.37 umol, 65.31% yield) as white solid. LC-MS (ES+, m/z): 541.3 [(M+H)⁺]; Rt=0.890 min.

### Step 2: (S)-5-cyclopropyl-6-ethyl-3-((3-methoxy-5-(2-(2-(methylamino)propanamido)ethyl)phenyl)amino)pyrazine-2-carboxamide

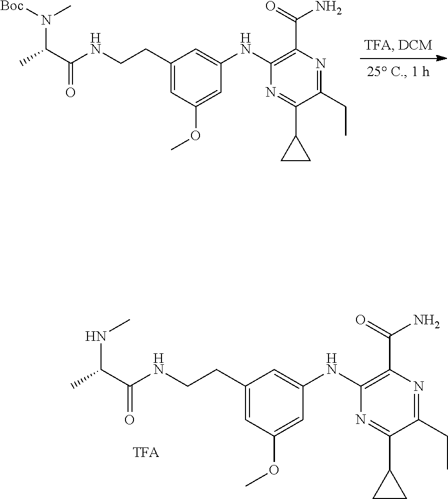

To solution of tert-butyl (S)-(1-((3-((3-carbamoyl-6-cyclopropyl-5-ethylpyrazin-2-yl)amino)-5-methoxyphenethyl)amino)-1-oxopropan-2-yl)(methyl) carbamate (330 mg, 610.37 umol, 1 eq) in DCM (40 mL) was added TFA (20 mL). The mixture was stirred at 25° C. for 1 h. LCMS indicated the reaction was completed. The crude mixture was concentrated under reduced pressure. Then, the mixture was put into next step directly. to afford crude mixture of (S)-5-cyclopropyl-6-ethyl-3-((3-methoxy-5-(2-(2-(methylamino)propanamido)ethyl)phenyl)amino)pyrazine-2-carboxamide (250 mg, 567.49 umol, 92.97% yield) as yellow oil. LC-MS (ES+, m/z): 441.3 [(M+H)⁺]; Rt=0.753 min.

### Step 3: (S,E)-5-cyclopropyl-3-((3-(2-(2-(4-(dimethylamino)-N-methylbut-2-enamido)propanamido)ethyl)-5-methoxyphenyl)amino)-6-ethylpyrazine-2-carboxamide

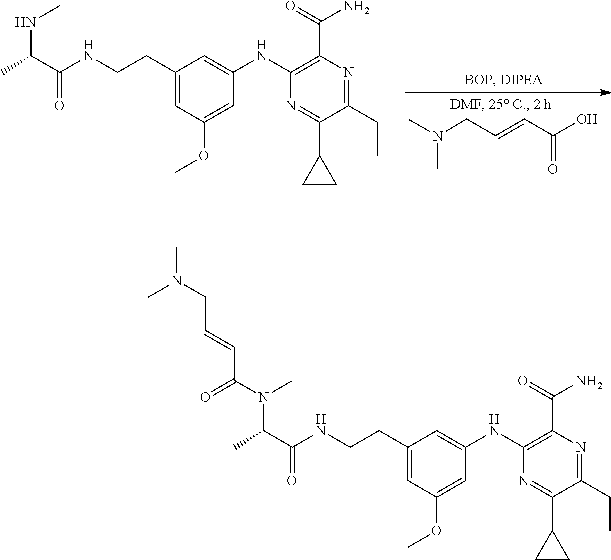

To a solution of (S)-5-cyclopropyl-6-ethyl-3-((3-methoxy-5-(2-(2-(methylamino)propanamido)ethyl)phenyl)amino)pyrazine-2-carboxamide (250 mg, 567.49 umol, 1 eq), (E)-4-(dimethylamino)but-2-enoic acid (187.97 mg, 1.13 mmol, 2 eq, HCl) in DMF (4 mL) was added BOP (376.48 mg, 851.23 umol, 1.5 eq), DIEA (733.44 mg, 5.67 mmol, 988.46 uL, 10 eq). The mixture was stirred at 25° C. for 2 hrs under N₂. LCMS indicated the reaction was completed. The crude mixture was purified by prep-HPLC column: Phenomenex Luna 80*30 mm*3 um; mobile phase: [water(TFA)-ACN]; B %: 20%-50%, 8 min. to afford (S,E)-5-cyclopropyl-3-((3-(2-(2-(4-(dimethylamino)-N-methylbut-2-enamido)propanamido)ethyl)-5-methoxyphenyl)amino)-6-ethylpyrazine-2-carboxamide (70 mg, 126.89 umol, 22.36% yield) as yellow solid. 1H NMR (400 MHz, DMSO-d6, TFA) δ=11.18-11.02 (m, 1H), 9.75 (br s, 1H), 8.15 (s, 1H), 7.92 (t, J=5.6 Hz, 1H), 7.84 (br s, 1H), 7.38 (br d, J=1.9 Hz, 1H), 6.86-6.78 (m, 1H), 6.78-6.75 (m, 1H), 6.65-6.48 (m, 1H), 6.40 (s, 1H), 5.02-4.54 (m, 1H), 3.92-3.81 (m, 2H), 3.77 (s, 3H), 3.35-3.25 (m, 2H), 2.96-2.87 (m, 4H), 2.82-2.64 (m, 9H), 2.39-2.27 (m, 1H), 1.32-1.19 (m, 6H), 1.15-1.07 (m, 4H). LC-MS (ES+, m/z): 552.3 [(M+H)⁺]; Rt=2.188 min; HRMS: 552.3289

### Example 64

### (Compound 156)

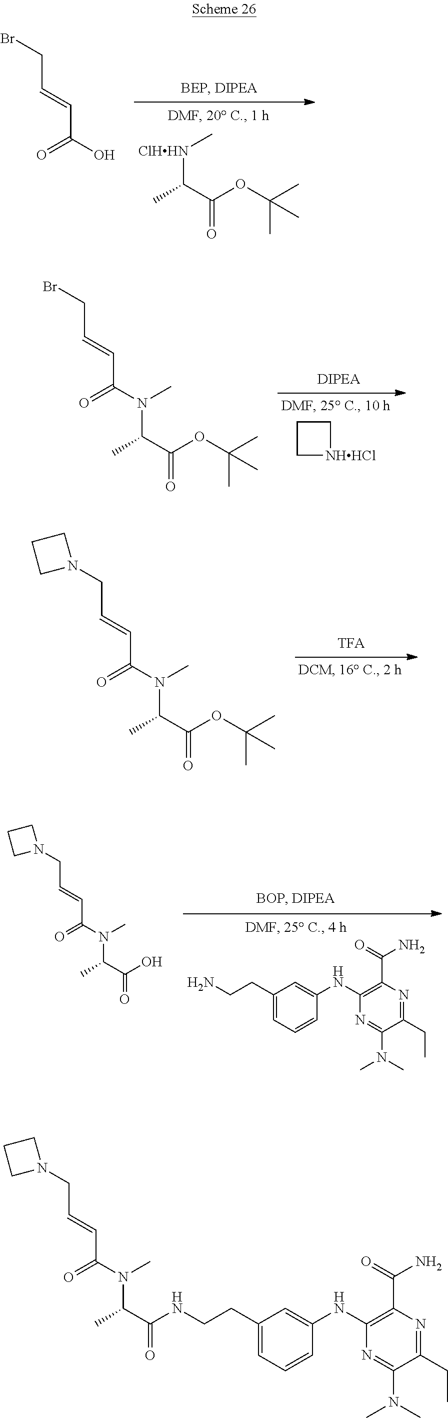

### Step 1: tert-butyl (E)-N-(4-bromobut-2-enoyl)-N-methyl-L-alaninate

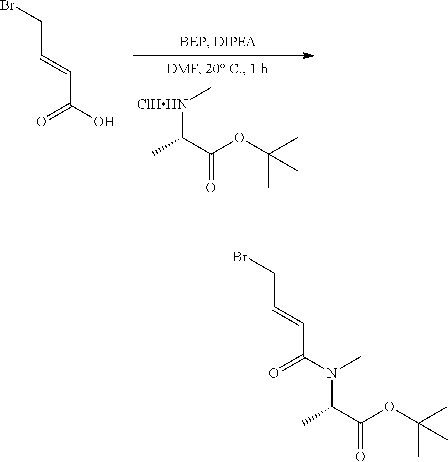

To a mixture of tert-butyl methyl-L-alaninate hydrochloride (9 g, 45.99 mmol, 1 eq) and (E)-4-bromobut-2-enoic acid (8.35 g, 50.59 mmol, 1.1 eq) in DMF (90 mL) was added 2-bromo-1-ethyl-pyridin-1-ium;tetrafluoroborate (18.89 g, 68.99 mmol, 1.5 eq) and DIEA (59.44 g, 459.92 mmol, 80.11 mL, 10 eq) in one portion at 20° C. under N₂. The mixture was stirred at 20° C. for 1 hr. LCMS indicated the reaction was completed. The residue was poured into water (200 mL). The aqueous phase was extracted with ethyl acetate (100 mL*3). The combined organic phase was washed with saturated brine (400 mL*1), dried with anhydrous Na₂SO₄, filtered and concentrated in vacuum. The residue was purified by column chromatography (SiO₂, Petroleum ether:Ethyl acetate=30/1 to 1/1). To afford the title compound tert-butyl (E)-N-(4-bromobut-2-enoyl)-N-methyl-L-alaninate (8 g, 26.13 mmol, 28.40% yield) as a yellow oil. LC-MS (ES+, m/z): 206.3 [(M+H)⁺]. Rt=1.837 min.

### Step 2: tert-butyl (E)-N-(4-(azetidin-1-yl)but-2-enoyl)-N-methyl-L-alaninate

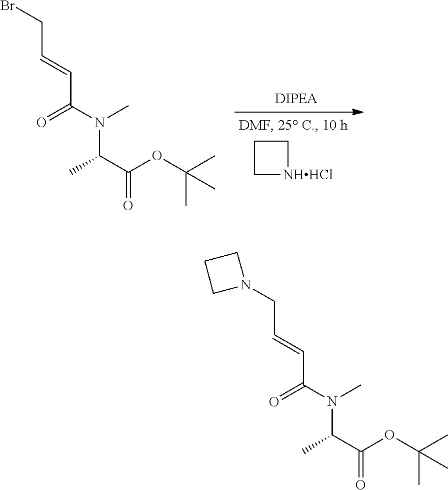

To a mixture of tert-butyl tert-butyl (E)-N-(4-bromobut-2-enoyl)-N-methyl-L-alaninate (8 g, 26.13 mmol, 1 eq) and azetidine hydrochloride (2.44 g, 26.13 mmol, 1 eq) in DMF (80 mL) was added DIPEA (10.13 g, 78.38 mmol, 3 eq) in one portion at 25° C. under N₂. The mixture was stirred at 25° C. for 10 hrs. LCMS indicated the reaction was completed. The reaction mixture was filtrated under reduced pressure to give a residue. The residue was purified by column chromatography (SiO₂, Petroleum ether:Ethyl acetate=1/1 to 1/1). To afford the title compound tert-butyl (E)-N-(4-(azetidin-1-yl)but-2-enoyl)-N-methyl-L-alaninate (550 mg, 1.95 mmol, 2.48% yield) as a yellow oil. LC-MS (ES+, m/z): 283.4 [(M+H)⁺]. Rt=0.546 min

### Step 3: (E)-N-(4-(azetidin-1-yl)but-2-enoyl)-N-methyl-L-alanine

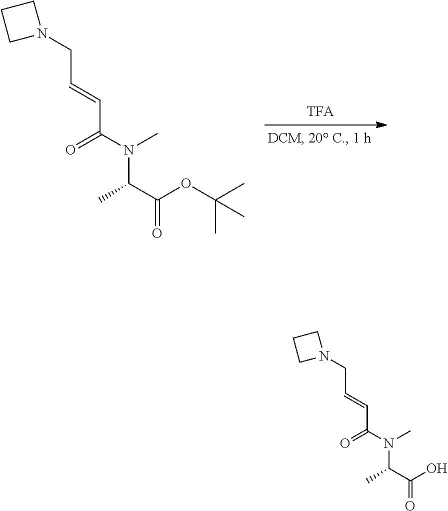 [SMILES: C[C@@H](C(=O)OC(C)(C)C)N(C)C(=O)/C=C/CN1CCC1 ; C[C@@H](C(=O)O)N(C)C(=O)/C=C/CN1CCC1]

To a mixture of tert-butyl (E)-N-(4-(azetidin-1-yl)but-2-enoyl)-N-methyl-L-alaninate (500 mg, 1.77 mmol, 1 eq) in DCM (1.5 mL) was added TFA (77.00 g, 675.30 mmol, 50.00 mL, 381.38 eq) in one portion at 20° C. under N₂. The mixture was stirred at 20° C. for 1 hr. LCMS indicated the reaction was completed. The reaction mixture was concentrated under reduced pressure to give a residue. To afford the title compound (E)-N-(4-(azetidin-1-yl)but-2-enoyl)-N-methyl-L-alanine (480 mg, crude) as a yellow oil. LC-MS (ES+, m/z): 227.3 [(M+H)⁺]. Rt=0.177 min.

### Step 4: (S,E)-3-((3-(2-(2-(4-(azetidin-1-yl)-N-methylbut-2-enamido)propanamido)ethyl)phenyl)amino)-5-(dimethylamino)-6-ethylpyrazine-2-carboxamide

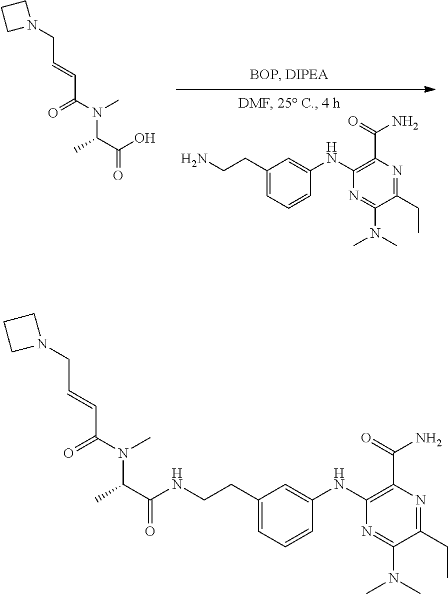 [SMILES: C[C@@H](C(=O)O)N(C)C(=O)/C=C/CN1CCC1 ; CCc1nc(C(N)=O)c(Nc2cccc(CCN)c2)nc1N(C)C ; CCc1nc(C(N)=O)c(Nc2cccc(CCNC(=O)[C@H](C)N(C)C(=O)/C=C/CN3CCC3)c2)nc1N(C)C]

To a mixture of (E)-N-(4-(azetidin-1-yl)but-2-enoyl)-N-methyl-L-alanine propanoic acid (109.44 mg, 483.66 umol, 2 eq) and 3-((3-(2-aminoethyl)phenyl)amino)-5-(dimethylamino)-6-ethylpyrazine-2-carboxamide (100 mg, 241.83 umol, 1 eq) in DMF (2 mL) was added BOP (160.43 mg, 362.74 umol, 1.5 eq) and DIPEA (312.55 mg, 2.42 mmol, 421.22 uL, 10 eq) in one portion at 25° C. under N₂. The mixture was stirred at 25° C. for 4 hrs LCMS indicated the reaction was completed. The reaction mixture was concentrated under reduced pressure to give a residue. The residue was purified by prep-HPLC (TFA condition). column: C18-1 150*30 mm*5 um; mobile phase: [water(TFA)-ACN]; B %: 10%-55%, 8 min. To afford the title compound (S,E)-3-((3-(2-(2-(4-(azetidin-1-yl)-N-methylbut-2-enamido)propanamido)ethyl)phenyl)amino)-5-(dimethylamino)-6-ethylpyrazine-2-carboxamide (25.1 mg, 45.85 umol, 18.96% yield) as a brown solid. ¹H NMR (400 MHz, DMSO-d₆, TFA) δ=11.22-11.07 (m, 1H), 10.02-9.71 (m, 1H), 8.11-7.86 (m, 1H), 7.79-7.72 (m, 1H), 7.56-7.42 (m, 3H), 7.26-7.15 (m, 1H), 6.82-6.67 (m, 2H), 6.52-6.19 (m, 1H), 4.97-4.56 (m, 1H), 4.14 (br d, J=6.2 Hz, 2H), 4.00-3.98 (m, 4H), 3.35-3.24 (m, 2H), 3.09-3.04 (m, 6H), 2.92-2.86 (m, 2H), 2.80-2.67 (m, 6H), 2.44-2.35 (m, 2H), 1.28-1.19 (m, 6H). ¹H NMR (400 MHz, D2O) δ=7.55-7.39 (m, 1H), 7.26-7.05 (m, 2H), 6.80 (br d, J=7.3 Hz, 1H), 6.58-6.40 (m, 1H), 6.38 (s, 1H), 4.85-4.41 (m, 1H), 4.31-4.07 (m, 2H), 4.05-3.86 (m, 2H), 3.84-3.59 (m, 2H), 3.53-3.27 (m, 2H), 3.05-2.93 (m, 6H), 2.79-2.62 (m, 7H), 2.58-2.29 (m, 2H), 1.32-1.12 (m, 6H) LC-MS (ES+, m/z): 537.3 [(M+H)⁺]. Rt=2.117 min. HRMS (EI): m/z [M]⁺ found: 537.3306.

### Example 66

### (Compound 158)

### (S,E)-5-(dimethylamino)-3-((3-(2-(2-(4-(dimethylamino)-N-methylbut-2-enamido)propanamido)ethyl)-5-fluorophenyl)amino)-6-ethylpyrazine-2-carboxamide

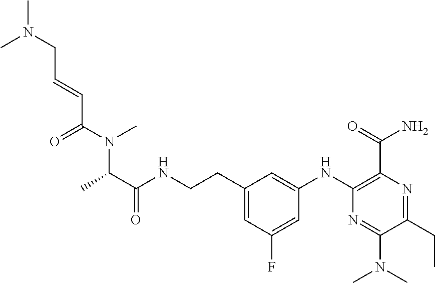 [SMILES: CCc1nc(C(N)=O)c(Nc2cc(F)cc(CCNC(=O)[C@H](C)N(C)C(=O)/C=C/CN(C)C)c2)nc1N(C)C]

### Step 1: tert-butyl (S)-(1-((3-((3-carbamoyl-6-(dimethylamino)-5-ethylpyrazin-2-yl)amino)-5-fluorophenethyl)amino)-1-oxopropan-2-yl)(methyl)carbamate

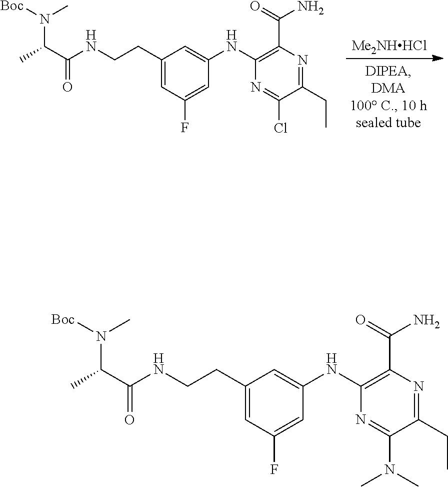

To a solution of tert-butyl (S)-(1-((3-((3-carbamoyl-6-chloro-5-ethylpyrazin-2-yl)amino)-5-fluorophenethyl)amino)-1-oxopropan-2-yl)(methyl)carbamate (500 mg, 956.03 umol, 1 eq) in DMA (5 mL) was added DIPEA (1.24 g, 9.56 mmol, 1.67 mL, 10 eq) and dimethylamine (779.58 mg, 9.56 mmol, 875.94 uL, 10 eq, HCl). The mixture was stirred at 100° C. for 10 hrs in sealed tube. LC-MS showed the reaction was completed. The reaction mixture was poured into H₂O (5 mL) and extracted with EA (10 mL*3). The combined organic layers were washed with saturated brine (10 mL*3), dried over anhydrous Na₂SO₄, filtered and concentrated under reduced pressure to give a residue. The residue was purified by column chromatography (SiO₂, Petroleum ether/Ethyl acetate=5/1 to 1/5) to afford tert-butyl (S)-(1-((3-((3-carbamoyl-6-(dimethylamino)-5-ethylpyrazin-2-yl)amino)-5-fluorophenethyl)amino)-1-oxopropan-2-yl)(methyl)carbamate (300 mg, 564.31 umol, 59.03% yield) as yellow oil. ¹H NMR (400 MHz, DMSO-d₆) δ=11.38-11.23 (m, 1H), 7.87-7.73 (m, 2H), 7.72-7.60 (m, 1H), 7.58-7.47 (m, 1H), 7.17-7.05 (m, 1H), 6.68-6.55 (m, 1H), 4.57-4.22 (m, 1H), 3.17 (d, J=5.3 Hz, 2H), 3.07 (s, 5H), 2.79-2.74 (m, 4H), 2.71-2.68 (m, 4H), 1.40-1.20 (m, 15H). LC-MS (ES⁺, m/z): 532.3 [(M+H)⁺]; Rt=0.886 min.

### Step 2: (S)-5-(dimethylamino)-6-ethyl-3-((3-fluoro-5-(2-(2-(methylamino)propanamido)ethyl)phenyl)amino)pyrazine-2-carboxamide

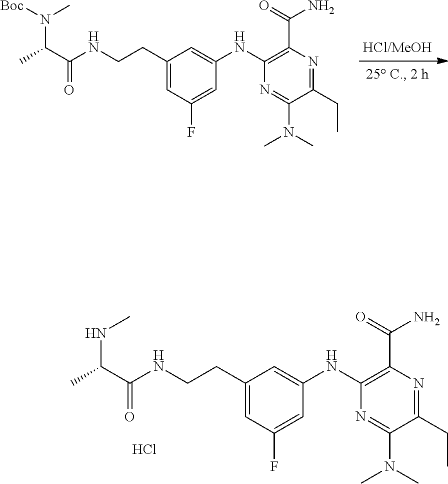

A mixture of tert-butyl (S)-(1-((3-((3-carbamoyl-6-(dimethylamino)-5-ethylpyrazin-2-yl)amino)-5-fluorophenethyl)amino)-1-oxopropan-2-yl)(methyl) carbamate (300 mg, 564.31 umol, 1 eq), in HCl/MeOH (4 M, 48.70 mL, 345.21 eq) was stirred at 25° C. for 2 hrs. LC-MS showed the reaction was completed. The reaction mixture was filtered and concentrated under reduced pressure to afford (S)-5-(dimethylamino)-6-ethyl-3-((3-fluoro-5-(2-(2-(methylamino)propanamido)ethyl)phenyl)amino)pyrazine-2-carboxamide (287.4 mg, crude, HCl) was obtained as a yellow oil (150 mg, crude) as yellow oil. LC-MS (ES⁺, m/z): 432.2 [(M+H)⁺]; Rt=0.755 min Note: HCl/MeOH (4 M): HCl was bubbled into a solution MeOH at 0° C. for 0.5 h.

### Step 3: (S,E)-5-(dimethylamino)-3-((3-(2-(2-(4-(dimethylamino)-N-methylbut-2-enamido)propanamido)ethyl)-5-fluorophenyl)amino)-6-ethylpyrazine-2-carboxamide

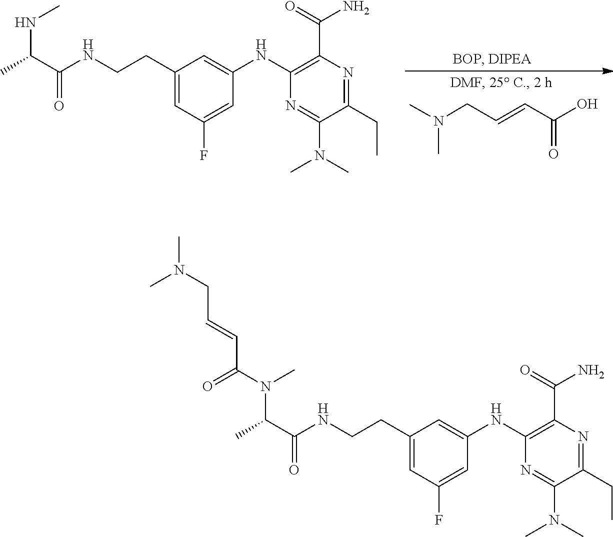

To a solution of (S)-5-(dimethylamino)-6-ethyl-3-((3-fluoro-5-(2-(2-(methylamino)propanamido)ethyl)phenyl)amino)pyrazine-2-carboxamide (287 mg, 665.11 umol, 1 eq) in DMF (4 mL) was added BOP (441.25 mg, 997.67 umol, 1.5 eq), DIPEA (1.60 g, 12.40 mmol, 2.16 mL, 18.64 eq) and (E)-4-(dimethylamino)but-2-enoic acid (352.36 mg, 2.73 mmol, 4.10 eq). The mixture was stirred at 25° C. for 2 hrs. LCMS showed the reaction was completed. The reaction mixture was filtered to give a residue. The residue was purified by prep-HPLC (column: Phenomenex Luna 80*30 mm*3 um; mobile phase: [water(TFA)-ACN]; B %: 20%-50%, 8 min) to afford (S,E)-5-(dimethylamino)-3-((3-(2-(2-(4-(dimethylamino)-N-methylbut-2-enamido) propanamido)ethyl)-5-fluorophenyl)amino)-6-ethylpyrazine-2-carboxamide (246 mg, 449.25 umol, 67.55% yield, 99.10% purity) as yellow solid. ¹H NMR (400 MHz, DMSO-d₆, TFA) δ=11.37-11.24 (m, 1H), 9.65 (br s, 1H), 8.14-7.86 (m, 1H), 7.79 (br s, 1H), 7.73-7.66 (m, 1H), 7.52 (br s, 1H), 7.08 (s, 1H), 6.86-6.75 (m, 1H), 6.65-6.46 (m, 2H), 4.96-4.55 (m, 1H), 3.91-3.86 (m, 2H), 3.33-3.24 (m, 2H), 3.10-3.06 (m, 6H), 2.89 (s, 2H), 2.77 (br d, J=7.3 Hz, 7H), 2.73-2.66 (m, 4H), 1.28-1.19 (m, 6H). LC-MS (ES⁺, m/z): 543.3 [(M+H)⁺]; Rt=2.200 min; 99.10% purity; HRMS: 543.3200.

### Example 67

### (Compound 159)

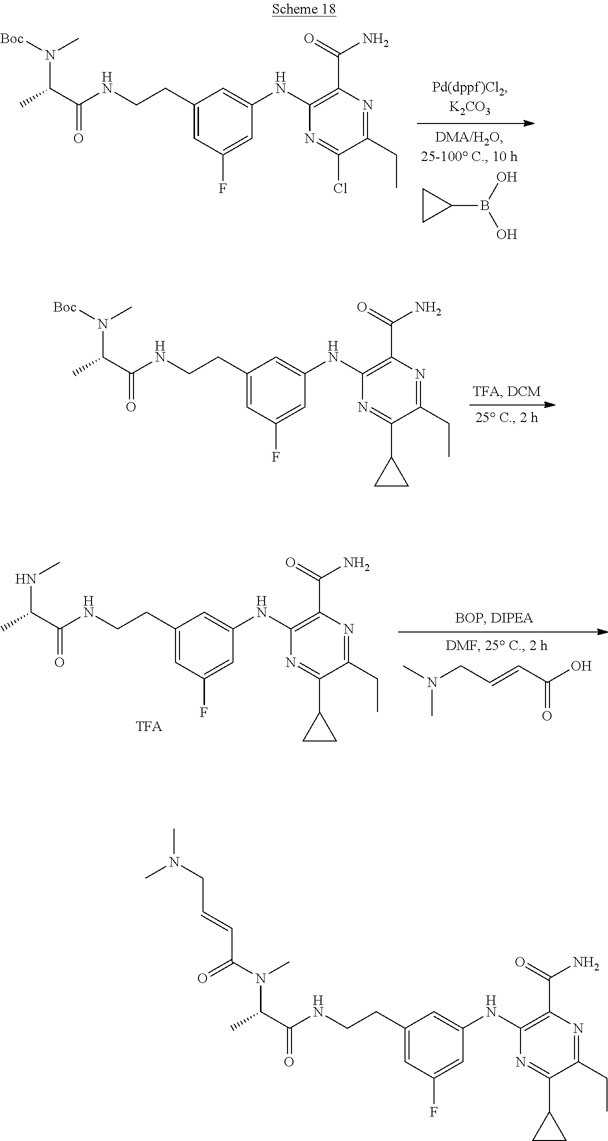

### (S,E)-5-cyclopropyl-3-((3-(2-(2-(4-(dimethylamino)-N-methylbut-2-enamido) propanamido)ethyl)-5-fluorophenyl)amino)-6-ethylpyrazine-2-carboxamide

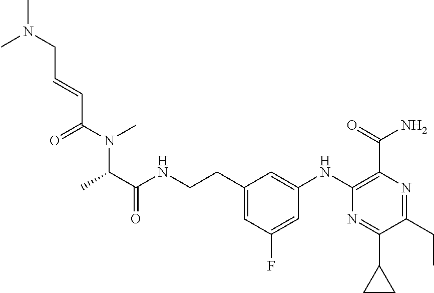 [SMILES: CCc1nc(C(N)=O)c(Nc2cc(F)cc(CCNC(=O)[C@H](C)N(C)C(=O)/C=C/CN(C)C)c2)nc1C1CC1]

### Step 1: tert-butyl (S)-(1-((3-((3-carbamoyl-6-cyclopropyl-5-ethylpyrazin-2-yl)amino)-5-fluorophenethyl)amino)-1-oxopropan-2-yl)(methyl)carbamate

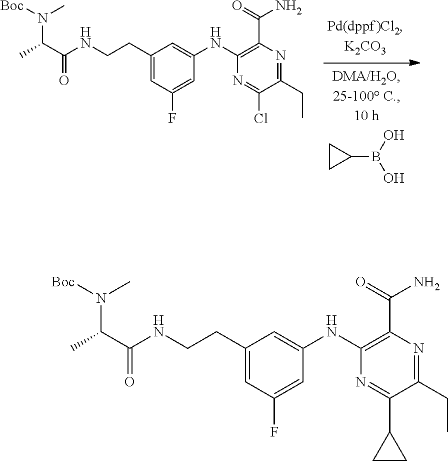

To a solution of tert-butyl (S)-(1-((3-((3-carbamoyl-6-chloro-5-ethylpyrazin-2-yl)amino)-5-fluorophenethyl)amino)-1-oxopropan-2-yl)(methyl) carbamate (500 mg, 956.03 umol, 1 eq) in DMA (4 mL) and H₂O (2 mL) was added Pd(dppf)Cl₂ (69.95 mg, 95.60 umol, 0.1 eq), cyclopropylboronic acid (821.20 mg, 9.56 mmol, 10 eq) and K₂CO₃ (396.39 mg, 2.87 mmol, 3 eq) at 25° C. The mixture was stirred at 100° C. for 10 hrs. LCMS showed the reaction was completed. The reaction mixture was poured into saturated EDTA (10 mL), EA (5 mL) and stirred 60 min, and then extracted with EA (5 mL*3). The combined organic layers were washed with saturated brine (10 mL*3), dried over anhydrous Na₂SO₄, filter and concentrated under reduced pressure to give a residue. The residue was purified by column chromatography (SiO₂, Petroleum ether/Ethyl acetate=5/1 to 1/1) to afford tert-butyl (S)-(1-((3-((3-carbamoyl-6-cyclopropyl-5-ethylpyrazin-2-yl)amino)-5-fluorophenethyl)amino)-1-oxopropan-2-yl)(methyl)carbamate (70 mg, 132.42 umol, 13.85% yield) as yellow oil. LC-MS (ES⁺, m/z): 529.3 [(M+H)⁺]; Rt=0.904 min.

### Step 2: (S)-5-cyclopropyl-6-ethyl-3-((3-fluoro-5-(2-(2-(methylamino)propanamido)ethyl)phenyl)amino)pyrazine-2-carboxamide

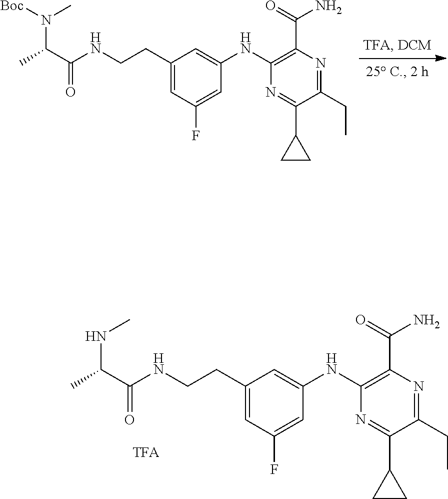

A mixture of tert-butyl (S)-(1-((3-((3-carbamoyl-6-cyclopropyl-5-ethylpyrazin-2-yl)amino)-5-fluorophenethyl)amino)-1-oxopropan-2-yl)(methyl) carbamate (310 mg, 586.44 umol, 1 eq) and TFA (2.45 g, 21.47 mmol, 1.59 mL, 36.61 eq) in DCM (5 mL) was stirred at 25° C. for 2 hrs. LCMS showed the reaction was completed. The reaction mixture was filtered and concentrated under reduced pressure to afford (S)-5-cyclopropyl-6-ethyl-3-((3-fluoro-5-(2-(2-(methylamino)propanamido)ethyl)phenyl)amino)pyrazine-2-carboxamide (440 mg, crude, TFA) as yellow solid. ¹H NMR (400 MHz, DMSO-d₆) δ=11.28-11.19 (m, 1H), 8.79-8.67 (m, 2H), 8.54-8.47 (m, 1H), 8.18 (br s, 1H), 7.93-7.84 (m, 1H), 7.63 (br d, J=11.9 Hz, 1H), 7.07-7.00 (m, 1H), 6.68 (br d, J=8.8 Hz, 1H), 3.70-3.64 (m, 1H), 3.51-3.35 (m, 2H), 2.93-2.90 (m, 1H), 2.79-2.72 (m, 2H), 2.70-2.67 (m, 1H), 2.45-2.43 (m, 3H), 1.30-1.26 (m, 6H), 1.13 (br s, 2H), 1.09-1.05 (m, 2H). LC-MS (ES⁺, m/z): 429.3 [(M+H)⁺]; Rt=0.744 min; 91.31% purity.

### Step 3: (S,E)-5-cyclopropyl-3-((3-(2-(2-(4-(dimethylamino)-N-methylbut-2-enamido)propanamido)ethyl)-5-fluorophenyl)amino)-6-ethylpyrazine-2-carboxamide

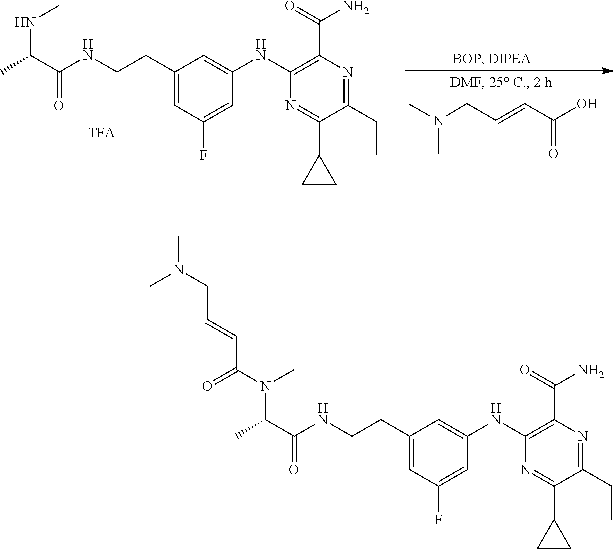

To a solution of (S)-5-cyclopropyl-6-ethyl-3-((3-fluoro-5-(2-(2-(methylamino)propanamido)ethyl)phenyl)amino)pyrazine-2-carboxamide (220 mg, 405.51 umol, 1 eq, TFA) and (E)-4-(dimethylamino)but-2-enoic acid in DMF (2 mL) was added BOP (269.02 mg, 608.27 umol, 1.5 eq), DIPEA (524.10 mg, 4.06 mmol, 706.33 uL, 10 eq). The mixture was stirred at 25° C. for 2 hrs. LCMS showed the reaction was completed The reaction mixture was filtered to give a residue. The residue was purified by prep-HPLC (column: Phenomenex Luna 80*30 mm*3 um; mobile phase: [water(TFA)-ACN]; B %: 20%-50%, 8 min) to afford (S,E)-5-cyclopropyl-3-((3-(2-(2-(4-(dimethylamino)-N-methylbut-2-enamido) propanamido)ethyl)-5-fluorophenyl)amino)-6-ethylpyrazine-2-carboxamide (60.81 mg, 106.44 umol, 26.25% yield, 94.46% purity) as yellow solid. ¹H NMR (400 MHz, DMSO-d₆) δ=11.23 (br s, 1H), 9.79-9.57 (m, 1H), 8.50-8.18 (m, 1H), 8.06-7.93 (m, 2H), 7.91-7.88 (m, 1H), 7.01-7.09 (m, 1H), 6.83-6.79 (m, 1H), 6.65-6.60 (m, 2H), 5.08-4.49 (m, 1H), 3.97-3.78 (m, 2H), 3.51-3.39 (m, 2H), 2.94-2.88 (m, 4H), 2.79-2.68 (m, 9H), 2.36-2.29 (m, 1H), 1.38-1.23 (m, 6H), 1.15-1.05 (m, 4H). LC-MS (ES+, m/z): 540.2 [(M+H)⁺]; Rt=2.261 min; 94.46 purity; HRMS: 540.3116.

### Example 68

### (Compound 160)

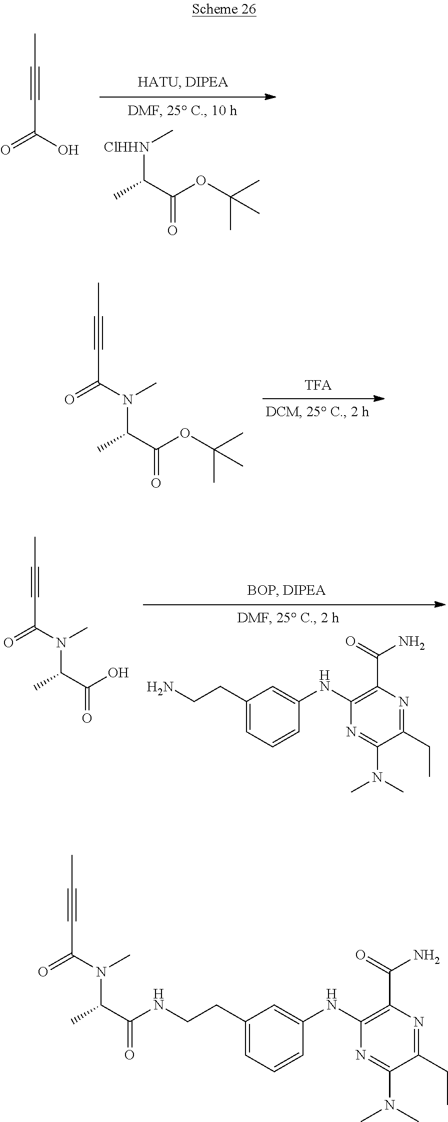

### (S)-5-(dimethylamino)-6-ethyl-3-((3-(2-(2-(N-methylbut-2-ynamido) propanamido)ethyl)phenyl)amino)pyrazine-2-carboxamide

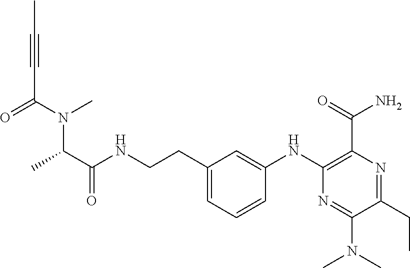 [SMILES: CC#CC(=O)N(C)[C@@H](C)C(=O)NCCc1cccc(Nc2nc(N(C)C)c(CC)nc2C(N)=O)c1]

### Step 1: tert-butyl N-(but-2-ynoyl)-N-methyl-L-alaninate

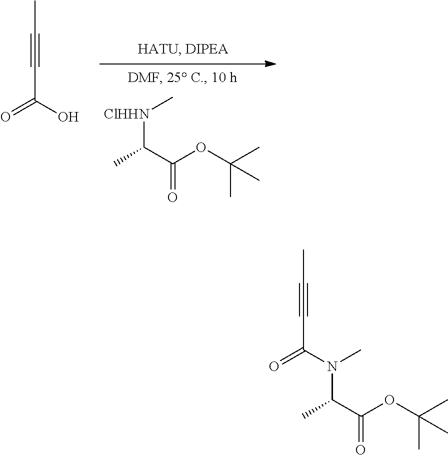

To a solution of tert-butyl methyl-L-alaninate (3 g, 15.33 mmol, 1 eq, HCl) and but-2-ynoic acid (1.42 g, 16.86 mmol, 1.1 eq) in DMF (30 mL) was added HATU (8.74 g, 23.00 mmol, 1.5 eq) and DIPEA (19.81 g, 153.31 mmol, 26.70 mL, 10 eq). The mixture was stirred at 25° C. for 10 hrs. LCMS showed the reaction was completed. The reaction was poured into water (30 mL) and extracted with EtOAc (40 mL*3). The organic layers were combined, washed with water (10 mL*3), sat. brine (10 mL*3), dried with anhydrous Na₂CO₃, filtered and concentrated to give crude product. The residue was purified by column chromatography (SiO₂, Petroleum ether/Ethyl acetate=5/1 to 1/1) to afford tert-butyl N-(but-2-ynoyl)-N-methyl-L-alaninate (3.2 g, 13.91 mmol, 90.76% yield, 97.96% purity) as yellow oil. ¹H NMR (400 MHz, DMSO-d₆) δ=4.98-4.67 (m, 1H), 3.17-3.05 (m, 2H), 2.73 (s, 1H), 2.02 (d, J=12.6 Hz, 3H), 1.45-1.25 (m, 12H); LC-MS (ES⁺, m/z): 226.1 [(M+H)⁺]; Rt=0.885 min;

### Step 2: 6-ethyl-3-((3-methoxy-5-(2-(3-propiolamidopropanamido)ethyl)phenyl)amino)-5-((tetrahydro-2H-pyran-4-yl)amino)pyrazine-2-carboxamide

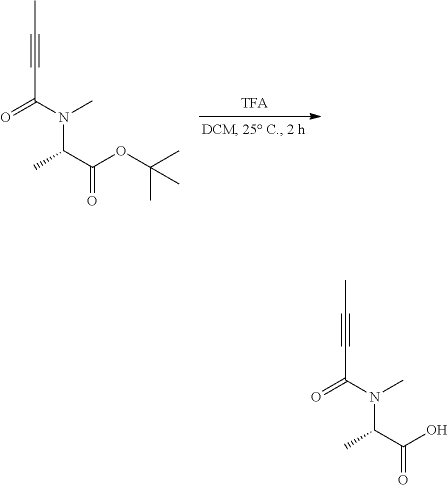 [SMILES: CC#CC(=O)N(C)[C@@H](C)C(=O)OC(C)(C)C ; CC#CC(=O)N(C)[C@@H](C)C(=O)O]

A mixture of tert-butyl N-(but-2-ynoyl)-N-methyl-L-alaninate (1.6 g, 7.10 mmol, 1 eq) and TFA (7.70 g, 67.53 mmol, 5 mL, 9.51 eq) in DCM (5 mL) was stirred at 25° C. for 2 hrs. LCMS showed the reaction was completed. The reaction mixture was filtered and concentrated under reduced pressure to afford N-(but-2-ynoyl)-N-methyl-L-alanine (1.84 g, crude, TFA) as yellow oil. ¹H NMR (400 MHz, DMSO-d₆) δ=12.91 (m, 1H), 5.04-4.75 (m, 1H), 3.07-2.51 (m, 3H), 2.03-2.00 (m, 3H), 1.38-1.28 (m, 3H); LC-MS (ES⁺, m/z): 170.0 [(M+H)+]; Rt=1.452 min; 98.93% purity; HRMS: 170.0775.

### Step 3: (S)-5-(dimethylamino)-6-ethyl-3-((3-(2-(2-(N-methylbut-2-ynamido) propanamido)ethyl)phenyl)amino)pyrazine-2-carboxamide

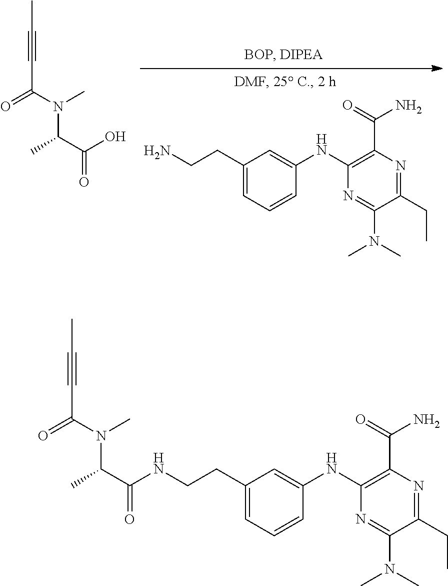 [SMILES: CC#CC(=O)N(C)[C@@H](C)C(=O)O ; CCc1nc(C(N)=O)c(Nc2cccc(CCN)c2)nc1N(C)C ; CC#CC(=O)N(C)[C@@H](C)C(=O)NCCc1cccc(Nc2nc(N(C)C)c(CC)nc2C(N)=O)c1]

To a solution of N-(but-2-ynoyl)-N-methyl-L-alanine (150 mg, 158.90 umol, 1 eq, TFA) and 3-((3-(2-aminoethyl)phenyl)amino)-5-(dimethylamino)-6-ethylpyrazine-2-carboxamide (104.37 mg, 317.80 umol, 2 eq) in DMF (1 mL) was added BOP (105.42 mg, 238.35 umol, 1.5 eq) and DIPEA (205.36 mg, 1.59 mmol, 276.77 uL, 10 eq). The mixture was stirred at 25° C. for 2 hrs. LCMS showed the reaction was completed. The reaction mixture was filtered to give a residue. The residue was purified by prep-HPLC (column: Phenomenex Luna 80*30 mm*3 um; mobile phase: [water(TFA)-ACN]; B %: 30%-60%, 8 min) to afford (S)-5-(dimethylamino)-6-ethyl-3-((3-(2-(2-(N-methylbut-2-ynamido)propanamido)ethyl)phenyl)amino)pyrazine-2-carboxamide (73 mg, 122.75 umol, 77.25% yield, 99.81% purity, TFA) as yellow solid. ¹H NMR (400 MHz, DMSO-d₆) δ=11.10 (d, J=2.7 Hz, 1H), 7.92 (s, 1H), 7.73 (br s, 1H), 7.56 (s, 1H), 7.53-7.40 (m, 2H), 7.20 (dt, J=1.8, 7.8 Hz, 1H), 6.78 (br d, J=7.5 Hz, 1H), 4.84 (dd, J=7.2, 18.9 Hz, 1H), 3.34-3.24 (m, 2H), 3.06 (s, 7H), 2.98 (s, 2H), 2.69-2.65 (m, 4H), 1.99 (d, J=16.8 Hz, 3H), 1.30-1.18 (m, 6H). LC-MS (ES⁺, m/z): 480.2 [(M+H)⁺]; Rt=2.607 min; HRMS: 480.2737.

### Example 70

### (Compound 162)

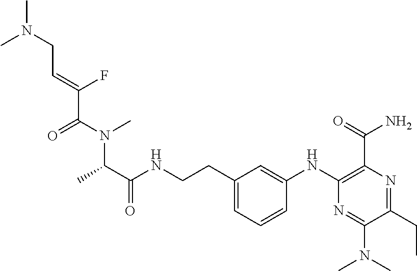 [SMILES: CCc1nc(C(N)=O)c(Nc2cccc(CCNC(=O)[C@H](C)N(C)C(=O)/C(F)=C/CN(C)C)c2)nc1N(C)C]

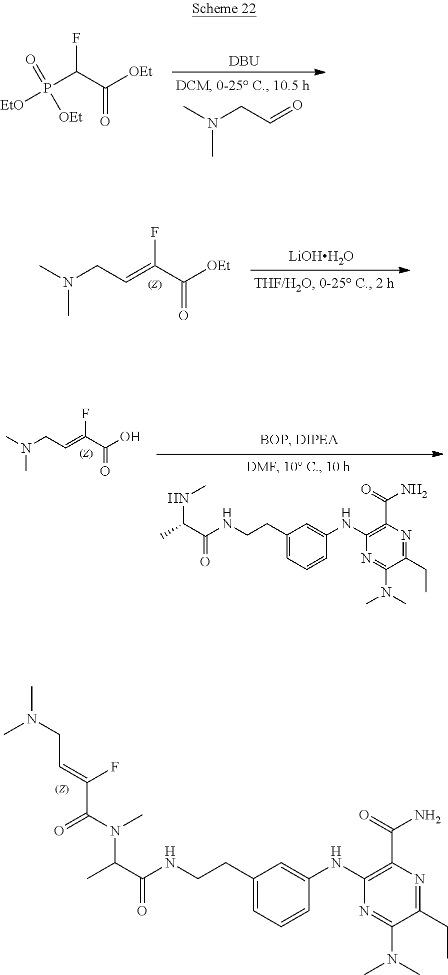

### (S,Z)-5-(dimethylamino)-3-((3-(2-(2-(4-(dimethylamino)-2-fluoro-N-methylbut-2-enamido)propanamido)ethyl)phenyl)amino)-6-ethylpyrazine-2-carboxamide

### Step 1: ethyl (Z)-4-(dimethylamino)-2-fluorobut-2-enoate

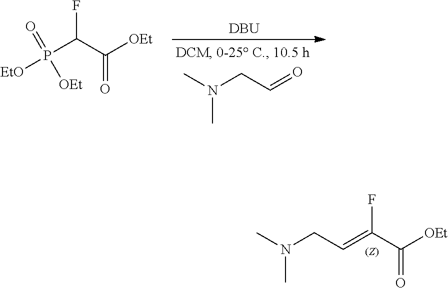

To a solution of ethyl 2-diethoxyphosphoryl-2-fluoro-acetate (10 g, 41.29 mmol, 8.40 mL, 1 eq) in DCM (100 mL) was added DBU (31.43 g, 206.46 mmol, 31.12 mL, 5 eq) at 0° C. After addition, the mixture was stirred at this temperature for 30 min, and then 2-(dimethylamino)acetaldehyde (10.48 g, 61.94 mmol, 1.5 eq, H₂SO₃) was added at 0° C. The resulting mixture was stirred at 25° C. for 10 hrs. HPLC showed the reaction was completed. The reaction mixture was quenched by addition sat. NH₄Cl (200 mL) at 0° C., and then extracted with DCM (100 mL*3). The combined organic layers were washed with sat. brine (50 mL*2), dried over anhydrous Na2SO4, filtered and concentrated under reduced pressure to give ethyl (Z)-4-(dimethylamino)-2-fluoro-but-2-enoate (4 g, crude) as yellow oil. LC-MS (ES+, m/z): 176.4 [(M+H)+]. Rt=0.247 min

### Step 2: (Z)-4-(dimethylamino)-2-fluorobut-2-enoic acid

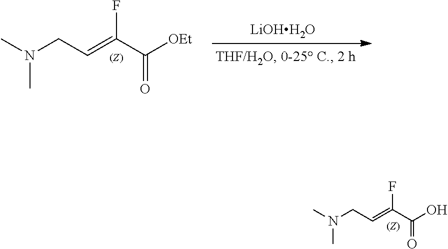

To a solution of ethyl (Z)-4-(dimethylamino)-2-fluoro-but-2-enoate (1 g, 5.71 mmol, 1 eq) in THF (10 mL) and H₂O (1 mL) was added LiOH·H₂O (718.55 mg, 17.12 mmol, 3 eq) at 0° C. After addition, the mixture was stirred at 25° C. for 2 hrs. LC-MS showed the reaction was completed. The reaction mixture was neutralized with 1M HCl and concentrated under reduced pressure to give a residue. The residue was purified by prep-HPLC (column: Phenomenex Luna C18 100*30 mm*5 um; mobile phase: [water(TFA)-ACN]; B %: 95%-70%, 10 min) to give compound (Z)-4-(dimethylamino)-2-fluorobut-2-enoic acid (620 mg, 4.21 mmol, 73.82% yield) as a white solid. LC-MS (ES+, m/z): 148.3 [(M+H)⁺]. Rt=0.183 min.

### Example 71

### (Compound 164)

### (S,E)-3-((3-(2-(2-(4-(bis(methyl-d3)amino)-N-methylbut-2-enamido)propanamido)ethyl)phenyl)amino)-6-ethyl-5-methylpyrazine-2-carboxamide

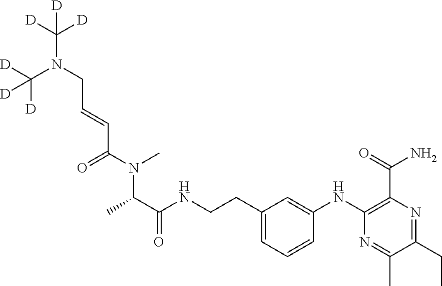 [SMILES: [2H]C([2H])([2H])N(C/C=C/C(=O)N(C)[C@@H](C)C(=O)NCCc1cccc(Nc2nc(C)c(CC)nc2C(N)=O)c1)C([2H])([2H])[2H]]

### Step 1: methyl (E)-4-(bis(methyl-d₃)amino)but-2-enoate

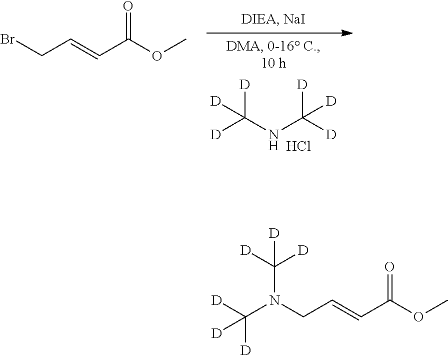

To a solution of DIEA (4.04 g, 31.28 mmol, 5.45 mL, 2.8 eq) bis(methyl-d₃)amine (1.96 g, 22.34 mmol, 2 eq) and NaI (5.86 g, 39.10 mmol, 3.5 eq) in DMA (10 mL) at 0° C., a solution of methyl (E)-4-bromobut-2-enoate (2 g, 11.17 mmol, 1.32 mL, 1 eq) in DMA (10 mL) was added dropwise to the solution. The mixture was stirred at 0° C. for 1 hr, then allowed to warm to 16° C. stirred for 9 hrs. LCMS showed the reaction was completed. The reaction was poured into water (40 ml) and extracted with DCM:MeOH=10:1 (30 mL*3). The organic layers were combined, washed with water (10 ml*2), saturated brine (20 mL*3), dried (Na₂SO₄), filtered and concentrated to give crude product. The crude product was purified by chromatography on silica thiol gel (DCM/MeOH=20/1) to give methyl (E)-4-(bis(methyl-ds)amino)but-2-enoate (240 mg, 1.61 mmol, 14.40% yield) as yellow oil. ¹H NMR (400 MHz, DMSO-d₆) δ=6.81 (td, J=6.9, 15.6 Hz, 1H), 6.25 (td, J=1.3, 15.6 Hz, 1H), 3.79 (dd, J=0.8, 6.8 Hz, 2H), 3.70 (s, 3H). LC-MS (ES⁺, m/z): 150.2 [(M+H)⁺]. Rt=0.447 min.

### Step 2: lithium (E)-4-(bis(methyl-d₃)amino)but-2-enoate

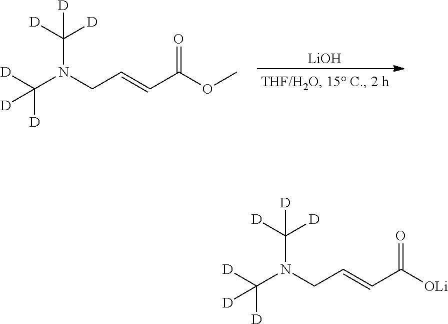

A mixture of methyl (E)-4-(bis(methyl-d₃)amino)but-2-enoate (240 mg, 1.61 mmol, 1 eq), LiOH·H₂O (202.46 mg, 4.83 mmol, 3 eq) and MeOH (1.5 mL) in H₂O (0.5 mL) was stirred at 15° C. for 2 hrs. LCMS indicated the reaction was complete. The mixture was concentrated under reduced pressure to give lithium (E)-4-(bis(methyl-d₃)amino)but-2-enoate (200 mg, 1.42 mmol, 88.11% yield) as yellow solid. LC-MS (ES⁺, m/z): 136.2 [(M+H)⁺]. Rt=0.242 min.

### Step 3: (S,E)-3-((3-(2-(2-(4-(bis(methyl-d₃)amino)-N-methylbut-2-enamido)propanamido)ethyl)phenyl)amino)-6-ethyl-5-methylpyrazine-2-carboxamide

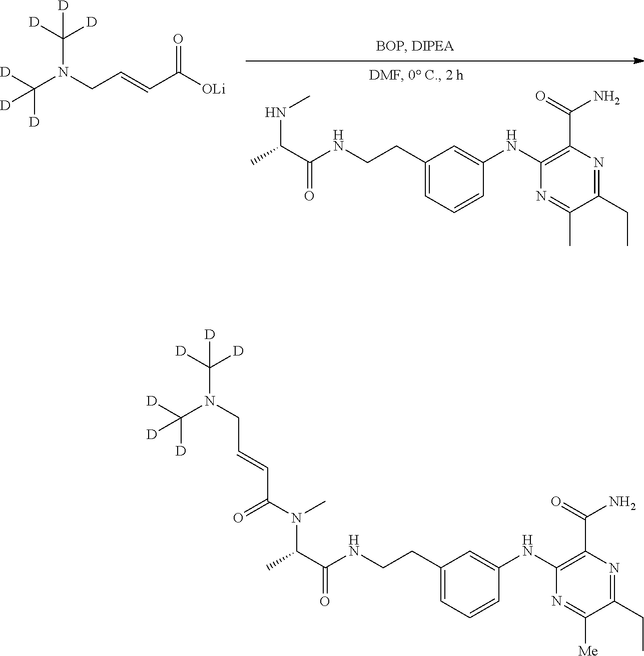

To a solution of lithium (E)-4-(bis(methyl-d₃)amino)but-2-enoate (176.02 mg, 1.25 mmol, 1.5 eq) and DIEA (1.07 g, 8.31 mmol, 1.45 mL, 10 eq) in DMF (2 mL) was added (S)-6-ethyl-5-methyl-3-((3-(2-(2-(methylamino)propanamido)ethyl)phenyl)amino)pyrazine-2-carboxamide (350 mg, 831.48 umol, 1 eq, HCl), and then BOP (551.62 mg, 1.25 mmol, 1.5 eq) was added at 0° C. The mixture was stirred at 0° C. for 2 hrs. LCMS indicated the reaction was complete. The reaction was concentrated. The crude was purified by prep-HPLC (column: C18-1 150*30 mm*5 um; mobile phase: [water(NH₄HCO₃)-MeCN]; B %: 25%-55%, 20 min) to give (S,E)-3-((3-(2-(2-(4-(bis(methyl-d3)amino)-N-methylbut-2-enamido)propanamido)ethyl)phenyl)amino)-6-ethyl-5-methylpyrazine-2-carboxamide (75.37 mg, 150.24 umol, 18.07% yield) as yellow solid. ¹H NMR (400 MHz, DMSO-d₆) 5=11.02 (s, 1H), 8.14 (s, 1H), 7.86-7.78 (m, 2H), 7.63 (br d, J=7.9 Hz, 1H), 7.49-7.39 (m, 1H), 7.22 (t, J=7.8 Hz, 1H), 6.81 (d, J=7.6 Hz, 1H), 6.65-6.49 (m, 1H), 6.46 (br d, J=15.0 Hz, 1H), 4.97 (br d, J=7.1 Hz, 1H), 3.38-3.22 (m, 2H), 3.04-2.89 (m, 2H), 2.83 (s, 2H), 2.77-2.67 (m, 5H), 2.49-2.48 (m, 3H), 1.28-1.16 (m, 6H); ¹H NMR (400 MHz, CDCl₃) δ=10.69 (s, 1H), 7.87 (br d, J=1.4 Hz, 1H), 7.63 (br d, J=7.4 Hz, 1H), 7.53 (br s, 1H), 7.25-7.20 (m, 1H), 6.91-6.79 (m, 2H), 6.45-6.24 (m, 2H), 5.47 (br s, 1H), 5.17 (q, J=7.0 Hz, 1H), 3.64-3.41 (m, 2H), 3.08 (br s, 2H), 2.86 (s, 3H), 2.81-2.72 (m, 4H), 2.54 (s, 3H), 1.32-1.26 (m, 6H). LC-MS (ES+, m/z): 502.3 [(M+H)⁺]; Rt=2.110 min; HRMS (EI): m/z [M]⁺ found: 502.3394; SFC: 96.74%.

### Example 72

### (Compound 204)

### (S,E)-3-((3-(2-(2-(4-(dimethylamino)-N-methylbut-2-enamido)propanamido)ethyl)phenyl)amino)-6-ethyl-5-(isopropyl(methyl)amino)pyrazine-2-carboxamide

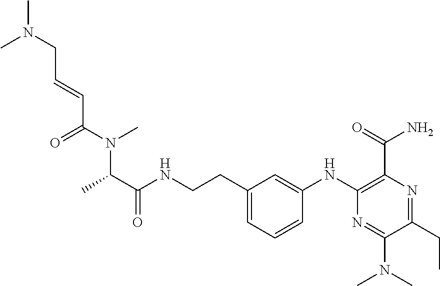 [SMILES: CCc1nc(C(N)=O)c(Nc2cccc(CCNC(=O)[C@H](C)N(C)C(=O)/C=C/CN(C)C)c2)nc1N(C)C]

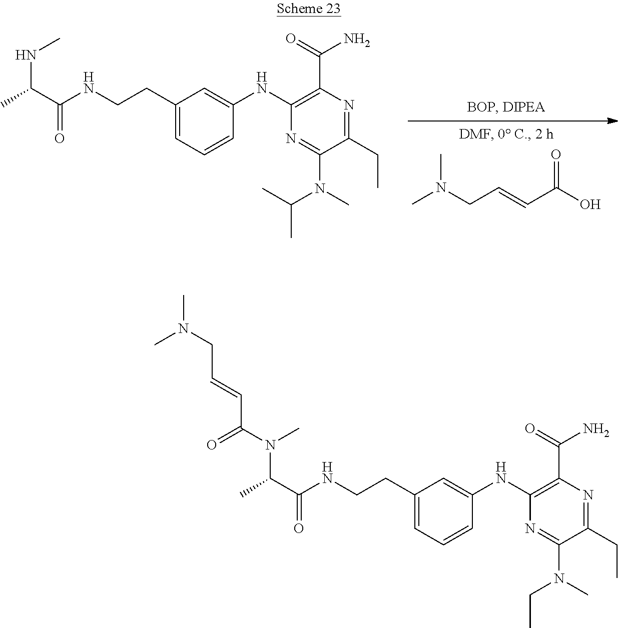

### Step 1: (S,E)-3-((3-(2-(2-(4-(dimethylamino)-N-methylbut-2-enamido)propanamido)ethyl)phenyl)amino)-6-ethyl-5-(isopropyl(methyl)amino)pyrazine-2-carboxamide

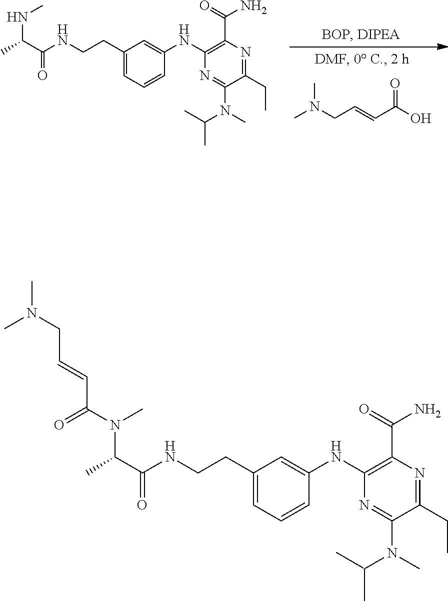

To a solution of (E)-4-(dimethylamino)but-2-enoic acid (118.46 mg, 917.19 umol, 1.5 eq) in DMF (2.5 mL) was added DIEA (790.27 mg, 6.11 mmol, 1.07 mL, 10 eq) and (S)-6-ethyl-5-(isopropyl(methyl)amino)-3-((3-(2-(2-(methylamino)propanamido)ethyl)phenyl)amino)pyrazine-2-carboxamide (270 mg, 611.46 umol, 1 eq), then BOP (405.65 mg, 917.19 umol, 1.5 eq) was added. The mixture was stirred at 0° C. for 2 hrs. LCMS indicated the reaction was complete. The reaction was concentrated. The crude was purified by prep-HPLC (column: Phenomenex Luna C18 150*30 mm*5 um; mobile phase: [water(TFA)-ACN]; B %: 15%-45%, 8 min) to give (S,E)-3-((3-(2-(2-(4-(dimethylamino)-N-methylbut-2-enamido)propanamido)ethyl)phenyl)amino)-6-ethyl-5-(isopropyl(methyl)amino) pyrazine-2-carboxamide (64.81 mg, 116.25 umol, 19.01% yield) as yellow solid. 1H NMR (400 MHz, DMSO-d₆) δ=11.15-11.06 (m, 1H), 9.67 (br s, 1H), 8.11-7.88 (m, 1H), 7.76 (br d, J=2.1 Hz, 1H), 7.55-7.50 (m, 1H), 7.49-7.42 (m, 2H), 7.24-7.16 (m, 1H), 6.86-6.75 (m, 2H), 6.65-6.46 (m, 1H), 4.99-4.54 (m, 1H), 4.25 (td, J=6.6, 13.2 Hz, 1H), 3.90-3.81 (m, 2H), 3.33-3.23 (m, 2H), 2.91-2.84 (m, 5H), 2.80-2.67 (m, 11H), 1.30-1.19 (m, 12H). LC-MS (ES+, m/z): 553.4 [(M+H)⁺]; Rt=2.247 min; HRMS (EI): m/z [M]⁺ found: 553.3696; SFC: 99.14%.

### Example 73

### (Compound 228)

### (S,E)-5-cyclopropyl-3-((3-(2-(2-(4-(dimethylamino)-N-methylbut-2-enamido)propanamido)ethyl)phenyl)amino)-6-methylpyrazine-2-carboxamide

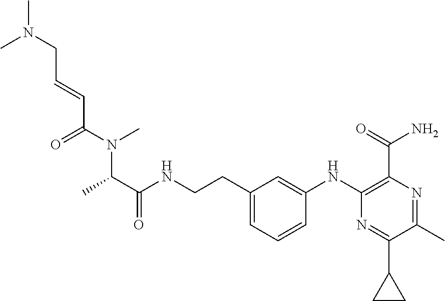

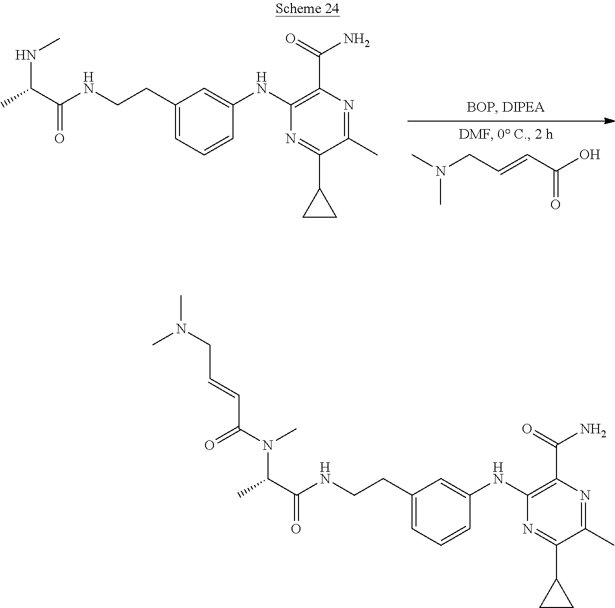

### Step 1: (S,E)-5-cyclopropyl-3-((3-(2-(2-(4-(dimethylamino)-N-methylbut-2-enamido)propanamido)ethyl)phenyl)amino)-6-methylpyrazine-2-carboxamide

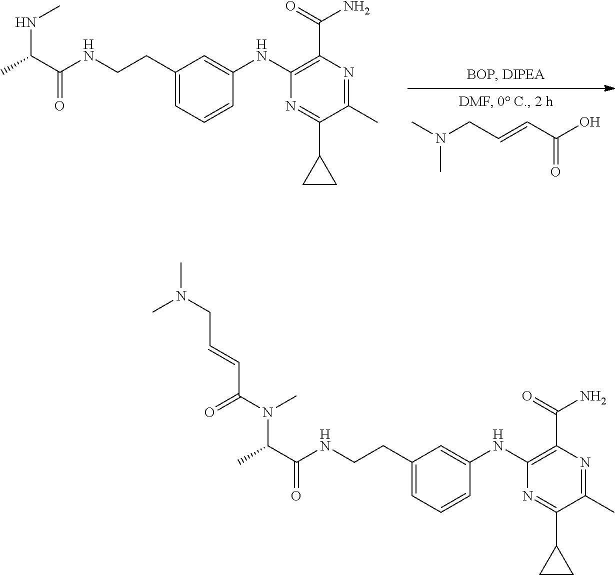

A mixture of (E)-4-(dimethylamino)but-2-enoic acid (573.80 mg, 3.46 mmol, 1.5 eq, HCl) in DMF (8 mL), DIPEA (2.99 g, 23.10 mmol, 10 eq), (S)-5-cyclopropyl-6-methyl-3-((3-(2-(2-(methylamino)propanamido)ethyl)phenyl)amino) pyrazine-2-carboxamide (1 g, 2.31 mmol, 1 eq, HCl) was added at 0° C., and then BOP (1.53 g, 3.46 mmol, 1.5 eq) was added. The mixture was stirred at 0° C. for 2 h. LCMS indicated the reaction was complete. The mixture was concentrated to dryness. The mixture was purified by prep-HPLC (column: Phenomenex luna C18 250*50 mm*10 um; mobile phase: [water(TFA)-ACN]; B %: 20%-50%, 10 min) to give (S,E)-5-cyclopropyl-3-((3-(2-(2-(4-(dimethylamino)-N-methylbut-2-enamido) propanamido)ethyl)phenyl)amino)-6-methylpyrazine-2-carboxamide (320 mg, 622.44 umol, 26.95% yield) as yellow solid. ¹H NMR (400 MHz, DMSO-d₆) δ=11.05 (s, 1H), 9.70 (br s, 1H), 8.20 (br s, 1H), 8.11-7.88 (m, 1H), 7.80 (br s, 1H), 7.54-7.36 (m, 2H), 7.22 (br t, J=7.8 Hz, 1H), 6.81 (q, J=8.0 Hz, 2H), 6.66-6.45 (m, 1H), 5.05-4.51 (m, 1H), 3.98-3.80 (m, 2H), 3.45-3.28 (m, 2H), 2.90 (s, 2H), 2.81-2.66 (m, 9H), 2.56 (s, 3H), 2.30-2.20 (m, 1H), 1.33-1.18 (m, 3H), 1.14-1.01 (m, 4H)(TFA, salt); LCMS (ES+, m/z): 508.2 [(M+H)⁺]; Rt=2.117 min; HRMS (EI): m/z [M]⁺ found: 508.3014

### Example 74

### (Compound 232)

### (S,E)-3-((3-(2-(2-(4-(dimethylamino)-N-methylbut-2-enamido)propanamido)ethyl)phenyl)amino)-5-(isopropyl(methyl)amino)-6-methylpyrazine-2-carboxamide

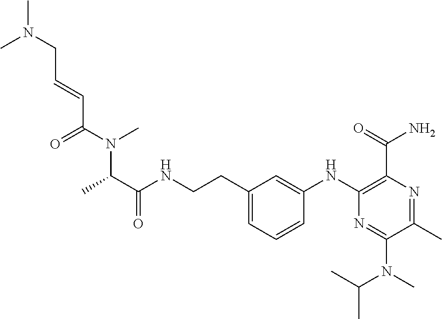

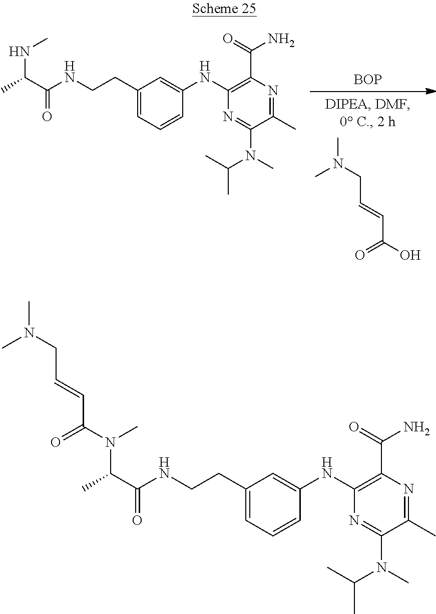

### Step 1: (S,E)-3-((3-(2-(2-(4-(dimethylamino)-N-methylbut-2-enamido)propanamido)ethyl)phenyl)amino)-5-(isopropyl(methyl)amino)-6-methylpyrazine-2-carboxamide

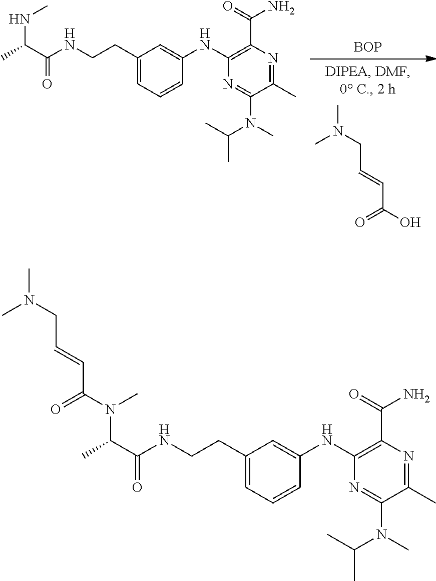

To a solution of (E)-4-(dimethylamino)but-2-enoic acid (1.49 g, 11.51 mmol, 1.2 eq) in DMF (40 mL) was added DIEA (12.39 g, 95.90 mmol, 16.70 mL, 10 eq), (S)-5-(isopropyl(methyl)amino)-6-methyl-3-((3-(2-(2-(methylamino)propanamido)ethyl)phenyl)amino) pyrazine-2-carboxamide (4.1 g, 9.59 mmol, 1 eq), and then added BOP (5.09 g, 11.51 mmol, 1.2 eq). The mixture was stirred at 0° C. for 2 hr. LCMS showed the reaction was completed. The reaction mixture was quenched by addition water (120 mL), and then extracted with EtOAc (90 mL*3). The combined organic layers were washed with saturated brine (100 mL*2), dried over Na₂SO₄, filtered and concentrated under reduced pressure to give a residue. The residue was purified by column chromatography (SiO₂, DCM/MeOH=30/1 to 5/1) to give (S,E)-3-((3-(2-(2-(4-(dimethylamino)-N-methylbut-2-enamido)propanamido)ethyl)phenyl)amino)-5-(isopropyl(methyl)amino)-6-methylpyrazine-2-carboxamide as yellow solid (96.13 mg, 198.40 umol, 32.58% yield). ¹H NMR (400 MHz, DMSO-d₆) δ=11.11 (s, 1H), 8.07 (br s, 1H), 7.81-7.77 (m, 1H), 7.55-7.51 (m, 1H), 7.46-7.41 (m, 2H), 7.22-7.16 (m, 1H), 6.80-6.75 (m, 1H), 6.62-6.49 (m, 2H), 5.02-4.94 (m, 1H), 4.34 (quin, J=6.6 Hz, 1H), 3.32-3.24 (m, 2H), 3.16-3.08 (m, 2H), 2.91-2.88 (m, 3H), 2.86-2.83 (m, 2H), 2.67 (br s, 3H), 2.44 (s, 3H), 2.24-2.20 (m, 6H), 1.24-1.16 (m, 9H). LC-MS (ES+, m/z): 539.4 [(M+H)+]; Rt=2.178 min; HRMS (EI): m/z [M]⁺ found: 539.3435.

### Example 76

### (Compound 205)

### (S,E)-3-((3-(2-(2-(4-(dimethylamino)-N-methylbut-2-enamido)propanamido)ethyl)phenyl)amino)-6-ethyl-5-(isopropylamino)pyrazine-2-carboxamide

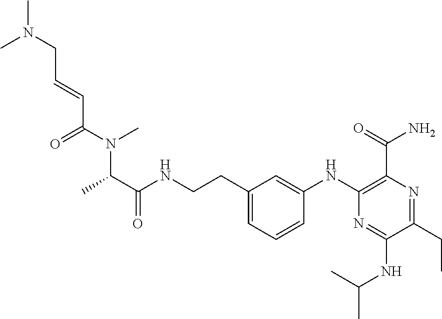 [SMILES: CCc1nc(C(N)=O)c(Nc2cccc(CCNC(=O)[C@H](C)N(C)C(=O)/C=C/CN(C)C)c2)nc1NC(C)C]

### Step 1: tert-butyl (3-((3-carbamoyl-5-ethyl-6-(isopropylamino) pyrazin-2-yl)amino)phenethyl)carbamate

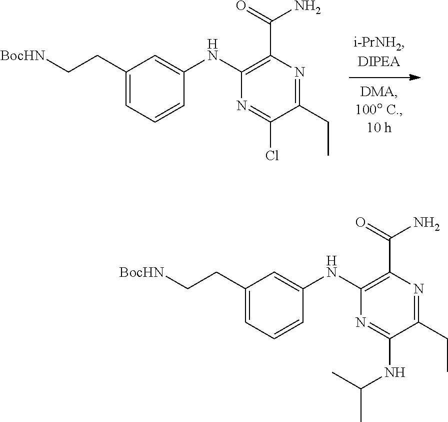 [SMILES: CCc1nc(C(N)=O)c(Nc2cccc(CCNC(=O)OC(C)(C)C)c2)nc1Cl ; CCc1nc(C(N)=O)c(Nc2cccc(CCNC(=O)OC(C)(C)C)c2)nc1NC(C)C]

To a solution of tert-butyl (3-((3-carbamoyl-6-chloro-5-ethylpyrazin-2-yl)amino)phenethyl)carbamate (200 mg, 476.30 umol, 1 eq) in DMA (2 mL) was added propan-2-amine (140.77 mg, 2.38 mmol, 5 eq) and DIPEA (615.58 mg, 4.76 mmol, 829.63 uL, 10 eq). The mixture was stirred at 100° C. for 10 hr. LCMS showed the reaction was completed. The reaction mixture was quenched by addition water (50 mL) at 25° C., and then extracted with EtOAc (30 mL*3). The combined organic layers were washed with saturated brine (20 mL*2), dried over Na₂SO₄, filtered and concentrated under reduced pressure to give a residue. The residue was purified by column chromatography (SiO₂, Petroleum ether/Ethyl acetate=10/1 to 1/1) to give tert-butyl (3-((3-carbamoyl-5-ethyl-6-(isopropylamino) pyrazin-2-yl)amino) phenethyl) carbamate (180 mg, 389.97 umol, 81.88% yield) as yellow solid. ¹H NMR (400 MHz, DMSO-d₆) δ=11.22 (s, 1H), 7.73-7.68 (m, 1H), 7.57-7.53 (m, 1H), 7.33-7.24 (m, 2H), 7.21-7.15 (m, 1H), 6.92 (t, J=5.3 Hz, 1H), 6.78-6.74 (m, 2H), 4.31-3.98 (m, 1H), 3.15-3.10 (m, 2H), 2.67-2.63 (m, 2H), 2.61-2.55 (m, 2H), 1.37 (s, 9H), 1.27 (d, J=6.6 Hz, 6H), 1.21-1.17 (m, 3H). LC-MS (ES⁺, m/z): 443.3 [(M+H)+]; Rt=0.887 min.

### Step 2: 3-((3-(2-aminoethyl)phenyl)amino)-6-ethyl-5-(isopropylamino)pyrazine-2-carboxamide

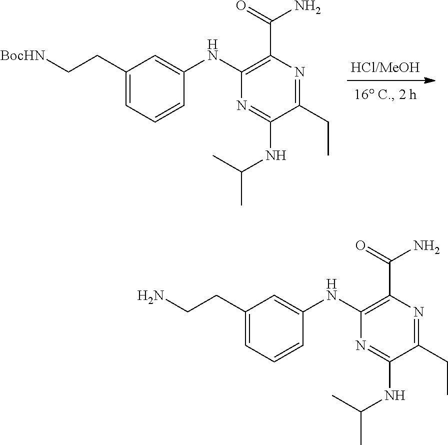 [SMILES: CCc1nc(C(N)=O)c(Nc2cccc(CCNC(=O)OC(C)(C)C)c2)nc1NC(C)C ; CCc1nc(C(N)=O)c(Nc2cccc(CCN)c2)nc1NC(C)C]

To tert-butyl (3-((3-carbamoyl-5-ethyl-6-(isopropylamino)pyrazin-2-yl)amino)phenethyl)carbamate (180 mg, 406.73 umol, 1 eq) was added HCl/MeOH (4 M, 101.68 uL). The mixture was stirred at 16° C. for 2 hr. LCMS showed the reaction was completed. The reaction mixture was concentrated under reduced pressure to give a residue. to give a residue. The crude product was triturated with EtOAc at 25° C. for 10 min to give 3-((3-(2-aminoethyl)phenyl)amino)-6-ethyl-5-(isopropylamino)pyrazine-2-carboxamide (130 mg, 367.71 umol, 90.41% yield) as yellow solid. 1H NMR (400 MHz, DMSO-d₆) δ=11.25 (br s, 1H), 8.08 (br s, 3H), 7.57 (s, 1H), 7.53 (br d, J=8.1 Hz, 1H), 7.24 (t, J=7.8 Hz, 2H), 6.86-6.82 (m, 1H), 6.82-6.74 (m, 1H), 4.29-4.18 (m, 1H), 3.05-2.96 (m, 2H), 2.90-2.83 (m, 2H), 2.61-2.55 (m, 2H), 1.28 (d, J=6.5 Hz, 6H), 1.19 (t, J=7.4 Hz, 3H); LC-MS (ES⁺, m/z): 343.2 [(M+H)+]; Rt=0.684 min

### Step 3: tert-butyl (S)-(1-((3-((3-carbamoyl-5-ethyl-6-(isopropylamino) pyrazin-2-yl)amino)phenethyl)amino)-1-oxopropan-2-yl)(methyl)carbamate

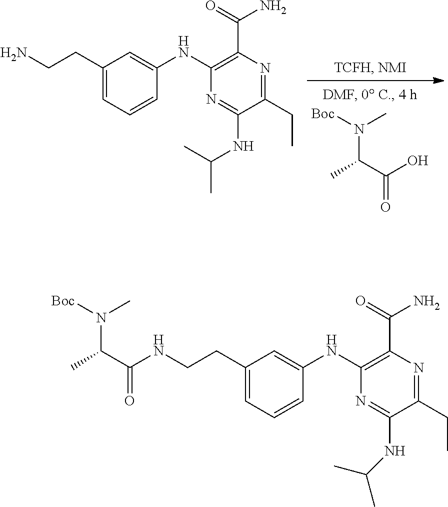

To a solution of 3-((3-(2-aminoethyl)phenyl)amino)-6-ethyl-5-(isopropylamino)pyrazine-2-carboxamide (130 mg, 379.63 umol, 1 eq), 1-methylimidazole (311.68 mg, 3.80 mmol, 302.60 uL, 10 eq) and N-(tert-butoxycarbonyl)-N-methyl-L-alanine (115.73 mg, 569.45 umol, 1.5 eq), 1-methylimidazole (311.68 mg, 3.80 mmol, 302.60 uL, 10 eq) in DMF (1.5 mL) was added TCFH (159.78 mg, 569.45 umol, 1.5 eq) at 0° C. The mixture was stirred at 0° C. for 4 hrs. LCMS showed the reaction was completed. The reaction mixture was quenched by addition water (50 mL) at 25° C., and then extracted with EtOAc (30 mL*3). The combined organic layers were washed with saturated brine (20 mL*2), dried over Na₂SO₄, filtered and concentrated under reduced pressure to give a residue. The residue was purified by column chromatography (SiO₂, Petroleum ether/Ethyl acetate=10/1 to 5/1) to give tert-butyl (S)-(1-((3-((3-carbamoyl-5-ethyl-6-(isopropylamino) pyrazin-2-yl)amino)phenethyl)amino)-1-oxopropan-2-yl)(methyl)carbamate (130 mg, 204.32 umol, 53.82% yield) as yellow solid. ¹H NMR (400 MHz, DMSO-d₆) δ=11.21 (s, 1H), 7.84-7.77 (m, 1H), 7.67 (s, 1H), 7.57-7.51 (m, 1H), 7.35 (br d, J=8.1 Hz, 1H), 7.24 (br d, J=2.4 Hz, 1H), 7.18 (t, J=7.8 Hz, 1H), 6.76 (t, J=8.3 Hz, 2H), 4.29-4.23 (m, 1H), 3.32-3.26 (m, 3H), 2.69 (s, 5H), 2.58 (q, J=7.4 Hz, 2H), 1.37 (br s, 9H), 1.28-1.17 (m, 12H), LC-MS (ES⁺, m/z): 528.4 [(M+H)⁺]; Rt=0.929 min.

### Step 4: (S)-6-ethyl-5-(isopropylamino)-3-((3-(2-(2-(methylamino)propanamido)ethyl)phenyl)amino)pyrazine-2-carboxamide

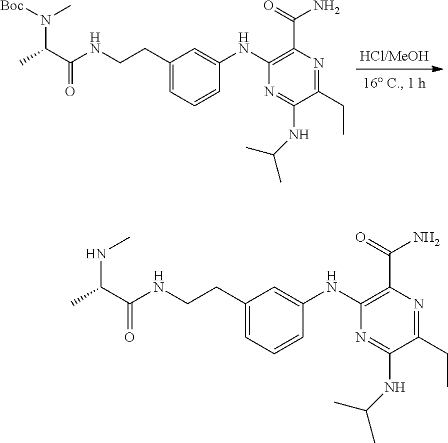

A mixture of tert-butyl (S)-(1-((3-((3-carbamoyl-5-ethyl-6-(isopropylamino)pyrazin-2-yl)amino)phenethyl)amino)-1-oxopropan-2-yl)(methyl)carbamate (130 mg, 246.37 umol, 1 eq) HCl/MeOH (4 M, 61.59 uL, 1 eq) was stirred at 16° C. for 1 hr. LCMS showed the reaction was completed. The reaction mixture was concentrated under reduced pressure to give a residue. The crude product was triturated with EtOAc at 25° C. for 10 min to give (S)-6-ethyl-5-(isopropylamino)-3-((3-(2-(2-(methylamino)propanamido)ethyl)phenyl)amino)pyrazine-2-carboxamide as yellow solid (100 mg, 215.39 umol, 87.43% yield). ¹H NMR (400 MHz, DMSO-d₆) δ=11.21 (br s, 1H), 9.24-9.15 (m, 1H), 8.85-8.76 (m, 1H), 8.64 (br t, J=5.4 Hz, 1H), 7.58 (s, 1H), 7.45 (br d, J=8.0 Hz, 1H), 7.20 (t, J=7.8 Hz, 1H), 6.80 (br d, J=7.4 Hz, 2H), 4.27 (br s, 1H), 3.74-3.64 (m, 1H), 3.43-3.37 (m, 1H), 2.85-2.67 (m, 4H), 2.59 (q, J=7.4 Hz, 3H), 2.42-2.40 (m, 2H), 1.32-1.17 (m, 12H); LC-MS (ES⁺, m/z): 428.3 [(M+H)⁺]; Rt=0.731 min.; LC-MS (ES⁺, m/z): 428.3 [(M+H)⁺]; Rt=0.731 min.

Note: HCl/EtOAc (4 M): HCl was bubbled into a solution EtOAc at 0° C. for 0.5 h. Then, the solution was weighed to obtained the HCl/EtOAc (4 M)

### Step 5: (S,E)-3-((3-(2-(2-(4-(dimethylamino)-N-methylbut-2-enamido)propanamido)ethyl)phenyl)amino)-6-ethyl-5-(isopropylamino) pyrazine-2-carboxamide

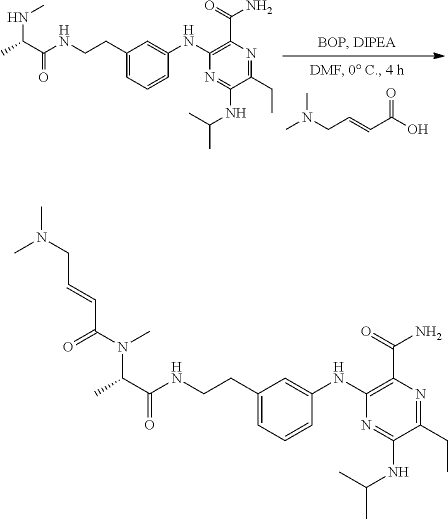

To a solution of (S)-6-ethyl-5-(isopropylamino)-3-((3-(2-(2-(methylamino) propanamido)ethyl)phenyl)amino)pyrazine-2-carboxamide (100 mg, 233.90 umol, 1 eq) in DMF (3 mL) was added BOP (124.14 mg, 280.67 umol, 1.2 eq) and DIPEA (302.29 mg, 2.34 mmol, 407.40 uL, 10 eq), (E)-4-(dimethylamino)but-2-enoic acid (36.25 mg, 280.67 umol, 1.2 eq). The mixture was stirred at 0° C. for 4 hrs. LC-MS indicated the mixture was complete. The reaction mixture was quenched by addition water (50 mL) at 25° C., and then extracted with EtOAc (30 mL*3). The combined organic layers were washed with saturated brine (20 mL*2), dried over Na₂SO₄, filtered and concentrated under reduced pressure to give a residue. The residue was purified by prep-HPLC (column: Phenomenex Luna C18 150*30 mm*5 um; mobile phase: [water(TFA)-ACN]; B %: 15%-45%, 8 min) to give desired compound to give (S,E)-3-((3-(2-(2-(4-(dimethylamino)-N-methylbut-2-enamido)propanamido)ethyl)phenyl)amino)-6-ethyl-5-(isopropylamino)pyrazine-2-carboxamide (13.13 mg, 23.96 umol, 10.24% yield) as yellow solid. ¹H NMR (400 MHz, DMSO-d₆) δ=11.25-11.18 (m, 1H), 9.90 (br s, 1H), 7.92 (br t, J=5.5 Hz, 1H), 7.64-7.52 (m, 2H), 7.44-7.37 (m, 1H), 7.25 (br s, 1H), 7.21-7.15 (m, 1H), 6.85-6.73 (m, 3H), 6.66-6.47 (m, 1H), 4.57 (q, J=6.7 Hz, 1H), 4.26 (qd, J=6.7, 13.6 Hz, 1H), 3.93-3.81 (m, 2H), 3.34-3.23 (m, 2H), 2.90 (s, 2H), 2.80-2.74 (m, 6H), 2.73-2.66 (m, 3H), 2.62-2.56 (m, 2H), 1.29-1.16 (m, 12H)(TFA, salt); LC-MS (ES⁺, m/z): 539.3 [(M+H)+]; Rt=2.117 min; 98.31% purity; HRMS: 539.3476.

### Example 77

### (Compound 219)

### (E)-5-cyclopropyl-3-((3-(2-(2-(4-(dimethylamino)-N-methylbut-2-enamido)acetamido)ethyl)phenyl)amino)-6-ethylpyrazine-2-carboxamide

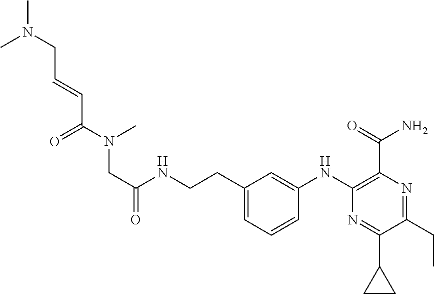 [SMILES: CCc1nc(C(N)=O)c(Nc2cccc(CCNC(=O)CN(C)C(=O)/C=C/CN(C)C)c2)nc1C1CC1]

### Step 1: tert-butyl (3-((3-carbamoyl-6-cyclopropyl-5-ethylpyrazin-2-yl)amino)phenethyl)carbamate

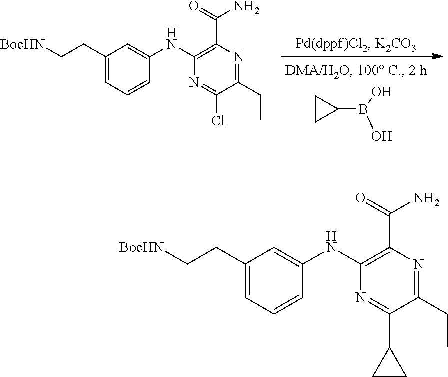 [SMILES: CCc1nc(C(N)=O)c(Nc2cccc(CCNC(=O)OC(C)(C)C)c2)nc1Cl ; OB(O)C1CC1 ; CCc1nc(C(N)=O)c(Nc2cccc(CCNC(=O)OC(C)(C)C)c2)nc1C1CC1]

To a solution of tert-butyl (3-((3-carbamoyl-6-chloro-5-ethylpyrazin-2-yl)amino) phenethyl) carbamate (1 g, 2.38 mmol, 1 eq) in DMA (10 mL) was added K₂CO₃ (1.15 g, 8.34 mmol, 3.5 eq) and Pd(dppf)Cl₂ (1.74 g, 2.38 mmol, 1 eq), cyclopropylboronic acid (245.48 mg, 2.86 mmol, 1.2 eq). The mixture was stirred at 100° C. for 2 hours under N₂. LCMS showed the reaction was completed. The reaction mixture was quenched by addition water (50 mL) at 25° C., The reaction mixture was poured into saturated EDTA (50 mL) and stirred 60 min, then extracted with EtOAc (30 mL*3). The combined organic layers were washed with saturated brine (20 mL*2), dried over Na₂SO₄, filtered and concentrated under reduced pressure to give a residue. The residue was purified by column chromatography (SiO₂, Petroleum ether/Ethyl acetate=10/1 to 5/1) to give tert-butyl (3-((3-carbamoyl-6-cyclopropyl-5-ethylpyrazin-2-yl)amino)phenethyl)carbamate as yellow solid (1 g, 2.04 mmol, 85.85% yield, 87% purity). ¹H NMR (400 MHz, DMSO-d₆) δ=11.06-11.00 (m, 1H), 8.13-8.09 (m, 1H), 7.81 (br d, J=1.5 Hz, 1H), 7.41 (s, 1H), 7.26 (s, 1H), 7.21 (t, J=7.8 Hz, 1H), 6.81 (br d, J=7.5 Hz, 1H), 4.07-3.97 (m, 1H), 3.19-3.12 (m, 2H), 2.92-2.85 (m, 2H), 2.67 (br t, J=7.3 Hz, 2H), 2.31-2.23 (m, 1H), 1.35-1.15 (m, 12H), 1.13-1.06 (m, 4H). LC-MS (ES⁺, m/z): 426.2 [(M+H)⁺]; Rt=0.928 min.

### Step 2: 3-((3-(2-aminoethyl)phenyl)amino)-5-cyclopropyl-6-ethylpyrazine-2-carboxamide hydrochloride

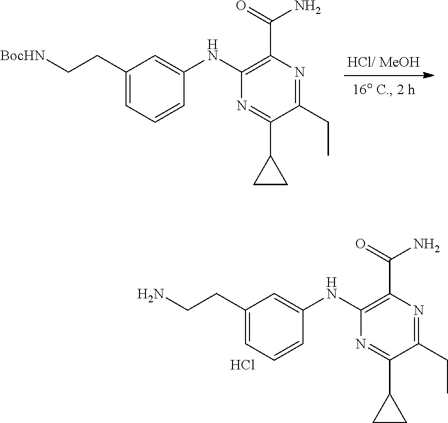 [SMILES: CCc1nc(C(N)=O)c(Nc2cccc(CCNC(=O)OC(C)(C)C)c2)nc1C1CC1 ; CCc1nc(C(N)=O)c(Nc2cccc(CCN)c2)nc1C1CC1 ; Cl]

To tert-butyl (3-((3-carbamoyl-6-cyclopropyl-5-ethylpyrazin-2-yl)amino) phenethyl) carbamate (1 g, 2.35 mmol, 1 eq) was added HCl/MeOH (4 M, 25.00 mL, 42.55 eq). The mixture was stirred at 16° C. for 2 hours. LCMS showed the reaction was completed, the reaction mixture was concentrated under reduced pressure to give 3-((3-(2-aminoethyl)phenyl)amino)-5-cyclopropyl-6-ethylpyrazine-2-carboxamide hydrochloride as yellow solid (0.9 g, crude, HCl). ¹H NMR (400 MHz, DMSO-d₆) δ=11.07 (s, 1H), 8.13 (br s, 4H), 7.88-7.78 (m, 1H), 7.59-7.48 (m, 1H), 7.46-7.40 (m, 1H), 7.33-7.22 (m, 1H), 6.95-6.81 (m, 1H), 3.10-2.99 (m, 2H), 2.96-2.83 (m, 4H), 2.37-2.21 (m, 1H), 1.27 (t, J=7.5 Hz, 3H), 1.14-1.03 (m, 4H)(HCl salt); LC-MS (ES⁺, m/z): 362.2 [(M+H)⁺]. Rt=0.711 min.

Note: HCl/MeOH (4 M): HCl gas was bubbled into a solution MeOH at 0° C. for 0.5 h.

### Step 3: (E)-5-cyclopropyl-3-((3-(2-(2-(4-(dimethylamino)-N-methylbut-2-enamido)acetamido)ethyl)phenyl)amino)-6-ethylpyrazine-2-carboxamide

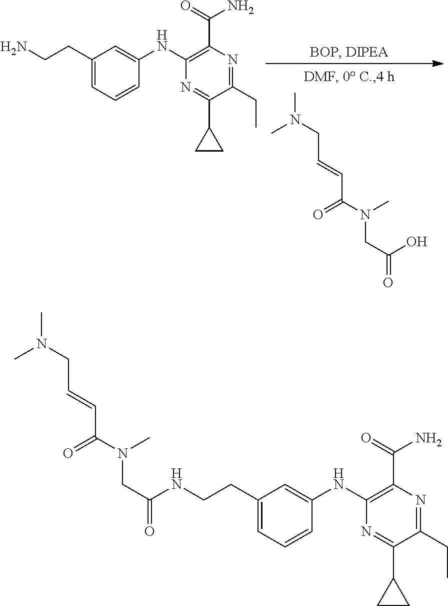

To a solution of 3-((3-(2-aminoethyl)phenyl)amino)-5-cyclopropyl-6-ethylpyrazine-2-carboxamide (80 mg, 245.85 umol, 1 eq) in DMF (0.5 mL) was added DIEA (317.73 mg, 2.46 mmol, 428.21 uL, 10 eq) (E)-N-(4-(dimethylamino)but-2-enoyl)-N-methylglycine (172.29 mg, 344.18 umol, 40% purity, 1.4 eq) and then BOP (152.23 mg, 344.18 umol, 1.4 eq). The mixture was stirred at 0° C. for 4 hours. LCMS indicated the reaction was completed. The reaction mixture was quenched by addition water (50 mL), and then extracted with EtOAc (30 mL*3). The combined organic layers were washed with saturated brine (20 mL*2), dried over Na₂SO₄, filtered and concentrated under reduced pressure to give a residue. The residue was purified by prep-HPLC (column: Phenomenex Luna C18 150*30 mm*5 um; mobile phase: [water(TFA)-ACN]; B %: 15%-35%, 8 min) to give desired compound (E)-5-cyclopropyl-3-((3-(2-(2-(4-(dimethylamino)-N-methylbut-2-enamido) acetamido)ethyl)phenyl)amino)-6-ethylpyrazine-2-carboxamide (10.37 mg, 20.43 umol, 8.31% yield, 100% purity) as yellow solid. ¹H NMR (400 MHz, DMSO-d₆) δ=11.05 (d, J=2.3 Hz, 1H), 9.69 (br d, J=1.5 Hz, 1H), 8.23-7.99 (m, 2H), 7.84-7.78 (m, 1H), 7.50-7.38 (m, 2H), 7.27-7.20 (m, 1H), 6.91-6.67 (m, 2H), 6.63-6.49 (m, 1H), 4.04-3.94 (m, 2H), 3.92-3.79 (m, 2H), 3.31-3.27 (m, 2H), 3.06-3.02 (m, 2H), 2.94-2.87 (m, 2H), 2.80-2.66 (m, 9H), 2.32-2.24 (m, 1H), 1.27 (t, J=7.5 Hz, 3H), 1.13-1.04 (m, 4H), LC-MS (ES+, m/z): 508.3 [(M+H)⁺]. Rt=2.182 min. HRMS (EI): m/z [M]⁺ found: 508.3000.

### Example 78

### (Compound 221)

### (S,E)-3-((3-(2-(2-(4-(dimethylamino)-N-methylbut-2-enamido)propanamido)ethyl)phenyl)amino)-6-ethyl-5-(ethyl(methyl)amino)pyrazine-2-carboxamide

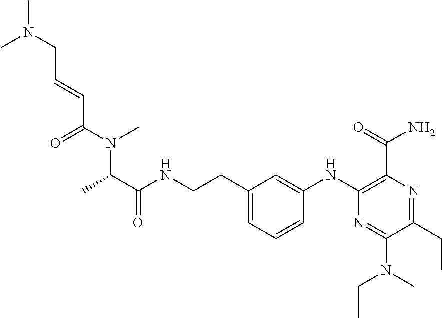 [SMILES: CCc1nc(C(N)=O)c(Nc2cccc(CCNC(=O)[C@H](C)N(C)C(=O)/C=C/CN(C)C)c2)nc1N(C)CC]

### Step 1: tert-butyl (3-((3-carbamoyl-5-ethyl-6-(ethyl(methyl)amino)pyrazin-2-yl)amino)phenethyl)carbamate

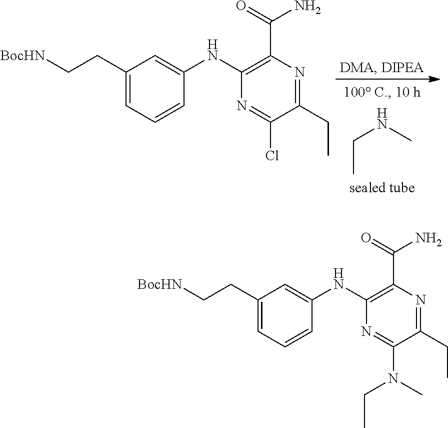

To a solution of tert-butyl (3-((3-carbamoyl-6-chloro-5-ethylpyrazin-2-yl)amino)phenethyl)carbamate in DMA (10 mL) was added DIPEA (1.54 g, 11.91 mmol, 2.07 mL, 10 eq) and N-methylethanamine (703.85 mg, 11.91 mmol, 1.02 mL, 10 eq) at 20° C. The mixture was stirred at 100° C. for 10 hr at sealed tube. LCMS showed the reaction was completed. The reaction was poured into water (15 mL) and extracted with EtOAc (20 mL*3). The organic layers were combined, washed with water (10 mL*3), saturated brine (10 mL*3), dried (Na₂SO₄), filtered and concentrated to give crude product. The residue was purified by column chromatography (SiO₂, Petroleum ether/Ethyl acetate=5/1 to 1/1) to afford tert-butyl (3-((3-carbamoyl-5-ethyl-6-(ethyl(methyl)amino)pyrazin-2-yl)amino)phenethyl)carbamate (430 mg, 971.63 umol, 81.60% yield) as a yellow solid. ¹H NMR (400 MHz, DMSO-d₆) δ=11.12-11.06 (m, 1H), 7.81-7.65 (m, 1H), 7.61-7.52 (m, 1H), 7.49-7.36 (m, 2H), 7.23-7.16 (m, 1H), 6.92-6.83 (m, 1H), 6.78 (d, J=7.4 Hz, 1H), 3.52-3.40 (m, 2H), 3.17-3.14 (m, 2H), 3.04 (s, 3H), 2.76-2.64 (m, 4H), 1.38-1.33 (m, 9H), 1.24-1.18 (m, 6H). LC-MS (ES+, m/z): 443.3 [(M+H)⁺]; Rt=0.908 min.

### Step 2: 3-((3-(2-aminoethyl)phenyl)amino)-6-ethyl-5-(ethyl(methyl)amino) pyrazine-2-carboxamide

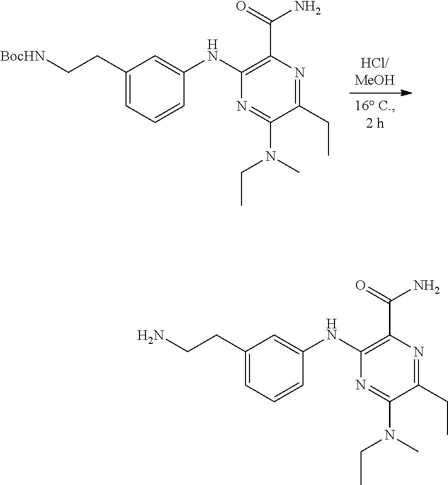 [SMILES: CCc1nc(C(N)=O)c(Nc2cccc(CCNC(=O)OC(C)(C)C)c2)nc1N(C)CC ; CCc1nc(C(N)=O)c(Nc2cccc(CCN)c2)nc1N(C)CC]

A mixture of tert-butyl (3-((3-carbamoyl-5-ethyl-6-(ethyl(methyl)amino) pyrazin-2-yl)amino)phenethyl)carbamate (400 mg, 903.85 umol, 1 eq) in HCl/MeOH (40 mL) was stirred at 16° C. for 2 hrs. LCMS showed the reaction was completed. The reaction mixture was filtered and concentrated under reduced pressure to afford 3-((3-(2-aminoethyl)phenyl)amino)-6-ethyl-5-(ethyl(methyl)amino)pyrazine-2-carboxamide (410 mg, crude) as yellow solid. ¹H NMR (400 MHz, DMSO-d₆) δ=11.14 (s, 1H), 7.75 (br s, 4H), 7.61-7.55 (m, 1H), 7.52 (s, 1H), 7.50-7.41 (m, 1H), 7.26 (t, J=7.8 Hz, 1H), 6.89-6.83 (m, 1H), 4.11 (br s, 2H), 3.46 (q, J=7.0 Hz, 2H), 3.04 (s, 3H), 2.88-2.81 (m, 2H), 2.74 (q, J=7.4 Hz, 2H), 1.25-1.18 (m, 6H). LC-MS (ES+, m/z): 343.1 [(M+H)⁺]; Rt=0.703 min.

HCl/MeOH (4 M): HCl was bubbled into a solution MeOH at 0° C. for 0.5 h.

### Step 3 tert-butyl (S)-(1-((3-((3-carbamoyl-5-ethyl-6-(ethyl(methyl)amino)pyrazin-2-yl)amino)phenethyl)amino)-1-oxopropan-2-yl)(methyl)carbamate

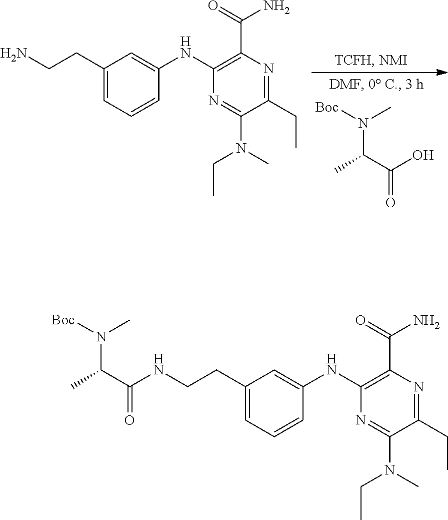

To a solution of 3-((3-(2-aminoethyl)phenyl)amino)-6-ethyl-5-(ethyl(methyl)amino)pyrazine-2-carboxamide (390 mg, 1.14 mmol, 1 eq) and N-(tert-butoxycarbonyl)-N-methyl-L-alanine (347.19 mg, 1.71 mmol, 1.5 eq) in DMF (4 mL) was added 1-methylimidazole (935.07 mg, 11.39 mmol, 907.84 uL, 10 eq), and then TCFH (479.32 mg, 1.71 mmol, 1.5 eq) at 0° C. The mixture was stirred at 0° C. for 3 hrs. LCMS showed the reaction was completed. The reaction was poured into water (10 mL) and extracted with EtOAc (15 mL*3). The organic layers were combined, washed with water (10 mL*3), saturated brine (10 mL*3), dried (Na₂SO₄), filtered and concentrated to give crude product. The residue was purified by column chromatography (SiO₂, Petroleum ether/Ethyl acetate=5/1 to 1/1) to afford tert-butyl (S)-(1-((3-((3-carbamoyl-5-ethyl-6-(ethyl(methyl)amino)pyrazin-2-yl)amino)phenethyl)amino)-1-oxopropan-2-yl)(methyl) carbamate (506 mg, 958.96 umol, 84.20% yield) as yellow solid. ¹H NMR (400 MHz, DMSO-d₆) δ=10.95-10.87 (m, 1H), 7.68-7.50 (m, 2H), 7.42-7.34 (m, 1H), 7.31-7.19 (m, 2H), 7.05-6.97 (m, 1H), 6.60 (d, J=7.5 Hz, 1H), 4.43-3.99 (m, 1H), 3.27 (q, J=7.0 Hz, 2H), 3.12-3.05 (m, 2H), 2.88-2.82 (m, 3H), 2.57-2.53 (m, 2H), 2.32-2.31 (m, 5H), 1.21-1.13 (m, 9H), 1.06-0.98 (m, 9H). LC-MS (ES+, m/z): 528.2 [(M+H)⁺]; Rt=0.890 min.

### Step 4: (S)-6-ethyl-5-(ethyl(methyl)amino)-3-((3-(2-(2-(methylamino)propanamido)ethyl)phenyl)amino)pyrazine-2-carboxamide

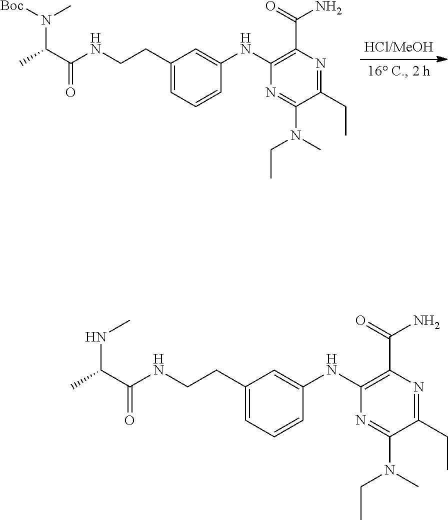

A mixture of tert-butyl (S)-(1-((3-((3-carbamoyl-5-ethyl-6-(ethyl(methyl)amino)pyrazin-2-yl)amino)phenethyl)amino)-1-oxopropan-2-yl)(methyl)carbamate (300 mg, 568.55 umol, 1 eq) in HCl/MeOH (30 mL) was stirred at 16° C. for 2 hr. LCMS showed the reaction was completed. The reaction mixture was filtered and concentrated under reduced pressure to afford (S)-6-ethyl-5-(ethyl(methyl)amino)-3-((3-(2-(2-(methylamino)propanamido)ethyl)phenyl)amino)pyrazine-2-carboxamide (298 mg, crude) as yellow oil. LC-MS (ES+, m/z): 428.2 [(M+H)+]; Rt=0.721 min.

Note: HCl was bubbled into a solution MeOH at 0° C. for 0.5 h.

### Step 5: (S,E)-3-((3-(2-(2-(4-(dimethylamino)-N-methylbut-2-enamido)propanamido)ethyl)phenyl)amino)-6-ethyl-5-(ethyl(methyl)amino)pyrazine-2-carboxamide

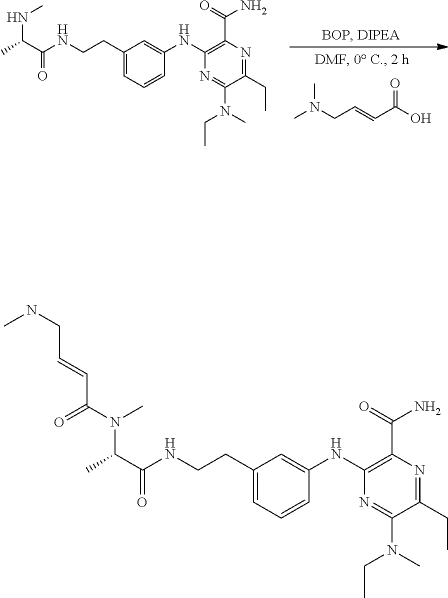

To a solution of (E)-4-(dimethylamino)but-2-enoic acid (131.41 mg, 1.02 mmol, 1.5 eq) in DMF (2.5 mL) was added DIPEA (876.63 mg, 6.78 mmol, 1.18 mL, 10 eq), (S)-6-ethyl-5-(ethyl(methyl)amino)-3-((3-(2-(2-(methylamino)propanamido)ethyl)phenyl)amino)pyrazine-2-carboxamide (290 mg, 678.30 umol, 1 eq) and then BOP (450.00 mg, 1.02 mmol, 1.5 eq) at 0° C. The mixture was stirred at 0° C. for 2 hours. LCMS indicated the reaction was completed. The reaction mixture was filtered to give a residue. The residue was purified by prep-HPLC column: Phenomenex Luna C18 75*30 mm*3 um; mobile phase: [water(TFA)-ACN]; B %: 20%-50%, 7 min. to afford (S,E)-3-((3-(2-(2-(4-(dimethylamino)-N-methylbut-2-enamido)propanamido)ethyl)phenyl)amino)-6-ethyl-5-(ethyl(methyl)amino)pyrazine-2-carboxamide (89.46 mg, 165.14 umol, 24.35% yield) as yellow solid. ¹H NMR (400 MHz, DMSO-d₆) δ=11.16-11.05 (m, 1H), 10.10-9.61 (m, 1H), 8.15-7.85 (m, 1H), 7.74 (br d, J=1.9 Hz, 1H), 7.59-7.50 (m, 1H), 7.50-7.40 (m, 2H), 7.26-7.16 (m, 1H), 6.87-6.75 (m, 2H), 6.65-6.46 (m, 1H), 5.00-4.54 (m, 1H), 3.91-3.82 (m, 2H), 3.50-3.41 (m, 2H), 3.35-3.24 (m, 2H), 3.04 (s, 3H), 2.89 (s, 2H), 2.80-2.66 (m, 11H), 1.30-1.17 (m, 9H)(TFA salt). LC-MS (ES+, m/z): 539.3 [(M+H)⁺]; Rt=2.205 min; HRMS (EI): m/z [M]⁺ found: 539.3488

### Example 79

### (Compound 226)

### (E)-3-((3-(2-(2-(4-(dimethylamino)-N-methylbut-2-enamido)acetamido)ethyl)phenyl)amino)-6-ethyl-5-methylpyrazine-2-carboxamide

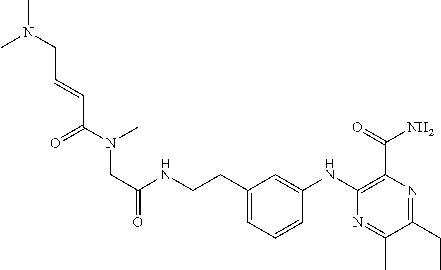 [SMILES: CCc1nc(C(N)=O)c(Nc2cccc(CCNC(=O)CN(C)C(=O)/C=C/CN(C)C)c2)nc1C]

### Step 1: tert-butyl_(E)-N-(4-(dimethylamino)but-2-enoyl)-N-methylglycinate

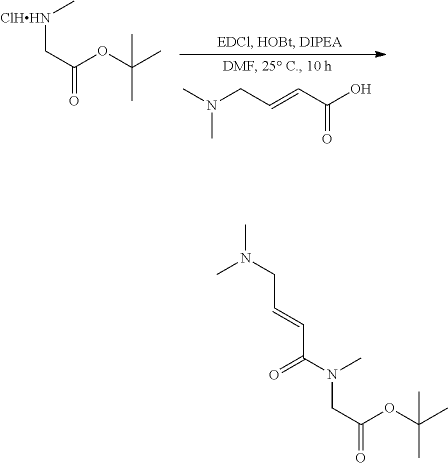

To a solution of (E)-4-(dimethylamino)but-2-enoic acid (1.52 g, 9.17 mmol, 2 eq, HCl) in DMF (5 mL) was added DIPEA (5.93 g, 45.85 mmol, 7.99 mL, 10 eq), tert-butyl methylglycinate (1.00 g, 4.58 mmol, 1 eq, HCl), HOBt (619.49 mg, 4.58 mmol, 1 eq) and EDCI (1.32 g, 6.88 mmol, 1.5 eq). The mixture was stirred at 25° C. for 10 hours. LCMS showed the reaction was completed. The reaction mixture was filtered and concentrated under reduced pressure to give a residue. The residue was purified by prep-HPLC column: C18 (250*50 mm*10 um); mobile phase: [water(NH₄HCO₃)-ACN]; B %: 15%-40%, 10 min. to afford tert-butyl (E)-N-(4-(dimethylamino)but-2-enoyl)-N-methylglycinate (540 mg, 2.11 mmol, 45.95% yield) as a brown oil. ¹H NMR (400 MHz, DMSO-d₆) δ=6.68-6.34 (m, 2H), 4.23-3.95 (m, 2H), 3.11-2.84 (m, 5H), 2.18-2.07 (m, 6H), 1.45-1.37 (m, 9H) LC-MS (ES+, m/z): 257.1 [(M+H)⁺]; Rt=1.329 min.

### Step 2: (E)-N-(4-(dimethylamino)but-2-enoyl)-N-methylglycine

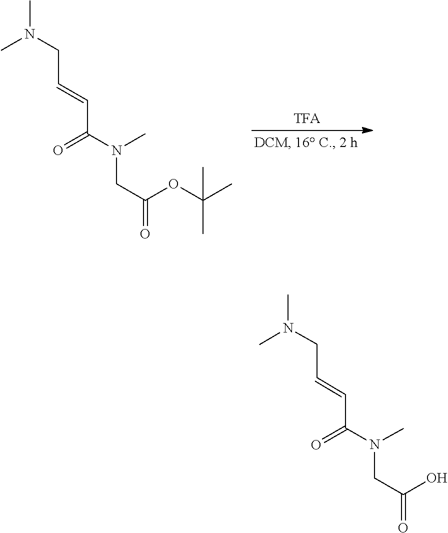 [SMILES: CN(C)C/C=C/C(=O)N(C)CC(=O)OC(C)(C)C ; CN(C)C/C=C/C(=O)N(C)CC(=O)O]

To a solution of tert-butyl (E)-N-(4-(dimethylamino)but-2-enoyl)-N-methylglycinate (270 mg, 1.05 mmol, 1 eq) in DCM (2 mL) was added TFA (3.08 g, 27.01 mmol, 2 mL, 25.65 eq). The mixture was stirred at 16° C. for 2 hours. LCMS showed the reaction was completed. The reaction mixture was concentrated under reduced pressure to afford (E)-N-(4-(dimethylamino)but-2-enoyl)-N-methylglycine (466 mg, crude) as a yellow oil. ¹H NMR (400 MHz, DMSO-d₆) δ=9.97 (br d, J=17.4 Hz, 1H), 6.97-6.48 (m, 2H), 4.28-4.02 (m, 2H), 3.95-3.82 (m, 2H), 3.10 (s, 3H), 2.76 (br d, J=13.4 Hz, 6H). LC-MS (ES+, m/z): 201.1 [(M+H)⁺]; Rt=0.089 min.

### Step 3: (E)-3-((3-(2-(2-(4-(dimethylamino)-N-methylbut-2-enamido)acetamido)ethyl)phenyl)amino)-6-ethyl-5-methylpyrazine-2-carboxamide

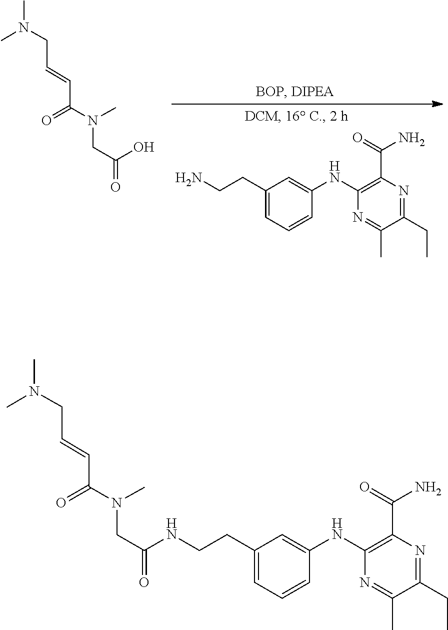 [SMILES: CN(C)C/C=C/C(=O)N(C)CC(=O)O ; CCc1nc(C(N)=O)c(Nc2cccc(CCN)c2)nc1C ; CCc1nc(C(N)=O)c(Nc2cccc(CCNC(=O)CN(C)C(=O)/C=C/CN(C)C)c2)nc1C]

To a solution of (E)-N-(4-(dimethylamino)but-2-enoyl)-N-methylglycine (200.66 mg, 400.84 umol, 40% purity, 1.5 eq) in DMF (1 mL) was added DIPEA (345.37 mg, 2.67 mmol, 465.46 uL, 10 eq), 3-((3-(2-aminoethyl)phenyl)amino)-6-ethyl-5-methylpyrazine-2-carboxamide (80 mg, 267.23 umol, 1 eq) and then BOP (177.28 mg, 400.84 umol, 1.5 eq). The mixture was stirred at 16° C. for 2 hours. LCMS indicated the reaction was completed. The reaction mixture was filtered to give a residue. The residue was purified by prep-HPLC column: C18-1150*30 mm*5 um; mobile phase: [water(TFA)-ACN]; B %: 5%-50%, 8 min to afford (E)-3-((3-(2-(2-(4-(dimethylamino)-N-methylbut-2-enamido)acetamido)ethyl)phenyl)amino)-6-ethyl-5-methylpyrazine-2-carboxamide (84.28 mg, 174.48 umol, 65.29% yield) as a yellow solid. ¹H NMR (400 MHz, DMSO-d₆) δ=11.09-11.04 (m, 1H), 9.84-9.67 (m, 1H), 8.21 (br s, 2H), 7.89-7.83 (m, 1H), 7.72-7.63 (m, 1H), 7.47-7.41 (m, 1H), 7.27-7.21 (m, 1H), 6.91-6.68 (m, 2H), 6.63-6.48 (m, 1H), 4.03-3.95 (m, 2H), 3.91-3.81 (m, 2H), 3.36-3.29 (m, 2H), 3.04 (s, 2H), 2.80-2.70 (m, 11H), 2.50-2.47 (m, 3H), 1.26-1.21 (m, 3H)(TFA salt). LC-MS (ES+, m/z): 482.3 [(M+H)⁺]; Rt=2.056 min. HRMS (EI): m/z [M+H]+: 482.2882.

### Example 80

### (Compound 227)

### (S,E)-3-((3-(2-(2-(4-(azetidin-1-yl)-N-methylbut-2-enamido)propanamido)ethyl)phenyl)amino)-6-ethyl-5-methylpyrazine-2-carboxamide

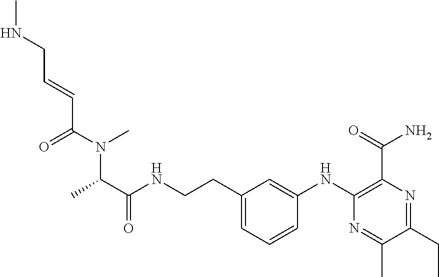 [SMILES: CCc1nc(C(N)=O)c(Nc2cccc(CCNC(=O)[C@H](C)N(C)C(=O)/C=C/CNC)c2)nc1C]

### Step 1 tert-butyl (S,E)-(4-((1-((3-((3-carbamoyl-5-ethyl-6-methylpyrazin-2-yl)amino)phenethyl)amino)-1-oxopropan-2-yl)(methyl)amino)-4-oxobut-2-en-1-yl)(methyl)carbamate

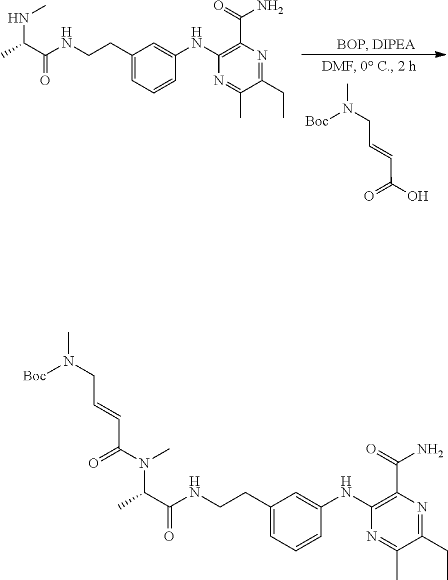 [SMILES: CCc1nc(C(N)=O)c(Nc2cccc(CCNC(=O)[C@H](C)NC)c2)nc1C ; CN(C/C=C/C(=O)O)C(=O)OC(C)(C)C ; CCc1nc(C(N)=O)c(Nc2cccc(CCNC(=O)[C@H](C)N(C)C(=O)/C=C/CN(C)C(=O)OC(C)(C)C)c2)nc1C]

To a solution of (E)-4-((tert-butoxycarbonyl)(methyl)amino)but-2-enoic acid (279.92 mg, 1.30 mmol, 2 eq) in DMF (3 mL) was added DIPEA (840.39 mg, 6.50 mmol, 1.13 mL, 10 eq), (S)-6-ethyl-5-methyl-3-((3-(2-(2-(methylamino)propanamido)ethyl)phenyl)amino)pyrazine-2-carboxamide (250 mg, 650.24 umol, 1 eq) and then BOP (431.38 mg, 975.36 umol, 1.5 eq) at 0° C. The mixture was stirred at 0° C. for 2 hours. LCMS indicated the reaction was completed. The reaction was poured into water (5 mL) and extracted with EtOAc (10 mL*3). The organic layers were combined, washed with water (10 mL*3), saturated brine (10 mL*3), dried (Na₂SO₄), filtered and concentrated to give crude product. The residue was purified by column chromatography (SiO₂, Petroleum ether/Ethyl acetate=5/1 to 1/1) to afford tert-butyl (S,E)-(4-((1-((3-((3-carbamoyl-5-ethyl-6-methylpyrazin-2-yl)amino)phenethyl)amino)-1-oxopropan-2-yl)(methyl)amino)-4-oxobut-2-en-1-yl)(methyl)carbamate (300 mg, 515.73 umol, 79.31% yield) as yellow solid. LC-MS (ES+, m/z): 582.3 [(M+H)+]; Rt=0.782 min.

### Step 2: (S,E)-6-ethyl-5-methyl-3-((3-(2-(2-(N-methyl-4-(methylamino)but-2-enamido)propanamido)ethyl)phenyl)amino)pyrazine-2-carboxamide

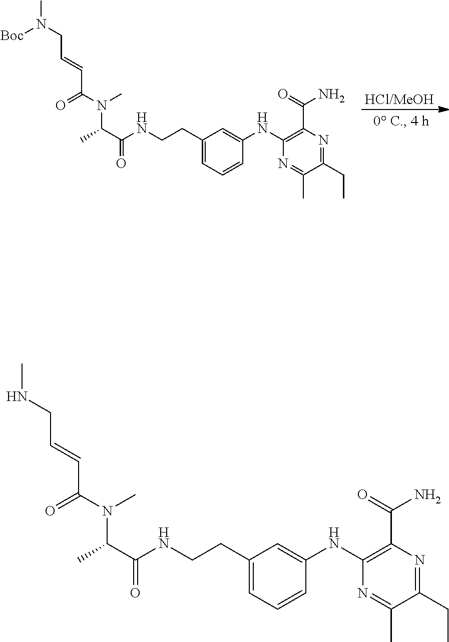

A mixture of tert-butyl (S,E)-(4-((1-((3-((3-carbamoyl-5-ethyl-6-methylpyrazin-2-yl)amino)phenethyl)amino)-1-oxopropan-2-yl)(methyl)amino)-4-oxobut-2-en-1-yl)(methyl)carbamate (280 mg, 481.34 umol, 1 eq) in HCl/MeOH (30 mL) was stirred at 0° C. for 4 hours. LCMS indicated the reaction was completed. The reaction mixture was filtered to give a residue. The residue was purified by prep-HPLC column: Phenomenex Luna C18 75*30 mm*3 um; mobile phase: [water(TFA)-ACN]; B %: 15%-45%, 7 min to afford (S,E)-6-ethyl-5-methyl-3-((3-(2-(2-(N-methyl-4-(methylamino)but-2-enamido)propanamido)ethyl)phenyl)amino)pyrazine-2-carboxamide (96.08 mg, 196.89 umol, 40.90% yield) as yellow solid. ¹H NMR (400 MHz, DMSO-d₆) δ=11.08-11.04 (m, 1H), 8.73-8.52 (m, 2H), 8.19-8.12 (m, 1H), 8.10-7.90 (m, 1H), 7.89-7.83 (m, 1H), 7.69-7.64 (m, 1H), 7.46-7.41 (m, 1H), 7.28-7.19 (m, 1H), 6.85-6.79 (m, 1H), 6.79-6.69 (m, 1H), 6.63-6.45 (m, 1H), 5.01-4.53 (m, 1H), 3.77-3.67 (m, 2H), 3.32-3.26 (m, 2H), 2.93-2.87 (m, 2H), 2.77-2.67 (m, 5H), 2.59-2.54 (m, 3H), 2.49-2.47 (m, 3H), 1.30-1.18 (m, 6H). LC-MS (ES+, m/z): 482.3 [(M+H)⁺]; Rt=2.084 min; HRMS (EI): m/z [M]⁺ found: 482.2899.

Note: HCl was bubbled into a solution MeOH at 0° C. for 0.5 h.

### Example 81

### (Compound 236)

### (R)-5-cyclopropyl-6-ethyl-3-((3-(2-(2-(N-methylacrylamido)propanamido)ethyl)phenyl)amino)pyrazine-2-carboxamide

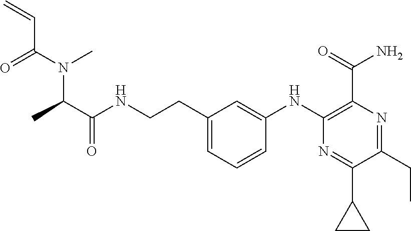 [SMILES: C=CC(=O)N(C)[C@H](C)C(=O)NCCc1cccc(Nc2nc(C3CC3)c(CC)nc2C(N)=O)c1]

### Step 1: tert-butyl (R)-(1-((3-((3-carbamoyl-6-cyclopropyl-5-ethylpyrazin-2-yl)amino)phenethyl)amino)-1-oxopropan-2-yl)(methyl)carbamate

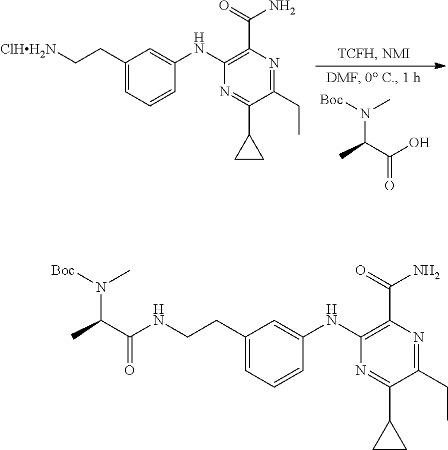

To a solution of 3-((3-(2-aminoethyl)phenyl)amino)-5-cyclopropyl-6-ethylpyrazine-2-carboxamide hydrochloride (360 mg, 1.11 mmol, 1 eq)N-(tert-butoxycarbonyl)-N-methyl-D-alanine (247.32 mg, 1.22 mmol, 1.1 eq) NMI (908.32 mg, 11.06 mmol, 881.86 uL, 10 eq) in DMF (4 mL) at 0° C., TCFH (465.61 mg, 1.66 mmol, 1.5 eq) was added. The mixture was stirred at 0° C. for 1 hour. LCMS showed the reaction was completed. The mixture was poured into water (15 mL) and extracted with EtOAc (10 mL*2). The organic layers was washed with water (10 mL*2), saturated brine (10 mL*2), dried over Na₂SO₄, filtered, concentrated under reduced pressure to give a residue. The crude product was purified by chromatography on silica gel Ethyl acetate:Petroleum ether=1:1) TLC (plate 1) to give tert-butyl (R)-(1-((3-((3-carbamoyl-6-cyclopropyl-5-ethylpyrazin-2-yl)amino)phenethyl)amino)-1-oxopropan-2-yl)(methyl)carbamate (520 mg, 1.02 mmol, 92.05% yield) as yellow oil. LC-MS (ES+, m/z): 511.4 [(M+H)+]. Rt=0.918 min.

### Step 2: (R)-5-cyclopropyl-6-ethyl-3-((3-(2-(2-(methylamino)propanamido)ethyl)phenyl)amino)pyrazine-2-carboxamide

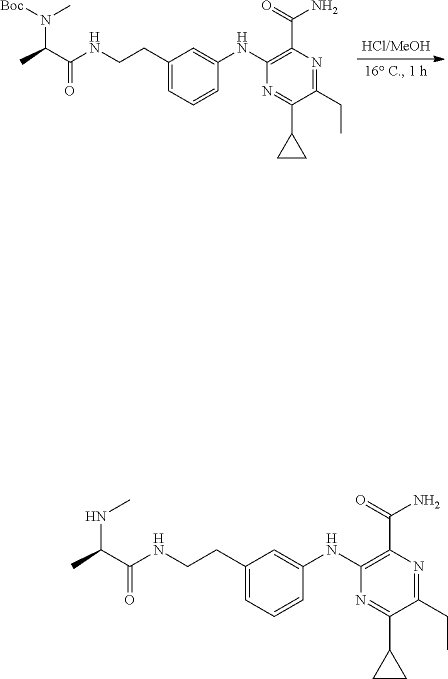

The mixture tert-butyl (R)-(1-((3-((3-carbamoyl-6-cyclopropyl-5-ethylpyrazin-2-yl)amino)phenethyl)amino)-1-oxopropan-2-yl)(methyl)carbamate (500 mg, 979.19 umol, 1 eq) and HCl/MeOH (4 M, 30 mL, 122.55 eq) was stirred at 16° C. for 1 hour. LCMS showed the reaction was completed. The mixture was concentrated under reduced pressure to give (R)-5-cyclopropyl-6-ethyl-3-((3-(2-(2-(methylamino)propanamido)ethyl)phenyl)amino)pyrazine-2-carboxamide (440 mg, crude) as red solid. LC-MS (ES+, m/z): 411.2 [(M+H)⁺]. Rt=0.699.

Note: HCl/MeOH (4 M): HCl was bubbled into a solution MeOH at 0° C. for 0.5 h.

### Step 3: (R)-5-cyclopropyl-6-ethyl-3-((3-(2-(2-(N-methylacrylamido)propanamido)ethyl)phenyl)amino)pyrazine-2-carboxamide

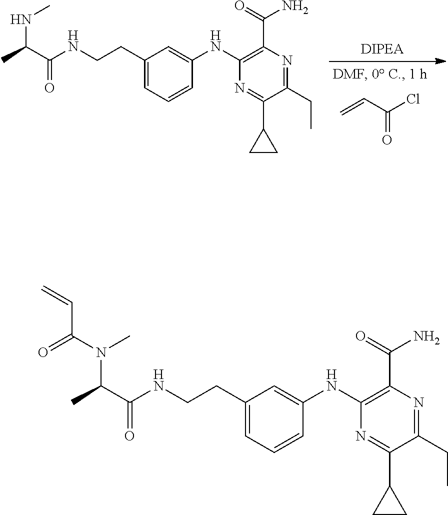

To a solution of (R)-5-cyclopropyl-6-ethyl-3-((3-(2-(2-(methylamino)propanamido)ethyl)phenyl)amino)pyrazine-2-carboxamide (440 mg, 1.07 mmol, 1 eq) DIPEA (692.64 mg, 5.36 mmol, 933.47 uL, 5 eq) in DMF (4 mL) at 0° C., acryloyl chloride (116.41 mg, 1.29 mmol, 104.88 uL, 1.2 eq) was added. The mixture was stirred at 0° C. for 1 hour. LCMS showed the reaction was completed. The mixture was filtered to give a residue. The crude was purified by prep-HPLC column: Phenomenex Luna C18 150*30 mm*5 um; mobile phase: [water(TFA)-ACN]; B %: 25%-55%, 8 min) to give (R)-5-cyclopropyl-6-ethyl-3-((3-(2-(2-(N-methylacrylamido)propanamido)ethyl)phenyl)amino)pyrazine-2-carboxamide (205.22 mg, 441.75 umol, 41.21% yield) as yellow solid. ¹H NMR (400 MHz, DMSO-d₆) 5=11.10-10.99 (m, 1H), 8.19-7.78 (m, 3H), 7.48-7.38 (m, 2H), 7.26-7.18 (m, 1H), 6.85-6.78 (m, 1H), 6.75-6.61 (m, 1H), 6.15-5.99 (m, 1H), 5.72-5.56 (m, 1H), 4.57 (br d, J=7.0 Hz, 1H), 3.39-3.23 (m, 2H), 2.96-2.82 (m, 4H), 2.76-2.66 (m, 3H), 2.34-2.25 (m, 1H), 1.31-1.17 (m, 6H), 1.14-1.04 (m, 4H); LC-MS (ES+, m/z): 465.3 [(M+H)+]; Rt=2.640 min; HRMS (EI): m/z [M]⁺ found: 465.2622.

### Example 82

### (Compound 207)

### (S,E)-3-((3-(2-(2-(4-(dimethylamino)-N-methylbut-2-enamido)propanamido)ethyl)phenyl)amino)-5,6-dimethylpyrazine-2-carboxamide

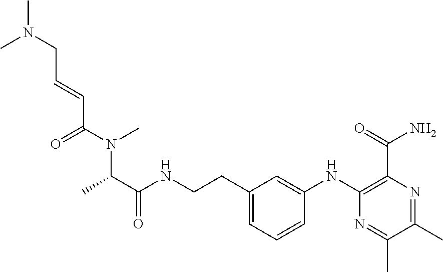

### Step 1: (S,E)-3-((3-(2-(2-(4-(dimethylamino)-N-methylbut-2-enamido)propanamido)ethyl)phenyl)amino)-5,6-dimethylpyrazine-2-carboxamide

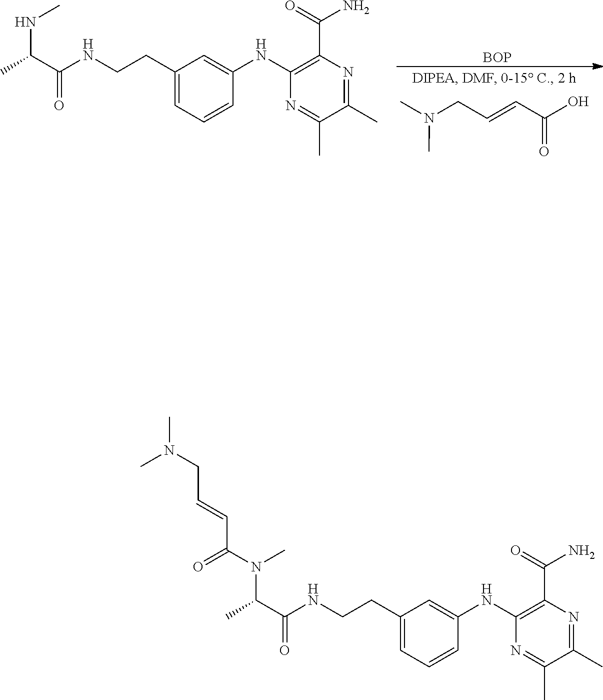

To a solution of (S)-5,6-dimethyl-3-((3-(2-(2-(methylamino)propanamido)ethyl)phenyl)amino)pyrazine-2-carboxamide (300 mg, 809.83 umol, 1 eq) (E)-4-(dimethylamino)but-2-enoic acid (201.18 mg, 1.21 mmol, 1.5 eq) BOP (537.26 mg, 1.21 mmol, 1.5 eq in DMF (3 mL) at 0° C., DIPEA (1.05 g, 8.10 mmol, 1.41 mL, 10 eq) was added. The mixture was stirred at 15° C. for 2 hrs. LCMS indicated the reaction was complete. The mixture was filtered to give a residue. The crude was purified by prep-HPLC column: Phenomenex Luna 80*30 mm*3 um; mobile phase: [water(TFA)-ACN]; B %: 10%-40%, 8 min) to give (S,E)-3-((3-(2-(2-(4-(dimethylamino)-N-methylbut-2-enamido)propanamido)ethyl)phenyl)amino)-5,6-dimethylpyrazine-2-carboxamide (121.2 mg, 250.46 umol, 30.93% yield, 99.52% purity) as brown solid. ¹H NMR (400 MHz, DMSO-d₆) δ=11.11-11.05 (m, 1H), 9.88-9.70 (m, 1H), 8.27-8.19 (m, 1H), 7.93 (br t, J=5.5 Hz, 1H), 7.84 (br s, 1H), 7.67 (br d, J=7.9 Hz, 1H), 7.43 (s, 1H), 7.27-7.20 (m, 1H), 6.87-6.76 (m, 2H), 6.67-6.43 (m, 1H), 4.57 (q, J=6.7 Hz, 1H), 3.93-3.81 (m, 2H), 3.37-3.27 (m, 2H), 2.91 (s, 2H), 2.81-2.75 (m, 6H), 2.74-2.66 (m, 3H), 2.48 (s, 3H), 2.43 (s, 3H), 1.31-1.22 (m, 3H)(TFA, salt); LC-MS (ES+, m/z): 482.3 [(M+H)+]. Rt=1.950 min; HRMS (EI): m/z [M]+ found: 482.2889.

### Example 83

### (Compound 214)

### (S,E)-3-((3-(2-(2-(4-(azetidin-1-yl)-N-methylbut-2-enamido)propanamido)ethyl)phenyl)amino)-5,6-dimethylpyrazine-2-carboxamide

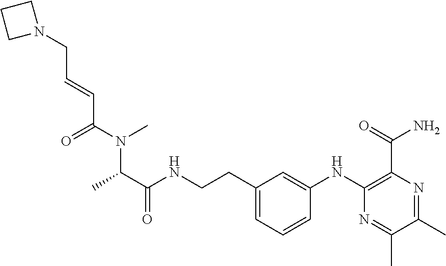 [SMILES: Cc1nc(Nc2cccc(CCNC(=O)[C@H](C)N(C)C(=O)/C=C/CN3CCC3)c2)c(C(N)=O)nc1C]

### Step 1: (S,E)-3-((3-(2-(2-(4-bromo-N-methylbut-2-enamido)propanamido)ethyl)phenyl)amino)-5,6-dimethylpyrazine-2-carboxamide

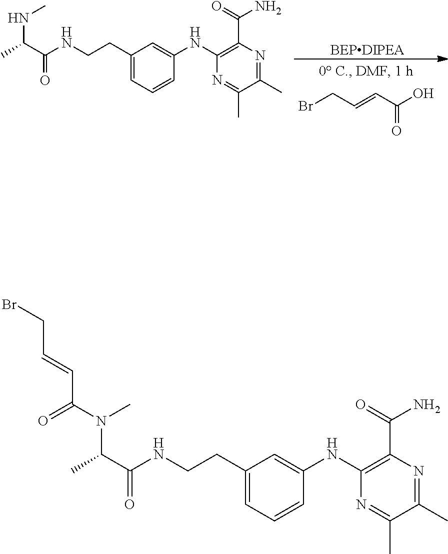

To a solution of (E)-4-bromobut-2-enoic acid (97.98 mg, 593.88 umol, 1.1 eq) DIPEA (697.77 mg, 5.40 mmol, 940.39 uL, 10 eq), BEP (221.78 mg, 809.83 umol, 1.5 eq) in DMF (2 mL) at 0° C., (S)-5,6-dimethyl-3-((3-(2-(2-(methylamino)propanamido)ethyl)phenyl)-amino)pyrazine-2-carboxamide (200 mg, 539.89 umol, 1 eq) was added. The mixture was stirred at 0° C. for 1 h. LCMS indicated the reaction was complete. The mixture was poured into water (10 mL) and extracted with EtOAc (5 mL*2). The organic layers was washed with water(5 mL*2), saturated brine (5 mL*2), dried over Na₂SO₄, filtered, concentrated under reduced pressure to give a residue. The crude product was purified by chromatography on silica gel Ethyl acetate:Petroleum ether=3:1) to give (S,E)-3-((3-(2-(2-(4-bromo-N-methylbut-2-enamido)propanamido)ethyl)phenyl)amino)-5,6-dimethylpyrazine-2-carboxamide (130 mg, 251.25 umol, 46.54% yield) as yellow solid. LC-MS (ES+, m/z): 517.3 [(M+H)+]. Rt=0.784 min.

### Step 2: (S,E)-3-((3-(2-(2-(4-(azetidin-1-yl)-N-methylbut-2-enamido)propanamido)ethyl)phenyl)amino)-5,6-dimethylpyrazine-2-carboxamide

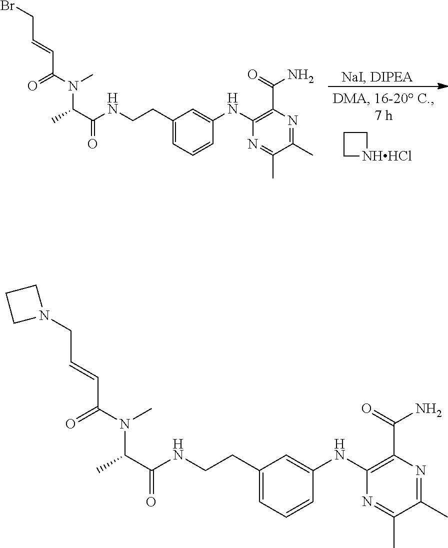

To a solution of azetidine hydrochloride (31.82 mg, 340.15 umol, 1.1 eq), NaI (139.05 mg, 927.68 umol, 3 eq), DIPEA (399.66 mg, 3.09 mmol, 538.62 uL, 10 eq) in DMA (2 mL) at 16° C., (S,E)-3-((3-(2-(2-(4-bromo-N-methylbut-2-enamido)propanamido)ethyl)phenyl)amino)-5,6-dimethylpyrazine-2-carboxamide (160 mg, 309.23 umol, 1 eq) was added, the mixture was stirred at 20° C. for 7 h. LCMS indicated the reaction was complete. The mixture was filtered to give a residue. The crude was purified by prep-HPLC column: Phenomenex Luna 80*30 mm*3 um; mobile phase: [water(TFA)-ACN]; B %: 10%-40%, 8 min) to give (S,E)-3-((3-(2-(2-(4-(azetidin-1-yl)-N-methylbut-2-enamido)propanamido)ethyl)phenyl)amino)-5,6-dimethylpyrazine-2-carboxamide (40 mg, 81.04 umol, 26.21% yield, 100% purity) as yellow solid. 1H NMR (400 MHz, DMSO-d₆) δ=11.15-11.01 (m, 1H), 9.98-9.82 (m, 1H), 8.29-8.17 (m, 1H), 8.13-7.90 (m, 1H), 7.88-7.78 (m, 1H), 7.74-7.60 (m, 1H), 7.49-7.38 (m, 1H), 7.30-7.17 (m, 1H), 6.90-6.63 (m, 2H), 6.57-6.31 (m, 1H), 5.00-4.54 (m, 1H), 4.14 (br s, 6H), 3.32-3.23 (m, 2H), 2.91 (s, 2H), 2.76-2.69 (m, 3H), 2.49-2.26 (m, 8H), 1.31-1.20 (m, 3H)(TFA, salt); LC-MS (ES+, m/z): 494.3 [(M+H)+]. Rt=1.970 min; HRMS (EI): m/z [M]+ found: 494.2851.

### Example 84

### (Compound 233)

### (S,E)-5-(dimethylamino)-3-((3-(2-(2-(4-(dimethylamino)-N-methylbut-2-enamido)propanamido)ethyl)phenyl)amino)-6-methylpyrazine-2-carboxamide

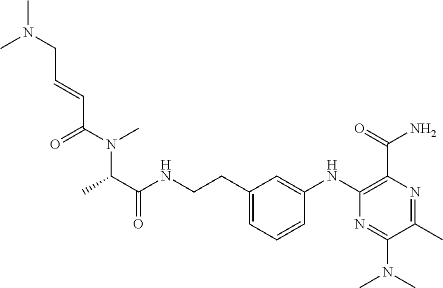

### Step 1: (E)-1-(4-(dimethylamino)-N-methylbut-2-enamido)cyclobutane-1-carboxylic acid

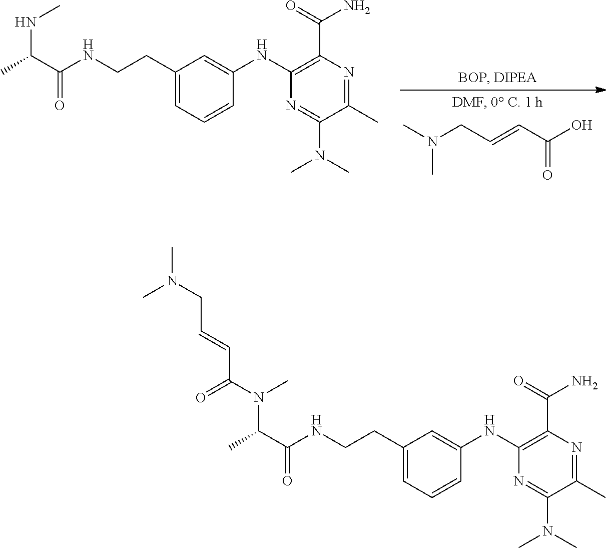

To a mixture of (E)-4-(dimethylamino)but-2-enoic acid (41.46 mg, 250.32 umol, 1 eq) in DMF (1 mL) was added DIPEA (323.51 mg, 2.50 mmol, 10 eq) and (S)-5-(dimethylamino)-6-methyl-3-((3-(2-(2-(methylamino)propanamido)ethyl)phenyl)amino)pyrazine-2-carboxamide (100 mg, 250.32 umol, 1 eq) finally added BOP (166.07 mg, 375.48 umol, 1.5 eq) in one portion at 0° C. under N₂. The mixture was stirred at 0° C. for 1 h. LCMS showed the reaction was completed. The residue was purified by prep-HPLC (column: Phenomenex C18 80*30 mm*3 um; mobile phase: [water(TFA)-ACN]; B %: 10%-40%, 7 min). Compound (S,E)-5-(dimethylamino)-3-((3-(2-(2-(4-(dimethylamino)-N-methylbut-2-enamido)propanamido)ethyl)phenyl)amino)-6-methylpyrazine-2-carboxamide (10.43 mg, 20.43 umol, 8.16% yield) was obtained as a white solid. ¹H NMR (400 MHz, DMSO-d₆) δ=11.15-11.10 (m, 1H), 9.81-9.69 (m, 1H), 8.12-7.90 (m, 1H), 7.83-7.79 (m, 1H), 7.55-7.53 (m, 1H), 7.51-7.41 (m, 2H), 7.20 (br t, J=7.7 Hz, 1H), 6.86-6.75 (m, 2H), 6.63-6.45 (m, 1H), 5.02-4.50 (m, 1H), 3.91-3.80 (m, 2H), 3.35-3.24 (m, 2H), 3.12-3.09 (m, 6H), 2.89 (s, 2H), 2.80-2.75 (m, 6H), 2.72-2.65 (m, 3H), 2.47 (s, 3H), 1.31-1.18 (m, 3H)(TFA salt). LC-MS (ES+, m/z): 511.3 [(M+H)⁺]. Rt=2.031 min; HRMS (EI): m/z [M]⁺ found: 511.3134.

### Example 85

### (Compound 230)

### (S,E)-3-((3-(2-(2-(4-(azetidin-1-yl)-N-methylbut-2-enamido)propanamido)ethyl)phenyl)amino)-5-(dimethylamino)-6-methylpyrazine-2-carboxamide

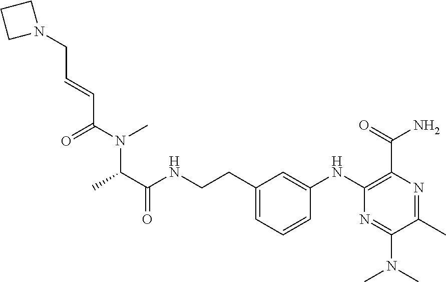 [SMILES: Cc1nc(C(N)=O)c(Nc2cccc(CCNC(=O)[C@H](C)N(C)C(=O)/C=C/CN3CCC3)c2)nc1N(C)C]

### Step 1: (S,E)-3-((3-(2-(2-(4-bromo-N-methylbut-2-enamido)propanamido)ethyl)phenyl)amino)-5-(dimethylamino)-6-methylpyrazine-2-carboxamide

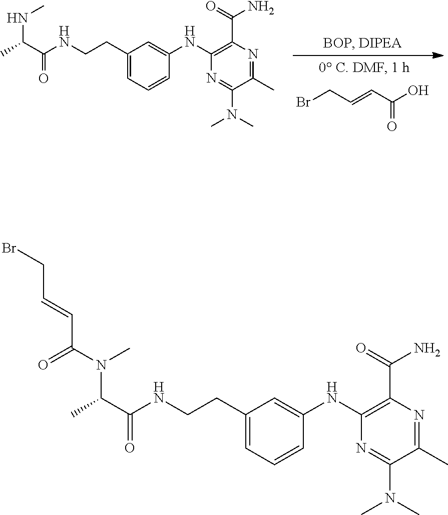

To a mixture of (E)-4-bromobut-2-enoic acid (45.43 mg, 275.35 umol, 1.1 eq) in DMF (1 mL) was added DIPEA (323.52 mg, 2.50 mmol, 10 eq) and (S)-5-(dimethylamino)-6-methyl-3-((3-(2-(2-(methylamino)propanamido)ethyl)phenyl)amino)pyrazine-2-carboxamide (100 mg, 250.32 umol, 1 eq), finally added BOP (166.07 mg, 375.48 umol, 1.5 eq) in one portion at 0° C. under N₂. The mixture was stirred at 0° C. for 1 hour. LCMS showed the reaction was completed. The residue was poured into water (20 mL). The aqueous phase was extracted with ethyl acetate (20 mL*3). The combined organic phase was washed with saturated brine (20 mL), dried with anhydrous Na₂SO₄, filtered and concentrated in vacuum. The residue was purified by column chromatography (SiO₂, Petroleum ether:Ethyl acetate=0:1). Compound (S,E)-3-((3-(2-(2-(4-bromo-N-methylbut-2-enamido)propanamido)ethyl)phenyl)amino)-5-(dimethylamino)-6-methylpyrazine-2-carboxamide (100 mg, 183.00 umol, 73.11% yield) was obtained as a yellow solid. LC-MS (ES+, m/z): 546.3 [(M+H)⁺]. Rt=0.804 min.

### Step 2: (S,E)-3-((3-(2-(2-(4-(azetidin-1-yl)-N-methylbut-2-enamido)propanamido)ethyl)phenyl)amino)-5-(dimethylamino)-6-methylpyrazine-2-carboxamide

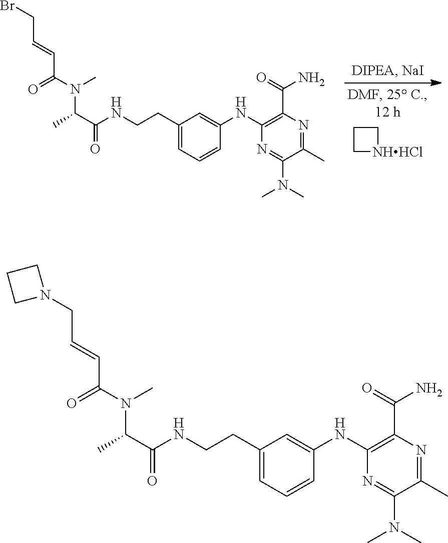

To a mixture of azetidine hydrochloride (18.83 mg, 201.30 umol, 1.1 eq) in DMA (2 mL) was added NaI (82.29 mg, 548.99 umol, 3 eq) and DIPEA (236.51 mg, 1.83 mmol, 10 eq), finally added (S,E)-3-((3-(2-(2-(4-bromo-N-methylbut-2-enamido)propanamido)ethyl)phenyl)amino)-5-(dimethylamino)-6-methylpyrazine-2-carboxamide (100 mg, 183.00 umol, 1 eq) in one portion at 25° C. under N₂. The mixture was stirred at 25° C. for 12 hrs. LCMS showed the reaction was completed. The residue was purified by prep-HPLC (column: Phenomenex C18 80*30 mm*3 um; mobile phase: [water (TFA)-ACN]; B %: 15%-45%, 7 min). Compound (S,E)-3-((3-(2-(2-(4-(azetidin-1-yl)-N-methylbut-2-enamido)propanamido)ethyl)phenyl)amino)-5-(dimethylamino)-6-methylpyrazine-2-carboxamide (10.27 mg, 19.65 umol, 10.74% yield) was obtained as a yellow solid. ¹H NMR (400 MHz, DMSO-d₆) δ=11.15-11.09 (m, 1H), 10.03-9.91 (m, 1H), 8.11-7.90 (m, 1H), 7.83-7.77 (m, 1H), 7.54 (s, 1H), 7.51-7.41 (m, 2H), 7.24-7.17 (m, 1H), 6.81-6.76 (m, 1H), 6.75-6.68 (m, 1H), 6.51-6.33 (m, 1H), 4.95-4.57 (m, 1H), 4.18-4.10 (m, 2H), 4.03-3.89 (m, 4H), 3.28 (td, J=6.6, 13.3 Hz, 2H), 3.10 (s, 6H), 2.89 (s, 2H), 2.74-2.64 (m, 3H), 2.47 (s, 3H), 2.42-2.30 (m, 2H), 1.29-1.19 (m, 3H)(TFA salt) LC-MS (ES+, m/z): 523.3 [(M+H)⁺]. Rt=2.046 min; HRMS (EI): m/z [M]⁺ found: 523.3141.

### Example 86

### (Compound 217)

### (S)-5-(dimethylamino)-6-methyl-3-((3-(2-(2-(N-methylbut-2-ynamido)propanamido)ethyl)phenyl)amino)pyrazine-2-carboxamide

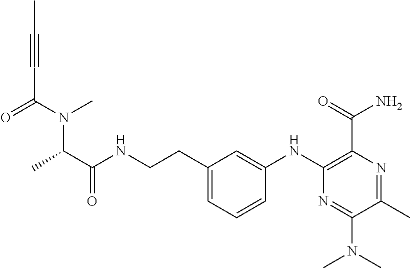 [SMILES: CC#CC(=O)N(C)[C@@H](C)C(=O)NCCc1cccc(Nc2nc(N(C)C)c(C)nc2C(N)=O)c1]

### Step 1: (S)-5-(dimethylamino)-6-methyl-3-((3-(2-(2-(N-methylbut-2-ynamido) propanamido)ethyl)phenyl)amino)pyrazine-2-carboxamide

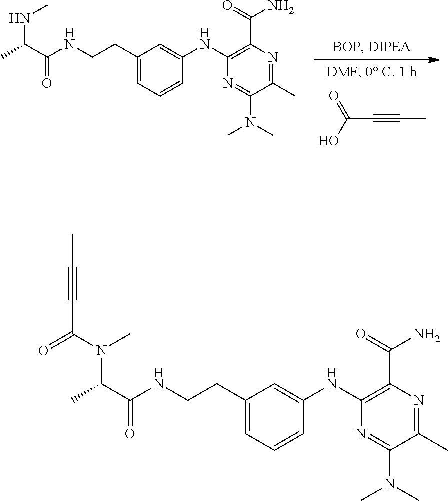

To a mixture of but-2-ynoic acid (46.30 mg, 550.70 umol, 1.1 eq) in DMF (2 mL) was added DIPEA (647.04 mg, 5.01 mmol, 10 eq) and (S)-5-(dimethylamino)-6-methyl-3-((3-(2-(2-(methylamino)propanamido)ethyl)phenyl)amino)pyrazine-2-carboxamide (200 mg, 500.64 umol, 1 eq), finally added BOP (332.14 mg, 750.96 umol, 1.5 eq) in one portion at 0° C. under N₂. The mixture was stirred at 0° C. for 1 hour. LCMS showed the reaction was completed. The residue was purified by prep-HPLC (column: Phenomenex Luna 80*30 mm*3 um; mobile phase: [water (TFA)-ACN]; B %: 25%-55%, 8 min). Compound (S)-5-(dimethylamino)-6-methyl-3-((3-(2-(2-(N-methylbut-2-ynamido)propanamido)ethyl)phenyl)amino)pyrazine-2-carboxamide (62.07 mg, 133.33 umol, 26.63% yield) was obtained as a yellow solid. ¹H NMR (400 MHz, DMSO-d₆) δ=11.16-11.08 (m, 1H), 8.07-7.89 (m, 1H), 7.80-7.75 (m, 1H), 7.60-7.55 (m, 1H), 7.50-7.44 (m, 1H), 7.43-7.38 (m, 1H), 7.24-7.17 (m, 1H), 6.79 (br d, J=7.5 Hz, 1H), 4.89-4.82 (m, 1H), 3.34-3.25 (m, 2H), 3.11-3.10 (m, 6H), 2.99 (s, 2H), 2.69 (s, 3H), 2.48 (s, 3H), 2.00 (d, J=17.3 Hz, 3H), 1.29-1.18 (m, 3H) LC-MS (ES+, m/z): 466.3 [(M+H)+]. Rt=2.455 min; HRMS (EI): m/z [M]⁺ found: 466.2534.

### Example 87

### (Compound 210)

### (E)-3-((3-(2-(1-(4-(dimethylamino)-N-methylbut-2-enamido)cyclobutane-1-carboxamido)ethyl)phenyl)amino)-6-ethyl-5-methylpyrazine-2-carboxamide

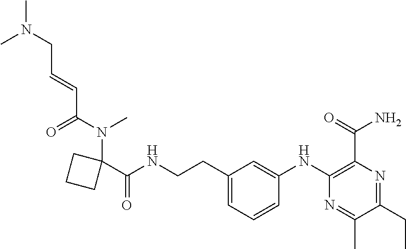 [SMILES: CCc1nc(C(N)=O)c(Nc2cccc(CCNC(=O)C3(N(C)C(=O)/C=C/CN(C)C)CCC3)c2)nc1C]

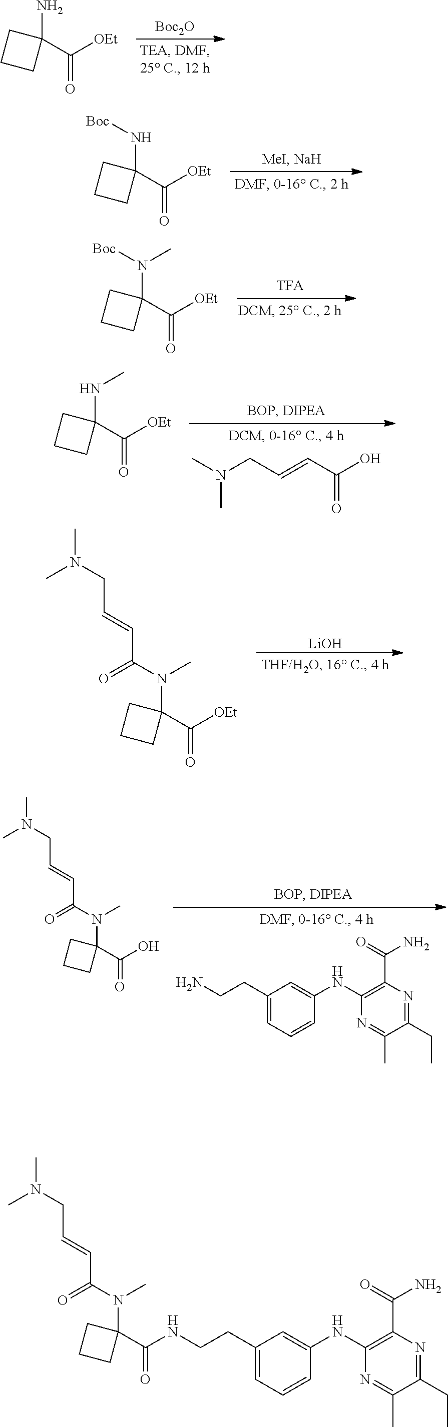

### Step 1: ethyl 1-((tert-butoxycarbonyl)amino)cyclobutane-1-carboxylate

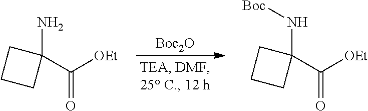 [SMILES: "Boc2O" ; CCOC(=O)C1(N)CCC1 ; CCOC(=O)C1(NC(=O)OC(C)(C)C)CCC1]

To a mixture of ethyl 1-aminocyclobutane-1-carboxylate (5 g, 27.83 mmol, 1 eq) in DMF (50 mL) was added TEA (3.38 g, 33.40 mmol, 1.2 eq) finally added Boc₂O (6.68 g, 30.62 mmol, 1.1 eq) in one portion at 25° C. The mixture was stirred at 25° C. for 12 hours. LCMS showed the reaction was completed. The residue was poured into water (30 mL). The aqueous phase was extracted with ethyl acetate (30 mL*3). The combined organic phase was washed with saturated brine (30 mL), dried with anhydrous Na₂SO₄, filtered and concentrated in vacuum. The residue was purified by column chromatography (SiO₂, Petroleum ether:Ethyl acetate=10:1). Compound ethyl 1-((tert-butoxycarbonyl)amino)cyclobutane-1-carboxylate (5 g, 15.62 mmol, 56.12% yield) was obtained as a yellow Oil. LC-MS (ES+, m/z): 244.2 [(M+H)+]. Rt=0.777 min.

### Step 2: ethyl 1-((tert-butoxycarbonyl)(methyl)amino)cyclobutane-1-carboxylate

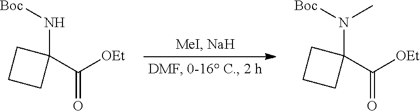 [SMILES: CCOC(=O)C1(NC(=O)OC(C)(C)C)CCC1 ; CCOC(=O)C1(N(C)C(=O)OC(C)(C)C)CCC1]

To a solution of Ethyl 1-((tert-butoxycarbonyl)amino)cyclobutane-1-carboxylate (5 g, 15.62 mmol, 1 eq) in DMF (50 mL) 0° C., NaH (937.03 mg, 23.43 mmol, 1.5 eq, 60% purity) was added. The mixture was stirred at 0° C. for 30 min, MeI (3.33 g, 23.43 mmol, 1.5 eq) was added, and then allowed to 16° C. for 1.5 hr. LCMS showed the reaction was completed. The residue was poured into saturated NH₄Cl (30 mL). The aqueous phase was extracted with ethyl acetate (30 mL*3). The combined organic phase was washed with saturated brine (30 mL*1), dried with anhydrous Na₂SO₄, filtered and concentrated in vacuum. The residue was purified by column chromatography (SiO₂, Dichloromethane:Methanol=20:1). Compound ethyl 1-((tert-butoxycarbonyl)(methyl)amino)cyclobutane-1-carboxylate (4 g, 11.97 mmol, 76.63% yield) was obtained as a white oil. LC-MS (ES+, m/z): 258.3 [(M+H)+]. Rt=0.848 min.

### Step 3: ethyl 1-(methylamino)cyclobutane-1-carboxylate

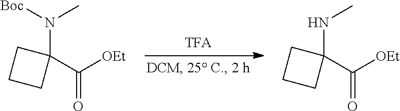 [SMILES: CCOC(=O)C1(N(C)C(=O)OC(C)(C)C)CCC1 ; CCOC(=O)C1(NC)CCC1]

To a mixture of ethyl 1-((tert-butoxycarbonyl)(methyl)amino)cyclobutane-1-carboxylate (2 g, 7.77 mmol, 1 eq) in DCM (14 mL), TFA (7 mL) was added 25° C. and stirred for 2 hrs. LCMS showed the reaction was completed. The residue was concentrated in vacuum. Compound ethyl 1-(methylamino)cyclobutane-1-carboxylate (3 g, crude) was obtained as a yellow oil. LC-MS (ES+, m/z): 158.2 [(M+H)⁺]; Rt=0.187 min.

### Step 4: ethyl (E)-1-(4-(dimethylamino)-N-methylbut-2-enamido)cyclobutane-1-carboxylate

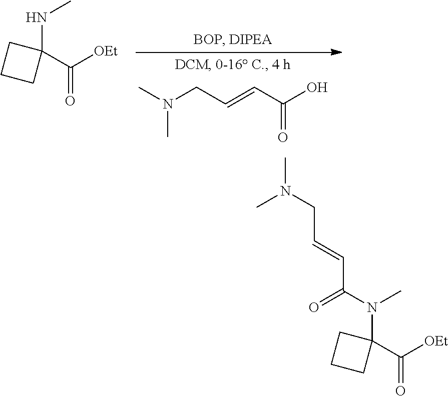

To a mixture of (E)-4-(dimethylamino)but-2-enoic acid (1.57 g, 9.50 mmol, 1.2 eq) in DMF (15 mL) was added BOP (5.25 g, 11.88 mmol, 1.5 eq) DIPEA (10.24 g, 79.19 mmol, 10 eq) finally added ethyl 1-(methylamino)cyclobutane-1-carboxylate (1.5 g, 7.92 mmol, 1 eq) in one portion at 0° C. under N₂, then heated to 16° C. and stirred for 4 hrs. LCMS showed the reaction was completed. The residue was poured into water (20 mL). The aqueous phase was extracted with ethyl acetate (30 mL*3). The combined organic phase was washed with saturated brine (30 mL*1), dried with anhydrous Na₂SO₄, filtered and concentrated in vacuum. The residue was purified by prep-HPLC (column: Waters Xbridge BEH C18 100*30 mm*10 um; mobile phase: [water(NH₄HCO₃)-ACN]; B %: 20%-50%, 8 min) to give ethyl (E)-1-(4-(dimethylamino)-N-methylbut-2-enamido)cyclobutane-1-carboxylate (230 mg, 857.09 umol, 10.82% yield) as a yellow oil. LC-MS (ES+, m/z): 269.1 [(M+H)⁺]. Rt=1.355 min.

### Step 5: (E)-1-(4-(dimethylamino)-N-methylbut-2-enamido)cyclobutane-1-carboxylic acid

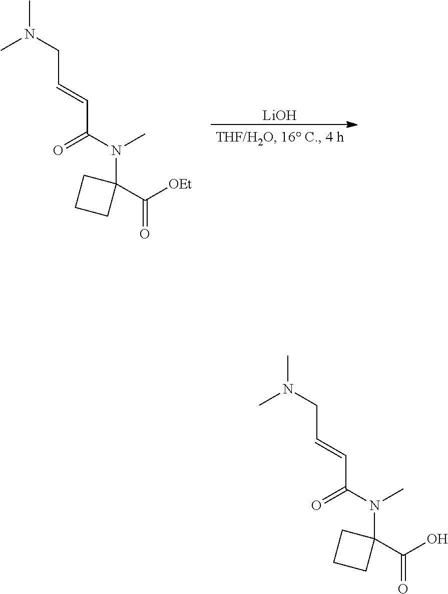 [SMILES: CCOC(=O)C1(N(C)C(=O)/C=C/CN(C)C)CCC1 ; "LiOH" ; CN(C)C/C=C/C(=O)N(C)C1(C(=O)O)CCC1]

To a mixture of ethyl (E)-1-(4-(dimethylamino)-N-methylbut-2-enamido)cyclobutane-1-carboxylate (200 mg, 745.29 umol, 1 eq) in THF (1.5 mL) H₂O (0.5 mL) was added LiOH (62.55 mg, 1.49 mmol, 2 eq) in one portion at 16° C. under N₂. The mixture was stirred at 16° C. for 4 hr. LCMS showed the reaction was completed. The residue was filtered and concentrated in vacuum. Compound (E)-1-(4-(dimethylamino)-N-methylbut-2-enamido)cyclobutane-1-carboxylic acid (300 mg, crude) was obtained as a white solid. LC-MS (ES+, m/z): 241.3 [(M+H)⁺]. Rt=0.453 min.

### Step 6: (E)-3-((3-(2-(1-(4-(dimethylamino)-N-methylbut-2-enamido)cyclobutane-1-carboxamido)ethyl)phenyl)amino)-6-ethyl-5-methylpyrazine-2-carboxamide

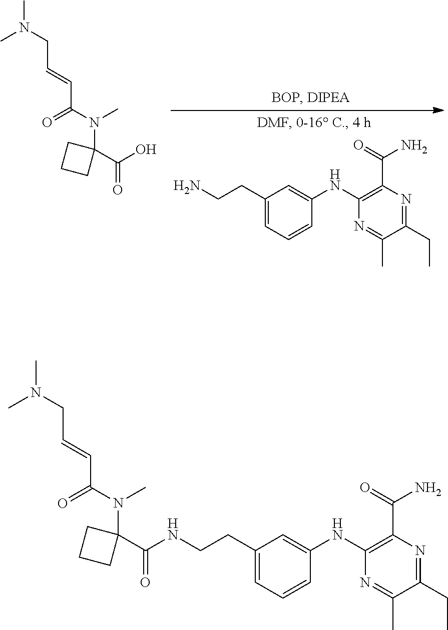 [SMILES: CN(C)C/C=C/C(=O)N(C)C1(C(=O)O)CCC1 ; CCc1nc(C(N)=O)c(Nc2cccc(CCN)c2)nc1C ; CCc1nc(C(N)=O)c(Nc2cccc(CCNC(=O)C3(N(C)C(=O)/C=C/CN(C)C)CCC3)c2)nc1C]

To a mixture of (E)-1-(4-(dimethylamino)-N-methylbut-2-enamido)cyclobutane-1-carboxylic acid (150 mg, 624.22 umol, 1 eq) in DMF (1.5 mL) was added BOP (414.12 mg, 936.34 umol, 1.5 eq) DIPEA (806.77 mg, 6.24 mmol, 10 eq) finally added 3-((3-(2-aminoethyl)phenyl)amino)-6-ethyl-5-methylpyrazine-2-carboxamide (93.44 mg, 312.11 umol, 0.5 eq) in one portion at 0° C., then allowed to warmed to 16° C. and stirred for 4 hrs. LCMS showed the reaction was completed. The residue was purified by prep-HPLC (column: Waters Xbridge Prep OBD C18 150*40 mm*10 um; mobile phase: [water(NH₄HCO₃)-ACN]; B %: 35%-65%, 8 min). Compound (E)-3-((3-(2-(1-(4-(dimethylamino)-N-methylbut-2-enamido)cyclobutane-1-carboxamido)ethyl)phenyl)amino)-6-ethyl-5-methylpyrazine-2-carboxamide (10.8 mg, 20.29 umol, 3.25% yield) was obtained as a yellow solid. ¹H NMR (400 MHz, DMSO-d₆) δ=11.07-11.02 (m, 1H), 8.17-8.11 (m, 1H), 7.94-7.82 (m, 1H), 7.68-7.63 (m, 1H), 7.56-7.32 (m, 2H), 7.24-7.15 (m, 1H), 6.81-6.75 (m, 1H), 6.56-6.45 (m, 1H), 6.37 (br s, 1H), 3.37-3.32 (m, 2H), 2.93 (br s, 2H), 2.79 (s, 2H), 2.78-2.66 (m, 5H), 2.49-2.49 (m, 3H), 2.45-2.36 (m, 2H), 2.18-2.03 (m, 8H), 1.76-1.50 (m, 2H), 1.22 (t, J=7.5 Hz, 3H). LC-MS (ES+, m/z): 522.3 [(M+H)⁺]. Rt=2.843 min; HRMS (EI): m/z [M]⁺ found: 522.3168.

### Example 88

### (Compound 213)

### (S)-5-(dimethylamino)-6-ethyl-3-((3-(2-(2-(N-methylacrylamido)propanamido)ethyl)phenyl)amino)pyrazine-2-carboxamide

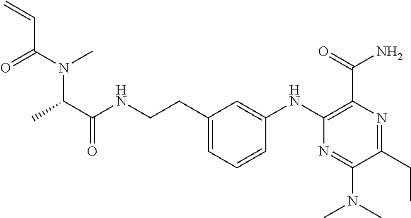 [SMILES: C=CC(=O)N(C)[C@@H](C)C(=O)NCCc1cccc(Nc2nc(N(C)C)c(CC)nc2C(N)=O)c1]

### Step 1: 3-((3-(2-aminoethyl)phenyl)amino)-5-(dimethylamino)-6-ethylpyrazine-2-carboxamide

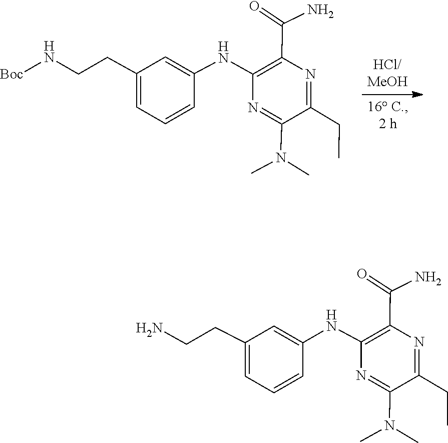 [SMILES: CCc1nc(C(N)=O)c(Nc2cccc(CCNC(=O)OC(C)(C)C)c2)nc1N(C)C ; CCc1nc(C(N)=O)c(Nc2cccc(CCN)c2)nc1N(C)C]

To a mixture of tert-butyl (3-((3-carbamoyl-6-(dimethylamino)-5-ethylpyrazin-2-yl)amino)phenethyl)carbamate (400 mg, 933.43 umol, 1 eq) in MeOH (7 mL) was added MeOH/HCl (933.43 umol, 30 mL, 1 eq). The mixture was stirred at 16° C. for 2 hrs. The crude was concentrated under reduced pressure to afford 3-((3-(2-aminoethyl)phenyl)amino)-5-(dimethylamino)-6-ethylpyrazine-2-carboxamide as yellow solid. LC-MS (ES+, m/z): 329.3 [(M+H)⁺]; Rt=0.646 min.

Note: HCl/MeOH (4 M): HCl was bubbled into a solution MeOH at 0° C. for 0.5 h. Then, the solution was weighed to obtained the HCl/MeOH (4 M).

### Step 2: tert-butyl (S)-(1-((3-((3-carbamoyl-6-(dimethylamino)-5-ethylpyrazin-2-yl)amino)phenethyl)amino)-1-oxopropan-2-yl)(methyl)carbamate

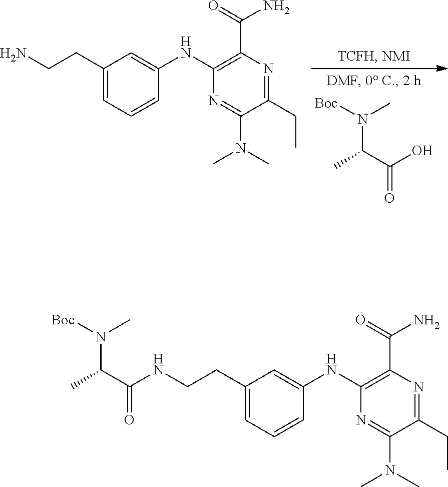

To a mixture of N-(tert-butoxycarbonyl)-N-methyl-L-alanine (631.22 mg, 3.11 mmol, 1.2 eq), 1-methylimidazole (2.12 g, 25.88 mmol, 2.06 mL, 10 eq), 3-((3-(2-aminoethyl)phenyl)amino)-5-(dimethylamino)-6-ethylpyrazine-2-carboxamide (850 mg, 2.59 mmol, 1 eq) and in DMF (6 mL) was added TCFH (1.09 g, 3.88 mmol, 1.5 eq) at 0° C. The mixture was stirred at 0° C. for 2 hrs. LCMS indicated the reaction was completed. The residue was poured into ice-water (w/w=1/1) (200 mL). The aqueous phase was extracted with ethyl acetate (100 mL*3). The combined organic phase was washed with saturated brine (100 mL*2), dried with anhydrous Na₂SO₄, filtered and concentrated in vacuum. The residue was purified by column chromatography (SiO₂, Petroleum ether:Ethyl acetate=1/1). to afford the title compound tert-butyl (S)-(1-((3-((3-carbamoyl-6-(dimethylamino)-5-ethylpyrazin-2-yl)amino)phenethyl)amino)-1-oxopropan-2-yl)(methyl)carbamate (1 g, 1.95 mmol, 91.34% yield) as a yellow oil. LC-MS (ES+, m/z): 514.4 [(M+H)⁺]; Rt=0.892 min.

### Step 3: (S)-5-(dimethylamino)-6-ethyl-3-((3-(2-(2-(methylamino)propanamido)ethyl)phenyl)amino)pyrazine-2-carboxamide

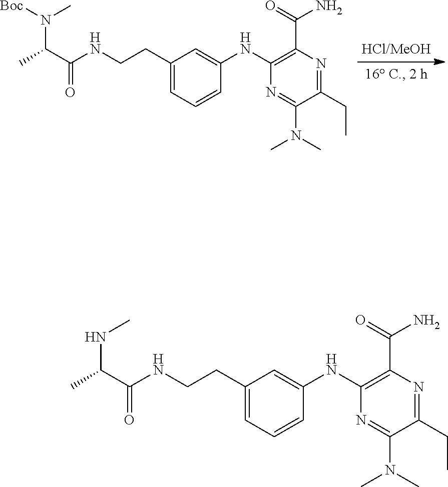

To a solution of tert-butyl (S)-(1-((3-((3-carbamoyl-6-(dimethylamino)-5-ethylpyrazin-2-yl)amino)phenethyl)amino)-1-oxopropan-2-yl)(methyl)carbamate (700 mg, 1.36 mmol, 1 eq) in HCl/MeOH (60 mL). The mixture was stirred at 16° C. for 2 hrs. LCMS indicated the reaction was completed. The crude was concentrated under reduced pressure to afford (S)-5-(dimethylamino)-6-ethyl-3-((3-(2-(2-(methylamino)propanamido)ethyl)phenyl)amino)pyrazine-2-carboxamide (700 mg, crude) as yellow solid. LC-MS (ES+, m/z): 414.3 [(M+H)⁺]; Rt=0.690 min.

Note: HCl/MeOH (4 M): HCl was bubbled into a solution MeOH at 0° C. for 0.5 h. Then, the solution was weighed to obtained the HCl/MeOH (4 M).

### Step 4: (S)-5-(dimethylamino)-6-ethyl-3-((3-(2-(2-(N-methylacrylamido)propanamido)ethyl)phenyl)amino)pyrazine-2-carboxamide

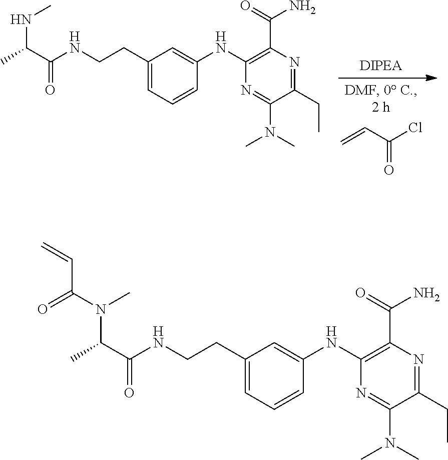

To a solution of (S)-5-(dimethylamino)-6-ethyl-3-((3-(2-(2-(methylamino)propanamido)ethyl)phenyl)amino)pyrazine-2-carboxamide (700 mg, 1.69 mmol, 1 eq) in DMF (2 mL) at ice bath. The resulting mixture was stirred at 0° C., DIEA (1.09 g, 8.46 mmol, 1.47 mL, 5 eq), and then acryloyl chloride (183.86 mg, 2.03 mmol, 165.64 uL, 1.2 eq) were added. The mixture was stirred at 0° C. for 2 hrs. LCMS indicated the reaction was completed. The crude mixture was purified by prep-HPLC column: Waters Xbridge BEH C18 100*30 mm*10 um; mobile phase: [water(NH₄HCO₃)-ACN]; B %: 30%-60%, 8 min to afford (S)-5-(dimethylamino)-6-ethyl-3-((3-(2-(2-(N-methylacrylamido)propanamido)ethyl)phenyl)amino)pyrazine-2-carboxamide (150 mg, 320.81 umol, 18.95% yield) as yellow solid. ¹H NMR (400 MHz, DMSO-d₆) δ=11.11 (s, 1H), 8.10-7.82 (m, 1H), 7.75 (br d, J=2.4 Hz, 1H), 7.56 (s, 1H), 7.46 (br s, 2H), 7.20 (t, J=7.8 Hz, 1H), 6.79 (d, J=7.6 Hz, 1H), 6.71 (dd, J=10.3, 16.5 Hz, 1H), 6.15-6.01 (m, 1H), 5.70-5.58 (m, 1H), 4.56 (br s, 1H), 3.30-3.22 (m, 2H), 3.07 (s, 6H), 2.86 (s, 2H), 2.79-2.67 (m, 5H), 1.27-1.15 (m, 6H). LC-MS (ES+, m/z): 468.3 [(M+H)⁺]; Rt=2.535 min; HRMS: 468.2704.

### Example 89

### (Compound 215)

### (S)-5-cyclopropyl-6-ethyl-3-((3-(2-(2-(2-fluoro-N-methylacrylamido)propanamido)ethyl)phenyl)amino)pyrazine-2-carboxamide

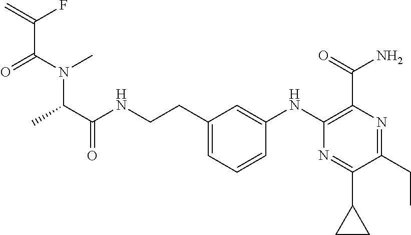 [SMILES: C=C(F)C(=O)N(C)[C@@H](C)C(=O)NCCc1cccc(Nc2nc(C3CC3)c(CC)nc2C(N)=O)c1]

### Step 1: (S)-5-cyclopropyl-6-ethyl-3-((3-(2-(2-(2-fluoro-N-methylacrylamido)propanamido)ethyl)phenyl)amino)pyrazine-2-carboxamide

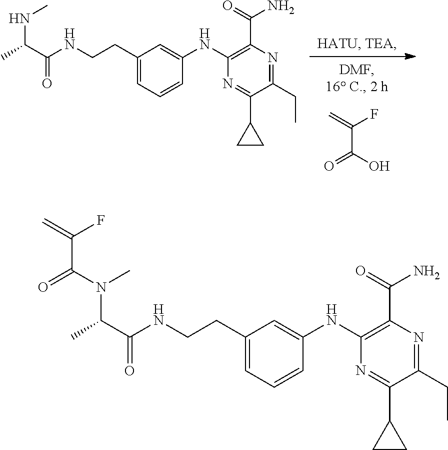

To a solution of (S)-5-cyclopropyl-6-ethyl-3-((3-(2-(2-(methylamino)propanamido)ethyl)phenyl)amino)pyrazine-2-carboxamide (80 mg, 194.88 umol, 1 eq) in DMF (0.2 mL) was added HATU (88.92 mg, 233.85 umol, 1.2 eq) and TEA (98.60 mg, 974.39 umol, 135.62 uL, 5 eq), 2-fluoroprop-2-enoic acid (21.06 mg, 233.85 umol, 1.2 eq). The mixture was stirred at 16° C. for 2 hours. LCMS indicated the reaction was completed. The reaction mixture was quenched by addition water (50 mL) at 25° C., and then extracted with EtOAc (30 mL*3). The combined organic layers were washed with saturated.brine (20 mL*2), dried over Na₂SO₄, filtered and concentrated under reduced pressure to give a residue. The residue was purified by prep-HPLC (column: Phenomenex Luna C18 150*30 mm*5 um; mobile phase: [water(TFA)-ACN]; B %: 30%-60%, 8 min) to give desired compound (S)-5-cyclopropyl-6-ethyl-3-((3-(2-(2-(2-fluoro-N-methylacrylamido)propanamido)ethyl)phenyl)amino)pyrazine-2-carboxamide (10.13 mg, 20.99 umol, 10.77% yield, 100% purity) as yellow solid. ¹H NMR (400 MHz, DMSO-d₆) δ=11.04 (s, 1H), 8.16-7.95 (m, 2H), 7.86-7.79 (m, 1H), 7.47-7.40 (m, 2H), 7.25-7.18 (m, 1H), 6.83-6.78 (m, 1H), 5.31-4.81 (m, 2H), 4.75-4.45 (m, 1H), 3.36-3.27 (m, 2H), 2.94-2.83 (m, 4H), 2.76-2.64 (m, 3H), 2.34-2.24 (m, 1H), 1.30-1.23 (m, 6H), 1.11-1.06 (m, 4H), LC-MS (ES+, m/z): 483.3 [(M+H)⁺]. Rt=2.756 min. HRMS (EI): m/z [M+H]+: 483.2490.

### Example 90

### (Compound 216)

### (S)-6-ethyl-5-methyl-3-((3-(2-(2-(N-methylacrylamido)propanamido)ethyl)phenyl)amino)pyrazine-2-carboxamide

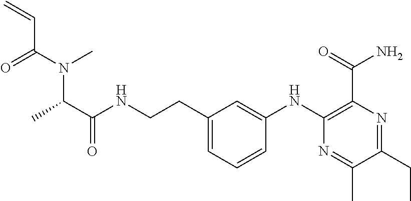 [SMILES: C=CC(=O)N(C)[C@@H](C)C(=O)NCCc1cccc(Nc2nc(C)c(CC)nc2C(N)=O)c1]

### Step 1: tert-butyl (S)-(1-((3-((3-carbamoyl-5-ethyl-6-methylpyrazin-2-yl)amino)phenethyl)amino)-1-oxopropan-2-yl)(methyl)carbamate

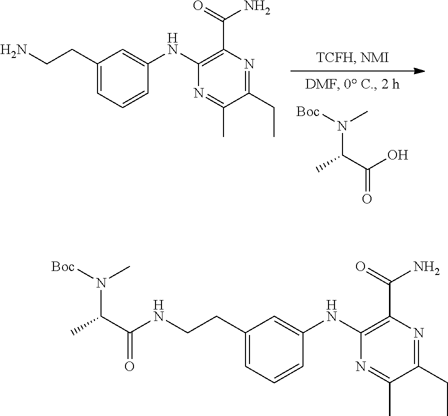

To a solution of 3-((3-(2-aminoethyl)phenyl)amino)-6-ethyl-5-methylpyrazine-2-carboxamide (450 mg, 1.50 mmol, 1 eq), N-(tert-butoxycarbonyl)-N-methyl-L-alanine (366.59 mg, 1.80 mmol, 1.2 eq) 1-methylimidazole (1.23 g, 15.03 mmol, 1.20 mL, 10 eq) in DMF (8 mL) at 0° C., TCFH (632.63 mg, 2.25 mmol, 1.5 eq) was added, the mixture was stirred at 0° C. for 2 hrs. LCMS showed the reaction was completed. The reaction was poured into H₂O (30 mL). The aqueous phase was extracted with ethyl acetate (10 mL*6). The combined organic phase was washed with saturated brine (30 mL), dried with anhydrous Na₂SO₄. Filtered and concentrated in vacuum. The residue was purified by silica gel chromatography (column height: 250 mm, diameter: 100 mm, 100-200 mesh silica gel, Petroleum ether/Ethyl acetate=1/1, 10/1) to afford tert-butyl (S)-(1-((3-((3-carbamoyl-5-ethyl-6-methylpyrazin-2-yl)amino)phenethyl)amino)-1-oxopropan-2-yl)(methyl)carbamate (1 g, 1.28 mmol, 85.12% yield) as a yellow solid. ¹H NMR (400 MHz, DMSO-d6) δ=11.06 (s, 1H), 8.14 (br d, J=2.0 Hz, 1H), 7.85 (br d, J=2.1 Hz, 1H), 7.83-7.76 (m, 1H), 7.67-7.60 (m, 1H), 7.45 (br s, 1H), 7.22 (t, J=7.8 Hz, 1H), 6.82 (d, J=7.7 Hz, 1H), 4.61-4.16 (d, 1H), 3.30 (br d, J=6.8 Hz, 2H), 2.94-2.89 (m, 2H), 2.78-2.73 (m, 2H), 2.69 (s, 3H), 2.51 (s, 3H), 1.36 (br s, 9H), 1.25-1.21 (m, 3H), 1.19-1.15 (m, 3H). LC-MS (ES+, m/z): 485.4 [(M+H)⁺]; Rt=0.877 min.

### Step 2: (S)-6-ethyl-5-methyl-3-((3-(2-(2-(methylamino)propanamido)ethyl)phenyl)amino)pyrazine-2-carboxamide

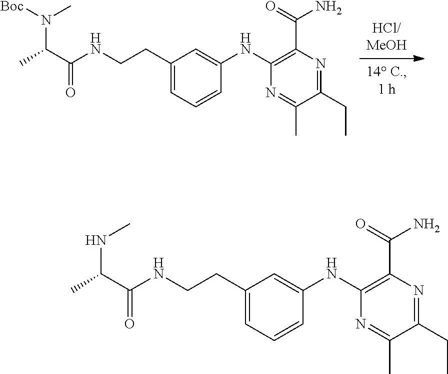

To a mixture of tert-butyl (S)-(1-((3-((3-carbamoyl-5-ethyl-6-methylpyrazin-2-yl)amino)phenethyl)amino)-1-oxopropan-2-yl)(methyl)carbamate (1 g, 1.28 mmol, 1 eq) in HCl/MeOH (4 M, 20 mL, 62.53 eq) at 14° C. for 1 hour. LCMS showed the reaction was completed. The mixture was concentrated in vacuum to afford (S)-6-ethyl-5-methyl-3-((3-(2-(2-(methylamino)propanamido)ethyl)phenyl)amino)pyrazine-2-carboxamide (430 mg, 1.12 mmol, 87.41% yield) as a yellow solid. LC-MS (ES+, m/z): 385.3 [(M+H)⁺]; Rt=0.671 min.

Note: HCl was bubbled into a solution MeOH at 0° C. for 0.5 h.

### Step 3: (S)-6-ethyl-5-methyl-3-((3-(2-(2-(N-methylacrylamido)propanamido)ethyl)phenyl)amino)pyrazine-2-carboxamide

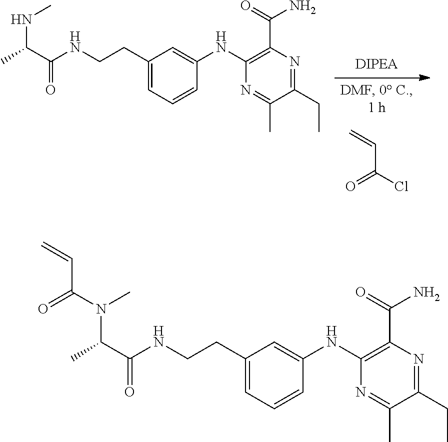

To a solution of (5)-6-ethyl-5-methyl-3-((3-(2-(2-(methylamino) propanamido)ethyl)phenyl)amino)pyrazine-2-carboxamide (500 mg, 1.19 mmol, 1 eq) and DIEA (767.59 mg, 5.94 mmol, 5 eq) in DMF (10 mL), acryloyl chloride (129.01 mg, 1.43 mmol, 1.2 eq) was added at 0° C. The mixture was stirred at 0° C. for 1 h. LCMS showed the reaction was completed. The reaction was poured into H₂O (50 mL). The aqueous phase was extracted with ethyl acetate (20 mL*3). The combined organic phase was washed with saturated brine (50 mL), dried with anhydrous Na₂SO₄. Filtered and concentrated in vacuum. The residue was purified by silica gel chromatography (column height: 250 mm, diameter: 100 mm, 100-200 mesh silica gel, Petroleum ether/Ethyl acetate=1/1, 10/1) to afford (S)-6-ethyl-5-methyl-3-((3-(2-(2-(N-methylacrylamido)propanamido)ethyl)phenyl)amino)pyrazine-2-carboxamide (241.3 mg, 550.26 umol, 46.32% yield) as a yellow solid. 1H NMR (400 MHz, CD₃CN-d₃) δ=10.88 (br s, 1H), 7.87 (br s, 1H), 7.65 (br d, J=8.0 Hz, 1H), 7.51 (br s, 1H), 7.22 (t, J=7.8 Hz, 1H), 6.83 (br d, J=7.5 Hz, 1H), 6.70-6.46 (m, 2H), 6.25-6.00 (m, 2H), 5.62 (br d, J=10.5 Hz, 1H), 5.02 (br d, J=7.0 Hz, 1H), 3.54-3.27 (m, 2H), 2.84-2.65 (m, 7H), 2.51 (s, 3H), 1.31-1.16 (m, 6H). LC-MS (ES+, m/z): 439.3 [(M+H)⁺]; Rt=2.488 min; HRMS (EI): m/z [M]⁺ found: 439.2481.

### Example 91

### (Compound 220)

### (E)-5-cyclopropyl-3-((3-(2-(1-(4-(dimethylamino)-N-methylbut-2-enamido)cyclopropane-1-carboxamido)ethyl)phenyl)amino)-6-ethylpyrazine-2-carboxamide

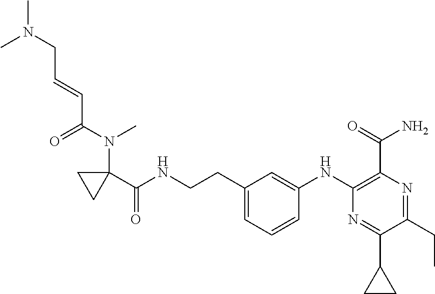 [SMILES: CCc1nc(C(N)=O)c(Nc2cccc(CCNC(=O)C3(N(C)C(=O)/C=C/CN(C)C)CC3)c2)nc1C1CC1]

### Step 1: tert-butyl (1-((3-((3-carbamoyl-6-cyclopropyl-5-ethylpyrazin-2-yl)amino) phenethyl) carbamoyl)cyclopropyl)(methyl)carbamate

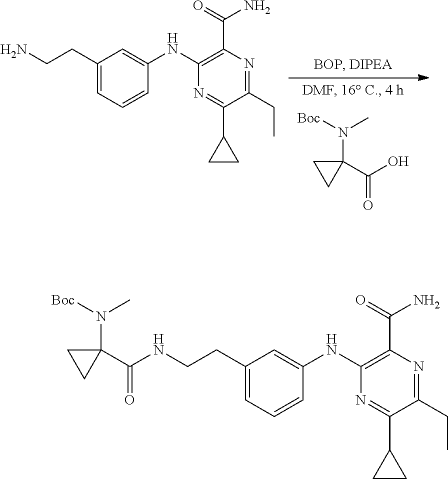 [SMILES: CCc1nc(C(N)=O)c(Nc2cccc(CCN)c2)nc1C1CC1 ; CN(C(=O)OC(C)(C)C)C1(C(=O)O)CC1 ; CCc1nc(C(N)=O)c(Nc2cccc(CCNC(=O)C3(N(C)C(=O)OC(C)(C)C)CC3)c2)nc1C1CC1]

To a solution of 3-((3-(2-aminoethyl)phenyl)amino)-5-cyclopropyl-6-ethylpyrazine-2-carboxamide (450 mg, 1.24 mmol, 1 eq, HCl) in DMF (0.5 mL) was added BOP (659.99 mg, 1.49 mmol, 1.2 eq) and DIEA (1.61 g, 12.44 mmol, 2.17 mL, 10 eq), 1-((tert-butoxycarbonyl)(methyl)amino)cyclopropane-1-carboxylic acid (803.00 mg, 3.73 mmol, 3 eq). The mixture was stirred at 16° C. for 4 hours. LCMS indicated the reaction was completed. The reaction mixture was quenched by addition water (50 mL), and then extracted with EtOAc (30 mL*3). The combined organic layers were washed with saturated brine (20 mL*2), dried over Na₂SO₄, filtered and concentrated under reduced pressure to give a residue. The residue was purified by column chromatography (SiO₂, Petroleum ether/Ethyl acetate=10/1 to 1/1), to give tert-butyl (1-((3-((3-carbamoyl-6-cyclopropyl-5-ethylpyrazin-2-yl)amino)phenethyl) carbamoyl)cyclopropyl)(methyl)carbamate (450 mg, 774.92 umol, 62.31% yield, 90% purity) as yellow solid. ¹H NMR (400 MHz, DMSO-d₆) δ=11.04 (s, 1H), 8.16-8.09 (m, 1H), 7.83 (s, 1H), 7.71 (s, 1H), 7.44-7.38 (m, 2H), 7.22 (t, J=8.0 Hz, 1H), 6.81 (d, J=7.7 Hz, 1H), 3.32-3.29 (m, 2H), 2.93-2.87 (m, 2H), 2.74-2.69 (m, 5H), 2.32-2.25 (m, 1H), 1.34-1.03 (m, 20H). LC-MS (ES+, m/z): 523.4 [(M+H)⁺]; Rt=0.886 min.

### Step 2: 5-cyclopropyl-6-ethyl-3-((3-(2-(1-(methylamino)cyclopropane-1-carboxamido)ethyl)phenyl)amino)pyrazine-2-carboxamide

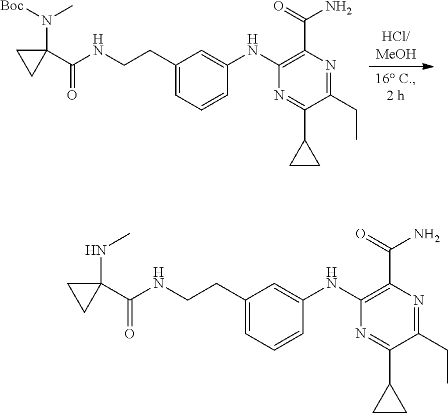 [SMILES: CCc1nc(C(N)=O)c(Nc2cccc(CCNC(=O)C3(N(C)C(=O)OC(C)(C)C)CC3)c2)nc1C1CC1 ; CCc1nc(C(N)=O)c(Nc2cccc(CCNC(=O)C3(NC)CC3)c2)nc1C1CC1]

To tert-butyl (1-((3-((3-carbamoyl-6-cyclopropyl-5-ethylpyrazin-2-yl)amino) phenethyl) carbamoyl)cyclopropyl)(methyl)carbamate (450 mg, 861.02 umol, 1 eq) was added HCl/MeOH (4 M, 10 mL). The mixture was stirred at 16° C. for 2 hours. LCMS indicated the reaction was completed. The reaction mixture was concentrated under reduced pressure to give 5-cyclopropyl-6-ethyl-3-((3-(2-(1-(methylamino)cyclopropane-1-carboxamido)ethyl)phenyl)amino) pyrazine-2-carboxamide (300 mg, 710.02 umol, 82.46% yield) as yellow solid. ¹H NMR (400 MHz, DMSO-d₆) δ=11.05 (s, 1H), 9.54 (br s, 2H), 8.13 (br s, 1H), 7.96-7.95 (m, 1H), 7.84-7.82 (m, 1H), 7.47 (br d, J=6.8 Hz, 1H), 7.39-7.38 (m, 1H), 7.22 (s, 1H), 6.79 (s, 1H), 3.34 (br s, 2H), 2.90 (d, J=7.5 Hz, 2H), 2.73-2.71 (m, 3H), 2.53-2.51 (m, 2H), 2.30-2.27 (m, 1H), 1.27-1.09 (m, 11H)(TFA salt). LC-MS (ES+, m/z): 423.3 [(M+H)⁺]; Rt=0.717 min. Note: HCl/MeOH (4 M): HCl gas was bubbled into a solution MeOH at 0° C. for 0.5 h.

### Step 3: (E)-5-cyclopropyl-3-((3-(2-(1-(4-(dimethylamino)-N-methylbut-2-enamido)cyclopropane-1-carboxamido)ethyl)phenyl)amino)-6-ethylpyrazine-2-carboxamide

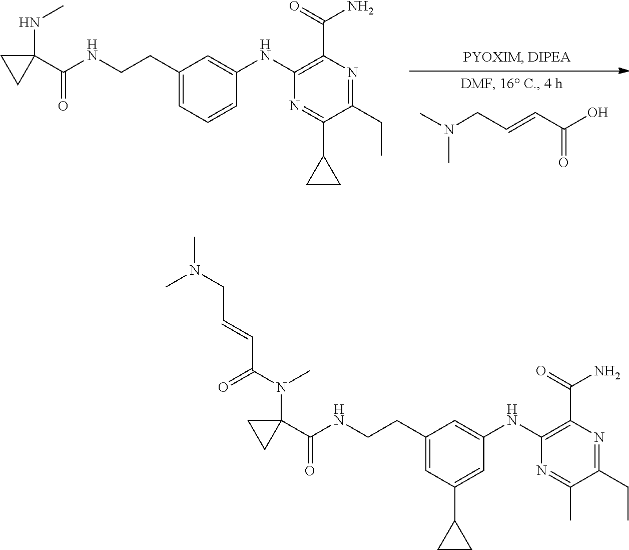

To a solution of 5-cyclopropyl-6-ethyl-3-((3-(2-(1-(methylamino)cyclopropane-1-carboxamido)ethyl)phenyl)amino)pyrazine-2-carboxamide (150 mg, 355.01 umol, 1 eq) in DMF (0.2 mL) was added PYOXIM (374.45 mg, 710.02 umol, 2 eq) and DIEA (458.83 mg, 3.55 mmol, 618.37 uL, 10 eq), (E)-4-(dimethylamino)but-2-enoic acid (91.70 mg, 710.02 umol, 2 eq). The mixture was stirred at 16° C. for 4 hours. LCMS indicated the reaction was completed. The reaction mixture was quenched by addition water (50 mL), and then extracted with EtOAc (30 mL*3). The combined organic layers were washed with saturated brine (20 mL*2), dried over Na₂SO₄, filtered and concentrated under reduced pressure to give a residue. The residue was purified by prep-HPLC (column: Waters Xbridge BEH C18 100*30 mm*10 um; mobile phase: [water (NH₄HCO₃)-ACN]; B %: 35%-55%, 8 min) to give desired compound (E)-5-cyclopropyl-3-((3-(2-(1-(4-(dimethylamino)-N-methylbut-2-enamido)cyclopropane-1-carboxamido)ethyl)phenyl)amino)-6-ethylpyrazine-2-carboxamide (10.17 mg, 18.96 umol, 5.34% yield, 99.49% purity) as yellow solid. ¹H NMR (400 MHz, DMSO-d₆) δ=11.08-11.01 (m, 1H), 8.12 (br s, 1H), 8.04 (t, J=5.7 Hz, 1H), 7.84-7.79 (m, 1H), 7.49-7.44 (m, 1H), 7.38-7.33 (m, 1H), 7.24-7.18 (m, 1H), 6.82-6.76 (m, 1H), 6.61-6.51 (m, 1H), 6.34-6.25 (m, 1H), 3.37-3.33 (m, 2H), 2.97-2.86 (m, 4H), 2.82 (s, 3H), 2.70 (br t, J=7.2 Hz, 2H), 2.32-2.27 (m, 1H), 2.15-2.06 (m, 6H), 1.30-1.02 (m, 11H), LC-MS (ES+, m/z): 423.3 [(M+H)⁺]. Rt=HRMS (EI): m/z [M]⁺ found: 534.3177.

### Example 92

### (Compound 223)

### (E)-3-((3-(2-(1-(4-(dimethylamino)-N-methylbut-2-enamido)cyclopropane-1-carboxamido)ethyl)phenyl)amino)-6-ethyl-5-methylpyrazine-2-carboxamide

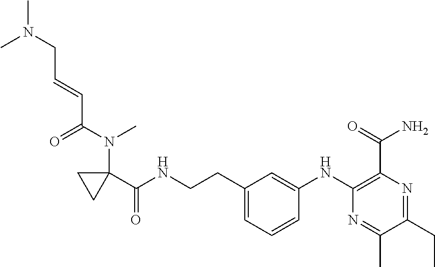 [SMILES: CCc1nc(C(N)=O)c(Nc2cccc(CCNC(=O)C3(N(C)C(=O)/C=C/CN(C)C)CC3)c2)nc1C]

### Step 1: ethyl 1-((tert-butoxycarbonyl)(methyl)amino)cyclopropane-1-carboxylate

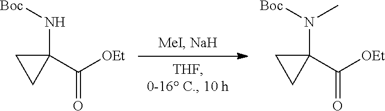 [SMILES: CCOC(=O)C1(NC(=O)OC(C)(C)C)CC1 ; CCOC(=O)C1(N(C)C(=O)OC(C)(C)C)CC1]

NaH (1.31 g, 32.71 mmol, 60% purity, 1.5 eq) was suspend in DMF (50 mL), and then cooled to 0° C., to a solution of ethyl 1-((tert-butoxycarbonyl)amino)cyclopropane-1-carboxylate (5 g, 21.81 mmol, 1 eq) in DMF (50 mL) was added dropwise, the resulting mixture was stirred until gas evolution ceased. MeI (4.64 g, 32.71 mmol, 2.04 mL, 1.5 eq) was added dropwise at the same temperature. The mixture was allowed to warm to 16° C. for 10 hrs under N₂. LCMS indicated the reaction was completed. The reaction was poured into saturated NH₄Cl solution (100 mL) and extracted with EtOAc (100 mL*2). The organic layers were combined, washed with water (100 mL*2), saturated brine (100 mL), dried (Na₂SO₄), filtered and concentrated to give crude product. The crude product was purified by chromatography on silica thiol gel (petroleum ether/EtOAc=4/1) to afford ethyl 1-((tert-butoxycarbonyl)(methyl)amino)cyclopropane-1-carboxylate (5 g, 16.44 mmol, 75.39% yield, 80% purity) as white solid. LC-MS (ES+, m/z): 144.2 [(M+H-100)⁺]; Rt=0.799 min;

### Step 2: 1-((tert-butoxycarbonyl)(methyl)amino)cyclopropane-1-carboxylic acid

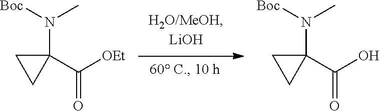 [SMILES: CCOC(=O)C1(N(C)C(=O)OC(C)(C)C)CC1 ; CN(C(=O)OC(C)(C)C)C1(C(=O)O)CC1]

To a solution of ethyl 1-((tert-butoxycarbonyl)(methyl)amino)cyclopropane-1-carboxylate (1 g, 4.11 mmol, 1 eq) in H₂O (1 mL) and MeOH (6 mL) was added LiOH·H₂O (1.72 g, 41.10 mmol, 10 eq). The mixture was stirred at 60° C. for 10 hours. LCMS indicated the reaction was completed. The crude mixture was concentrated under reduced pressure to afford crude mixture of 1-((tert-butoxycarbonyl)(methyl)amino)cyclopropane-1-carboxylic acid (1 g, crude) as white solid. LC-MS (ES+, m/z): 116.2 [(M+H-100)+]; Rt=0.679 min.

### Step 3: tert-butyl (1-((3-((3-carbamoyl-5-ethyl-6-methylpyrazin-2-yl)amino)phenethyl)carbamoyl)cyclopropyl)(methyl)carbamate

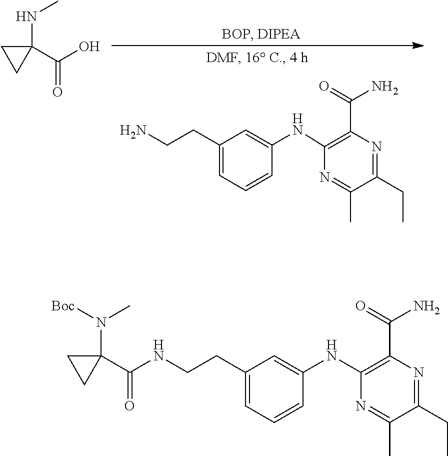

To a solution of 1-((tert-butoxycarbonyl)(methyl)amino)cyclopropane-1-carboxylic acid (287.60 mg, 1.34 mmol, 2 eq) in DMF (1 mL) was added BOP (443.21 mg, 1.00 mmol, 1.5 eq), DIEA (863.43 mg, 6.68 mmol, 1.16 mL, 10 eq), 3-((3-(2-aminoethyl)phenyl)amino)-6-ethyl-5-methylpyrazine-2-carboxamide (200 mg, 668.07 umol, 1 eq). The mixture was stirred at 16° C. for 4 hours under N₂. LCMS indicated the reaction was completed. The reaction was poured into water (50 mL) and extracted with EtOAc (30 mL*2). The organic layers were combined, washed with water (20 mL*2), saturated brine (10 mL), dried (Na₂SO₄), filtered and concentrated to give crude product. The crude product was purified by chromatography on silica thiol gel (petroleum ether/EtOAc=1/1) to afford tert-butyl (1-((3-((3-carbamoyl-5-ethyl-6-methylpyrazin-2-yl)amino)phenethyl)carbamoyl)cyclopropyl)(methyl)carbamate (150 mg, 302.05 umol, 45.21% yield) as yellow solid. LC-MS (ES+, m/z): 497.2 [(M+H)⁺]; Rt=0.891 min;

### Step 4: 6-ethyl-5-methyl-3-((3-(2-(1-(methylamino)cyclopropane-1-carboxamido)ethyl)phenyl)amino)pyrazine-2-carboxamide

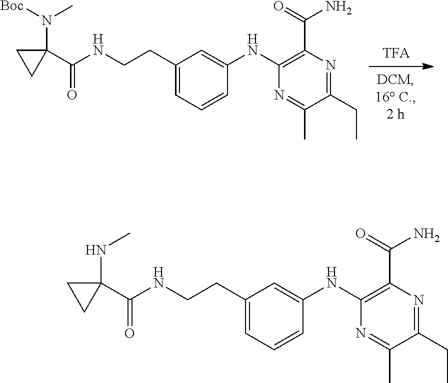 [SMILES: CCc1nc(C(N)=O)c(Nc2cccc(CCNC(=O)C3(N(C)C(=O)OC(C)(C)C)CC3)c2)nc1C ; CCc1nc(C(N)=O)c(Nc2cccc(CCNC(=O)C3(NC)CC3)c2)nc1C]

To a solution of tert-butyl (1-((3-((3-carbamoyl-5-ethyl-6-methylpyrazin-2-yl)amino)phenethyl)carbamoyl)cyclopropyl)(methyl)carbamate (150 mg, 302.05 umol, 1 eq) in DCM (20 mL) was added TFA (15.40 g, 135.06 mmol, 10 mL, 447.14 eq). The mixture was stirred at 16° C. for 2 hours. LCMS indicated the reaction was completed. The crude mixture was concentrated under reduced pressure to afford crude mixture of 6-ethyl-5-methyl-3-((3-(2-(1-(methylamino)cyclopropane-1-carboxamido)ethyl)phenyl)amino)pyrazine-2-carboxamide (150 mg, crude) as yellow solid. LC-MS (ES+, m/z): 397.3 [(M+H)⁺]; Rt=0.526 min.

### Step 5: (E)-3-((3-(2-(1-(4-(dimethylamino)-N-methylbut-2-enamido)cyclopropane-1-carboxamido)ethyl)phenyl)amino)-6-ethyl-5-methylpyrazine-2-carboxamide

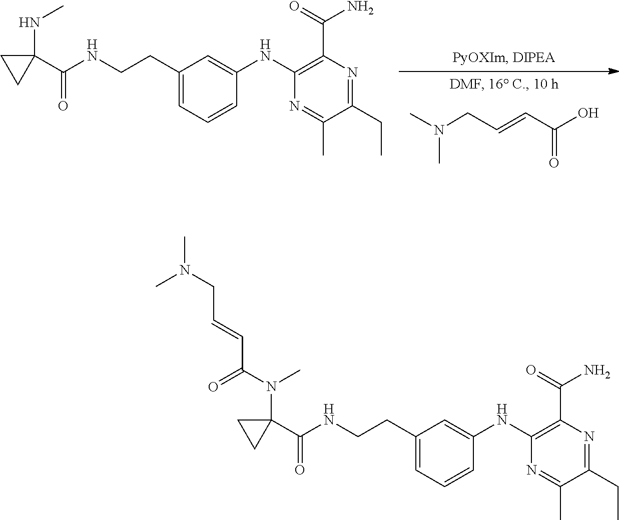

To a solution of 6-ethyl-5-methyl-3-((3-(2-(1-(methylamino)cyclopropane-1-carboxamido)ethyl)phenyl)amino)pyrazine-2-carboxamide (200 mg, 504.43 umol, 1 eq), (E)-4-(dimethylamino)but-2-enoic acid (167.09 mg, 1.01 mmol, 2 eq, HCl) in DMF (2 mL) was added DIEA (651.94 mg, 5.04 mmol, 878.63 uL, 10 eq), PyOXim (399.04 mg, 756.65 umol, 1.5 eq). The mixture was stirred at 16° C. for 10 hrs. LCMS indicated the reaction was completed. The reaction was poured into water (50 mL) and extracted with EtOAc (30 mL*2). The organic layers were combined, washed with water (20 mL*2), saturated brine (10 mL), dried (Na₂SO₄), filtered and concentrated to give crude product. The crude mixture was purified by prep-HPLC column: Waters Xbridge BEH C18 100*30 mm*10 um; mobile phase: [water(NH4HCO3)-ACN]; B %: 25%-55%, 8 min to afford (E)-3-((3-(2-(1-(4-(dimethylamino)-N-methylbut-2-enamido)cyclopropane-1-carboxamido)ethyl)phenyl)amino)-6-ethyl-5-methylpyrazine-2-carboxamide (10 mg, 19.70 umol, 3.91% yield) as yellow solid. 1H NMR (400 MHz, CDCl₃) δ −10.64 (s, 1H), 7.80 (br d, J=3.4 Hz, 1H), 7.61 (br d, J=8.0 Hz, 1H), 7.45 (s, 1H), 7.18-7.15 (m, 1H), 6.89-6.79 (m, 1H), 6.72 (d, J=7.5 Hz, 1H), 6.34 (d, J=15.1 Hz, 1H), 6.10 (br s, 1H), 5.45 (br d, J=2.8 Hz, 1H), 3.60-3.36 (m, 2H), 2.98 (br t, J=6.4 Hz, 2H), 2.81 (s, 3H), 2.75-2.64 (m, 4H), 2.46 (s, 3H), 2.15 (s, 6H), 1.84-1.48 (m, 2H), 1.20 (t, J=7.5 Hz, 3H), 1.12-0.98 (m, 2H). LC-MS (ES+, m/z): 508.3 [(M+H)⁺]; Rt=2.109 min; HRMS (EI): m/z [M+H]+: 508.2993.

### Example 93

### (Compound 224)

### (S,E)-5-cyclopropyl-6-ethyl-3-((3-(2-(2-(N-methyl-4-(methylamino)but-2-enamido)propanamido)ethyl)phenyl)amino)pyrazine-2-carboxamide

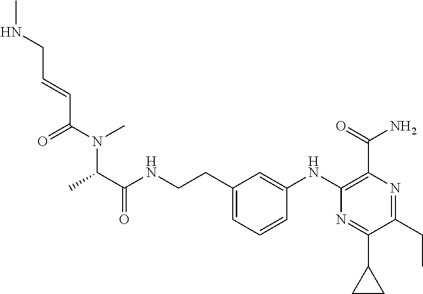 [SMILES: CCc1nc(C(N)=O)c(Nc2cccc(CCNC(=O)[C@H](C)N(C)C(=O)/C=C/CNC)c2)nc1C1CC1]

### Step 1: tert-butyl (S)-(1-((3-((3-carbamoyl-6-cyclopropyl-5-ethylpyrazin-2-yl)amino) phenethyl)amino)-1-oxopropan-2-yl)(methyl)carbamate

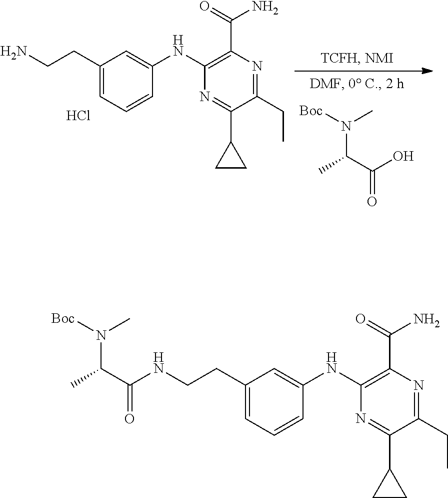

To a solution of 1-methylimidazole (1.51 g, 18.44 mmol, 1.47 mL, 10 eq), N-(tert-butoxycarbonyl)-N-methyl-L-alanine (449.68 mg, 2.21 mmol, 1.20 eq), 3-((3-(2-aminoethyl)phenyl)amino)-5-cyclopropyl-6-ethylpyrazine-2-carboxamide hydrochloride (600 mg, 1.84 mmol, 1 eq) in DMF (5 mL), TCFH (620.81 mg, 2.21 mmol, 1.2 eq) was added at 0° C. The mixture was stirred at 0° C. for 2 hrs. LCMS showed the reaction was completed. The reaction mixture was quenched by addition water (50 mL) at 25° C., and then extracted with EtOAc (30 mL*3). The combined organic layers were washed with saturated brine (20 mL*2), dried over Na₂SO₄, filtered and concentrated under reduced pressure to give a residue. The residue was purified by column chromatography (SiO₂, Petroleum ether/Ethyl acetate=10/1 to 5/1) to give tert-butyl (S)-(1-((3-((3-carbamoyl-6-cyclopropyl-5-ethylpyrazin-2-yl)amino) phenethyl)amino)-1-oxopropan-2-yl)(methyl)carbamate as yellow solid (400 mg, 721.86 umol, 39.15% yield, 92.15% purity). ¹H NMR (400 MHz, DMSO-d6) δ=11.05 (s, 1H), 8.14-8.09 (m, 1H), 7.98-7.93 (m, 1H), 7.82-7.80 (m, 1H), 7.42 (br s, 2H), 7.21 (t, J=7.9 Hz, 1H), 6.81 (d, J=7.5 Hz, 1H), 4.27 (br s, 1H), 4.02 (q, J=7.1 Hz, 2H), 2.93-2.88 (m, 4H), 2.71 (br s, 3H), 2.31-2.24 (m, 1H), 1.36-1.16 (m, 15H), 1.10-1.05 (m, 4H); LC-MS (ES⁺, m/z): 511.4 [(M+H)⁺]; Rt=0.889 min

### Step 2: tert-butyl (S,E)-(4-((1-((3-((3-carbamoyl-6-cyclopropyl-5-ethylpyrazin-2-yl)amino) phenethyl)amino)-1-oxopropan-2-yl)(methyl)amino)-4-oxobut-2-en-1-yl)(methyl)carbamate

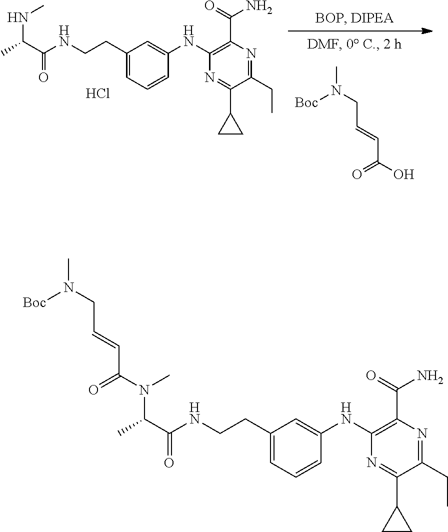 [SMILES: CCc1nc(C(N)=O)c(Nc2cccc(CCNC(=O)[C@H](C)NC)c2)nc1C1CC1 ; Cl ; CN(C/C=C/C(=O)O)C(=O)OC(C)(C)C ; CCc1nc(C(N)=O)c(Nc2cccc(CCNC(=O)[C@H](C)N(C)C(=O)/C=C/CN(C)C(=O)OC(C)(C)C)c2)nc1C1CC1]

To a solution of (S)-5-cyclopropyl-6-ethyl-3-((3-(2-(2-(methylamino)propanamido)ethyl)phenyl)amino)pyrazine-2-carboxamide hydrochloride (300 mg, 671.18 umol, 1 eq, HCl) and (E)-4-((tert-butoxycarbonyl)(methyl)amino)but-2-enoic acid (175.36 mg, 814.70 umol, 1.21 eq) in DMF (3 mL) was added DIEA (433.73 mg, 3.36 mmol, 584.54 uL, 5 eq) and then BOP (296.85 mg, 671.18 umol, 1 eq) at 0° C. The mixture was stirred at 0° C. for 2 hour. LC-MS showed the reaction was completed. The reaction mixture was quenched by addition water (100 mL) and then extracted with EtOAc (50 mL*3). The combined organic layers were washed with saturated brine (30 mL*2), dried over Na₂SO₄, filtered and concentrated under reduced pressure to give a residue. The residue was purified by prep-TLC (SiO₂, PE:EtOAc=0:1) to give tert-butyl (S,E)-(4-((1-((3-((3-carbamoyl-6-cyclopropyl-5-ethylpyrazin-2-yl)amino)phenethyl)amino)-1-oxopropan-2-yl)(methyl)amino)-4-oxobut-2-en-1-yl)(methyl)carbamate as yellow gum (200 mg, 329.09 umol, 49.03% yield). LC-MS (ES+, m/z): 608.5 [(M+H)⁺]. Rt=0.894 min.

### Step 3: (S,E)-5-cyclopropyl-6-ethyl-3-((3-(2-(2-(N-methyl-4-(methylamino)but-2-enamido)propanamido)ethyl)phenyl)amino)pyrazine-2-carboxamide

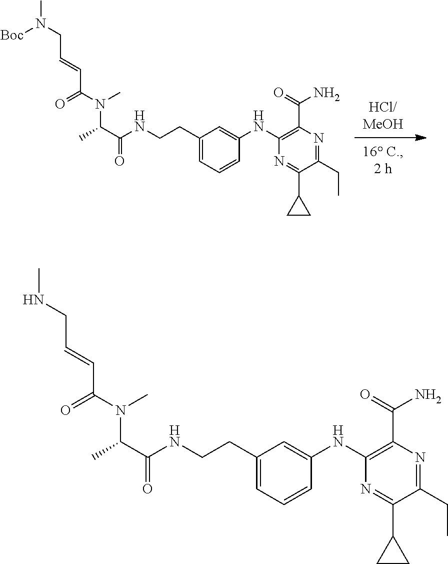

To a solution of tert-butyl (S,E)-(4-((1-((3-((3-carbamoyl-6-cyclopropyl-5-ethylpyrazin-2-yl)amino)phenethyl)amino)-1-oxopropan-2-yl)(methyl)amino)-4-oxobut-2-en-1-yl)(methyl)carbamate (200 mg, 329.09 umol, 1 eq) was added HCl/MeOH (4 M, 82.27 uL, 1 eq). The mixture was stirred at 16° C. for 2 hours. LC-MS showed the reaction was completed. The reaction mixture was concentrated under reduced pressure to give a residue. The residue was purified by prep-HPLC (column: Phenomenex Luna C18 150*30 mm*5 um; mobile phase: [water(TFA)-ACN]; B %: 15%-45%, 8 min) to give compound (S,E)-5-cyclopropyl-6-ethyl-3-((3-(2-(2-(N-methyl-4-(methylamino)but-2-enamido)propanamido)ethyl)phenyl)amino)pyrazine-2-carboxamide as a yellow solid (60.74 mg, 97.33 umol, 29.57% yield, 99.61% purity, TFA). ¹H NMR (400 MHz, DMSO-d₆) δ=11.04 (s, 1H), 8.81 (br s, 2H), 8.22-7.87 (m, 2H), 7.86-7.77 (m, 1H), 7.49-7.37 (m, 2H), 7.21 (br t, J=7.7 Hz, 1H), 6.85-6.70 (m, 2H), 6.64-6.46 (m, 1H), 5.01-4.55 (m, 1H), 3.80-3.66 (m, 2H), 3.37 (br s, 2H), 2.96-2.84 (m, 4H), 2.77-2.65 (m, 3H), 2.63-2.53 (m, 3H), 2.34-2.23 (m, 1H), 1.31-1.19 (m, 6H), 1.14-1.03 (m, 4H)(TFA salt). LC-MS (ES+, m/z): 508.3 [(M+H)⁺]. Rt=2.202 min;

Note: HCl/MeOH (4 M): HCl gas was bubbled into a solution MeOH at 0° C. for 0.5 h.

### Example 94

### (Compound 229)

### (E)-3-((3-(2-(2-(4-(dimethylamino)-N-methylbut-2-enamido)-2-methylpropanamido)ethyl)phenyl)amino)-6-ethyl-5-methylpyrazine-2-carboxamide

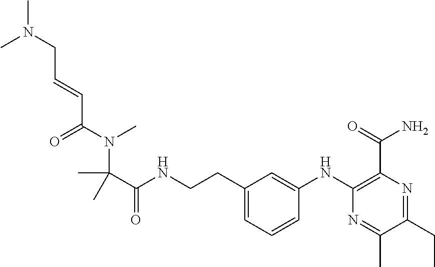 [SMILES: CCc1nc(C(N)=O)c(Nc2cccc(CCNC(=O)C(C)(C)N(C)C(=O)/C=C/CN(C)C)c2)nc1C]

### Step 1: 6-ethyl-5-methyl-3-((3-(2-(2-methyl-2-(methylamino)propanamido)ethyl)phenyl)amino)pyrazine-2-carboxamide

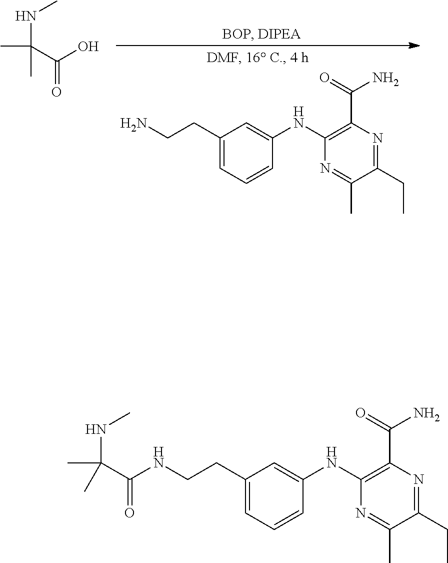

To a mixture of 3-((3-(2-aminoethyl)phenyl)amino)-6-ethyl-5-methylpyrazine-2-carboxamide (100 mg, 334.03 umol, 1 eq) and 2-methyl-2-(methylamino)propanoic acid (39.13 mg, 334.03 umol, 1 eq) in DMF (1 mL) was added BOP (221.61 mg, 501.05 umol, 1.5 eq) and DIPEA (431.72 mg, 3.34 mmol, 581.83 uL, 10 eq) in one portion at 16° C. under N₂. The mixture was stirred at 16° C. for 4 hours. LCMS showed the reaction was completed. The residue was poured into ice-water (w/w=1/1) (10 mL). The aqueous phase was extracted with ethyl acetate (5 mL*3). The combined organic phase was washed with saturated brine (5 mL*2), dried with anhydrous Na₂SO₄, filtered and concentrated in vacuum. The residue was purified by prep-TLC (SiO2, Dichloromethane:Methanol=20:1). To afford the title compound 6-ethyl-5-methyl-3-((3-(2-(2-methyl-2-(methylamino)propanamido)ethyl)phenyl)amino) pyrazine-2-carboxamide (100 mg, 250.94 umol, 75.12% yield) as a yellow oil. LC-MS (ES+, m/z): 399.3 [(M+H)⁺]; Rt=0.653 min.

### Step 2: (E)-3-((3-(2-(2-(4-(dimethylamino)-N-methylbut-2-enamido)-2-methylpropanamido)ethyl)phenyl)amino)-6-ethyl-5-methylpyrazine-2-carboxamide

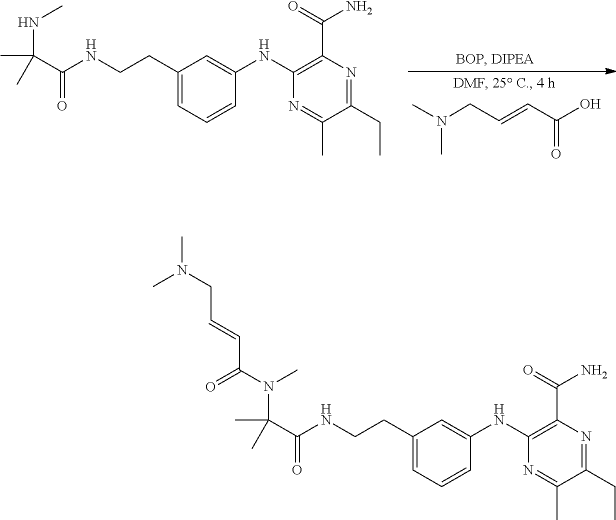

To a mixture of 6-ethyl-5-methyl-3-((3-(2-(2-methyl-2-(methylamino)propanamido)ethyl)phenyl)amino)pyrazine-2-carboxamide (100 mg, 250.94 umol, 1 eq) and (E)-4-(dimethylamino)but-2-enoic acid (64.82 mg, 501.88 umol, 2 eq) in DMF (0.2 mL) was added BOP (166.48 mg, 376.41 umol, 1.5 eq) and DIEA (324.32 mg, 2.51 mmol, 437.09 uL, 10 eq) in one portion at 25° C. The mixture was stirred at 25° C. for 4 hours. LCMS showed the reaction was completed. The residue was poured into water (10 mL) and extracted with ethyl acetate (5 mL*5). The combined organic phase was washed with saturated brine (5 mL*3), dried with anhydrous Na₂SO₄, filtered and concentrated in vacuum. The residue was purified by prep-HPLC (TFA condition). column: C18-1 150*30 mm*5 um; mobile phase: [water(TFA)-ACN]; B %: 5%-50%, 8 min. To afford the title compound (E)-3-((3-(2-(2-(4-(dimethylamino)-N-methylbut-2-enamido)-2-methylpropanamido)ethyl)phenyl)amino)-6-ethyl-5-methylpyrazine-2-carboxamide (6.18 mg, 11.84 umol, 4.72% yield) as a yellow solid. 1H NMR (400 MHz, DMSO-d₆) δ=11.11-10.92 (m, 1H), 9.61 (br s, 1H), 8.21-8.11 (m, 1H), 7.89-7.82 (m, 1H), 7.71-7.62 (m, 1H), 7.47 (br d, J=4.5 Hz, 1H), 7.40-7.30 (m, 1H), 7.26-7.16 (m, 1H), 6.81 (d, J=7.5 Hz, 1H), 6.78 (br d, J=2.1 Hz, 1H), 6.48 (td, J=7.2, 14.9 Hz, 1H), 3.89-3.82 (m, 2H), 3.25-3.18 (m, 2H), 2.99 (s, 3H), 2.78-2.70 (m, 8H), 2.65 (br t, J=7.3 Hz, 2H), 2.51 (br s, 3H), 1.31 (s, 6H), 1.24 (t, J=7.4 Hz, 3H)(TFA, salt). ¹H NMR (400 MHz, D₂O) δ=7.47-7.34 (m, 2H), 7.28 (br d, J=5.5 Hz, 1H), 7.00-6.93 (m, 1H), 6.83-6.71 (m, 1H), 6.56-6.37 (m, 1H), 3.86 (d, J=7.4 Hz, 2H), 3.41 (br t, J=6.8 Hz, 2H), 3.00 (s, 3H), 2.88-2.83 (m, 6H), 2.76 (br d, J=6.9 Hz, 2H), 2.74-2.68 (m, 2H), 2.45 (br d, J=6.1 Hz, 3H), 1.30 (s, 6H), 1.20 (t, J=7.5 Hz, 3H). LC-MS (ES+, m/z): 510.3 [(M+H)⁺]; Rt=2.104 min. HRMS (EI): m/z [M]+ found: 510.3177

### Example 95

### (Compound 234)

### (R,E)-3-((3-(2-(2-(4-(dimethylamino)-N-methylbut-2-enamido)propanamido)ethyl)phenyl)amino)-6-ethyl-5-methylpyrazine-2-carboxamide

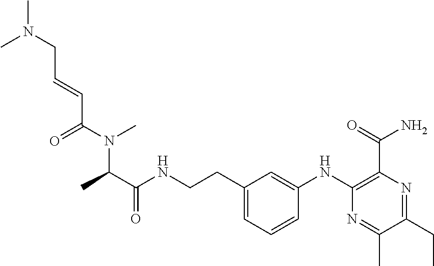 [SMILES: CCc1nc(C(N)=O)c(Nc2cccc(CCNC(=O)[C@@H](C)N(C)C(=O)/C=C/CN(C)C)c2)nc1C]

### Step 1: (R)-6-ethyl-5-methyl-3-((3-(2-(2-(methylamino)propanamido)ethyl)phenyl)amino)pyrazine-2-carboxamide

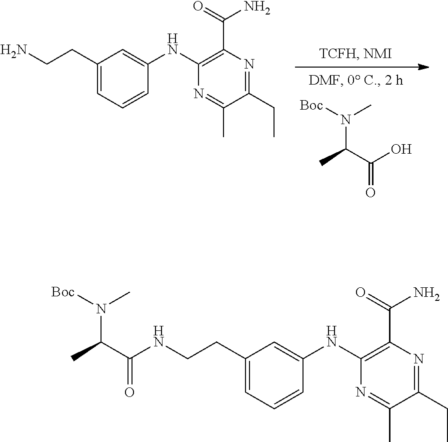 [SMILES: CCc1nc(C(N)=O)c(Nc2cccc(CCN)c2)nc1C ; C[C@H](C(=O)O)N(C)C(=O)OC(C)(C)C ; CCc1nc(C(N)=O)c(Nc2cccc(CCNC(=O)[C@@H](C)N(C)C(=O)OC(C)(C)C)c2)nc1C]

To a mixture of N-(tert-butoxycarbonyl)-N-methyl-D-alanine (651.72 mg, 3.21 mmol, 1.2 eq) in DMIF (8 mL) was added 1-methylimidazole (2.19 g, 26.72 mmol, 2.13 mL, 10 eq) and 3-((3-(2-aminoethyl)phenyl)amino)-6-ethyl-5-methylpyrazine-2-carboxamide (800 mg, 2.67 mmol, 1 eq), finally added TCFH (1.12 g, 4.01 mmol, 1.5 eq) in one portion at 0° C. under N₂. The mixture was stirred at 0° C. for 2 hours. LCMS showed the reaction was completed. The residue was poured into water (20 mL). The aqueous phase was extracted with ethyl acetate (20 mL*3). The combined organic phase was washed with saturated brine (20 mL), dried with anhydrous Na₂SO₄, filtered and concentrated in vacuum. The residue was purified by column chromatography (SiO₂, Petroleum ether:Ethyl acetate=1:1). Compound tert-butyl (R)-(1-((3-((3-carbamoyl-5-ethyl-6-methylpyrazin-2-yl)amino)phenethyl)amino)-1-oxopropan-2-yl)(methyl)carbamate (1.2 g, 2.48 mmol, 92.67% yield) was obtained as a yellow solid. LC-MS (ES+, m/z): 485.4 [(M+H)⁺]; Rt=0.885 min.

### Step 2: (R)-6-ethyl-5-methyl-3-((3-(2-(2-(methylamino)propanamido)ethyl)phenyl)amino)pyrazine-2-carboxamide

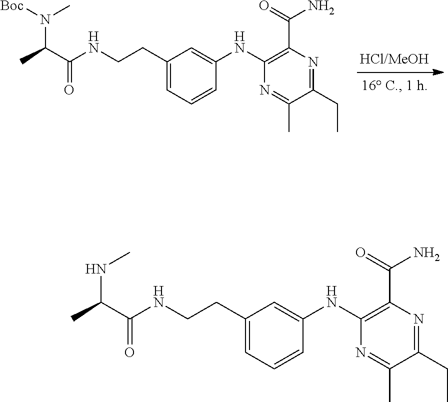

To a mixture of tert-butyl (R)-(1-((3-((3-carbamoyl-5-ethyl-6-methylpyrazin-2-yl)amino)phenethyl)amino)-1-oxopropan-2-yl)(methyl)carbamate (500 mg, 1.03 mmol, 1 eq) in HCl/MeOH (10 mL) in one portion at 16° C. The mixture was stirred at 16° C. for 1 hour. LCMS showed the reaction was completed. The residue was filtered and concentrated in vacuum. Compound (R)-6-ethyl-5-methyl-3-((3-(2-(2-(methylamino)propanamido)ethyl)phenyl)amino)pyrazine-2-carboxamide (400 mg, crude) was obtained as a yellow solid. LC-MS (ES+, m/z): 358.4 [(M+H)⁺]; Rt=0.666 min.

Note: HCl/MeOH (4 M): HCl was bubbled into a solution MeOH at 0° C. for 0.5 h.

### Step 3: (R,E)-3-((3-(2-(2-(4-(dimethylamino)-N-methylbut-2-enamido)propanamido)ethyl)phenyl)amino)-6-ethyl-5-methylpyrazine-2-carboxamide

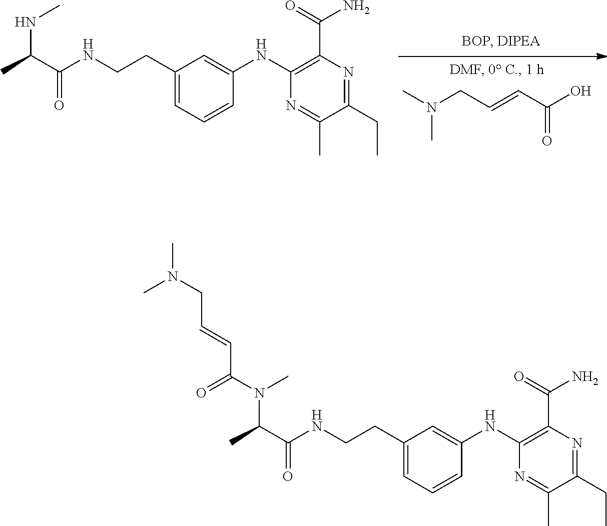

To a mixture of (E)-4-(dimethylamino)but-2-enoic acid (206.77 mg, 1.25 mmol, 1.2 eq) in DMF (4 mL) was added DIPEA (1.34 g, 10.40 mmol, 10 eq) and (R)-6-ethyl-5-methyl-3-((3-(2-(2-(methylamino)propanamido)ethyl)phenyl)amino) pyrazine-2-carboxamide (400 mg, 1.04 mmol, 1 eq) finally added BOP (690.21 mg, 1.56 mmol, 1.5 eq) in one portion at 0° C. under N₂. The mixture was stirred at 0° C. for 1 hour. LCMS showed the reaction was completed. The residue was purified by prep-HPLC (column: C18-1 150*30 mm*5 um; mobile phase: [water (TFA)-ACN]; B %: 1%-45%, 8 min). Compound (R,E)-3-((3-(2-(2-(4-(dimethylamino)-N-methylbut-2-enamido)propanamido)ethyl)phenyl)amino)-6-ethyl-5-methylpyrazine-2-carboxamide (62.76 mg, 125.11 umol, 12.03% yield) was obtained as a yellow solid. ¹H NMR (400 MHz, DMSO-d₆) δ=11.06 (br s, 1H), 9.85-9.70 (m, 1H), 8.20-8.11 (m, 1H), 8.03-7.84 (m, 2H), 7.69-7.64 (m, 1H), 7.45-7.41 (m, 1H), 7.26-7.19 (m, 1H), 6.86-6.77 (m, 2H), 6.64-6.47 (m, 1H), 5.00-4.53 (m, 1H), 3.92-3.80 (m, 2H), 3.37-3.26 (m, 2H), 2.90 (s, 2H), 2.80-2.75 (m, 11H), 2.52-2.50 (m, 3H), 1.31-1.20 (m, 6H). ¹H NMR (400 MHz, D₂O) δ=7.29-7.23 (m, 2H), 7.20-7.14 (m, 1H), 6.87-6.82 (m, 1H), 6.73-6.66 (m, 1H), 6.60-6.48 (m, 1H), 4.85-4.51 (m, 1H), 3.83-3.71 (m, 2H), 3.49-3.34 (m, 2H), 2.83-2.80 (m, 6H), 2.79-2.76 (m, 2H), 2.74-2.57 (m, 5H), 2.34-2.28 (m, 3H), 1.32-1.10 (m, 6H) LC-MS (ES+, m/z): 496.3 [(M+H)⁺]. Rt=2.079 min; HRMS (EI): m/z [M]⁺ found: 496.3013.

### Example 96

### (Compound 203)

### (S)-5-(dimethylamino)-3-((3-(2-(2-(4-(dimethylamino)-N-methylbut-2-ynamido)propanamido)ethyl)phenyl)amino)-6-ethylpyrazine-2-carboxamide

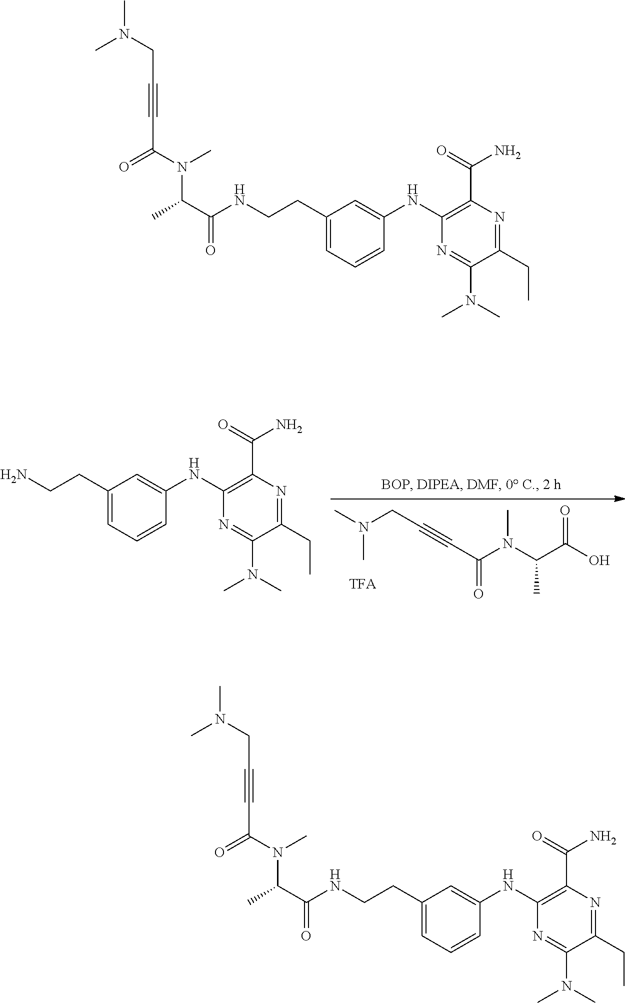

To a solution of N-(4-(dimethylamino)but-2-ynoyl)-N-methyl-L-alanine (198.69 mg, 608.99 umol, 2 eq, TFA), BOP (202.01 mg, 456.74 umol, 1.5 eq), DIEA (393.54 mg, 3.04 mmol, 530.38 uL, 10 eq) in DMF (2.5 mL) was added 3-((3-(2-aminoethyl)phenyl)amino)-5-(dimethylamino)-6-ethylpyrazine-2-carboxamide (100 mg, 304.50 umol, 1 eq). The mixture was stirred at 0° C. for 2 hrs. LCMS indicated the reaction was complete. The reaction was concentrated. The crude was purified by prep-HPLC (column: C18-1 150*30 mm*5 um; mobile phase: [water(TFA)-ACN]; B %: 10%-55%, 8 min) then it was further purified by prep-HPLC column: Phenomenex Luna C18 100*30 mm*5 um; mobile phase: [water(TFA)-MeOH]; B %: 45%-75%, 10 min to give (S)-5-(dimethylamino)-3-((3-(2-(2-(4-(dimethylamino)-N-methylbut-2-ynamido) propanamido)ethyl)phenyl)amino)-6-ethylpyrazine-2-carboxamide (6.67 mg, 12.76 umol, 4.19% yield) as yellow solid. 1H NMR (400 MHz, DMSO-d₆) δ=11.11 (d, J=3.9 Hz, 1H), 10.85-10.60 (m, 1H), 8.23-7.97 (m, 1H), 7.74 (br s, 1H), 7.55 (br d, J=6.1 Hz, 1H), 7.52-7.43 (m, 2H), 7.21 (dt, J=3.5, 7.8 Hz, 1H), 6.79 (td, J=1.4, 7.6 Hz, 1H), 4.93-4.79 (m, 1H), 4.24 (br d, J=16.0 Hz, 2H), 3.34-3.27 (m, 2H), 3.26-3.03 (m, 8H), 2.79-2.66 (m, 11H), 1.34-1.21 (m, 6H)(TFA salt). LC-MS (ES+, m/z): 523.3 [(M+H)+]; Rt=2.813 min; HRMS (EI): m/z [M]⁺ found: 523.3130; SFC: 95.58%.

### Example 97

### (Compound 208)

### (S)-5-cyclopropyl-6-ethyl-3-((3-(2-(2-(N-methylacrylamido)propanamido)ethyl)phenyl)amino)pyrazine-2-carboxamide

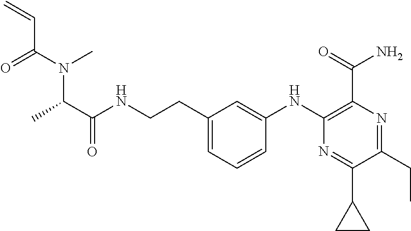 [SMILES: C=CC(=O)N(C)[C@@H](C)C(=O)NCCc1cccc(Nc2nc(C3CC3)c(CC)nc2C(N)=O)c1]

### Step 1: (S)-5-cyclopropyl-6-ethyl-3-((3-(2-(2-(N-methylacrylamido)propanamido)ethyl)phenyl)amino)pyrazine-2-carboxamide

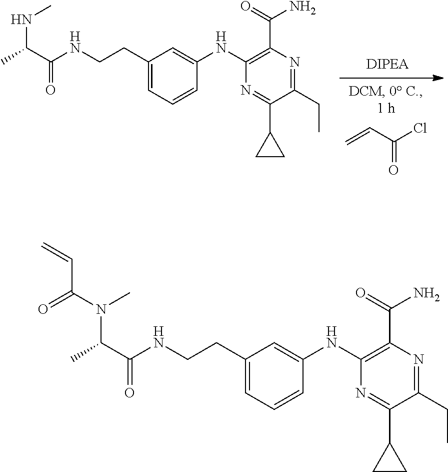

To a solution of give (S)-5-cyclopropyl-6-ethyl-3-((3-(2-(2-(methylamino)propanamido)ethyl)phenyl)amino) pyrazine-2-carboxamide hydrochloride (300 mg, 671.18 umol, 1 eq, HCl) in THF (5 mL) and DMF (3 mL) was added DIEA (433.73 mg, 3.36 mmol, 584.54 uL, 5 eq) and prop-2-enoyl chloride (72.90 mg, 805.42 umol, 65.67 uL, 1.2 eq) at 0° C. The mixture was stirred at 0° C. for 1 h. LC-MS showed the reaction was completed. The reaction mixture was quenched by addition water (50 mL) at 0° C., and then extracted with EtOAc (30 mL*3). The combined organic layers were washed with saturated brine (20 mL*2), dried over Na₂SO₄, filtered and concentrated under reduced pressure to give a residue. The residue was combined with another batch was purified by prep-HPLC (column: Phenomenex Luna C18 150*30 mm*5 um; mobile phase: [water(TFA)-ACN]; B %: 30%-60%, 8 min) to give compound (S)-5-cyclopropyl-6-ethyl-3-((3-(2-(2-(N-methylacrylamido)propanamido)ethyl)phenyl)amino)pyrazine-2-carboxamide as a yellow solid (328.65 mg, 674.20 umol). ¹H NMR (400 MHz, DMSO-d₆) δ=11.04 (br s, 1H), 8.22-7.75 (m, 3H), 7.51-7.36 (m, 2H), 7.27-7.16 (m, 1H), 6.87-6.77 (m, 1H), 6.75-6.58 (m, 1H), 6.16-5.96 (m, 1H), 5.72-5.53 (m, 1H), 5.14-4.44 (m, 1H), 3.57-3.17 (m, 2H), 2.99-2.78 (m, 4H), 2.76-2.65 (m, 3H), 2.34-2.24 (m, 1H), 1.32-1.16 (m, 6H), 1.14-1.02 (m, 4H); LC-MS (ES⁺, m/z): 465.2 [(M+H)⁺]. Rt=2.527 min.

### Example 98

### (Compound 209)

### (S)-5-cyclopropyl-6-ethyl-3-((3-(2-(2-(N-methylbut-2-ynamido)propanamido)ethyl)phenyl)amino)pyrazine-2-carboxamide

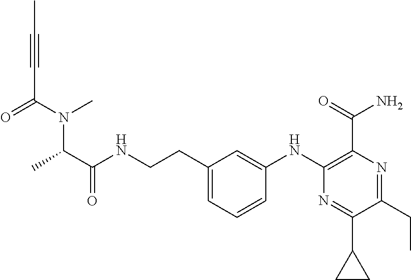 [SMILES: CC#CC(=O)N(C)[C@@H](C)C(=O)NCCc1cccc(Nc2nc(C3CC3)c(CC)nc2C(N)=O)c1]

### Step 1: (S)-5-cyclopropyl-6-ethyl-3-((3-(2-(2-(N-methylbut-2-ynamido)propanamido)ethyl)phenyl)amino)pyrazine-2-carboxamide

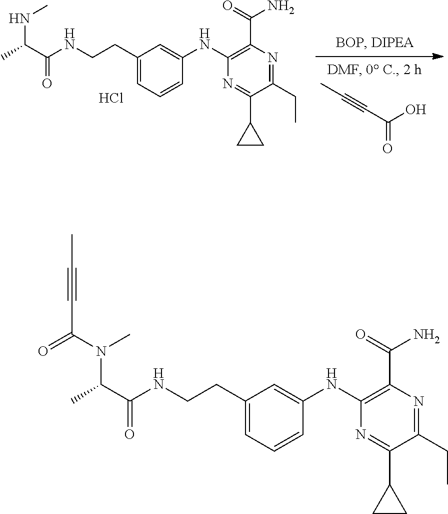

To a solution of but-2-ynoic acid (61.44 mg, 730.80 umol, 1.2 eq), DIEA (787.09 mg, 6.09 mmol, 1.06 mL, 10 eq), (S)-5-cyclopropyl-6-ethyl-3-((3-(2-(2-(methylamino)propanamido)ethyl)phenyl)amino)pyrazine-2-carboxamide hydrochloride (250 mg, 609.00 umol, 1 eq) in DMF (1 mL) at 0° C., BOP (323.22 mg, 730.80 umol, 1.2 eq) was added. The mixture was stirred at 0° C. for 2 hrs. LCMS showed the reaction was completed. The reaction mixture was quenched by addition water (50 mL) at 25° C., and then extracted with EtOAc (30 mL*3). The combined organic layers were washed with saturated brine (20 mL*2), dried over Na₂SO₄, filtered and concentrated under reduced pressure to give a residue. The residue was purified by prep-HPLC (column: Phenomenex Luna C18 150*30 mm*5 um; mobile phase: [water(TFA)-ACN]; B %: 30%-60%, 8 min) to give (S)-5-cyclopropyl-6-ethyl-3-((3-(2-(2-(N-methylbut-2-ynamido) propanamido)ethyl)phenyl)amino)pyrazine-2-carboxamide as yellow solid (96.13 mg, 198.40 umol, 32.58% yield, 98.36% purity). ¹H NMR (400 MHz, DMSO-d₆) δ=11.05 (d, J=3.1 Hz, 1H), 8.12 (br s, 1H), 8.06-7.92 (m, 1H), 7.83 (br s, 1H), 7.45-7.39 (m, 2H), 7.21 (dt, J=1.6, 7.7 Hz, 1H), 6.80 (br d, J=7.5 Hz, 1H), 4.90-4.79 (m, 1H), 3.37 (br d, J=0.9 Hz, 2H), 2.98-2.86 (m, 3H), 2.74-2.67 (m, 4H), 2.32-2.24 (m, 1H), 1.99 (d, J=15.6 Hz, 3H), 1.30-1.18 (m, 6H), 1.10-1.05 (m, 4H) LC-MS (ES⁺, m/z): 477.3 [(M+H)+]; Rt=2.705 min; 98.36% purity; HRMS: 477.2558.

### Example 99

### (Compound 212)

### (S)-5,6-dimethyl-3-((3-(2-(2-(N-methylbut-2-ynamido)propanamido)ethyl)phenyl)amino)pyrazine-2-carboxamide

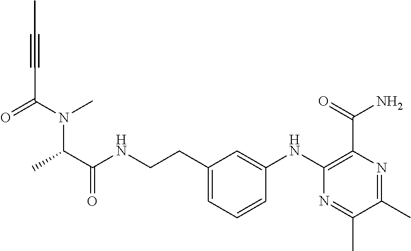 [SMILES: CC#CC(=O)N(C)[C@@H](C)C(=O)NCCc1cccc(Nc2nc(C)c(C)nc2C(N)=O)c1]

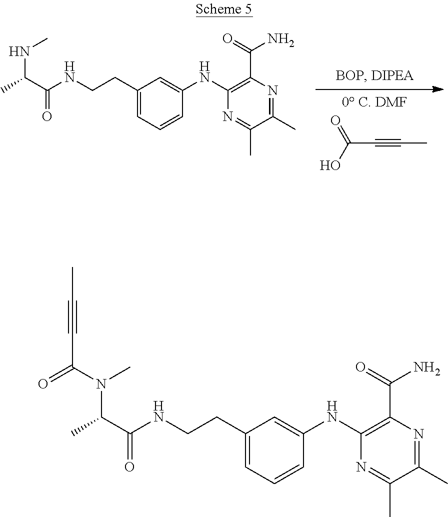

### Step 1: (S)-5,6-dimethyl-3-((3-(2-(2-(N-methylbut-2-ynamido)propanamido)ethyl)phenyl)amino)pyrazine-2-carboxamide

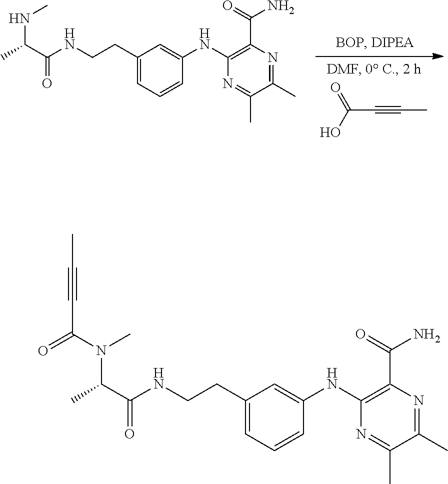

To a solution of but-2-ynoic acid (98.30 mg, 1.17 mmol, 1.2 eq) in DMF (3 mL) was added DIPEA (1.26 g, 9.74 mmol, 1.70 mL, 10 eq) and (S)-5,6-dimethyl-3-((3-(2-(2-(methylamino)propanamido)ethyl)phenyl)amino)pyrazine-2-carboxamide (360 mg, 974.40 umol, 1 eq), then BOP (646.44 mg, 1.46 mmol, 1.5 eq) was added. The mixture was stirred at 0° C. for 2 hrs. LCMS indicated the reaction was complete. The reaction was concentrated. The crude was purified by prep-HPLC column: Phenomenex Luna 80*30 mm*3 um; mobile phase: [water(TFA)-ACN]; B %: 35%-65%, 8 min to give (S)-5,6-dimethyl-3-((3-(2-(2-(N-methylbut-2-ynamido)propanamido)ethyl)phenyl)amino)pyrazine-2-carboxamide (135 mg, 309.27 umol, 31.74% yield) as yellow solid. ¹H NMR (400 MHz, DMSO-d₆) δ=11.06 (d, J=1.4 Hz, 1H), 8.21 (br s, 1H), 8.10-7.90 (m, 1H), 7.82 (br s, 1H), 7.65 (br d, J=8.0 Hz, 1H), 7.44 (br s, 1H), 7.22 (dt, J=1.6, 7.8 Hz, 1H), 6.82 (br d, J=7.5 Hz, 1H), 4.96-4.77 (m, 1H), 3.39-3.23 (m, 2H), 2.99 (s, 2H), 2.81-2.60 (m, 3H), 2.47 (s, 3H), 2.42 (s, 3H), 2.00 (d, J=15.3 Hz, 3H), 1.33-1.17 (m, 3H). LC-MS (ES+, m/z): 437.1 [(M+H)⁺]; Rt=2.401 min; HRMS (EI): m/z [M]⁺ found: 437.2285; SFC: 100%.

### Example 100

### (Compound 235)

### (S)-5-cyclopropyl-6-methyl-3-((3-(2-(2-(N-methylbut-2-ynamido)propanamido)ethyl)phenyl)amino)pyrazine-2-carboxamide

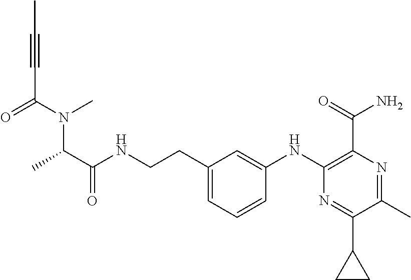 [SMILES: CC#CC(=O)N(C)[C@@H](C)C(=O)NCCc1cccc(Nc2nc(C3CC3)c(C)nc2C(N)=O)c1]

### Step 1: (S)-5-cyclopropyl-6-methyl-3-((3-(2-(2-(N-methylbut-2-ynamido)propanamido)ethyl)phenyl)amino)pyrazine-2-carboxamide

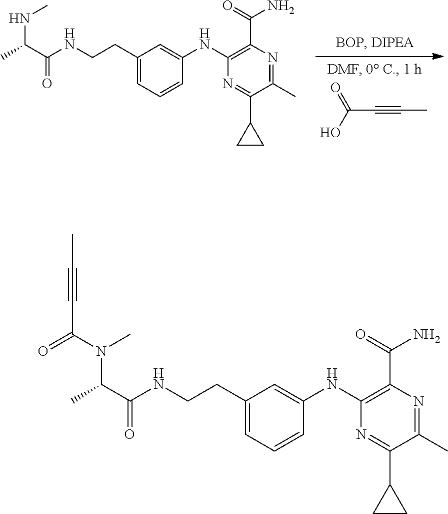

To a solution of but-2-ynoic acid (63.61 mg, 756.65 umol, 1.5 eq) DIPEA (651.94 mg, 5.04 mmol, 878.63 uL, 10 eq), (S)-5-cyclopropyl-6-methyl-3-((3-(2-(2-(methylamino)propanamido)ethyl)phenyl)amino)pyrazine-2-carboxamide (200 mg, 504.43 umol, 1 eq) in DMF (2 mL) at 0° C., BOP (334.65 mg, 756.65 umol, 1.5 eq) was added. The mixture was stirred at 0° C. for 1 h. LCMS indicated the reaction was complete. The mixture was filtered to give a residue. The crude was purified by prep-HPLC column: C18-1 150*30 mm*5 um; mobile phase: [water(TFA)-ACN]; B %: 20%-65%, 8 min) to afford (S)-5-cyclopropyl-6-methyl-3-((3-(2-(2-(N-methylbut-2-ynamido)propanamido)ethyl)phenyl)amino)pyrazine-2-carboxamide (97.03 mg, 209.50 umol, 41.53% yield, 99.87% purity) as yellow solid. 1H NMR (400 MHz, DMSO-d₆) δ=11.06 (d, J=3.0 Hz, 1H), 8.18 (br s, 1H), 8.07-7.91 (m, 1H), 7.83-7.76 (m, 1H), 7.48-7.40 (m, 2H), 7.26-7.19 (m, 1H), 6.85-6.78 (m, 1H), 4.85 (qd, J=7.1, 18.7 Hz, 1H), 3.39-3.24 (m, 2H), 3.00-2.67 (m, 5H), 2.56 (s, 3H), 2.30-2.20 (m, 1H), 2.04-1.97 (m, 3H), 1.31-1.18 (m, 3H), 1.14-1.05 (m, 4H); LCMS (ES+, m/z): 463.3 [(M+H)+]; Rt=2.573 min; HRMS (EI): m/z [M]⁺ found: 463.2424

### Example 101

### (Compound 222)

### (S)-5-cyclopropyl-6-methyl-3-((3-(2-(2-(N-methylacrylamido)propanamido)ethyl)phenyl)amino)pyrazine-2-carboxamide

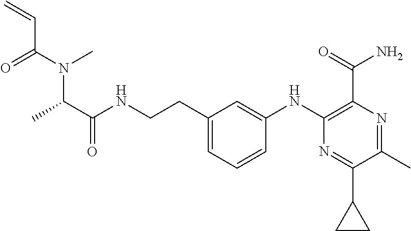 [SMILES: C=CC(=O)N(C)[C@@H](C)C(=O)NCCc1cccc(Nc2nc(C3CC3)c(C)nc2C(N)=O)c1]

### Step 1: (S)-5-cyclopropyl-6-methyl-3-((3-(2-(2-(N-methylacrylamido)propanamido)ethyl)phenyl)amino)pyrazine-2-carboxamide

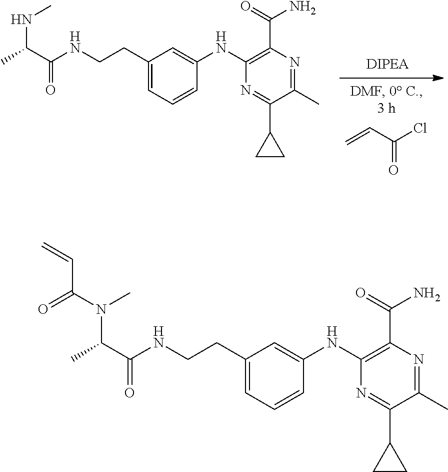

To a solution of (S)-5-cyclopropyl-6-methyl-3-((3-(2-(2-(methylamino)propanamido)ethyl)phenyl)amino) pyrazine-2-carboxamide (1 g, 2.52 mmol, 1 eq) and DIPEA (3.26 g, 25.20 mmol, 4.39 mL, 10 eq) in DMF (10 mL) was added acryloyl chloride (273.93 mg, 3.02 mmol, 246.78 uL, 1.2 eq) at 0° C. The mixture was stirred at 0° C. for 3 hrs. LCMS showed the reaction was completed. The reaction mixture was filtered to give a residue. The residue was purified by prep-HPLC column: C18-1 150*30 mm*5 um; mobile phase: [water(TFA)-ACN]; B %: 15%-60%, 8 min to afford (S)-5-cyclopropyl-6-methyl-3-((3-(2-(2-(N-methylacrylamido)propanamido)ethyl)phenyl)amino)pyrazine-2-carboxamide (307.32 mg, 606.96 umol, 24.09% yield) as yellow solid. ¹H NMR (400 MHz, DMSO-d₆) δ=11.07-11.02 (m, 1H), 8.23-8.13 (m, 1H), 8.11-7.72 (m, 2H), 7.49-7.37 (m, 2H), 7.25-7.17 (m, 1H), 6.85-6.77 (m, 1H), 6.75-6.60 (m, 1H), 6.17-5.97 (m, 1H), 5.71-5.55 (m, 1H), 4.99-4.56 (m, 1H), 3.40-3.23 (m, 2H), 2.91-2.78 (m, 2H), 2.74-2.67 (m, 3H), 2.56-2.53 (m, 3H), 2.28-2.19 (m, 1H), 1.26-1.06 (m, 7H). LC-MS (ES+, m/z): 451.3 [(M+H)⁺]; Rt=2.507 min; HRMS (EI): m/z [M]⁺ found: 451.2243.

### Example 102

### (Compound 218)

### (S)-5-(dimethylamino)-6-methyl-3-((3-(2-(2-(N-methylacrylamido)propanamido)ethyl)phenyl)amino)pyrazine-2-carboxamide

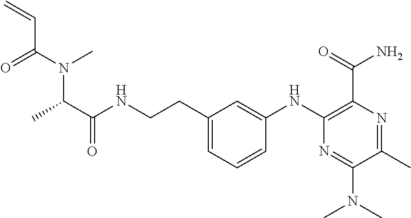 [SMILES: C=CC(=O)N(C)[C@@H](C)C(=O)NCCc1cccc(Nc2nc(N(C)C)c(C)nc2C(N)=O)c1]

### Step 1: (S)-5-(dimethylamino)-6-methyl-3-((3-(2-(2-(N-methylacrylamido)propanamido)ethyl)phenyl)amino)pyrazine-2-carboxamide

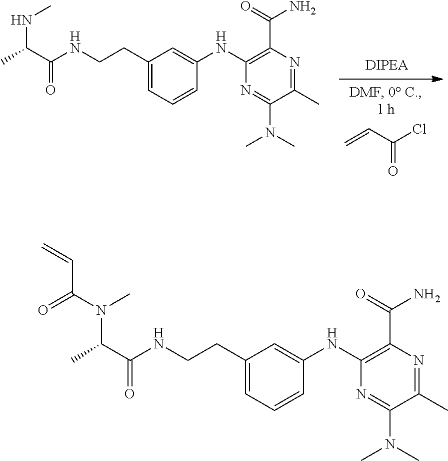

To a mixture of (S)-5-(dimethylamino)-6-methyl-3-((3-(2-(2-(methylamino) propanamido)ethyl)phenyl)amino)pyrazine-2-carboxamide (900 mg, 2.25 mmol, 1 eq) in DMF (5 mL) was added DIPEA (2.91 g, 22.53 mmol, 10 eq) finally added acryloyl chloride (244.68 mg, 2.70 mmol, 1.2 eq) in one portion at 0° C. under N₂. The mixture was stirred at 0° C. for 1 hour. LCMS showed the reaction was completed. The residue was purified by prep-HPLC (column: Phenomenex Luna 80*30 mm*3 um; mobile phase: [water (TFA)-ACN]; B %: 25%-55%, 8 min). Compound (S)-5-(dimethylamino)-6-methyl-3-((3-(2-(2-(N-methylacrylamido) propanamido)ethyl)phenyl)amino)pyrazine-2-carboxamide (301.25 mg, 664.22 umol, 29.48% yield) was obtained as a yellow solid. ¹H NMR (400 MHz, DMSO-d₆) δ=11.17-11.11 (m, 1H), 8.12-7.77 (m, 2H), 7.61-7.55 (m, 1H), 7.52-7.40 (m, 2H), 7.22 (t, J=7.8 Hz, 1H), 6.81 (d, J=7.5 Hz, 1H), 6.73 (br dd, J=10.4, 16.8 Hz, 1H), 6.16 (br d, J=2.0 Hz, 1H), 5.73-5.60 (m, 1H), 5.10-4.50 (m, 1H), 3.44-3.22 (m, 2H), 3.13 (s, 6H), 2.88 (s, 2H), 2.77-2.68 (m, 3H), 2.50 (s, 3H), 1.31-1.17 (m, 3H). LC-MS (ES+, m/z): 454.2 [(M+H)⁺]. Rt=2.389 min; HRMS (EI): m/z [M]⁺ found: 454.2576.

### Example 103

### (Compound 231)

### (S)-5-(isopropyl(methyl)amino)-6-methyl-3-((3-(2-(2-(N-methylacrylamido)propanamido)ethyl)phenyl)amino)pyrazine-2-carboxamide

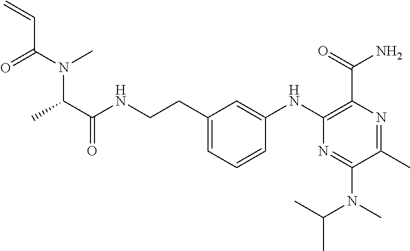 [SMILES: C=CC(=O)N(C)[C@@H](C)C(=O)NCCc1cccc(Nc2nc(N(C)C(C)C)c(C)nc2C(N)=O)c1]

Step 1: (S)-5-(isopropyl(methyl)amino)-6-methyl-3-((3-(2-(2-(N-methylacrylamido)propanamido)ethyl)phenyl)amino)pyrazine-2-carboxamide

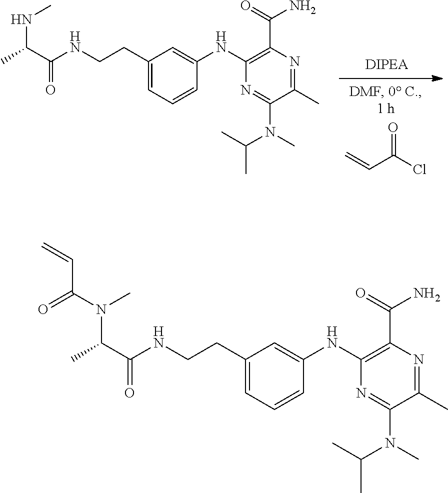

To a solution of (S)-5-(isopropyl(methyl)amino)-6-methyl-3-((3-(2-(2-(methylamino)propanamido)ethyl)phenyl)amino)pyrazine-2-carboxamide (1.3 g, 3.04 mmol, 1 eq) in DMF (3 mL) was added DIEA (1.96 g, 15.20 mmol, 2.65 mL, 5 eq), and then prop-2-enoyl chloride (330.24 mg, 3.65 mmol, 297.52 uL, 1.2 eq) at 0° C. The mixture was stirred at 0° C. for 1 h under N₂. LCMS indicated the reaction was completed. The crude mixture was purified by prep-HPLC column: column: C18 (250*50 mm*10 um); mobile phase: [water(NH₄HCO₃)-ACN]; B %: 35%-65%, 10 min to afford (S)-5-(isopropyl(methyl)amino)-6-methyl-3-((3-(2-(2-(N-methylacrylamido)propanamido)ethyl)phenyl)amino)pyrazine-2-carboxamide (200 mg, 415.29 umol, 13.66% yield) as white solid. 1H NMR (400 MHz, DMSO-d6) δ=11.11 (s, 1H), 8.08-7.84 (m, 1H), 7.78 (br d, J=1.9 Hz, 1H), 7.54 (s, 1H), 7.47-7.40 (m, 2H), 7.20 (t, J=7.8 Hz, 1H), 6.79 (d, J=7.5 Hz, 1H), 6.76-6.62 (m, 1H), 6.15-6.01 (m, 1H), 5.71-5.57 (m, 1H), 4.99 (q, J=7.1 Hz, 1H), 4.40-4.30 (m, 1H), 3.32-3.23 (m, 2H), 2.90 (s, 3H), 2.86 (s, 2H), 2.74-2.66 (m, 3H), 2.45 (s, 3H), 1.26-1.18 (m, 9H). LC-MS (ES+, m/z): 482.3 [(M+H)⁺]; Rt=2.601 min; HRMS (EI): m/z [M]⁺ found: 482.2842.

### Example 104

### (Compound 225)

### (S)-5-(ethyl(methyl)amino)-6-methyl-3-((3-(2-(2-(N-methylacrylamido)propanamido)ethyl)phenyl)amino)pyrazine-2-carboxamide

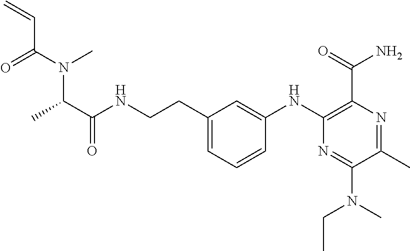 [SMILES: C=CC(=O)N(C)[C@@H](C)C(=O)NCCc1cccc(Nc2nc(N(C)CC)c(C)nc2C(N)=O)c1]

### Step 1: (S)-5-(ethyl(methyl)amino)-6-methyl-3-((3-(2-(2-(N-methylacrylamido)propanamido)ethyl)phenyl)amino)pyrazine-2-carboxamide

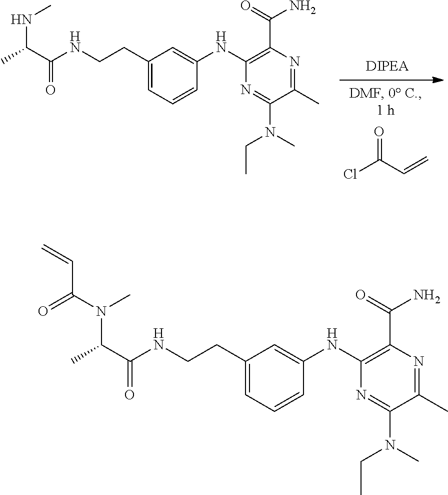

To a solution of (S)-5-(ethyl(methyl)amino)-6-methyl-3-((3-(2-(2-(methylamino)propanamido)ethyl)phenyl)amino)pyrazine-2-carboxamide (1.1 g, 2.66 mmol, 1 eq) in DMF (3 mL) was added DIEA (1.72 g, 13.30 mmol, 2.32 mL, 5 eq), and then prop-2-enoyl chloride (288.92 mg, 3.19 mmol, 260.28 uL, 1.2 eq) at 0° C. The mixture was stirred at 0° C. for 1 h. LCMS indicate the reaction was completed. The reaction was poured into water (100 mL) and extracted with EtOAc (100 mL*2). The organic layers were combined, washed with water (100 mL*2), saturated brine (100 mL), dried (Na₂SO₄), filtered and concentrated to give crude product. The crude mixture was purified by prep-HPLC column: Phenomenex C18 80*30 mm*3 um; mobile phase: [water(TFA)-ACN]; B %: 20%-50%, 7 min. to afford (S)-5-(ethyl(methyl)amino)-6-methyl-3-((3-(2-(2-(N-methylacrylamido)propanamido)ethyl)phenyl)amino)pyrazine-2-carboxamide (300 mg, 641.63 umol, 24.12% yield) as yellow solid. 1H NMR (400 MHz, DMSO-d₆) δ=11.11 (s, 1H), 8.09-7.84 (m, 1H), 7.82-7.73 (m, 1H), 7.55 (s, 1H), 7.46-7.36 (m, 2H), 7.20 (t, J=7.8 Hz, 1H), 6.79 (d, J=7.6 Hz, 1H), 6.75-6.62 (m, 1H), 6.16-6.01 (m, 1H), 5.70-5.59 (m, 1H), 4.99-4.58 (m, 1H), 3.51 (q, J=7.0 Hz, 2H), 3.35-3.24 (m, 2H), 3.09 (s, 3H), 2.86 (s, 2H), 2.75-2.65 (m, 3H), 2.47 (s, 3H), 1.28-1.18 (m, 6H). LC-MS (ES+, m/z): 468.3 [(M+H)⁺]; Rt=2.493 min; HRMS (EI): m/z [M+H]+: 468.2710.

### Additional Exemplary Compounds

The representative compounds prepared or can be prepared from readily available starting materials using the general methods and procedures described herein are depicted in Table 1.

### Example B1

### Flt3 In Vitro Inhibitory Activity

The proliferation inhibitory effect was investigated in human AML cell lines MOLM-13 (AddexBio: C0003003)[REF: Pumbed: 9305600] and MV-4-11 (ATCC: CRL-9591)[REF PMID: 3496132], and CML cell line K-652 (ATCC: CRL-3343) [REF PMID: 163658]. Molm-13 is heterozygous for FLT3-ITD mutation, while MV4-11 is homozygous for FLT3-ITD [PMID: 12529668]. K562 does not harbor any FLT3 mutations. Cells were maintained in RPMI-1640 medium (ThermoFisher catalog no. 61870036) supplemented with 10% of Heat Inactivated FBS (ThermoFisher catalog no. A31605) and 1% Pen-Strep (ThermoFisher catalog no. 10378016) and cultured at 37° C. in a humidified incubator with 5% CO₂. Cell lines were grown by adhering to culture flasks, and the cells were maintained in a range 70%-80% confluency.

ATP is present in all metabolically active cells and is considered as a marker for cell viability and proliferation. Metabolic cell activity was determined using the CellTiter-Glo kit (Promega catalog no. G7572), an ATP monitoring system based on the production of luminescence by the reaction of ATP with added UltraGlo® recombinant luciferase (Kawano et al., 2016, PLOS One, 8; 11(7):e0158888), according to the supplier's experimental recommendations. The assay was based on a 96 well plate format.

Test compounds were dissolved at 10 mM in DMSO (Sigma catalog no. D8418; purity ≥99.9%) and stored at −20° C. Eight concentrations of test compound were assessed in duplicate in an individual test occasion in parallel in the selected cell lines. Gilteratinib and FF-10101 were used as reference compounds and tested in duplicate at eight concentrations. 100% of proliferation is represented by the untreated cells (0.2% DMSO).

On the day of the experiment (T0), cells were quantified with the cell viability analyser NucleoCounter (Chemometec NC-200) and diluted with fresh medium to obtain the cell density of about 10,000 cells per 200 μL medium. 200 μL of cell suspension were added into wells in the 96 well plate.

After 2 hours from seeding cells, compounds were added using a D300e compound dispenser (Life Sciences Tecan D300) at desired concentrations with shaking between each compound dispensed. Cell plates containing 200 μL/well suspension were incubated at 37° C. in a humidified incubator with 5% CO₂.

On day T4, plates containing the samples were equilibrated at room temperature for approximately 15 min, and then 40 μL/well of the Promega CellTiterGlo® reagent was added. Contents were mixed for 5 min on an orbital shaker to induce cell lysis, and then incubated at room temperature for an additional 10 m in the dark to stabilize the luminescent signal. Luminescence was read by using a GloMax GM300 plate reader (Promega) using the luminescence for 96 well plate standard protocol.

Data was expressed as % of inhibition compared to the 0.2% DMSO negative control, and is calculated as follows: % inhibition=100−[(RLU sample)×100/(RLU average controls*)], where * indicates the average for 0.2% DMSO.

Results were analysed by GraphPad (Prism) and IC₅₀ values were calculated by non-linear regression using four parameter-logistic equation. IC₅₀ values measured on day T4 (4 days) are reported in the Table 3 below: *****≤1 nM, **** 1-10 nM, *** 10-100 nM, ** 100-500 nM, *>500 nM.

Compounds of the disclosure provided the following IC₅₀ (4 days) values:

TABLE 3
Representative compounds and their IC50 values

| Compd | | | | | |
|---|---|---|---|---|---|
| ID | Structure | MW | MOLM-13 | MV-4:11 | K-562 |
| 137 | 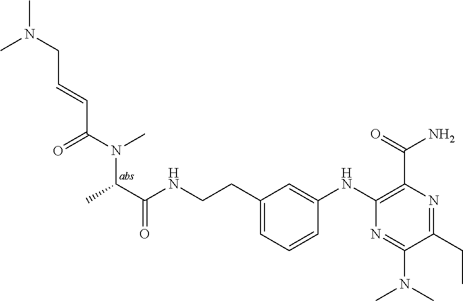 [SMILES: CCc1nc(C(N)=O)c(Nc2cccc(CCNC(=O)[C@H](C)N(C)C(=O)/C=C/CN(C)C)c2)nc1N(C)C] | 524.66 | ***** | ***** | * |
| 138 | 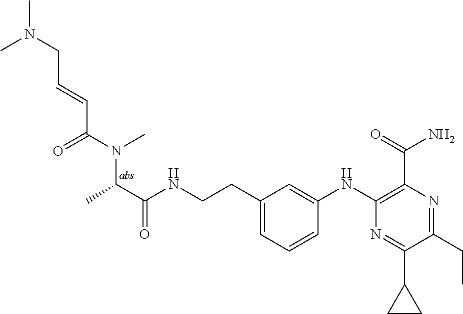 [SMILES: CCc1nc(C(N)=O)c(Nc2cccc(CCNC(=O)[C@H](C)N(C)C(=O)/C=C/CN(C)C)c2)nc1C1CC1] | 521.65 | **** | ***** | * |
| 142A | 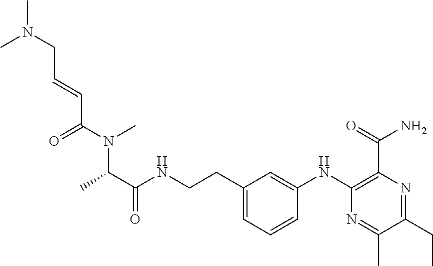 [SMILES: CCc1nc(C(N)=O)c(Nc2cccc(CCNC(=O)[C@H](C)N(C)C(=O)/C=C/CN(C)C)c2)nc1C] | 495.63 | **** | **** | * |
| 143 | 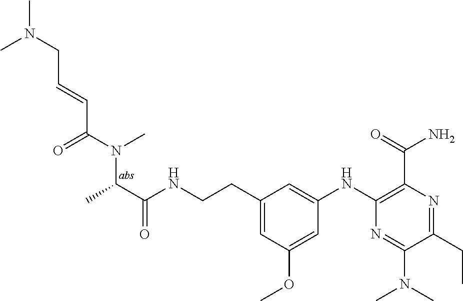 [SMILES: CCc1nc(C(N)=O)c(Nc2cc(CCNC(=O)[C@H](C)N(C)C(=O)/C=C/CN(C)C)cc(OC)c2)nc1N(C)C] | 554.68 | ***** | ***** | * |
| 144 | 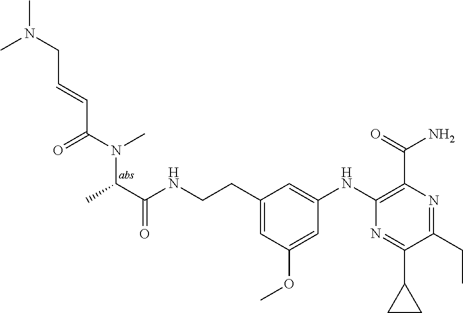 [SMILES: CCc1nc(C(N)=O)c(Nc2cc(CCNC(=O)[C@H](C)N(C)C(=O)/C=C/CN(C)C)cc(OC)c2)nc1C1CC1] | 551.68 | ***** | ***** | * |
| 156 | 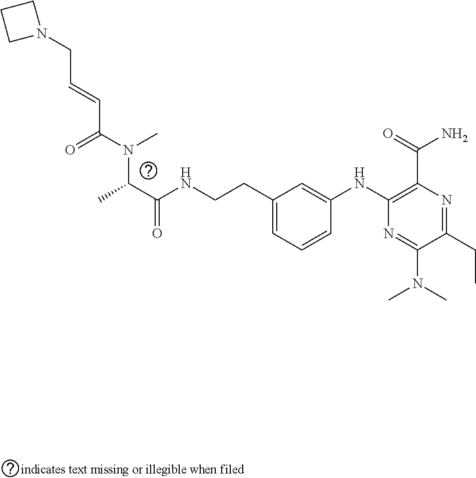 [SMILES: CCc1nc(C(N)=O)c(Nc2cccc(CCNC(=O)[C@H](C)N(C)C(=O)/C=C/CN3CCC3)c2)nc1N(C)C] | 536.67 | ***** | ***** | * |
| 158 | 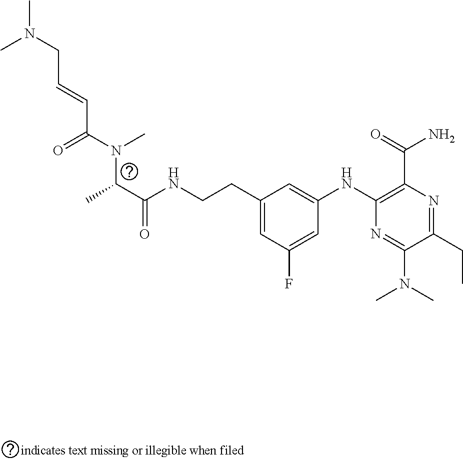 [SMILES: CCc1nc(C(N)=O)c(Nc2cc(F)cc(CCNC(=O)[C@H](C)N(C)C(=O)/C=C/CN(C)C)c2)nc1N(C)C] | 542.65 | ***** | ***** | * |
| 159 | 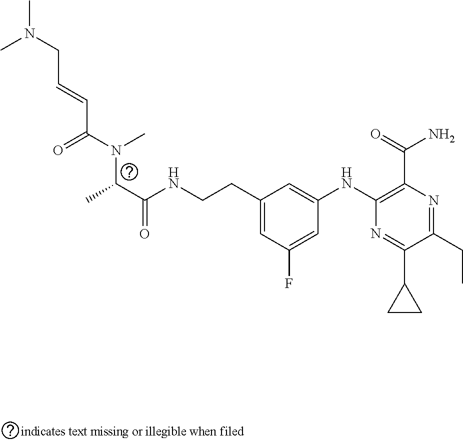 [SMILES: CCc1nc(C(N)=O)c(Nc2cc(F)cc(CCNC(=O)[C@H](C)N(C)C(=O)/C=C/CN(C)C)c2)nc1C1CC1] | 539.64 | **** | ***** | * |
| 160 | 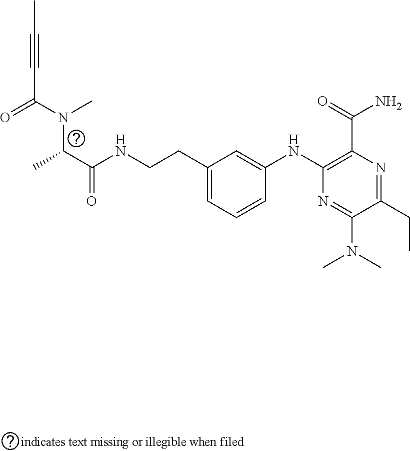 [SMILES: CC#CC(=O)N(C)[C@@H](C)C(=O)NCCc1cccc(Nc2nc(N(C)C)c(CC)nc2C(N)=O)c1] | 479.57 | *** | **** | * |
| 162 | 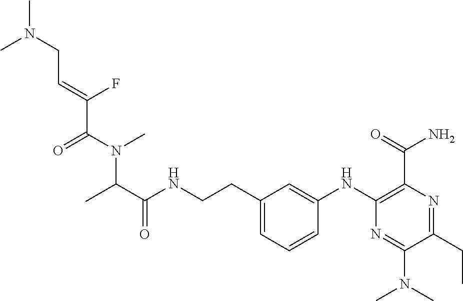 [SMILES: CCc1nc(C(N)=O)c(Nc2cccc(CCNC(=O)C(C)N(C)C(=O)/C(F)=C/CN(C)C)c2)nc1N(C)C] | 542.65 | ** | *** | * |
| 163 | 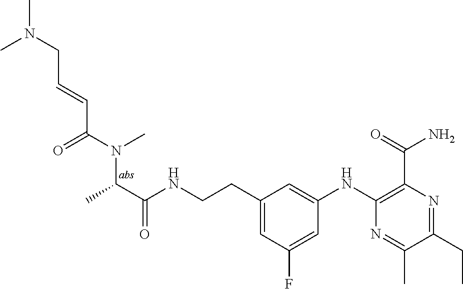 [SMILES: CCc1nc(C(N)=O)c(Nc2cc(F)cc(CCNC(=O)[C@H](C)N(C)C(=O)/C=C/CN(C)C)c2)nc1C] | 513.61 | **** | **** | * |
| 164 | 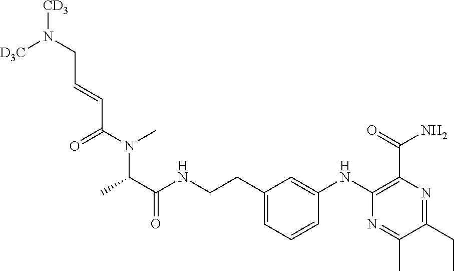 [SMILES: [2H]C([2H])([2H])N(C/C=C/C(=O)N(C)[C@@H](C)C(=O)NCCc1cccc(Nc2nc(C)c(CC)nc2C(N)=O)c1)C([2H])([2H])[2H]] | 501.65 | | | |
| 202 | 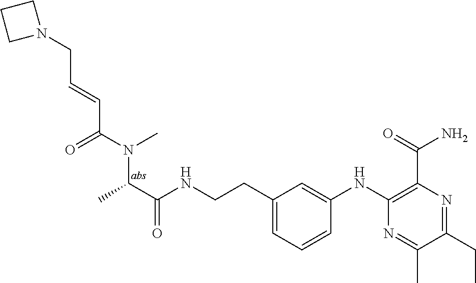 [SMILES: CCc1nc(C(N)=O)c(Nc2cccc(CCNC(=O)[C@H](C)N(C)C(=O)/C=C/CN3CCC3)c2)nc1C] | 507.63 | **** | **** | * |
| 203 | 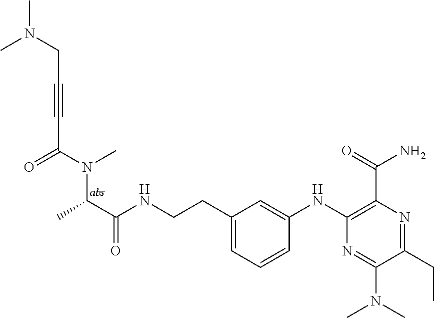 [SMILES: CCc1nc(C(N)=O)c(Nc2cccc(CCNC(=O)[C@H](C)N(C)C(=O)C#CCN(C)C)c2)nc1N(C)C] | 522.64 | **** | ***** | * |
| 204 | 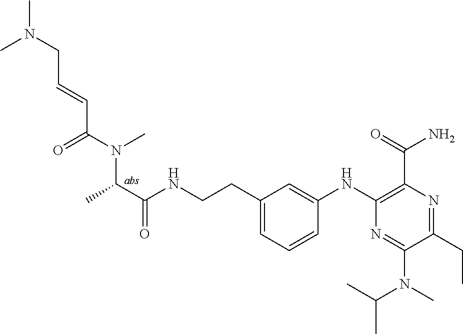 [SMILES: CCc1nc(C(N)=O)c(Nc2cccc(CCNC(=O)[C@H](C)N(C)C(=O)/C=C/CN(C)C)c2)nc1N(C)C(C)C] | 552.71 | ***** | ***** | * |
| 205 | 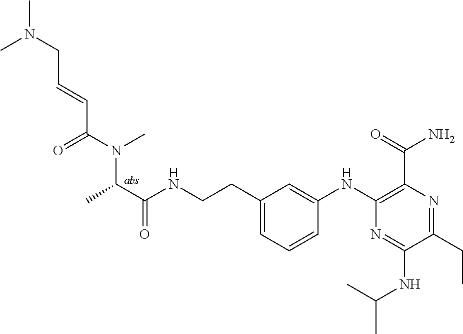 [SMILES: CCc1nc(C(N)=O)c(Nc2cccc(CCNC(=O)[C@H](C)N(C)C(=O)/C=C/CN(C)C)c2)nc1NC(C)C] | 538.68 | ***** | ***** | * |
| 207 | 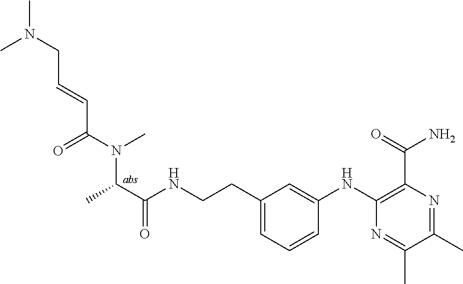 | 481.59 | *** | *** | * |
| 208 | 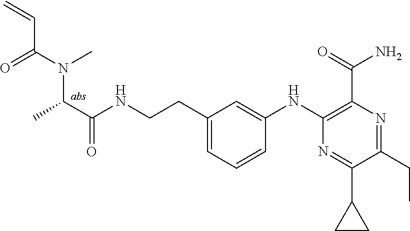 [SMILES: C=CC(=O)N(C)[C@@H](C)C(=O)NCCc1cccc(Nc2nc(C3CC3)c(CC)nc2C(N)=O)c1] | 464.56 | *** | **** | * |
| 209 | 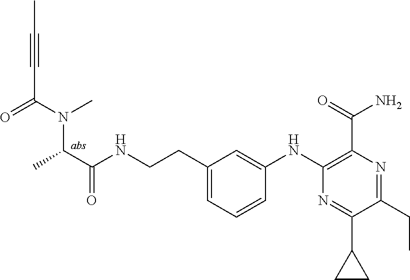 [SMILES: CC#CC(=O)N(C)[C@@H](C)C(=O)NCCc1cccc(Nc2nc(C3CC3)c(CC)nc2C(N)=O)c1] | 476.57 | *** | **** | * |
| 211 | 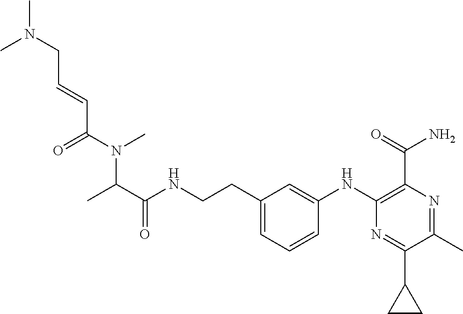 | 507.63 | *** | **** | * |
| 213 | 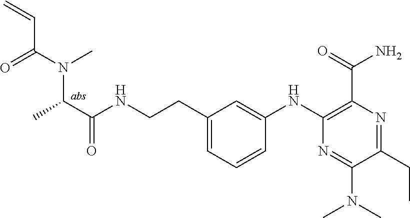 [SMILES: C=CC(=O)N(C)[C@@H](C)C(=O)NCCc1cccc(Nc2nc(N(C)C)c(CC)nc2C(N)=O)c1] | 467.56 | **** | ***** | * |
| 214 | 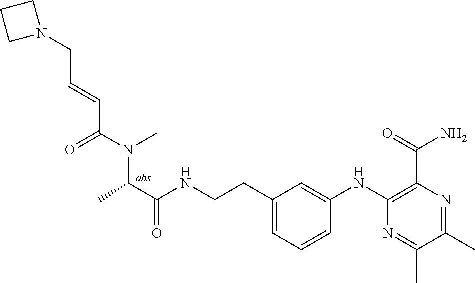 [SMILES: Cc1nc(Nc2cccc(CCNC(=O)[C@H](C)N(C)C(=O)/C=C/CN3CCC3)c2)c(C(N)=O)nc1C] | 493.6 | *** | *** | * |
| 215 | 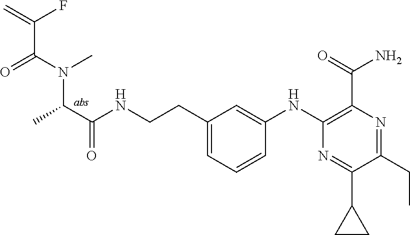 [SMILES: C=C(F)C(=O)N(C)[C@@H](C)C(=O)NCCc1cccc(Nc2nc(C3CC3)c(CC)nc2C(N)=O)c1] | 482.55 | * | * | * |
| 216 | 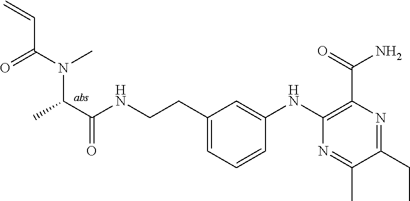 [SMILES: C=CC(=O)N(C)[C@@H](C)C(=O)NCCc1cccc(Nc2nc(C)c(CC)nc2C(N)=O)c1] | 438.52 | *** | *** | * |
| 217 | 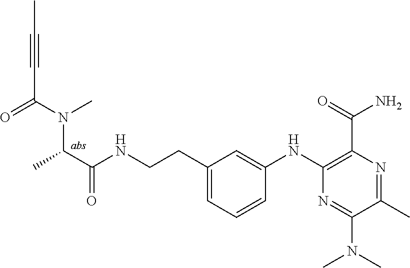 [SMILES: CC#CC(=O)N(C)[C@@H](C)C(=O)NCCc1cccc(Nc2nc(N(C)C)c(C)nc2C(N)=O)c1] | 465.55 | *** | *** | * |
| 218 | 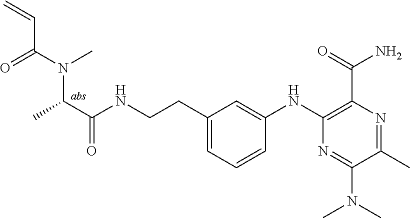 [SMILES: C=CC(=O)N(C)[C@@H](C)C(=O)NCCc1cccc(Nc2nc(N(C)C)c(C)nc2C(N)=O)c1] | 453.54 | *** | **** | * |
| 219 | 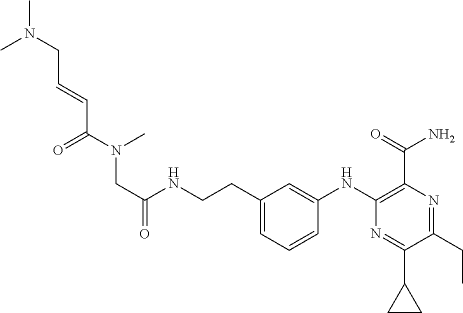 [SMILES: CCc1nc(C(N)=O)c(Nc2cccc(CCNC(=O)CN(C)C(=O)/C=C/CN(C)C)c2)nc1C1CC1] | 507.63 | ***** | ***** | * |
| 220 | 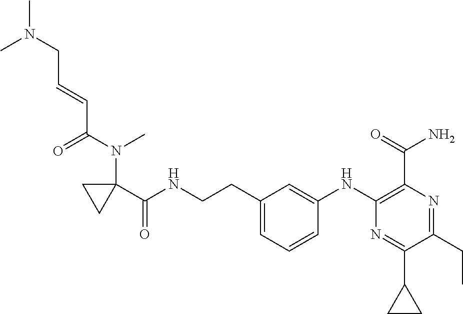 [SMILES: CCc1nc(C(N)=O)c(Nc2cccc(CCNC(=O)C3(N(C)C(=O)/C=C/CN(C)C)CC3)c2)nc1C1CC1] | 533.67 | *** | **** | * |
| 221 | 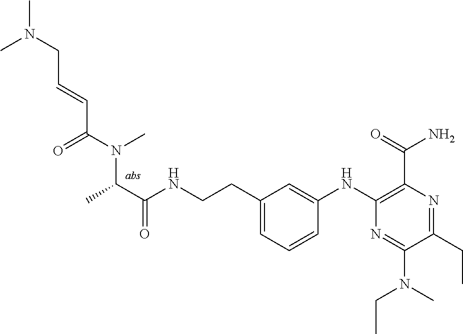 [SMILES: CCc1nc(C(N)=O)c(Nc2cccc(CCNC(=O)[C@H](C)N(C)C(=O)/C=C/CN(C)C)c2)nc1N(C)CC] | 538.68 | ***** | ***** | * |
| 222 | 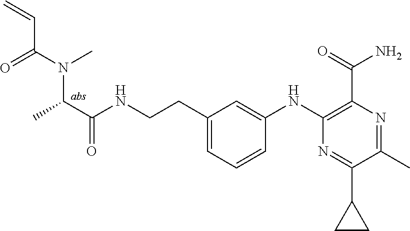 [SMILES: C=CC(=O)N(C)[C@@H](C)C(=O)NCCc1cccc(Nc2nc(C3CC3)c(C)nc2C(N)=O)c1] | 450.53 | *** | *** | * |
| 223 | 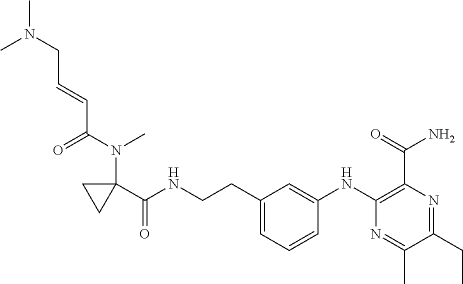 [SMILES: CCc1nc(C(N)=O)c(Nc2cccc(CCNC(=O)C3(N(C)C(=O)/C=C/CN(C)C)CC3)c2)nc1C] | 507.63 | *** | **** | * |
| 224 | 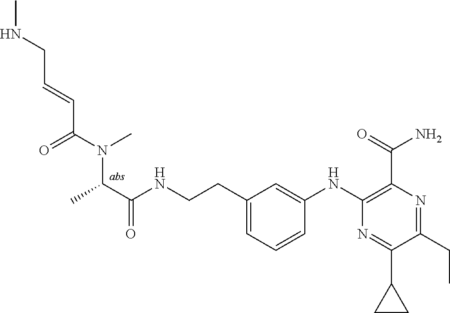 [SMILES: CCc1nc(C(N)=O)c(Nc2cccc(CCNC(=O)[C@H](C)N(C)C(=O)/C=C/CNC)c2)nc1C1CC1] | 507.63 | **** | ***** | * |
| 225 | 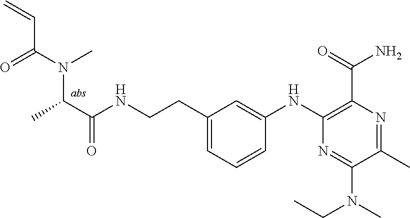 [SMILES: C=CC(=O)N(C)[C@@H](C)C(=O)NCCc1cccc(Nc2nc(N(C)CC)c(C)nc2C(N)=O)c1] | 467.56 | *** | **** | * |
| 226 | 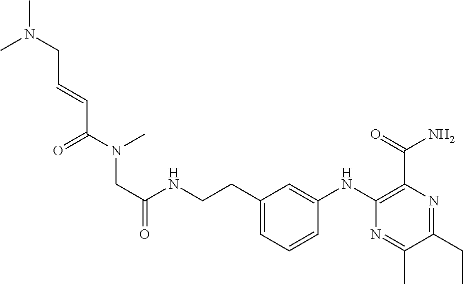 [SMILES: CCc1nc(C(N)=O)c(Nc2cccc(CCNC(=O)CN(C)C(=O)/C=C/CN(C)C)c2)nc1C] | 481.59 | **** | ***** | * |
| 227 | 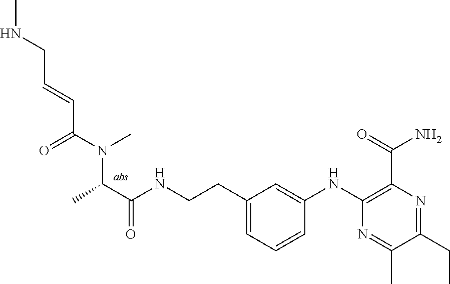 [SMILES: CCc1nc(C(N)=O)c(Nc2cccc(CCNC(=O)[C@H](C)N(C)C(=O)/C=C/CNC)c2)nc1C] | 481.59 | **** | **** | * |
| 228 | 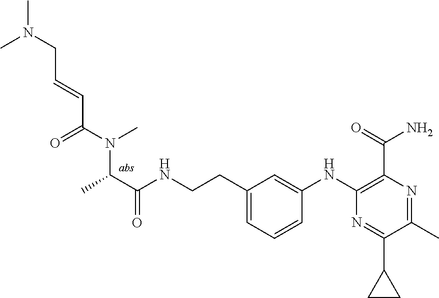 | 507.63 | **** | **** | * |
| 230 | 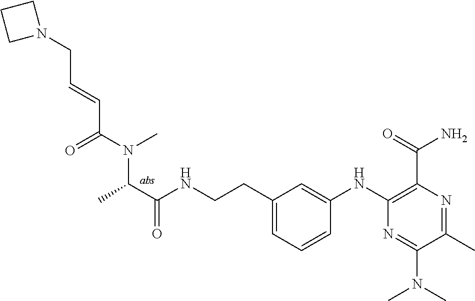 [SMILES: Cc1nc(C(N)=O)c(Nc2cccc(CCNC(=O)[C@H](C)N(C)C(=O)/C=C/CN3CCC3)c2)nc1N(C)C] | 522.64 | **** | ***** | * |
| 231 | 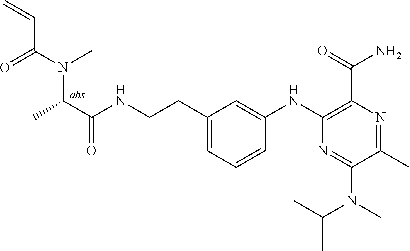 [SMILES: C=CC(=O)N(C)[C@@H](C)C(=O)NCCc1cccc(Nc2nc(N(C)C(C)C)c(C)nc2C(N)=O)c1] | 481.59 | *** | **** | * |
| 232 | 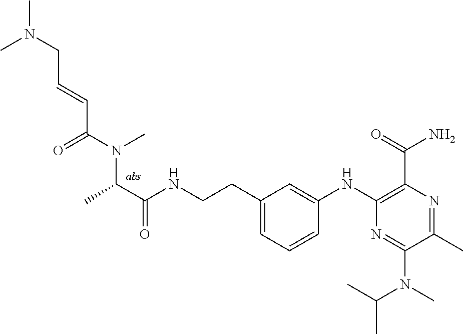 | 538.68 | **** | ***** | * |
| 233 | 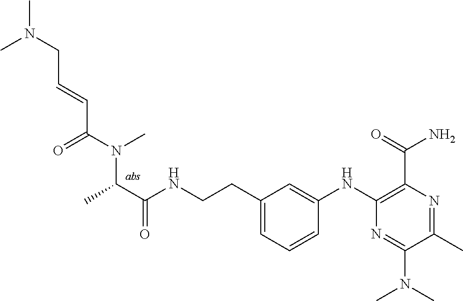 | 510.63 | **** | ***** | * |
| 234 | 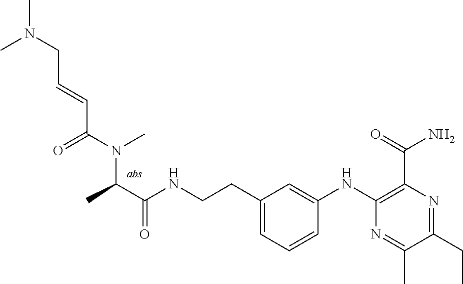 [SMILES: CCc1nc(C(N)=O)c(Nc2cccc(CCNC(=O)[C@@H](C)N(C)C(=O)/C=C/CN(C)C)c2)nc1C] | 495.62 | * | *** | * |
| 236 | 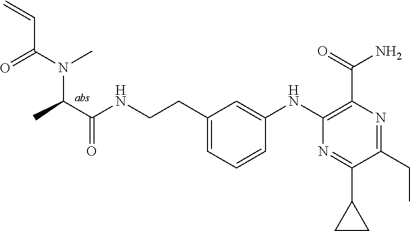 [SMILES: C=CC(=O)N(C)[C@H](C)C(=O)NCCc1cccc(Nc2nc(C3CC3)c(CC)nc2C(N)=O)c1] | 464.56 | *** | *** | * |
| 237 | 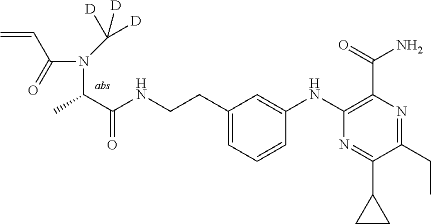 [SMILES: [2H]C([2H])([2H])N(C(=O)C=C)[C@@H](C)C(=O)NCCc1cccc(Nc2nc(C3CC3)c(CC)nc2C(N)=O)c1] | 467.58 | *** | **** | * |
| 238 | 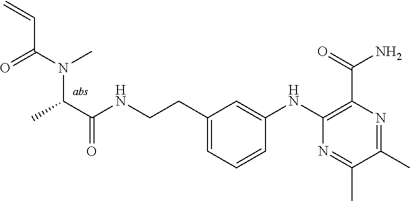 [SMILES: C=CC(=O)N(C)[C@@H](C)C(=O)NCCc1cccc(Nc2nc(C)c(C)nc2C(N)=O)c1] | 424.5 | * | * | * |
| 239 | 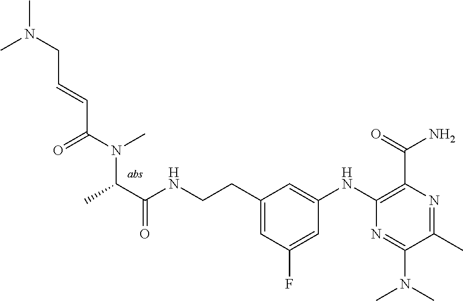 | 528.62 | *** | **** | * |
| 240 | 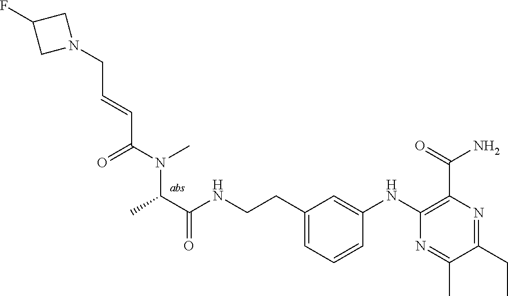 [SMILES: CCc1nc(C(N)=O)c(Nc2cccc(CCNC(=O)[C@H](C)N(C)C(=O)/C=C/CN3CC(F)C3)c2)nc1C] | 525.62 | * | *** | * |
| 241 | 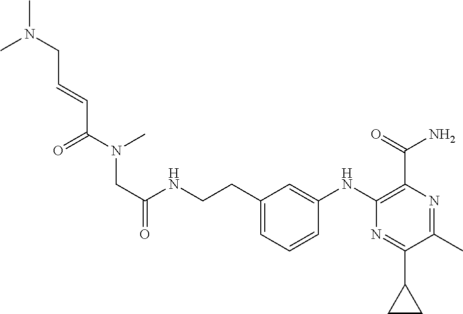 | 493.6 | **** | **** | * |
| 242 | 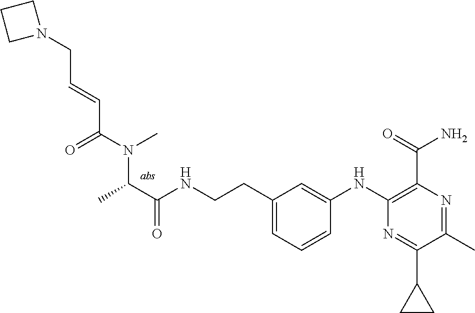 [SMILES: Cc1nc(C(N)=O)c(Nc2cccc(CCNC(=O)[C@H](C)N(C)C(=O)/C=C/CN3CCC3)c2)nc1C1CC1] | 519.64 | **** | **** | * |
| 243 | 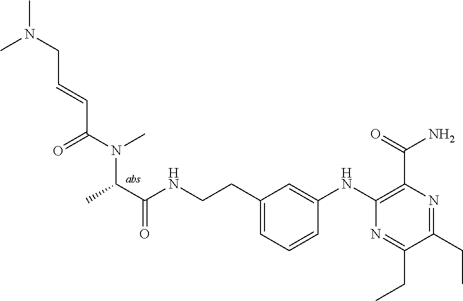 [SMILES: CCc1nc(Nc2cccc(CCNC(=O)[C@H](C)N(C)C(=O)/C=C/CN(C)C)c2)c(C(N)=O)nc1CC] | 509.64 | **** | ***** | * |
| 244 | 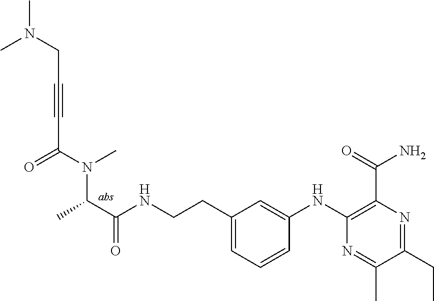 [SMILES: CCc1nc(C(N)=O)c(Nc2cccc(CCNC(=O)[C@H](C)N(C)C(=O)C#CCN(C)C)c2)nc1C] | 493.6 | **** | ***** | * |
| 245 | 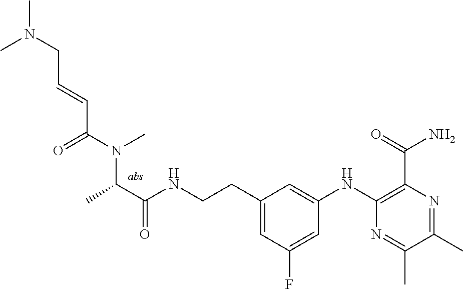 | 499.58 | *** | **** | * |
| 246 | 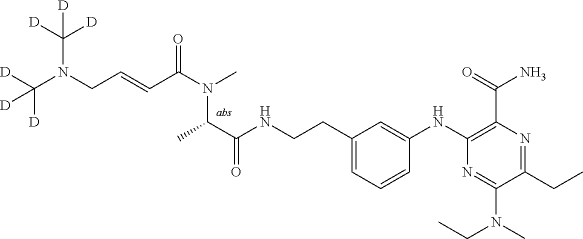 [SMILES: [2H]C([2H])([2H])N(C/C=C/C(=O)N(C)[C@@H](C)C(=O)NCCc1cccc(Nc2nc(N(C)CC)c(CC)nc2C(N)=O)c1)C([2H])([2H])[2H]] | 544.72 | ***** | ***** | * |
| 247 | 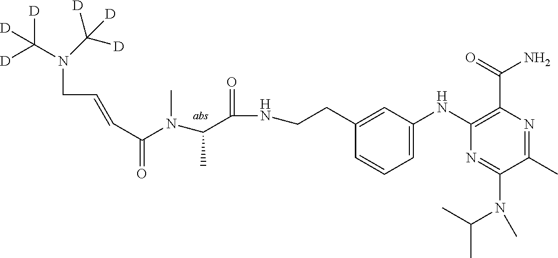 [SMILES: [2H]C([2H])([2H])N(C/C=C/C(=O)N(C)[C@@H](C)C(=O)NCCc1cccc(Nc2nc(N(C)C(C)C)c(C)nc2C(N)=O)c1)C([2H])([2H])[2H]] | 544.72 | **** | ***** | * |
| 248 | 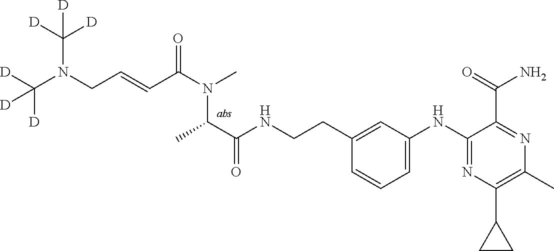 [SMILES: [2H]C([2H])([2H])N(C/C=C/C(=O)N(C)[C@@H](C)C(=O)NCCc1cccc(Nc2nc(C3CC3)c(C)nc2C(N)=O)c1)C([2H])([2H])[2H]] | 513.66 | **** | **** | * |
| 249 | 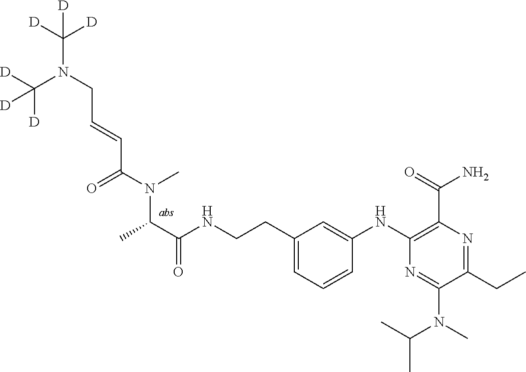 [SMILES: [2H]C([2H])([2H])N(C/C=C/C(=O)N(C)[C@@H](C)C(=O)NCCc1cccc(Nc2nc(N(C)C(C)C)c(CC)nc2C(N)=O)c1)C([2H])([2H])[2H]] | 558.75 | ***** | ***** | * |
| 250 | 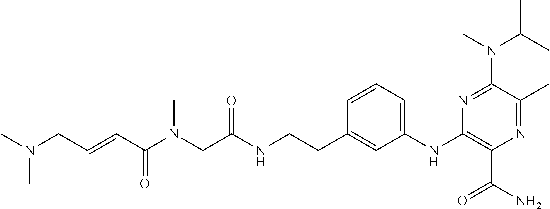 [SMILES: Cc1nc(C(N)=O)c(Nc2cccc(CCNC(=O)CN(C)C(=O)/C=C/CN(C)C)c2)nc1N(C)C(C)C] | 524.66 | **** | ***** | * |
| 251 | 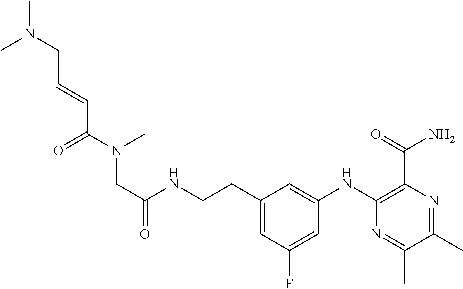 | 485.55 | *** | **** | * |
| 252 | 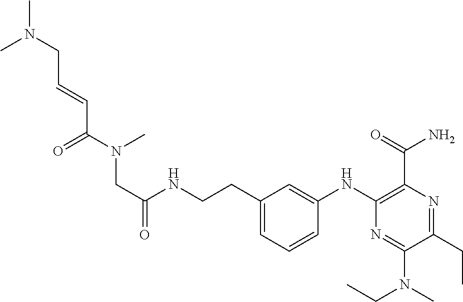 [SMILES: CCc1nc(C(N)=O)c(Nc2cccc(CCNC(=O)CN(C)C(=O)/C=C/CN(C)C)c2)nc1N(C)CC] | 524.66 | ***** | ***** | * |
| 253 | 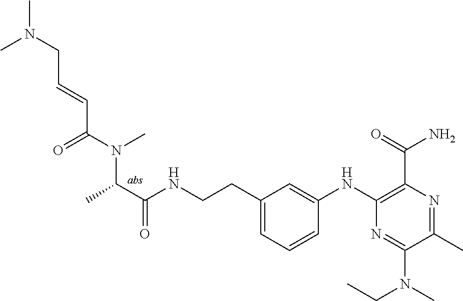 | 524.66 | **** | ***** | * |
| 254 | 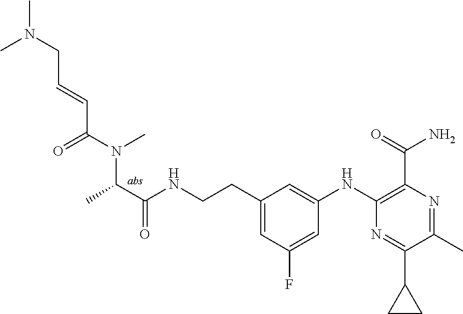 | 525.62 | **** | ***** | * |
| 255 | 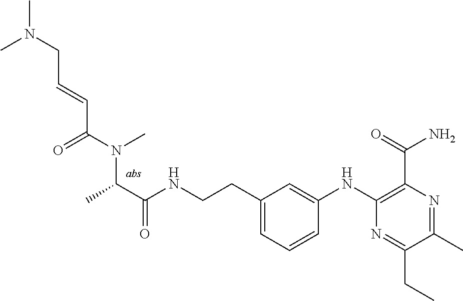 | 495.62 | *** | **** | * |
| 301 | 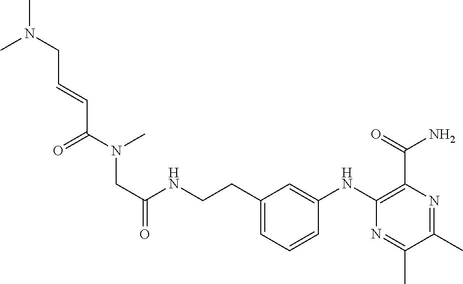 | 467.56 | *** | *** | * |
| 302 | 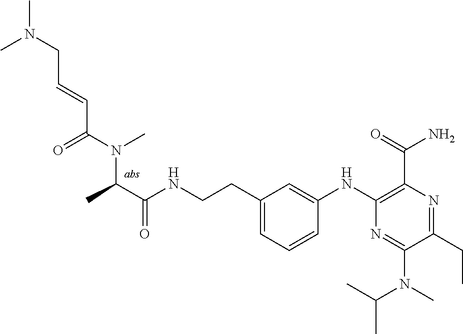 [SMILES: CCc1nc(C(N)=O)c(Nc2cccc(CCNC(=O)[C@@H](C)N(C)C(=O)/C=C/CN(C)C)c2)nc1N(C)C(C)C] | 552.71 | **** | ***** | * |
| 303 | 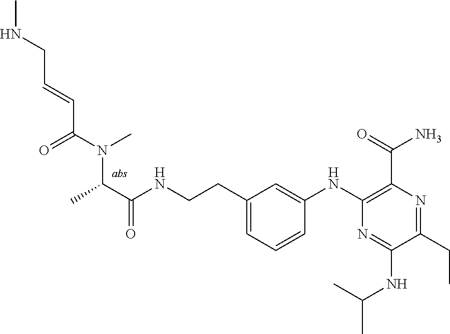 [SMILES: CCc1nc(C(N)=O)c(Nc2cccc(CCNC(=O)[C@H](C)N(C)C(=O)/C=C/CNC)c2)nc1NC(C)C] | 524.66 | ***** | ***** | * |
| 304 | 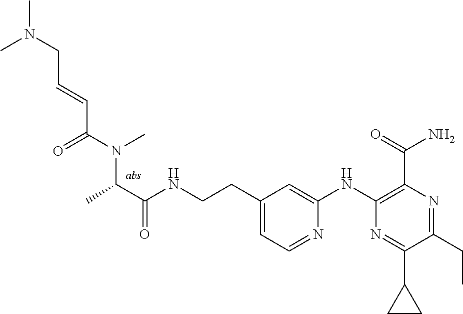 [SMILES: CCc1nc(C(N)=O)c(Nc2cc(CCNC(=O)[C@H](C)N(C)C(=O)/C=C/CN(C)C)ccn2)nc1C1CC1] | 522.64 | ** | ** | * |
| 305 | 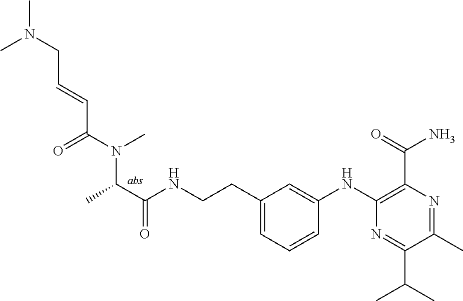 | 509.64 | **** | **** | * |
| 306 | 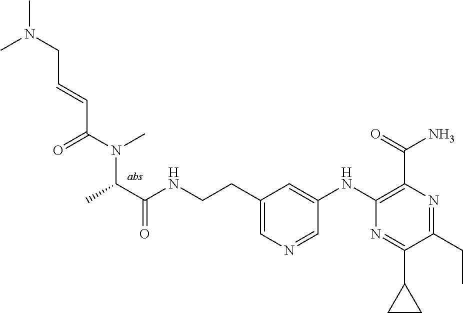 [SMILES: CCc1nc(C(N)=O)c(Nc2cncc(CCNC(=O)[C@H](C)N(C)C(=O)/C=C/CN(C)C)c2)nc1C1CC1] | 522.64 | **** | **** | * |
| 307 | 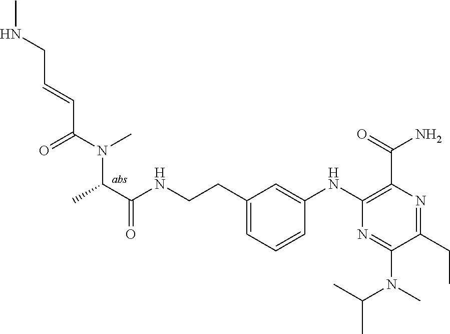 [SMILES: CCc1nc(C(N)=O)c(Nc2cccc(CCNC(=O)[C@H](C)N(C)C(=O)/C=C/CNC)c2)nc1N(C)C(C)C] | 538.68 | ***** | ***** | * |
| 308 | 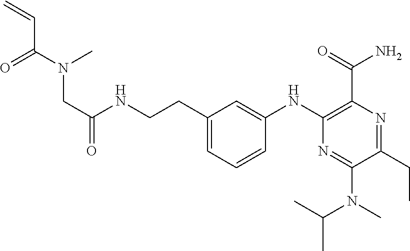 [SMILES: C=CC(=O)N(C)CC(=O)NCCc1cccc(Nc2nc(N(C)C(C)C)c(CC)nc2C(N)=O)c1] | 481.59 | **** | **** | * |
| 309 | 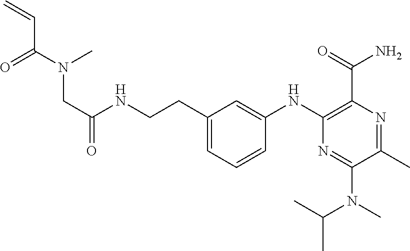 [SMILES: C=CC(=O)N(C)CC(=O)NCCc1cccc(Nc2nc(N(C)C(C)C)c(C)nc2C(N)=O)c1] | 467.56 | ** | **** | * |
| 310 | 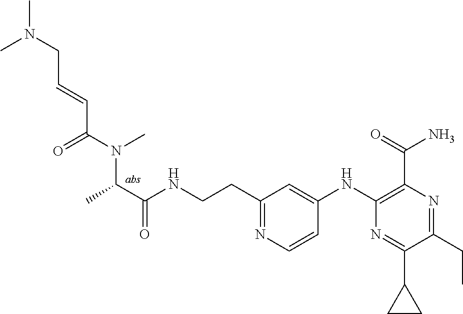 [SMILES: CCc1nc(C(N)=O)c(Nc2ccnc(CCNC(=O)[C@H](C)N(C)C(=O)/C=C/CN(C)C)c2)nc1C1CC1] | 522.64 | **** | ***** | * |
| 311 | 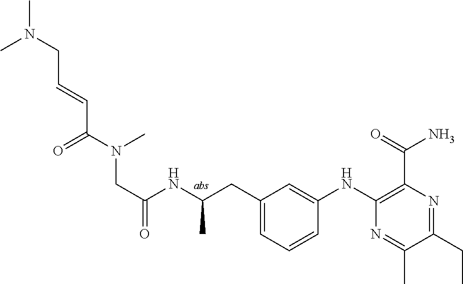 [SMILES: CCc1nc(C(N)=O)c(Nc2cccc(C[C@@H](C)NC(=O)CN(C)C(=O)/C=C/CN(C)C)c2)nc1C] | 495.62 | ** | *** | * |
| 312 | 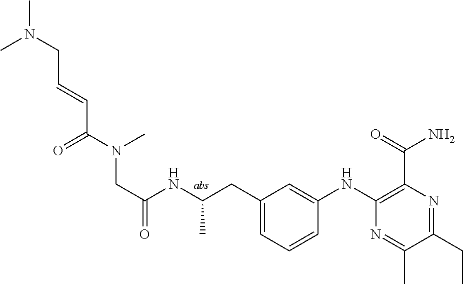 [SMILES: CCc1nc(C(N)=O)c(Nc2cccc(C[C@H](C)NC(=O)CN(C)C(=O)/C=C/CN(C)C)c2)nc1C] | 495.62 | ** | *** | * |
| 313 | 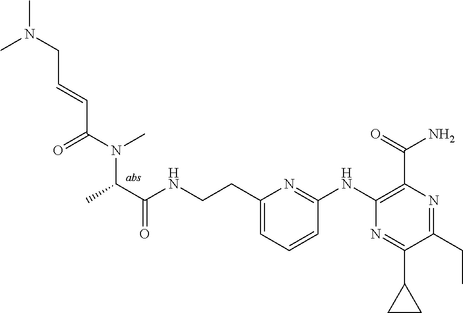 [SMILES: CCc1nc(C(N)=O)c(Nc2cccc(CCNC(=O)[C@H](C)N(C)C(=O)/C=C/CN(C)C)n2)nc1C1CC1] | 522.64 | ** | *** | * |
| 314 | 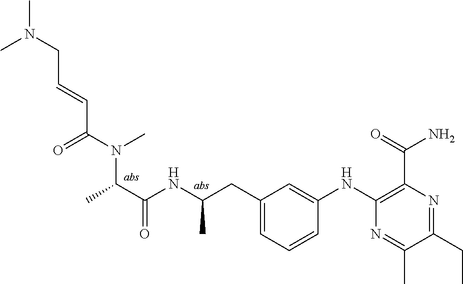 [SMILES: CCc1nc(C(N)=O)c(Nc2cccc(C[C@@H](C)NC(=O)[C@H](C)N(C)C(=O)/C=C/CN(C)C)c2)nc1C] | 509.64 | ** | *** | * |
| 315 | 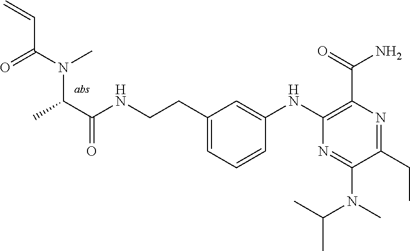 [SMILES: C=CC(=O)N(C)[C@@H](C)C(=O)NCCc1cccc(Nc2nc(N(C)C(C)C)c(CC)nc2C(N)=O)c1] | 495.62 | **** | **** | * |
| 316 | 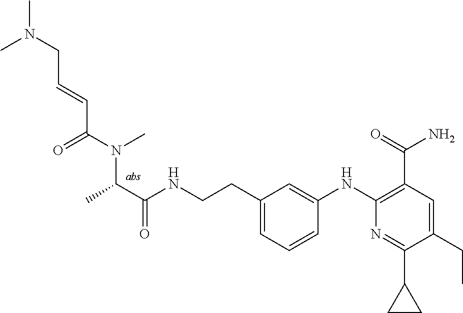 [SMILES: CCc1cc(C(N)=O)c(Nc2cccc(CCNC(=O)[C@H](C)N(C)C(=O)/C=C/CN(C)C)c2)nc1C1CC1] | 520.67 | ** | *** | * |
| 317 | 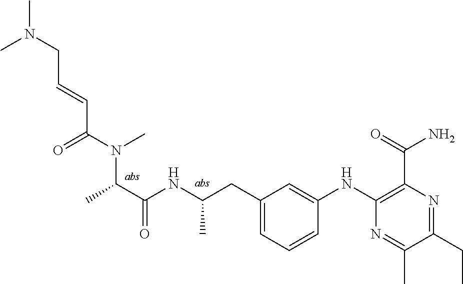 [SMILES: CCc1nc(C(N)=O)c(Nc2cccc(C[C@H](C)NC(=O)[C@H](C)N(C)C(=O)/C=C/CN(C)C)c2)nc1C] | 509.64 | *** | **** | * |
| 318 | 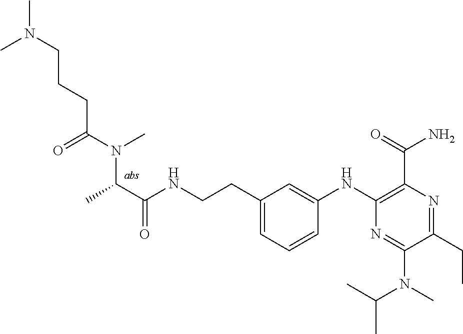 [SMILES: CCc1nc(C(N)=O)c(Nc2cccc(CCNC(=O)[C@H](C)N(C)C(=O)CCCN(C)C)c2)nc1N(C)C(C)C] | 554.73 | *** | **** | * |
| 319 | 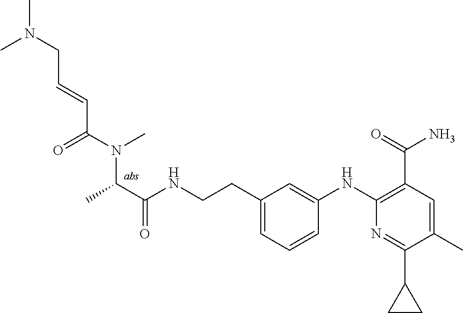 | 506.64 | *** | *** | * |
| 320 | 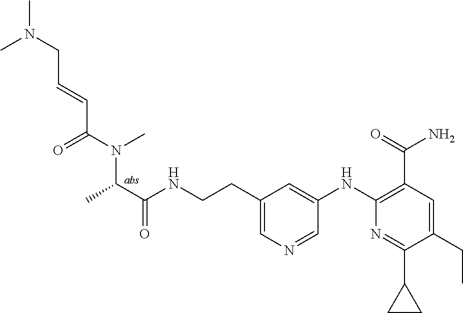 [SMILES: CCc1cc(C(N)=O)c(Nc2cncc(CCNC(=O)[C@H](C)N(C)C(=O)/C=C/CN(C)C)c2)nc1C1CC1] | 521.65 | ** | *** | * |
| 321 | 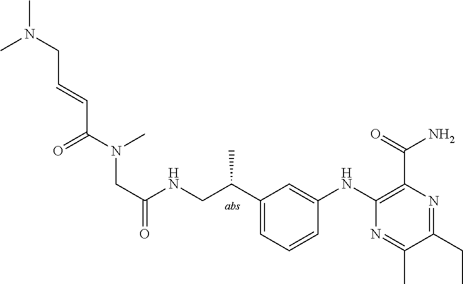 [SMILES: CCc1nc(C(N)=O)c(Nc2cccc([C@@H](C)CNC(=O)CN(C)C(=O)/C=C/CN(C)C)c2)nc1C] | 495.62 | **** | **** | * |
| 322 | 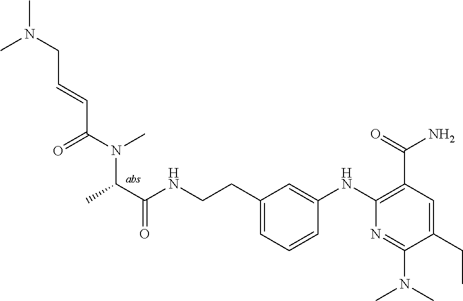 [SMILES: CCc1cc(C(N)=O)c(Nc2cccc(CCNC(=O)[C@H](C)N(C)C(=O)/C=C/CN(C)C)c2)nc1N(C)C] | 523.67 | ** | *** | * |
| 323 | 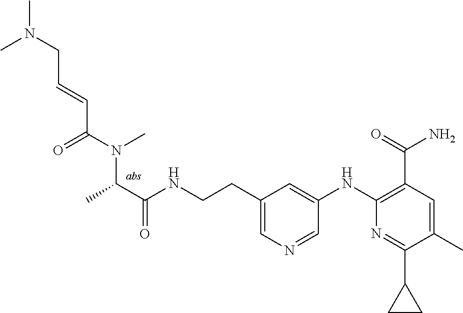 | 507.63 | ** | *** | * |
| 324 | 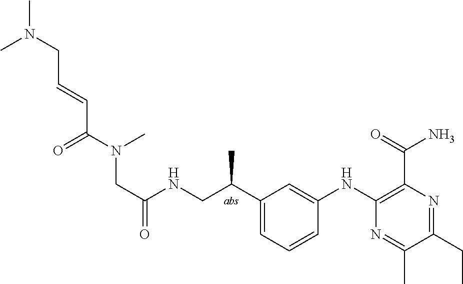 [SMILES: CCc1nc(C(N)=O)c(Nc2cccc([C@H](C)CNC(=O)CN(C)C(=O)/C=C/CN(C)C)c2)nc1C] | 495.62 | ** | *** | * |
| 325 | 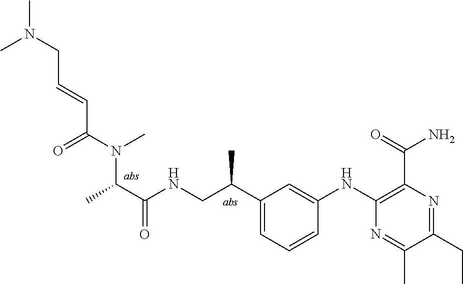 [SMILES: CCc1nc(C(N)=O)c(Nc2cccc([C@H](C)CNC(=O)[C@H](C)N(C)C(=O)/C=C/CN(C)C)c2)nc1C] | 509.64 | ** | ** | * |
| 326 | 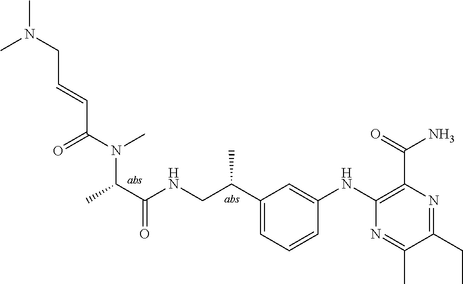 [SMILES: CCc1nc(C(N)=O)c(Nc2cccc([C@@H](C)CNC(=O)[C@H](C)N(C)C(=O)/C=C/CN(C)C)c2)nc1C] | 509.64 | | | |
| 327 | 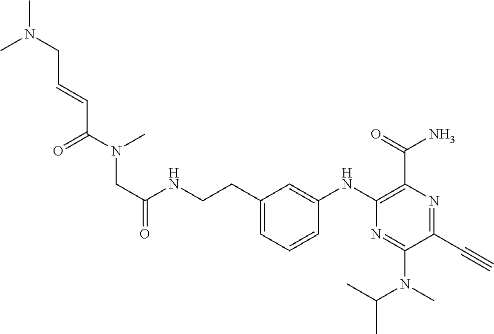 [SMILES: C#Cc1nc(C(N)=O)c(Nc2cccc(CCNC(=O)CN(C)C(=O)/C=C/CN(C)C)c2)nc1N(C)C(C)C] | 534.65 | | | |
| 328 | 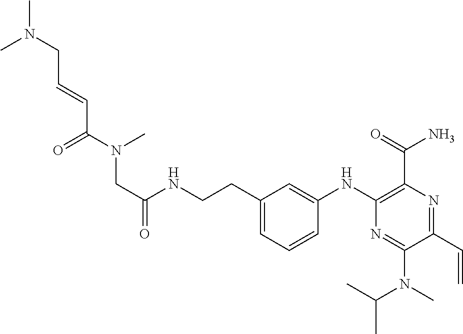 [SMILES: C=Cc1nc(C(N)=O)c(Nc2cccc(CCNC(=O)CN(C)C(=O)/C=C/CN(C)C)c2)nc1N(C)C(C)C] | 536.67 | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

At least some of the chemical names of compounds provided herein as given and set forth in this application, may have been generated on an automated basis by use of a commercially available chemical naming software program, and have not been independently verified In the instance where the indicated chemical name and the depicted structure differ, the depicted structure will control. In the chemical structures where a chiral center exists in a structure but no specific stereochemistry is shown for the chiral center, both enantiomers associated with the chiral structure are encompassed by the structure.

What is claimed is:

1. A method of treating cancer in a patient in need thereof comprising administering to the patient a compound according to Formula (P4-I): 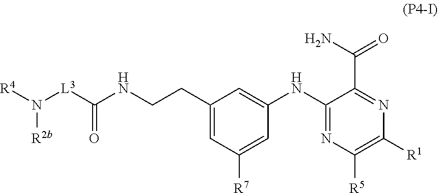
or a stereoisomer thereof, or a pharmaceutically acceptable salt thereof, wherein
L³ is substituted or unsubstituted C₁-C₄ alkylene, 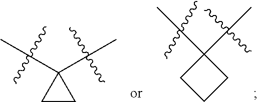 [SMILES: *C1(*)CC1 ; *C1(*)CCC1]
Cy is substituted or unsubstituted cycloalkyl or substituted or unsubstituted heterocycloalkyl;
R¹ is substituted or unsubstituted C₁₋₆ alkyl, substituted or unsubstituted alkoxy, substituted or unsubstituted cycloalkyl, or substituted or unsubstituted heterocycloalkyl;
R^{2b} is H or C₁-C₄ alkyl;
R⁴ is —C(O)—C(R^{6a})═C(R^{6b})(R^{6c});
R⁵ is Cy, substituted or unsubstituted C₁₋₆ alkyl, substituted or unsubstituted haloalkyl, substituted or unsubstituted C₁₋₆ alkoxy, substituted or unsubstituted haloalkoxy, or substituted or unsubstituted alkylamino;
each R^{6a} and R^{6b} is independently H, halo, CN, or C₁₋₆ alkyl; or R^{6a} and R^{6b} are joined together to form a bond;
R^{6c} is H, halo, CN, or C₁₋₆ alkyl, wherein the C₁₋₆ alkyl is unsubstituted or substituted with one or more groups selected from substituted or unsubstituted amino and substituted or unsubstituted heterocycloalkyl having 1-2 heteroatoms independently selected from nitrogen, oxygen, and sulfur;
each R⁷ is independently H, halo, CN, substituted or unsubstituted C₁₋₆ alkyl, substituted or unsubstituted alkoxy, or substituted or unsubstituted heterocycloalkyl;
in combination with a menin inhibitor.

2. The method according to claim 1, wherein the compound is according to Formula (P2-I): 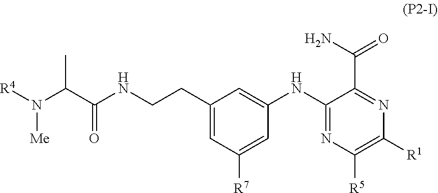
or a stereoisomer thereof, or a pharmaceutically acceptable salt thereof.

3. The method according to any one of claims 1-2; wherein R⁷ is H, Me, Et, Cl, F, or OMe.

4. The method according to any one of claims 1-2; wherein R⁷ is H.

5. The method according to any one of claims 1-2; wherein R⁷ is F.

6. The method according to any one of claims 1-5; wherein R¹ is Me or Et.

7. The method according to any one of claims 1-6; wherein R⁵ is cyclopropyl, Me, Et, N(Me)₂, or N(i-Pr)(Me).

8. The method according to claim 1, wherein the compound is according to Formula (XLIIIa) or (XLIIIc): 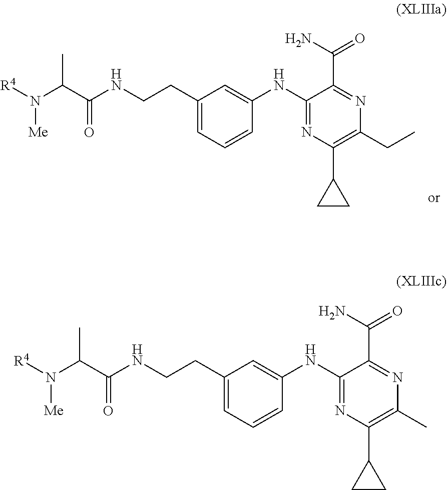
or a stereoisomer thereof, or a pharmaceutically acceptable salt thereof.

9. The method according to claim 1, wherein the compound is according to Formula (XLIVa) or (XLIVc): 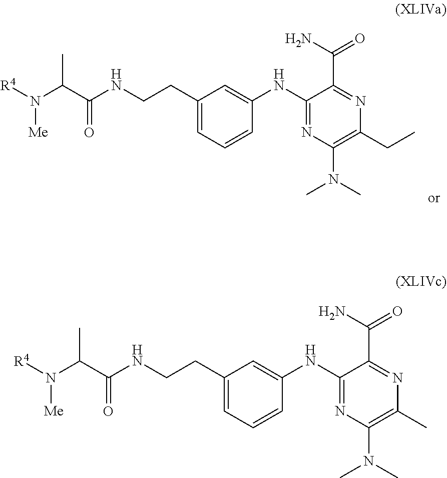
or a stereoisomer thereof, or a pharmaceutically acceptable salt thereof.

10. The method according to claim 1, wherein the compound is according to Formula (XLVIa), (XLVIc), (XLVIe), (XLVIm), (XLVIo) or (XLVIq): 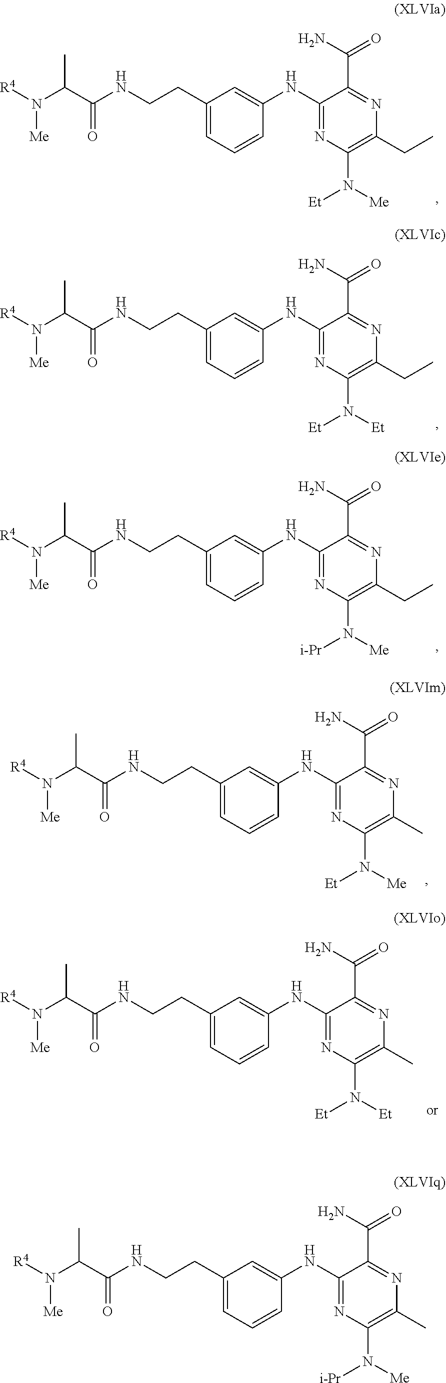
or a stereoisomer thereof, or a pharmaceutically acceptable salt thereof.

11. The method according to claim 1, wherein the compound is according to Formula (XLVIIIa), (XLVIIIc), (XLVIIIe), or (XLVIIIg): 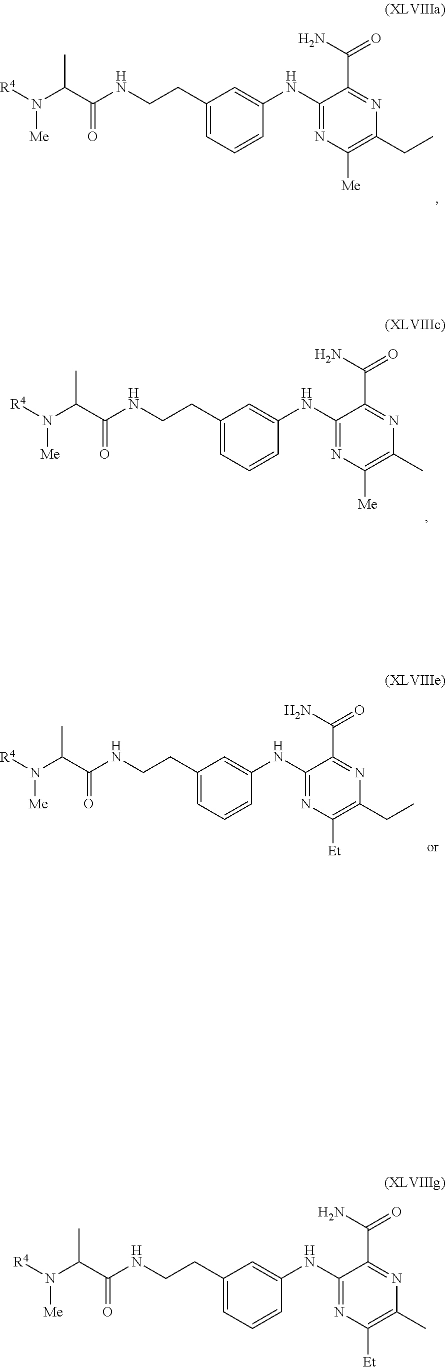
or a stereoisomer thereof, or a pharmaceutically acceptable salt thereof.

12. The method according to claim 1, wherein the compound is according to Formula (LXa), (LXc), or (LXe): 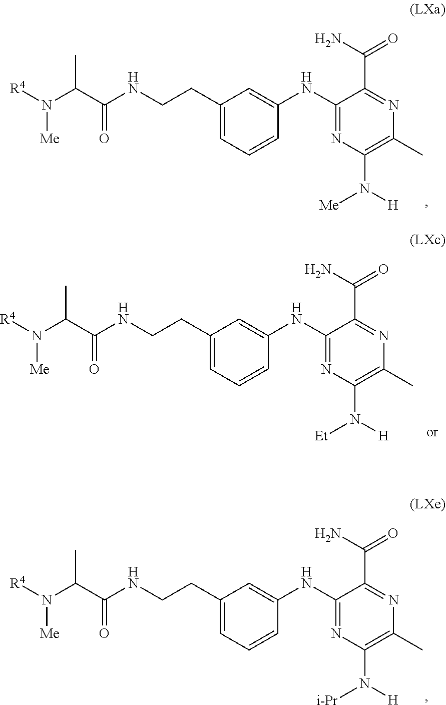
or a stereoisomer thereof, or a pharmaceutically acceptable salt thereof.

13. The method according to claim 1, wherein the compound is according to Formula (LXIa), or (LXIIa): 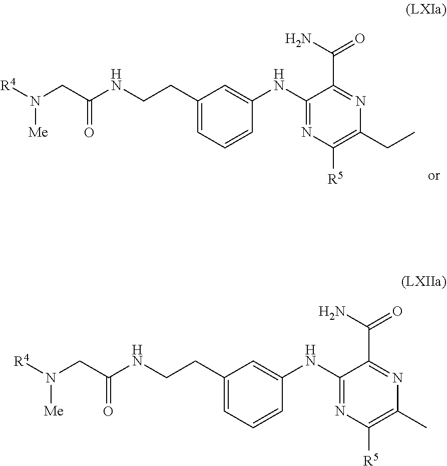
or a stereoisomer thereof, or a pharmaceutically acceptable salt thereof; wherein R⁵ is cyclopropyl, Me, Et, N(Me)₂, or N(i-Pr)(Me).

14. The method according to claim 1, wherein the compound is according to Formula (LXIIIa), or (LXIIb): 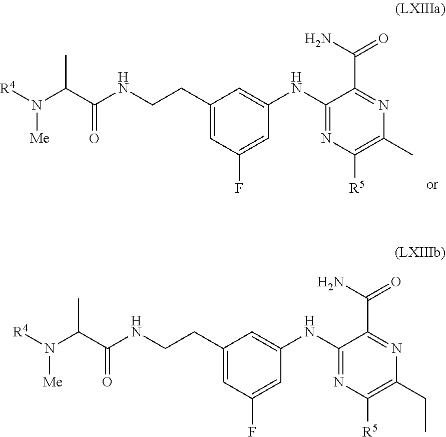
or a stereoisomer thereof, or a pharmaceutically acceptable salt thereof; wherein R⁵ is cyclopropyl, Me, Et, N(Me)₂, or N(i-Pr)(Me).

15. The method according to any one of claims 1-15, wherein each of R^{6a}, R^{6b}, and R^{6c} is H.

16. The method according to any one of claims 1-15, wherein each of R^{6a} and R^{6b} is H or F; and R^{6c} is substituted or unsubstituted alkyl.

17. The method according to any one of claims 1-15, wherein each of R^{6a} and R^{6b} is H; and R^{6c} is alkyl substituted with amino, alkylamino or dialkylamino.

18. The method according to any one of claims 1-15, wherein each of R^{6a} and R^{6b} is H; and R^{6c} is —CH₂NMe₂, or —CH₂NHMe.

19. The method according to any one of claims 1-15, wherein R^{6a} and R^{6b} form a bond; and R^{c} is H or substituted or unsubstituted alkyl.

20. The method according to any one of claims 1-15, wherein each of R^{6a} and R^{6b} is H; and R^{6c} is —(CH₂)_{q}-heterocycloalkyl; and q is 1, 2, 3, or 4.

21. The method according to any one of claims 1-15, wherein each of R^{6a} and R^{6b} is H; and R^{6c} is (CH₂)_{q}-heterocycloalkyl; q is 1; and heterocycloalkyl is substituted or unsubstituted azetidinyl, pyrrolidinyl, piperidinyl, or azepinyl.

22. The method according to any one of claims 1-15, wherein each of R^{6a}, and R^{6b} is H or Me; and R^{6c} is —CH₂-azetidin-1-yl, —CH₂-pyrrolidin-1-yl, or —CH₂-piperidin-1-yl.

23. The method according to claim 1, wherein the compound is selected from any one of compounds listed in Table 1, or a pharmaceutically acceptable stereoisomer, salt, or solvate thereof.

24. The method according to claim 1, wherein the menin inhibitor is a covalent menin inhibitor.

25. The method according to claim 1, wherein the menin inhibitor is according to formula (I): 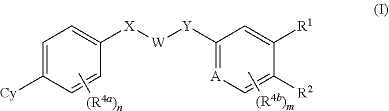
or a pharmaceutically acceptable salt thereof,
wherein:
A is C or N;
Cy is substituted or unsubstituted 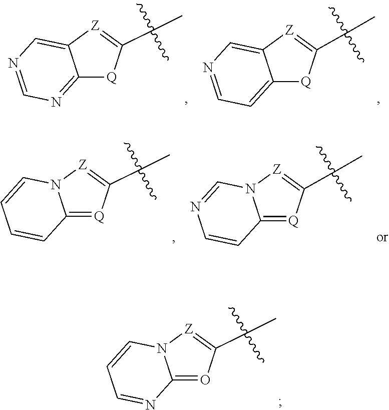
Q is N, —N(H)—, —O—, or —S—;
Z is —CR^{5a}═ or —N═;
X is —NR^{3a}—, —C(R^{3b})₂—, or —O—;
Y is a single bond, —NR^{3a}—, —C(R^{3b})₂—, or —O—;
W is —C(O)—, —S(O)—, or —S(O)₂—;
one of R¹ and R² is Cy²-N(H)C(O)—C(R^{6a})═C(R^{6b})(R^{6c}) or CH₂—Cy²-N(H)C(O)—C(R^{6a})═C(R^{6b})(R^{6c}); and the other is H, C₁₋₆ alkyl, C₁₋₆ haloalkyl, halo, or CN;
Cy² is an optionally substituted group selected from phenyl, pyridyl, or a 4-7 membered heterocycloalkyl ring having 1-2 heteroatoms independently selected from nitrogen, oxygen, or sulfur;
each R^{3a} and R^{3b} is independently H or C₁₋₆ alkyl;
each R^{4a} and R^{4b} is independently H, halo, CN, OR, —N(R)₂, —C(O)N(R)₂, —NRC(O)R, —SO₂R, —C(O)R, —CO₂R, or an optionally substituted group selected from C₁₋₆ alkyl, C₃₋₇ cycloalkyl, a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur;
each R is independently H, or an optionally substituted group selected from C₁₋₆ aliphatic, phenyl, an 8-10 membered bicyclic aryl ring, a 4-7 membered saturated or partially unsaturated heterocyclic ring having 1-2 heteroatoms independently selected from nitrogen, oxygen, or sulfur, and a 5-6 membered heteroaryl ring having 1-4 heteroatoms independently selected from nitrogen, oxygen, or sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, or sulfur;
R^{5a} is H, C₁₋₆ alkyl, C₁₋₆ haloalkyl, halo, or CN;
each R^{6a} and R^{6b} is independently H or C₁₋₆ alkyl; or R^{6a} and R^{6b} are joined together to form a bond;
R^{6c} is H or substituted or unsubstituted C₁₋₆ alkyl;
m is 1, 2, or 3; and n is 1, 2, 3, or 4.

26. The method according to claim 1, wherein the menin inhibitor is according to formula (XXI): 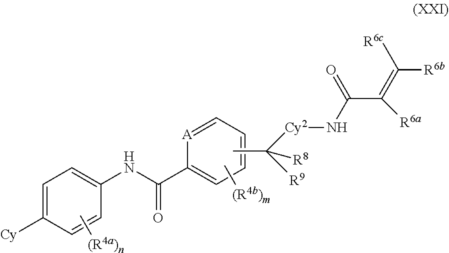
or a pharmaceutically acceptable salt thereof,
wherein each R⁸ and R⁹ is independently H, C₁₋₆ alkyl, C₁₋₆ haloalkyl, halo, or CN.

27. The method according to claim 1, wherein the menin inhibitor is according to formula (XIVa), (XIVb), or (XIVc): 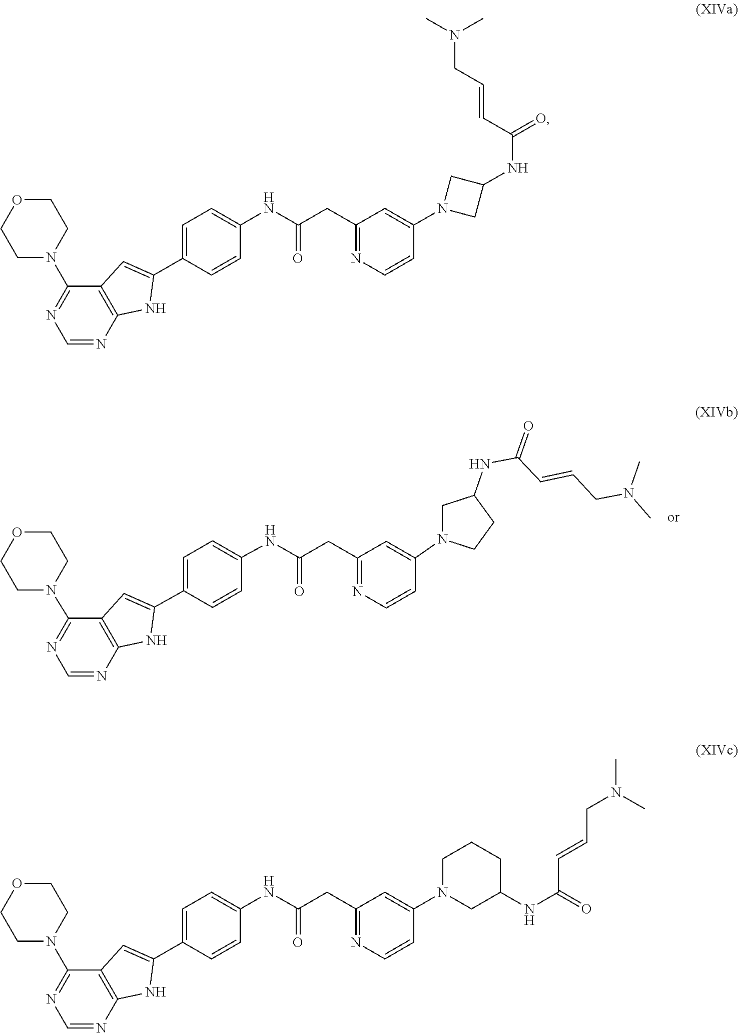 [SMILES: CN(C)C/C=C/C(=O)NC1CN(c2ccnc(CC(=O)Nc3ccc(-c4cc5c(N6CCOCC6)ncnc5[nH]4)cc3)c2)C1 ; CN(C)C/C=C/C(=O)NC1CCN(c2ccnc(CC(=O)Nc3ccc(-c4cc5c(N6CCOCC6)ncnc5[nH]4)cc3)c2)C1 ; CN(C)C/C=C/C(=O)NC1CCCN(c2ccnc(CC(=O)Nc3ccc(-c4cc5c(N6CCOCC6)ncnc5[nH]4)cc3)c2)C1]
or a pharmaceutically acceptable salt thereof.

28. The method according to claim 1, wherein the menin inhibitor is according to formula (XV): 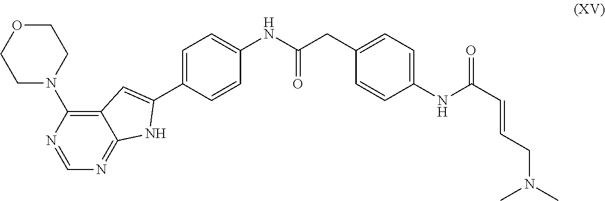 [SMILES: CN(C)C/C=C/C(=O)Nc1ccc(CC(=O)Nc2ccc(-c3cc4c(N5CCOCC5)ncnc4[nH]3)cc2)cc1]
or a pharmaceutically acceptable salt thereof.

29. The method according to claim 1, wherein the menin inhibitor is according to formula (XVI): 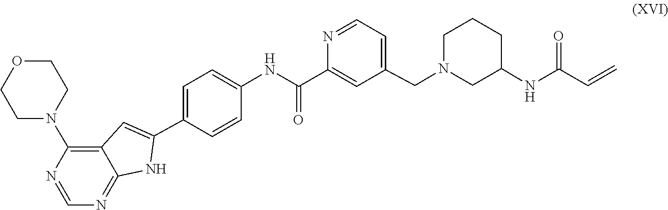 [SMILES: C=CC(=O)NC1CCCN(Cc2ccnc(C(=O)Nc3ccc(-c4cc5c(N6CCOCC6)ncnc5[nH]4)cc3)c2)C1]
or a pharmaceutically acceptable salt thereof.

30. The method according to claim 1, wherein the menin inhibitor is according to formula (XVII): 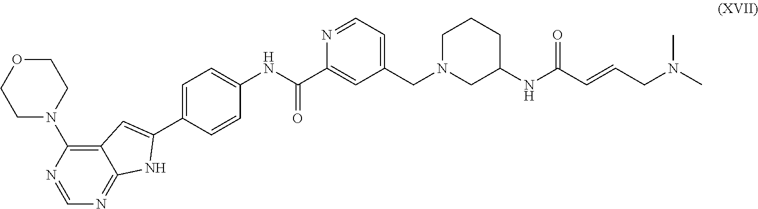 [SMILES: CN(C)C/C=C/C(=O)NC1CCCN(Cc2ccnc(C(=O)Nc3ccc(-c4cc5c(N6CCOCC6)ncnc5[nH]4)cc3)c2)C1]
or a pharmaceutically acceptable salt thereof.

31. The method according to claim 1, wherein the menin inhibitor is according to formula (XXVIIa), (XXVIIb), or (XXVIIc): 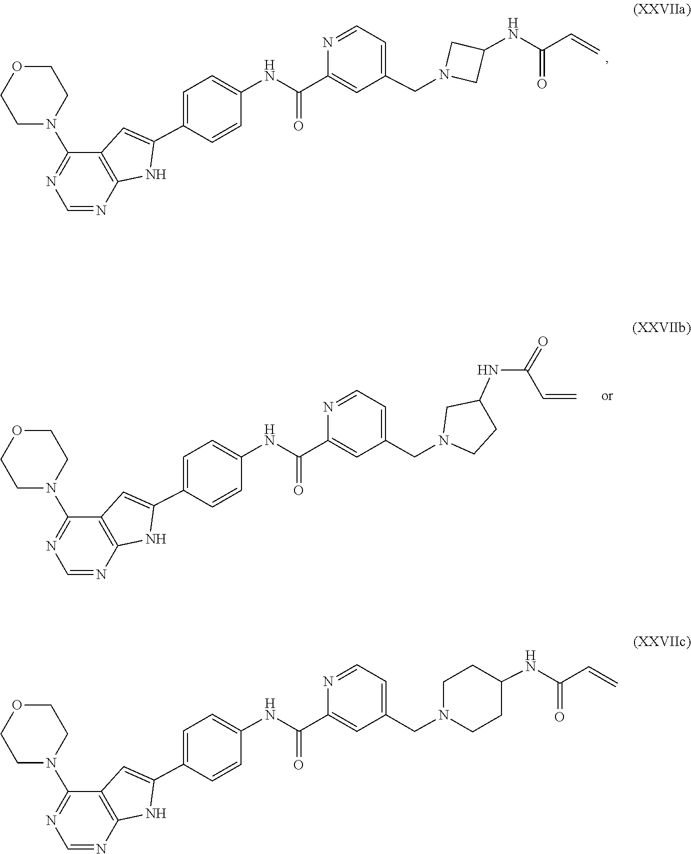 [SMILES: C=CC(=O)NC1CN(Cc2ccnc(C(=O)Nc3ccc(-c4cc5c(N6CCOCC6)ncnc5[nH]4)cc3)c2)C1 ; C=CC(=O)NC1CCN(Cc2ccnc(C(=O)Nc3ccc(-c4cc5c(N6CCOCC6)ncnc5[nH]4)cc3)c2)C1 ; C=CC(=O)NC1CCN(Cc2ccnc(C(=O)Nc3ccc(-c4cc5c(N6CCOCC6)ncnc5[nH]4)cc3)c2)CC1]
or a pharmaceutically acceptable salt thereof.

32. The method according to claim 1, wherein the menin inhibitor is according to formula (XXVIIIa), (XXVIIIb), or (XXVIIc): 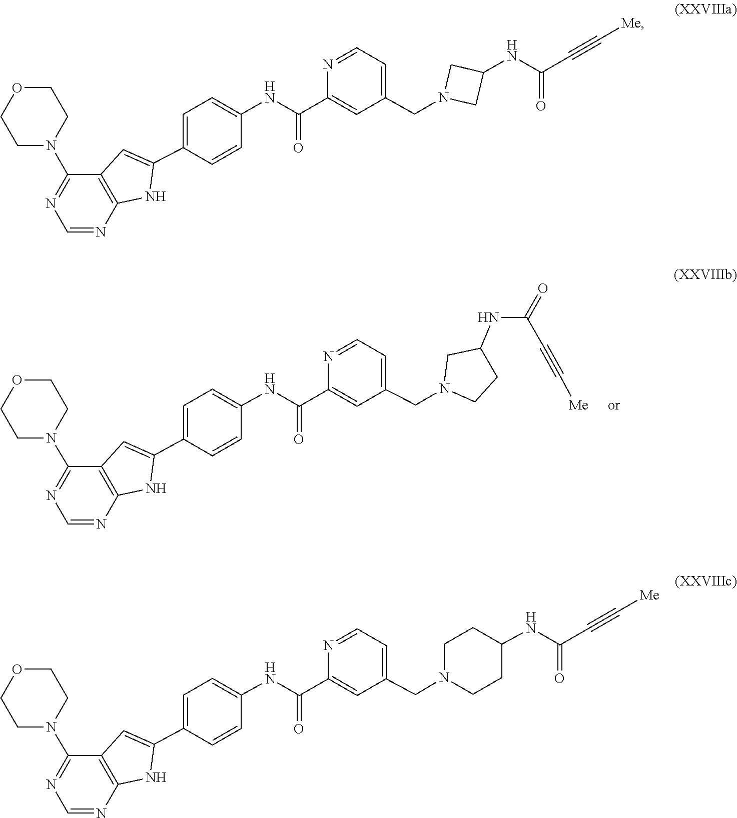 [SMILES: CC#CC(=O)NC1CN(Cc2ccnc(C(=O)Nc3ccc(-c4cc5c(N6CCOCC6)ncnc5[nH]4)cc3)c2)C1 ; CC#CC(=O)NC1CCN(Cc2ccnc(C(=O)Nc3ccc(-c4cc5c(N6CCOCC6)ncnc5[nH]4)cc3)c2)C1 ; CC#CC(=O)NC1CCN(Cc2ccnc(C(=O)Nc3ccc(-c4cc5c(N6CCOCC6)ncnc5[nH]4)cc3)c2)CC1]
or a pharmaceutically acceptable salt thereof.

33. The method according to claim 1, wherein the menin inhibitor is according to formula (XXIXa), (XXIXb), or (XXIXc): 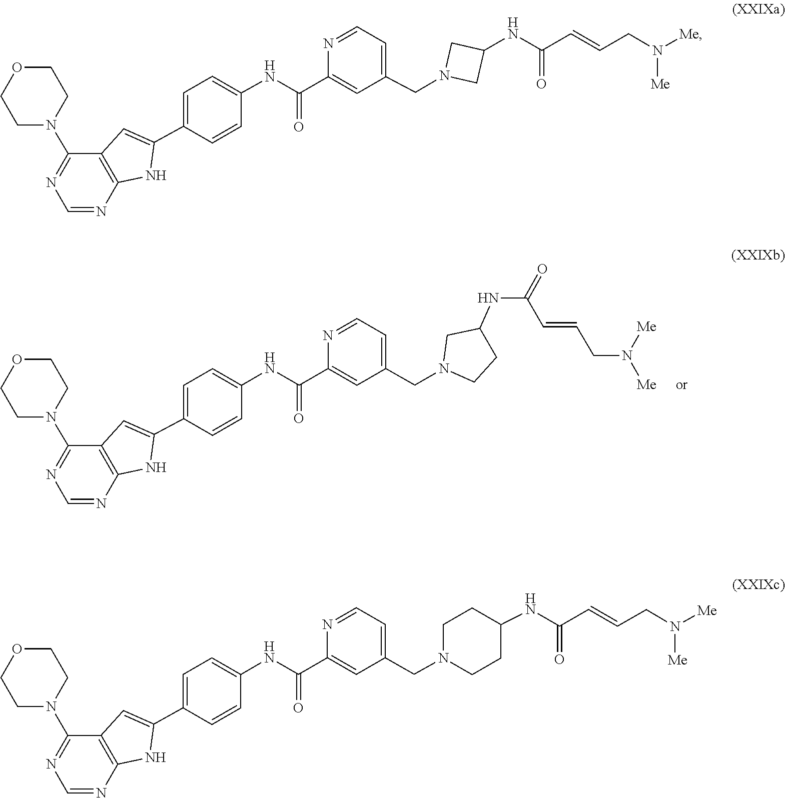 [SMILES: CN(C)C/C=C/C(=O)NC1CN(Cc2ccnc(C(=O)Nc3ccc(-c4cc5c(N6CCOCC6)ncnc5[nH]4)cc3)c2)C1 ; CN(C)C/C=C/C(=O)NC1CCN(Cc2ccnc(C(=O)Nc3ccc(-c4cc5c(N6CCOCC6)ncnc5[nH]4)cc3)c2)C1 ; CN(C)C/C=C/C(=O)NC1CCN(Cc2ccnc(C(=O)Nc3ccc(-c4cc5c(N6CCOCC6)ncnc5[nH]4)cc3)c2)CC1]
or a pharmaceutically acceptable salt thereof.

34. The method according to claim 1, wherein the menin inhibitor is according to formula (XLa), (XLb), or (XLc): 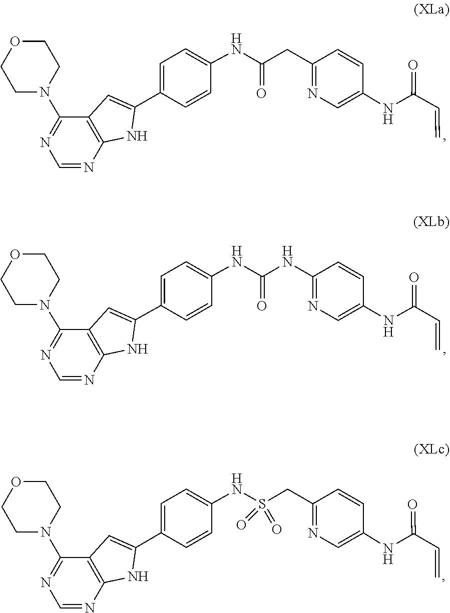 [SMILES: C=CC(=O)Nc1ccc(CC(=O)Nc2ccc(-c3cc4c(N5CCOCC5)ncnc4[nH]3)cc2)nc1 ; C=CC(=O)Nc1ccc(NC(=O)Nc2ccc(-c3cc4c(N5CCOCC5)ncnc4[nH]3)cc2)nc1 ; C=CC(=O)Nc1ccc(CS(=O)(=O)Nc2ccc(-c3cc4c(N5CCOCC5)ncnc4[nH]3)cc2)nc1]
or a pharmaceutically acceptable salt thereof.

35. The method according to claim 1, wherein the menin inhibitor is according to formula (XLIa), (XLIb), or (XLIc): 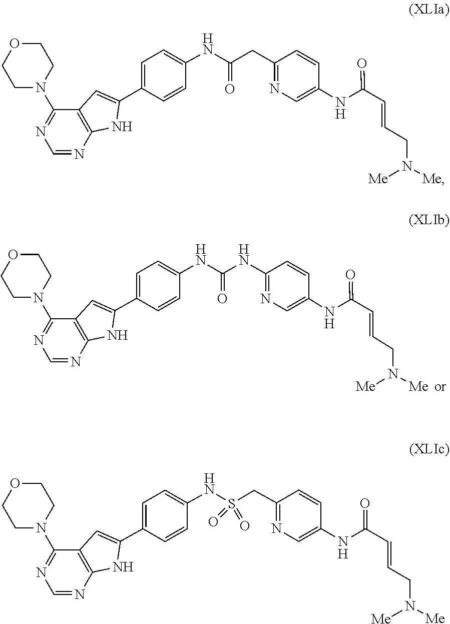 [SMILES: CN(C)C/C=C/C(=O)Nc1ccc(CC(=O)Nc2ccc(-c3cc4c(N5CCOCC5)ncnc4[nH]3)cc2)nc1 ; CN(C)C/C=C/C(=O)Nc1ccc(NC(=O)Nc2ccc(-c3cc4c(N5CCOCC5)ncnc4[nH]3)cc2)nc1 ; CN(C)C/C=C/C(=O)Nc1ccc(CS(=O)(=O)Nc2ccc(-c3cc4c(N5CCOCC5)ncnc4[nH]3)cc2)nc1]
or a pharmaceutically acceptable salt thereof.

36. The method according to claim 1, wherein the menin inhibitor is according to formula (XLIIa), (XLIIb), or (XLIIc): 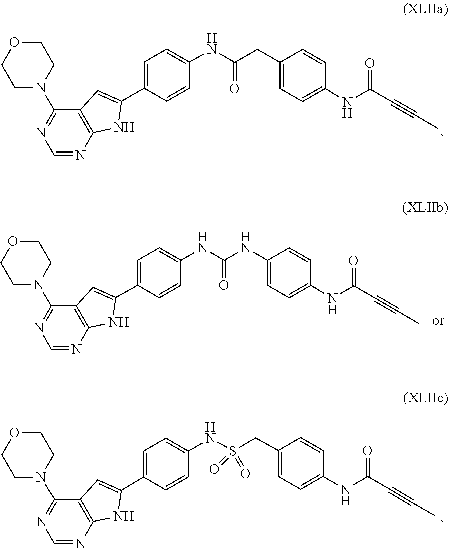 [SMILES: CC#CC(=O)Nc1ccc(CC(=O)Nc2ccc(-c3cc4c(N5CCOCC5)ncnc4[nH]3)cc2)cc1 ; CC#CC(=O)Nc1ccc(NC(=O)Nc2ccc(-c3cc4c(N5CCOCC5)ncnc4[nH]3)cc2)cc1 ; CC#CC(=O)Nc1ccc(CS(=O)(=O)Nc2ccc(-c3cc4c(N5CCOCC5)ncnc4[nH]3)cc2)cc1]
or a pharmaceutically acceptable salt thereof.

37. The method according to claim 1, wherein the menin inhibitor is according to formula (XLLIIIa), (XLIIIb), or (XLIIIc): 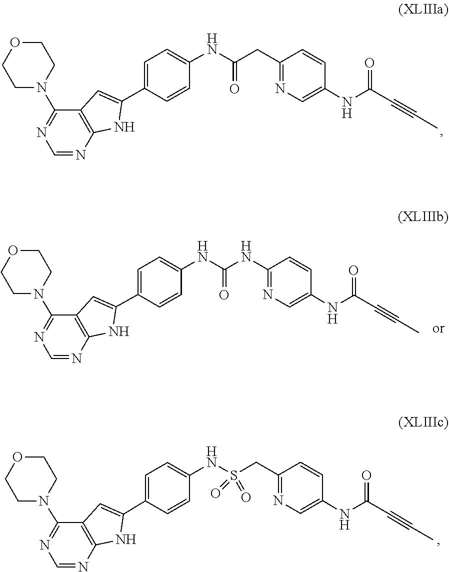 [SMILES: CC#CC(=O)Nc1ccc(CC(=O)Nc2ccc(-c3cc4c(N5CCOCC5)ncnc4[nH]3)cc2)nc1 ; CC#CC(=O)Nc1ccc(NC(=O)Nc2ccc(-c3cc4c(N5CCOCC5)ncnc4[nH]3)cc2)nc1 ; CC#CC(=O)Nc1ccc(CS(=O)(=O)Nc2ccc(-c3cc4c(N5CCOCC5)ncnc4[nH]3)cc2)nc1]
or a pharmaceutically acceptable salt thereof.

38. The method according to claim 1, wherein the menin inhibitor is selected from Compound 3, Compound 6, Compound 10, and Compound 13: 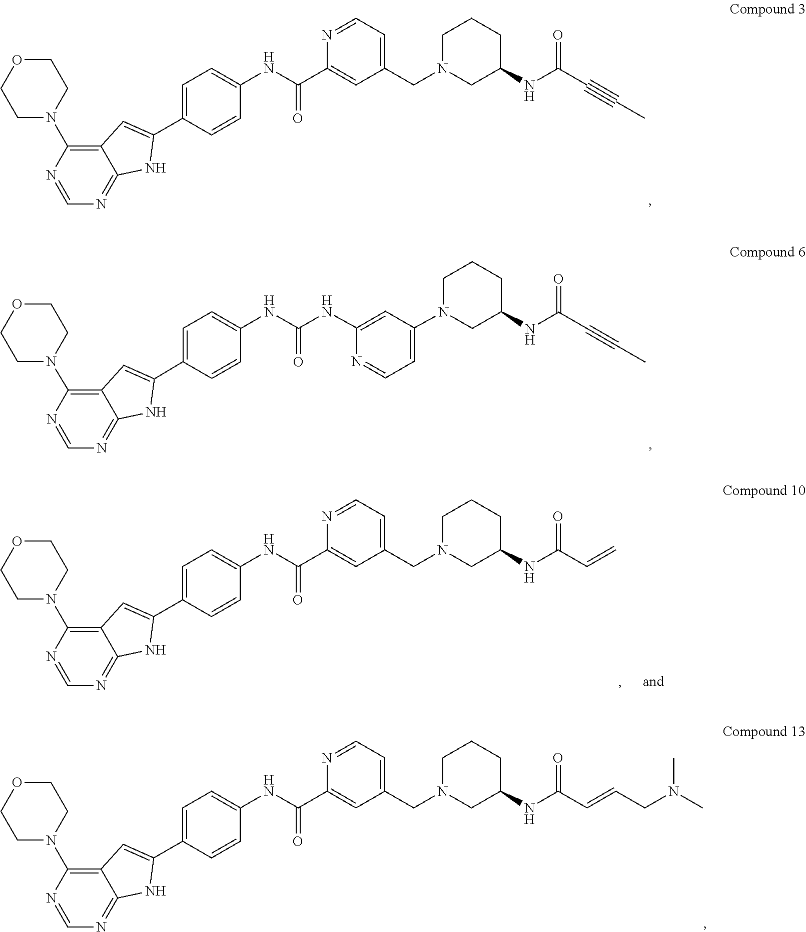 [SMILES: CCCC(=O)N[C@@H]1CCCN(Cc2ccnc(C(=O)Nc3ccc(-c4cc5c(N6CCOCC6)ncnc5[nH]4)cc3)c2)C1 ; CC#CC(=O)N[C@@H]1CCCN(c2ccnc(NC(=O)Nc3ccc(-c4cc5c(N6CCOCC6)ncnc5[nH]4)cc3)c2)C1 ; C=CC(=O)N[C@@H]1CCCN(Cc2ccnc(C(=O)Nc3ccc(-c4cc5c(N6CCOCC6)ncnc5[nH]4)cc3)c2)C1 ; CN(C)C/C=C/C(=O)N[C@@H]1CCCN(Cc2ccnc(C(=O)Nc3ccc(-c4cc5c(N6CCOCC6)ncnc5[nH]4)cc3)c2)C1]
or a salt thereof.

39. The method according to claim 1, wherein the menin inhibitor is 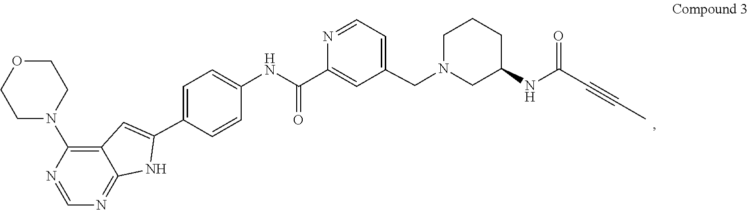 [SMILES: CC#CC(=O)N[C@@H]1CCCN(Cc2ccnc(C(=O)Nc3ccc(-c4cc5c(N6CCOCC6)ncnc5[nH]4)cc3)c2)C1]
or a salt thereof.

40. The method according to claim 1, wherein the menin inhibitor is 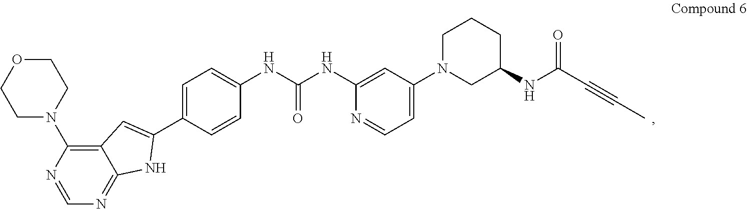 [SMILES: CC#CC(=O)N[C@@H]1CCCN(c2ccnc(NC(=O)Nc3ccc(-c4cc5c(N6CCOCC6)ncnc5[nH]4)cc3)c2)C1]
or a salt thereof.

41. The method according to claim 1, wherein the menin inhibitor is 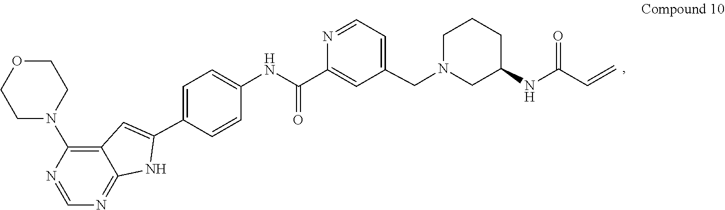 [SMILES: C=CC(=O)N[C@@H]1CCCN(Cc2ccnc(C(=O)Nc3ccc(-c4cc5c(N6CCOCC6)ncnc5[nH]4)cc3)c2)C1]
or a salt thereof.

42. The method according to claim 1, wherein the menin inhibitor is 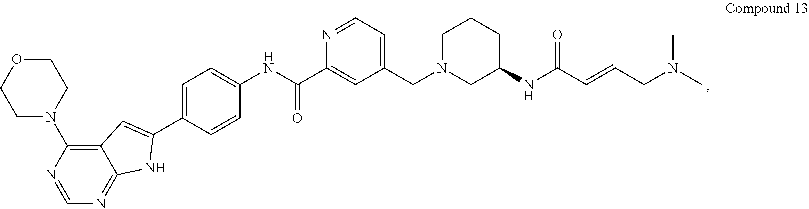 [SMILES: CN(C)C/C=C/C(=O)N[C@@H]1CCCN(Cc2ccnc(C(=O)Nc3ccc(-c4cc5c(N6CCOCC6)ncnc5[nH]4)cc3)c2)C1]
or a salt thereof.

43. The method of any one of claims 1-42, wherein the cancer is a hematologic cancer.

44. The method of claim 43, wherein the hematologic cancer is a leukemia, a lymphoma, a myeloma, a non-Hodgkin's lymphoma, a Hodgkin's lymphoma, or a B-cell malignancy.

45. The method of claim 43, wherein the B-cell malignancy is diffuse large B-cell lymphoma (DLBCL).

46. The method of claim 45, wherein DLBCL is activated B-cell diffuse large B-cell lymphoma (ABC-DLBCL).

47. The method of claim 45, wherein the B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof.

48. The method of any one of the claims 45-47, wherein the B-cell malignancy is a relapsed or refractory B-cell malignancy.

49. The method of claim 48, wherein the relapsed or refractory B-cell malignancy is diffuse large B-cell lymphoma (DLBCL).

50. The method of claim 49, wherein the relapsed or refractory DLBCL is activated B-cell diffuse large B-cell lymphoma (ABC-DLBCL).

51. The method of claim 48, wherein the relapsed or refractory B-cell malignancy is chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof.

52. The method of any one of the claims 45-47, wherein the B-cell malignancy is a metastasized B-cell malignancy.

53. The method of claim 52, wherein the metastasized B-cell malignancy is diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenström's macroglobulinemia, or a combination thereof.

54. The method of any one of claims 1-42, wherein the cancer is a sarcoma or carcinoma.

55. The method of claim 54, wherein the cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

56. The method of claim 55, wherein the cancer is a breast cancer.

57. The method of claim 56, wherein the breast cancer is ductal carcinoma in situ, lobular carcinoma in situ, invasive or infiltrating ductal carcinoma, invasive or infiltrating lobular carcinoma, inflammatory breast cancer, triple-negative breast cancer, paget disease of the nipple, phyllodes tumor, angiosarcoma or invasive breast carcinoma.

58. The method of claim 55, wherein the cancer is a colon cancer.

59. The method of claim 58, wherein the colon cancer is adenocarcinoma, gastrointestinal carcinoid tumors, gastrointestinal stromal tumors, primary colorectal lymphoma, leiomyosarcoma, melanoma, squamous cell-carcinoma, mucinous adenocarcinoma, or Signet ring cell adenocarcinoma.

60. The method of any one of the claims 54-59, wherein the cancer is a relapsed or refractory cancer.

61. The method of claim 60, wherein the relapsed or refractory cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

62. The method of any one of the claims 54-59, wherein the cancer is a metastasized cancer.

63. The method of claim 62, wherein the metastasized cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

64. The method of any one of claims 1-63, wherein the combination is administered once a day, two times per day, three times per day, four times per day, or five times per day.

65. The method of any one of claims 1-64, wherein the menin inhibitor is administered at a dosage of about 40 mg/day to about 1000 mg/day.

66. The method of any one of claims 1-65, wherein the menin inhibitor is administered orally.

67. The method of any one of claims 1-66, wherein the compound of formula (P4-I) and the menin inhibitor are administered in separate compositions.

68. The method of any one of claims 1-67, wherein the compound of formula (P4-I) and the menin inhibitor are administered simultaneously, sequentially or intermittently.

69. The method of any one of claims 1-68, further comprising administering an additional anticancer agent.

70. A pharmaceutical combination comprising:
a) a compound according to formula (P4-I); and
b) an menin inhibitor; and
c) a pharmaceutically-acceptable excipient.

71. The pharmaceutical combination of claim 70, wherein the compound according to formula (P4-I) and the menin inhibitor are administered in separate compositions.

72. The pharmaceutical combination of claim 70 or 71 that is formulated for a route of administration selected from oral administration, parenteral administration, buccal administration, nasal administration, topical administration, or rectal administration.

73. A method for treating an proliferative disease or condition comprising administering to a patient in need thereof a therapeutically effective amount of the pharmaceutical combination of claim 70 or 71.

74. A method for treating a proliferative disease or condition comprising administering to a patient in need thereof the pharmaceutical combination of claim 70 or 71.

75. A method for treating a cancer comprising administering to a patient in need thereof a therapeutically effective amount of the pharmaceutical combination of claim 70 or 71.

76. The method of claim 75, wherein the cancer is a hematological malignancy.

77. The method of claim 76, wherein the hematological malignancy is acute lymphocytic leukemia (ALL), acute myeloid leukemia (AML), chronic lymphocytic leukemia (CLL), chronic myeloid leukemia (CML), prolymphocytic leukemia (PLL), large granular lymphocytic (LGL), hairy cell leukemia (HCL), mast-cell leukemia (MCL) or myelodysplastic syndrome (MDS).
